(19) United States
(12) Patent Application Publication
(10) Pub. No.: US 2016/0355503 A1
(43) Pub. Date: Dec. 8, 2016

(54) NITROGEN HEXACYCLE COMPOUNDS AS INHIBITORS OF p97 COMPLEX

(71) Applicant: Cleave Biosciences, Inc., Burlingame, CA (US)

(72) Inventors: Han-Jie Zhou, Foster City, CA (US); David Wustrow, Los Gatos, CA (US)

(21) Appl. No.: 15/173,294

(22) Filed: Jun. 3, 2016

Related U.S. Application Data
(60) Provisional application No. 62/171,134, filed on Jun. 4, 2015.

(51) Int. Cl. C07D 403/04

(52) U.S. Cl. CPC C07D 403/04

(57) ABSTRACT

Nitrogen hexacycle compounds having an arylalkyl amine substituent at the P4 position and a substituted 5:6 bicyclic group at the P2 position of the nitrogen hexacycle as well as optional aliphatic, functional and/or aromatic components substituted at other positions of the nitrogen hexacycle, the aryl alkyl group and the 5:6 bicyclic group are disclosed. These compounds are inhibitors of the AAA proteasome complex containing p97 and are effective medicinal agents for treatment of diseases associated with p97 bioactivity such as cancer.

### CLAIM OF PRIORITY

This application claims the benefit of priority under 35 U.S.C. §119(e) to U.S. Provisional Patent Application Ser. No. 62/171,134, filed on Jun. 4, 2015, the benefit of priority of which is claimed hereby, and which is incorporated by reference herein in its entirety.

### BACKGROUND OF THE INVENTION

The AAA (ATPase Associated with a variety of Activities) ATPase p97 having the descriptive name, Valosin containing protein, is conserved across all eukaryotes and is essential for life in budding yeast (Giaever, G., et. al. Nature (2002) 418, 387-391) and mice (Muller, J. M. et al. Biochem. Biophys. Res. Commun. (2007) 354, 459-465). Humans bearing reduction-of-function alleles of p97 are afflicted with a syndrome that includes inclusion body myopathy and frontotemporal lobar degeneration (Weihl, C. et al. Hum. Mol. Genet. (2006) 15, 189-199). Loss-of-function studies in model organisms indicate that p97 plays a critical role in a broad array of cellular processes including Golgi membrane reassembly (Rabouille, C. et al. Cell (1995) 82, 905-914), membrane transport (Ye, Y. et al Nature (2001) 414, 652-656; Ye, Y. et al. Nature (2004) 429, 841-847) degradation of misfolded membrane and secretory proteins by the ubiquitin-proteasome system (UPS) (Golbik, R. et al. Biol. Chem. (1999) 380, 1049-1062; Richly, H. et al. Cell (2005) 120, 73-84), regulation of myofibril assembly (Janiesch, P. C. et al. Nat. Cell Biol. (2007) 9, 379-390), and cell division (Cao, K. et al. Cell (2003) 115, 355-367). The broad range of cellular functions for this protein is thought to derive from its ability to unfold proteins or disassemble protein complexes. The mechanochemical activity of p97 is linked to substrate proteins by an array of at least 14 UBX domain adapters that bind p97, as well as the non-UBX domain adaptors Ufdl and Np14 (Meyer, H. H. et al. EMBO J. (2000) 19, 2181-2192).

The sequence of p97 reveals three domains (N-domain, D1 ATPase domain, and D2 ATPase domain) joined by linker regions. X-ray crystallography of p97 revealed that it forms a homohexamer of 97 kilodalton subunits that assemble to form two stacked rings. The two rings are formed by the ATPase domains (Huyton, T. et al., Struct. Biol. (2003) 144, 337-348; DeLaBarre, B. et al. Nat. Struct. Biol. (2003) 10, 856-863). The ‘top’ ring is formed by a hexamer of the D1 domains, whereas the ‘bottom’ ring is formed by a hexamer of the D2 domains. The N-domain extends outward from the D1 domain ring. Although it is clear that the D2 domain hydrolyzes ATP in vitro, the level of D1-specific ATPase activity reported by different investigators varies. Nevertheless, genetic studies in yeast suggest that ATP hydrolysis by both the D1 and D2 domains is essential for the function of p97 (Song, C. et al. J. Biol. Chem. (2003) 278, 3648-3655; Ye, Y. et al. J. Cell Biol. (2004) 162, 71-84). Binding of ATP to the D1 domain is also required for assembly of p97 (Wang, Q. et al. Biochem. Biophys. Res. Commun. (2003) 300, 253-260). Although ATP hydrolysis by the D2 domain is not required for assembly of p97 hexamer, it is thought that ATP hydrolysis by the D2 domain is a substrate conversion, resulting in their unfolding or dissociation from bound partners.

A prominent cellular function for p97 that has received considerable scrutiny is its role in the turnover of misfolded secretory proteins via the UPS (ubiquitin proteasome system). In this process, which is known as ERAD (for endoplasmic reticulum-associated degradation), proteins that fail to fold within the ER are retrotranslocated in a p97-dependent manner into the cytoplasm where they are degraded by the UPS (Ye, Y. et al. Nature (2004) 429, 841-847). In this process, p97 is thought to mediate extraction of substrates from the ER membrane. The complex p97 is also required for the turnover of cytosolic substrates of the UPS (Janiesch, P. C. et al. Nat. Cell Biol. (2007) 9, 379-390; Cao, K. et al. Cell (2003) 115, 355-367; Fu, X. et al. J. Cell Biol. (2003) 163, 21-26), although its role in turnover of cytosolic proteins is less understood.

The Valosin containing protein, p97, represents a suitable target for cancer therapeutics. The complex p97 and its function are essential for continued cellular viability, and so drugs that inhibit it should be antiproliferative. In other words, inhibition of p97 will cause undesirable protein concentration within the target cell. A consequential cellular reaction is often apoptosis or at least amelioration of cellular growth and mitosis. Also, p97 is known to be overproduced in multiple cancers (Yamamoto, S. et al. Ann. Surg. Oncol. (2005) 12, 925-934; Yamamoto, S. et al. Clin. Cancer Res. (2004) 10, 5558-5565; Yamamoto, S. et al. Ann. Surg. Oncol. (2004) 11, 697-704; Yamamoto, S. et al. Ann. Surg. Oncol. (2004) 11, 165-172) suggesting that its activity may be rate-limiting for the development of at least some cancers. p97 is known to be essential for ERAD (Carvalho, P. et al. Cell (2006) 126, 361-373), and recent studies suggest that cancer cells may be particularly dependent upon ERAD (Boelens, J. et al. In Vivo (2007) 21, 215-226). Furthermore, p97 has been linked to the turnover of IIcB and consequent activation of NF-kB (Dai, R. M. et al. J. Biol. Chem. (1998) 273, 3562-3573). NF-kB activity is important for the survival of some tumor cells, particularly in multiple myeloma (Keats, J. J. et. al. Cancer Cell (2007) 12, 131-144; Annunziata, C. M. et. al. Cancer Cell (2007) 12, 115-130). It has been suggested that bortezomib is active in multiple myeloma due to its ability to block turnover of proteins via the ERAD pathway and its ability to block turnover of IkB, thereby squelching the activity of NF-kB. Given that p97 is implicated in both ERAD and IlcB turnover but otherwise has a more restricted role in the UPS compared to the proteasome itself, drugs that target p97 may retain much of the efficacy of bortezomib but with less toxicity.

### GOALS OF THE INVENTION

Thus, there is a need to develop compounds suitable for inhibition of p97 activity and for methods of inhibiting the activity of p97 using such compounds. There is a need to develop such compounds for use in treatment of neoplastic malconditions.

### SUMMARY OF THE INVENTION

These and other needs are met by aspects of the present invention, one of which is directed to a six member ring scaffold having a nitrogen hexacycle as the ring scaffold or core and optionally having one or more substituents bonded to the ring. In various embodiments, the substituents are not bonded to the nitrogens of the nitrogen hexacycle scaffold. In another aspect of the invention, the nitrogen hexacycle compounds of the invention have an ability to inhibit Valosin containing protein p97 and to ameliorate, diminish, shrink, moderate and/or eliminate cells exhibiting neoplastic tendencies and/or abnormal function. In a further aspect of the invention, such compounds inhibit the ATPase activity of p97. Another aspect of the invention concerns treatment of malconditions and/or disease such as cancer through use of such compounds.

An aspect of the invention is directed to the nitrogen hexacycle scaffold, having an aryl alkylamine substituent at position 4 (P4) of the nitrogen hexacycle scaffold, a 5:6 bicyclic group at the position 2 (P2) of the nitrogen hexacycle scaffold and optional single or multiple aliphatic, functional and/or aromatic components as additional substituents on the nitrogen hexacycle scaffold as well as on the P2 and P4 groups.

More specifically, an aspect of the invention is a nitrogen hexacycle compound of Formula I.

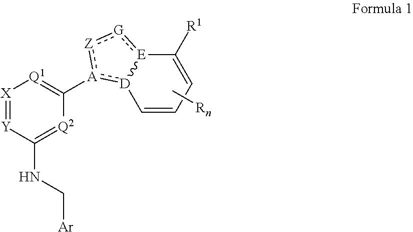

For Formula I, the designations of the symbols A, D, E, G, Q's, R's, X, Y, Z and Ar include the following.

One of X and Y is nitrogen and the other is CR².

One of Q¹ and Q² is nitrogen and the other is CR^{2′} or nitrogen.

The bicyclic ADEGZ ring is positioned at P2 of the scaffold that is 1,3,5-triazine and the P3 position of the scaffold that is 1,2,4 triazine and at the position between Q¹ and Q² for all other scaffold embodiments. These positions are all as shown by Formula I. This bicyclic ring is designated herein as the P2 group. The five member ring of this P2 group is partially saturated or aromatic. The squiggle bond between D and E is a single bond if either of D and E is N. The squiggle bond between D and E is a double bond if both of D and E are carbon. If either D or E is nitrogen, then the P2 group is aromatic. If both of D and E are carbon, then the P2 group has a partially saturated five member ring fused to a six member benzo ring or the P2 group is aromatic. The dotted bonds of the five member ring may be single or double bonds depending on the valence nature of the bonded atom.

A, D and E each are independently carbon or nitrogen, the carbon being an sp² carbon; and A may also be CR⁶, the carbon of CR⁶ being an sp³ carbon.

When one of D and E is nitrogen the squiggle bond between them is a single bond. When both of D and E are carbon, the squiggle bond between them is a double bond.

G and Z are each independently nitrogen, NR³, CR⁴, C(R⁵)₂, oxygen or sulfur, the carbon of CR⁴ being an sp² carbon and the carbon of C(R⁵)₂ being an sp³ carbon, provided that:
when one of G and Z is oxygen or sulfur, each of A, D and E is an sp² carbon;
G and Z are not together a combination of oxygen and sulfur and are not both oxygen or both sulfur;
when G and Z are both C(R⁵)₂, A is CR⁶ or N and D and E are both sp² carbons;
when G and Z are both CR⁴, A is CR⁶ or N and D and E are both sp² carbons.

R¹ is selected from hydrogen, an aliphatic group optionally substituted by a functional group or a functional group.

R², R^{2′}, Rₙ, R⁴ and R⁵'s are each independently selected from hydrogen, an aliphatic group optionally substituted by a functional group, an optionally substituted aromatic group and a functional group.

R³ is hydrogen, an aliphatic group optionally substituted by a functional group and a functional group.

R⁶ is hydrogen or an alkyl group of 1 to 3 carbons.

n is zero or an integer of 1 or 2.

Ar is a substituted or unsubstituted aryl or heteroaryl group positioned at the P4 position of the 1,3,5- or the P5 position of the 1,2,4-nitrogen hexacycle.

Both positions are indicated by Formula I. This Ar group is designated herein as the Ar group or the P4 group. The substituent of the Ar group is an aliphatic group optionally substituted by a functional group or a functional group.

R¹ may be hydrogen, an aliphatic group optionally substituted by a functional group or a functional group. Preferably, R¹ is not hydrogen and may be a functional group or an aliphatic group optionally substituted by a functional group. More preferably, R¹ is not hydrogen or an aliphatic group but may be a functional group. Even more preferably, R1 is any of the functional components that are polar and preferably are hydrogen bonding groups as set forth in Definitions section of the the Detailed Description. The presence of R¹ as other than hydrogen at the 4 position of the ADEGZ bicyclic ring at P2 (1,3,5-Triazine,pyridine, pyrimidine), P3 (1,2,4-Triazine,pyridine, pyrimidine) is an aspect for the development of the biological inhibition of the p97 enzyme complex by the nitrogen hexacycle compounds of Formula I.

As an aliphatic group, R¹ preferably is selected from linear, branched or cyclic alkyl of 1 to 6 carbons, linear, branched or cyclic alkenyl of 2 to 6 carbons, linear, branched or cyclic alkoxyalkyl or alkoxyalkenyl of 2 to 6 carbons, the thia analog thereof, linear, branched or cyclic alkanoyl or alkenoyl of 2 to 6 carbons, linear, branched or cyclic alkanoylalkyl or alkenoylalkyl or 3 to 7 carbons, linear, branched or cyclic alkanoyloxy or alkanoyloxy of 2 to 6 carbons, or linar, branched or cyclic alkanoyloxyalkyl or alkenoyloxyalkyl of 3 to 7 carbons.

More preferably under the polar and preferably hydrogen bonding aspect, R¹ is boronic acid, boronic ester, carboxylic acid, carboxylic ester, carboxamide, sulfonic acid, sulfonic ester, sulfonamide, aminocarbonyl alkyl, aminosulfonylalkyl, amine, monoalkyl amine, hydroxyl, hydroxyalkyl, alkoxy, nitrile, nitro, methylenyl or ethylenyl carboxylic acid or sulfonic acid, methylenyl or ethylenyl carboxylic or sulfonic ester, methylenyl or ethylenyl carboxamide or sulfonamide.

Preferably, R² and R^{2′} are each independently hydrogen or an aliphatic group or a functional group as set forth in the Detailed Description.

More preferably under the aliphatic aspect, R² and R^{2′} are each independently selected from halogen, straight or branched alkyl of 1 to 6 carbons, substituted aminomethyl, amine, mono, di or trialkyl amine of 1 to 6 carbons, nitrile, perfluoroalkyl of 1 to 3 carbons, alkoxy of 1 to 6 carbons.

More preferably under the functional group aspect, R² and R^{2′} are each independently selected from boronic acid, boronic ester, carboxylic acid, carboxylic ester, carboxamide, sulfonic acid, sulfonic ester, sulfonamide, aminocarbonyl alkyl, aminosulfonylalkyl, amine, monoalkyl amine, hydroxyl, hydroxyalkyl, alkoxy, cyano, nitro, methylenyl or ethylenyl carboxylic acid or sulfonic acid, methylenyl or ethylenyl carboxylic or sulfonic ester, methylenyl or ethylenyl carboxamide or sulfonamide wherein the ester groups each are 1 to 6 linear, branched or cyclic alkyl groups and the alkyl groups are linear, branched or cyclic and 1 to 6 carbons.

Most preferably, R² and R^{2′} are each independently selected from hydrogen, linear, branched or cyclic alkyl of 1 to 6 carbons, boronic acid, boronic ester, carboxylic acid, carboxylic ester, carboxamide, sulfonic acid, sulfonic ester, sulfonamide, aminocarbonyl alkyl, aminosulfonylalkyl with 1 to 6 carbons in the ester and alkyl groups.

More preferably, the substituents Rₙ, R⁴ and the R⁵'s may be independently selected from hydrogen, halogen, straight or branched alkyl of 1 to 6 carbons, carboxylic acid, carboxamide, substituted aminomethyl, sulfonic acid, sulfonamide, amine, mono, di or trialkyl amine of 1 to 6 carbons, nitrile, N-alkyl carboxamide of 1 to 6 carbons in the alkyl group, perfluoroalkyl of 1 to 3 carbons, alkoxy of 1 to 6 carbons, boronic acid or boronic ester having 1 to 3 carbons in the ester group. Preferably one of the R⁵'s is hydrogen.

More preferably, R³ is hydrogen, alkyl of 1 to 6 carbons, perfluoroalkyl of 1 to 6 carbons or alkylcarbonyl group of 2 to 6 carbons.

Especially more preferably, Rₙ, R⁴ and the R⁵'s are each independently hydrogen, straight or branched alkyl of 1 to 6 carbons, nitrile or halogen. Most preferably Rₙ, R⁴ and the R⁵'s are each independently hydrogen or methyl, with hydrogen being the preferred version of these two substituents for each of Rₙ and and one of R⁵.

Especially more preferably, R³ is hydrogen or alkyl of 1 to 6 carbons and most preferably, R³ is hydrogen or methyl, especially hydrogen, and most especially methyl.

More preferably, n is 0 or 1, most preferably n is 0.

More preferably Y is N and X is CR².

When R¹ is hydrogen or an aliphatic group, R² R^{2′} and Ar may be formulated as described above, or alternatively, R² and R^{2′} are each independently selected from boronic acid, boronic ester, carboxyl, sulfonoxy, carboxamide, sulfonamide, aminocarbonyl alkyl, aminosulfonylalkyl, amine, monoalkyl amine, hydroxyl, hydroxyalkyl, alkoxy, cyano, nitro, carboxylic ester, sulfonic ester, methylenyl or ethylenyl carboxylic acid or sulfonic acid, methylenyl or ethylenyl carboxylic or sulfonic ester, methylenyl or ethylenyl carboxamide or sulfonamide or alternatively, the Ar group may be substituted by any of the polar, hydrogen bonding functional groups listed in this paragraph for R¹. In this alternative, one or both of R² and one or both of R^{2′} and the substituent for Ar may be one of the polar, hydrogen bonding groups listed in this paragraph.

For all of the foregoing substitutions, the maximum number of boronic acid or ester groups present on the nitrogen hexacycle compounds of Formula I is one (1).

An additional aspect of the invention is directed to a pharmaceutical composition of a pharmaceutically acceptable carrier and the above described nitrogen hexacycle compounds of Formula I, especially as set forth in the following Detailed Description.

Another aspect of the invention is directed to a method of decreasing Valosin containing protein (p97) activity or decreasing degradation of a proteasome system substrate, especially a ubiquitin substrate, by administration to a patient in need an effective therapeutic amount of the foregoing nitrogen hexacycle ring scaffold, more specifically the above described nitrogen hexacycle compounds of Formula I.

Yet another aspect of the invention is directed to the treatment of neoplastic malconditions, cancer and other malconditions associated with p97 by administration to a patient in need, the foregoing pharmaceutical composition.

### DETAILED DESCRIPTION OF THE INVENTION DEFINITIONS

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “may” in the context of this application means “is permitted to” or “is able to” and is a synonym for the term “can.” The term “may” as used herein does not mean possibility or chance.

The term “disease” or “disorder” or “malcondition” are used interchangeably, and are used to refer to diseases or conditions wherein the p97 complex plays a role in the biochemical mechanisms involved in the disease or malcondition or symptom(s) thereof such that a therapeutically beneficial effect can be achieved by acting on the p97 complex. “Acting on” the p97 complex, or “modulating” the p97 complex, can include binding to the p97 complex and/or inhibiting the bioactivity of the p97 complex and/or allosterically regulating the bioactivity of the p97 complex in vivo.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, less than the total number of carbon atoms in the universe and bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite.

In general, “substituted” refers to an organic group as defined herein in which one or more bonds to a hydrogen atom contained therein are replaced by one or more bonds to a non-hydrogen atom. More particularly, the term “chemical substituent” refers to any and all aliphatic, aromatic and functional groups listed in this section that can be appended to an organic molecule. A functional group is an inorganic moiety such as halogen, sulfate, nitro, amino and the like as well as monocarbon functional groups such as carboxyl, carbonyl, carboxamide that are ordinary and typical optional substituents of organic molecules. In the context of this invention, recitation of this term without indication of specific groups constitutes the definition given above. Recitation of this term in combination with a Markush recitation of specific groups constitutes a subgenus of the understanding conveyed by the foregoing definition. The term “substituent” generally means any appropriate group named below that has an “yl”, “y” or “o” ending to designate that it is appended, attached or covalently bonded to another moiety such as but not limited to an aromatic framework. Examples include but are not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR′, OC(O)N(R′)₂, B(OH)₂, B(OR′″)₂ with R′″ being C1 to C6 alkyl, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

In various embodiments, J can be halo, nitro, cyano, OR, NR₂, or R, or is C(O)OR, C(O)NR₂, OC(O)OR, OC(O)NR₂, N(R)C(O)OR, N(R)C(O)NR₂ or thio/thiono analogs thereof. By “thio/thiono analogs thereof”, with respect to a group containing an O, is meant that any or all O atoms in the group can be replaced by an S atom; e.g., for group C(O)OR, a “thio/thiono analog thereof” includes C(S)OR, C(O)SR, and C(S)SR; e.g., for group OC(O)NR₂, a “thio/thiono analog thereof” includes SC(O)NR₂, OC(S)NR₂, and SC(S)NR₂; and so forth.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

For all substituents, the first atom of the molecular formula of the substituent is the atom bonding the substituent to its corresponding moiety, eg, for the functional group, N(R^{a})C(O)R^{a}, the N is bonded to the corresponding moiety substituted by this group. If the substituent is described in words, such as alkyenylamine, the phrase ending in “enyl” indicates the carbon atom bonding the substituent to its corresponding moiety. For substituents that display a single bonding site, such as carboxylic acid, sulfonic acid, fluoro, methyl and the like, the bonding arrangement is the expected arrangement.

“Aliphatic substituent, group or component” refers to any organic group that is non-aromatic. Included are acyclic and cyclic organic compounds composed of carbon, hydrogen and optionally of oxygen, nitrogen, sulfur and other heteroatoms. This term encompasses all of the following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched and linear versions and such groups optionally substituted with functional groups, as these groups and others meeting this definition of “aliphatic” are defined below.

“Aromatic substituent, group or component” refers to any and all aromatic groups including but not limited to aryl, aralkyl, heteroalkylaryl, heteroalkylheteroaryl and heteroaryl groups. The term “aromatic” is general in that it encompasses all compounds containing aryl groups optionally substituted with functional groups (all carbon aromatic groups) and all compounds containing heteroaryl groups optionally substituted with functional groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

Unless stated otherwise specifically in the specification, an alkyl group is optionally substituted by one or more of substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SW, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an (alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

Unless stated otherwise specifically in the specification, an alkenyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR, —SW, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., C2-C10 alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C2-C5 alkynyl). The alkynyl is attached to the rest of the molecule by a single bond, for example, ethynyl, propynyl, butynyl, pentynyl, hexynyl, and the like.

Unless stated otherwise specifically in the specification, an alkynyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SW, —OC(O)—R^{a}, —N(R^{a})₂, —C(O)R^{a}, —C(O)OR^{a}, —C(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Alkynyl-cycloalkyl” refers to refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a (C═O)H radical.

“Carboxyl” refers to a (C═O)OH radical.

“Cyano” refers to a CN radical.

“Cycloalkyl” refers to a or polycyclic radical that contains only carbon and hydrogen, and may be saturated, or partially unsaturated. Cycloalkyl groups include groups having from 3 to 10 ring atoms (i.e., C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. In some embodiments, it is a C₃-C₈ cycloalkyl radical. In some embodiments, it is a C₃-C₅ cycloalkyl radical. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

Unless stated otherwise specifically in the specification, a cycloalkyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Cycloalkyl-alkenyl” refers to a (cycloalkyl) alkenyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heterocycloalkyl” refers to a (cycloalkyl) heterocycyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heteroaryl” refers to a (cycloalkyl) heteroaryl radical where cycloalkyl and heterocycloalkyl are as disclosed herein and which are optionally substituted by one or more of the substituents described as suitable substituents for heterocycloalkyl and cycloalkyl respectively.

“Alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

“Substituted alkoxy” refers to alkoxy wherein the alkyl constituent is substituted (i.e., —O-(substituted alkyl)).

Unless stated otherwise specifically in the specification, the alkyl moiety of an alkoxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —C(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Alkoxycarbonyl” refers to a group of the formula (alkoxy)(C═O)-attached through the carbonyl carbon wherein the alkoxy group has the indicated number of carbon atoms. Thus a C1-C6 alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group. In some embodiments, C₁-C₄ alkoxy, is an alkoxy group which encompasses both straight and branched chain alkoxy groups of from 1 to 4 carbon atoms.

“Substituted alkoxycarbonyl” refers to the group (substituted alkyl)-O—C(O)— wherein the group is attached to the parent structure through the carbonyl functionality.

Unless stated otherwise specifically in the specification, the alkyl moiety of an alkoxycarbonyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—, and (heterocycloalkyl)-C(O)—, wherein the group is attached to the parent structure through the carbonyl functionality. In some embodiments, it is a C₁-C₁₀ acyl radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Acyloxy” refers to a R(C═O)O— radical wherein “R” is alkyl, aryl, heteroaryl, heteroalkyl, or heterocycloalkyl, which are as described herein. In some embodiments, it is a C₁-C₄ acyloxy radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl,—OR^{a}, —SR^{a},—OC(O)—R^{a}, —N(R^{a})₂, —C(O)R^{a}, —C(O)OR^{a},—OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2-S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, unless stated otherwise specifically in the specification. When a—N(R^{a})₂ group has two Ra other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl.

Unless stated otherwise specifically in the specification, an amino group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SW,—OC(O)—R^{a}, —N(R^{a})₂, —C(O)R^{a}, —C(O)OR^{a}, OC(O)N(R^{a})₂,—C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl and each of these moieties may be optionally substituted as defined herein.

“Substituted amino” also refers to N-oxides of the groups—NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

“Amide” or “amido” refers to a chemical moiety with formula —C(O)N(R)₂ or NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. In some embodiments it is a C₁-C₄ amido or amide radical, which includes the amide carbonyl in the total number of carbons in the radical. The R₂ of —N(R)₂ of the amide may optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

“Aryl” refers to a conjugated pi radical with six or ten ring atoms which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. The term includes or -ring polycyclic (i.e., rings which share adjacent pairs of ring atoms) groups.

Unless stated otherwise specifically in the specification, an aryl moiety is optionally substituted by one or more substituents as defined above. Such substituents further are independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl,—OR^{a}, —SW,—OC(O)—R^{a}, —N(R^{a})₂, —C(O)R^{a}, —C(O)OR^{a}, —OC(O)N(R^{a})₂,—C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Aralkyl” or “arylalkyl” refers to an (aryl)alkyl radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

Unless stated otherwise specifically in the specification, an ester group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SW,—OC(O)—R^{a}, —N(R^{a})₂, —C(O)R^{a}, —C(O)OR^{a}, —OC(O)N(R^{a})₂,—C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Functional substituent, group or component” refers to a substituent capable of displaying functionality such as hydroxyl, ester, amide, amine, enamine, halogen, cyano, thio, oxidized sulfur, nitrogen or phosphorus groups, alkoxy, olefinic, aldehyde, ketone, carboxylic acid, anhydride, urethane, urea, imine, amidine, hydroxylimine, hydroxylamine, nitrile, organometallic, and any other group capable of displaying dipole interaction and/or reactivity. See Basic Principles of Organic Chemistry, Roberts & Casario, W. A. Benjamin, publisher New York, N.Y. 1965, Chapter 10. Additional examples include hydroxy, halo, cyano, trifluoromethyl, trifluoromethoxy, nitro, trimethylsilanyl,—OR^{a}, —SR^{a},—OC(O)—R^{a}, —N(R^{a})₂, —C(O)R^{a}, —B(OH)₂, —B(OR′)₂ with R′ being C1-C6 alkyl, —C(O)OR^{a}, —C(O)N(R^{a})₂, —C(O)N(R^{a})₂, —N(R^{a})C(O)OR^{a}, —N(R^{a})C(O)R^{a},—N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), —R^{a}—N(R^{a})₂ or PO₃(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl, heteroarylalkyl or any combination thereof. Preferably W is hydrogen or linear, branched or cyclic alkyl of 1 to 6 carbons; more preferably hydrogen, methyl or ethyl; most preferably hydrogen.

A subcategory of the term “functional component” includes the foregoing groups that are polar and preferably are hydrogen bonding. The term “polar functional component” constitutes this subcategory and includes the foregoing examples except for olefinic groups and other non-polar groups. These non-polar groups are excluded from the term “polar functional component.”

The term “polar” means that the so designated group exhibits a dipole moment and/or significant electronegativity or electropositivity so that electromagnetic attraction between such polar groups occurs.

The term hydrogen bonding means that the group either will form a pseudobond with a polarized group containing hydrogen or is such a polarized group containing hydrogen.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given, e.g. C₁-C₄ heteroalkyl which refers to the chain length in total, which in this example is 4 atoms long. For example, a —CH₂OCH₂CH₃ radical is referred to as a “C₄” heteroalkyl, which includes the heteroatom center in the atom chain length description. Connection to the rest of the molecule may be through either a heteroatom or a carbon in the heteroalkyl chain.

A heteroalkyl group may be substituted with one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl,—OR^{a}, —SR^{a},—OC(O)—R^{a}, —N(R^{a})₂, —C(O)R^{a}, —C(O)OR^{a},—C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl respectively.

“Heteroalkylheterocycloalkyl” refers to an -(heteroalkyl)heterocycloalkyl radical where heteroalkyl and heteroaryl are as disclosed herein and which are optionally substituted by one or more of the substituents described as suitable substituents for heteroalkyl and heterocycloalkyl respectively.

“Heteroalkylcycloalkyl” refers to an -(heteroalkyl) cycloalkyl radical where heteroalkyl and cycloalkyl are as disclosed herein and which are optionally substituted by one or more of the substituents described as suitable substituents for heteroalkyl and cycloalkyl respectively.

“Heteroaryl” refers to a 5, 6 or 10-membered aromatic radical (e.g., C₅-C₁₃ heteroaryl) that includes one or more ring heteroatoms selected from nitrogen, oxygen and sulfur, and which may be a, bicyclic, tricyclic or tetracyclic ring system. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be or non-. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to adeninyl, azabenzimidazolyl, azaindolyl, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinykisothiazolyl, imidazolyl, imidazopyridinyl, isoxazolopyridinyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thianaphthalenyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl), xanthinyl, guaninyl, quinoxalinyl, and quinazolinyl groups.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Unless stated otherwise specifically in the specification, a heteraryl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a},—OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

Substituted heteroaryl also includes ring systems substituted with one or more oxide (—O—) substituents, such as pyridinyl N-oxides.

“Heterocyclic” refers to any or polycyclic moiety comprising at least one heteroatom selected from nitrogen, oxygen and sulfur. As used herein, heterocyclyl moieties can be aromatic or nonaromatic. The moieties heteroaryl and heterocyclyl alkyl are members of the heterocyclic group.

Unless stated otherwise, heterocyclic moieties are optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl,—OR^{a}, —SR^{a},—OC(O)—R^{a}, —N(R^{a})₂,—C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heteroarylalkyl” refers to a moiety having an aryl moiety, as described herein, connected to an alkylene moiety, as described herein, wherein the connection to the remainder of the molecule is through the alkylene group.

“Heterocyclylalkyl” refers to a stable 5, 6 or 10-membered non-aromatic ring radical having from one to six heteroatoms selected from nitrogen, oxygen and sulfur. Unless stated otherwise specifically in the specification, the heterocycloalkyl radical is a, bicyclic, tricyclic or tetracyclic ring system, which may include or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

Unless stated otherwise specifically in the specification, a heterocycloalkyl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl,—OR^{a}, —SR^{a},—OC(O)—R^{a}, —N(R^{a})₂, —C(O)R^{a}, —C(O)OR^{a},—C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heterocyclylalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂.

“Sulfanyl” refers to the groups: —S-(optionally substituted alkyl), —S-(optionally substituted aryl),—S-(optionally substituted heteroaryl), and —S-(optionally substituted heterocycloalkyl).

“Sulfinyl” refers to the groups: —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocycloalkyl).

“Sulfonyl” refers to the groups: —S(O₂)—H, —S(O₂)-(optionally substituted alkyl),—S(O₂)-(optionally substituted amino),—S(O₂)-(optionally substituted aryl),—S(O₂)-(optionally substituted heteroaryl), and—S(O₂)-(optionally substituted heterocycloalkyl).

“Sulfonamidyl” or “sulfonamido” refers to a S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in NRR of the S(═O)₂—NRR radical may be taken together with the nitrogen to which it is attached to form a 4-, 5-, 6-, or 7-membered ring. In some embodiments, it is a C₁-C₁₀ sulfonamido, wherein each R in sulfonamido contains 1 carbon, 2 carbons, 3 carbons, or 4 carbons total. A sulfonamido group is optionally substituted by one or more of the substituents described for alkyl, cycloalkyl, aryl, heteroaryl respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl respectively.

“Azido” refers to an N₃ group. An “azide” can be an organic azide or can be a salt of the azide (N₃) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

“Urethane” (“carbamoyl” or “carbamyl”) includes N- and O-urethane groups, i.e., —NRC(O)OR and OC(O)NR₂ groups, respectively.

“Sulfonamide” (or “sulfonamido”) includes S- and N-sulfonamide groups, i.e.,—SO₂NR₂ and NRSO₂R groups, respectively. Sulfonamide groups therefore include but are not limited to sulfamoyl groups (—SO₂NH₂). An organosulfur structure represented by the formula S(O)(NR) is understood to refer to a sulfoximine, wherein both the oxygen and the nitrogen atoms are bonded to the sulfur atom, which is also bonded to two carbon atoms.

“Amidine” or “amidino” includes groups of the formula —C(NR)NR₂. Typically, an amidino group is C(NH)NH₂.

“Guanidine” or “guanidino” includes groups of the formula —NRC(NR)NR₂. Typically, a guanidino group is NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The term “amino protecting group” or “N-protected” as used herein refers to those groups intended to protect an amino group against undesirable reactions during synthetic procedures and which can later be removed to reveal the amine.

Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

### Compounds

The invention is directed to compounds that inhibit ATPase Associated with a variety of Activities (AAA), the ATPase having the descriptive name Valosin containing protein, also known as p97, as well as methods to treat or prevent a disease or condition in a subject that would benefit by inhibition of p97. The compounds embodying of the invention incorporate a nitrogen hexacycle ring scaffold optionally substituted by aliphatic, functional and/or aromatic substituents. Preferably, the scaffold has an amino alkylaryl or heteroaryl group that is substituted or unsubstituted at P4 position and a bicyclic group at P2 position. More preferably, the scaffold is substituted by a substituted or unsubstituted benzyl amine group at the P4 position and a bicyclic 5:6 ring at the P2 position. The scaffold ring as well as the alkylaryl and heteroaromatic groups may be substituted by multiple aliphatic, functional and/or aromatic groups described in the foregoing Definitions section.

A preferred embodiment of the six member scaffold of the invention is a nitrogen hexacycle compound of Formula I:

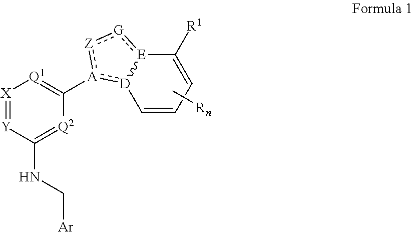

The variable groups X, Y, Q¹, Q², A, D, E, G and Z are as defined in the Summary of the Invention. The generic through the most preferred descriptions of Rₙ, and R¹ through R⁶ are given above in the Summary of the Invention. The preferred, more preferred and especially preferred descriptions of these substitutents are also given in the Summary and are repeated here. These degrees of preference for the substituents are repeated in the Claims.

The general designations for X and Y are such that one is N and the other is CR². The general designations for Q¹ and Q² provide that one is nitrogen and the other is CR^{2′} or nitrogen so that at least one of Wand Q² is always nitrogen and both can be nitrogen. The selections for X, Y, Wand Q² provide a nitrogen hexacycle scaffold with the proviso that one of X and Y and one of Q¹ and Q² is always N. The preferred scaffold designation is both of Wand Q² as N. A preferred designation of X and Y under this Q designation is Y as CR² and X as N. A more preferred designation of X and Y under this Q designation is Y as N and X as CR².

The variations in Q¹, Q², X and Y of Formula I produce the following nitrogen hexacycle scaffold skeleton embodiments of Formula I, namely RT 1-RT 6. The arrows in the upper right corner and bottom of each embodiment mark respectively the locations of the P2 and P4 groups. All substituents for the scaffold and the P2 and P 4 groups including R¹ through R⁶ as well as the P2 and P4 groups are considered present on each of these skeleton embodiments as described above for Formula I and all preferred, more preferred and most preferred designations of these groups and substituents.

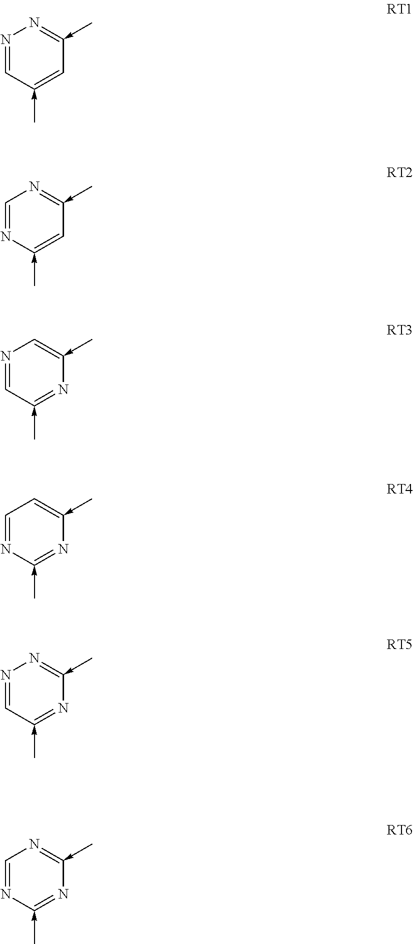

The preferred group for Ar is phenyl or substituted phenyl wherein the substituent is fluoro, trifluoromethyl, boronic acid, boronic alkyl ester with a C1 to C6 alkyl, carboxylic acid, carboxylic alkyl ester with a C1 to C6 alkyl, carboxyamide, sulfonic acid, sulfonamide. The preferred substituent is fluoro, boronic acid, boronic ester, carboxylic acid, carboxamid, sulfonic acid, sulfonic ester. The more preferred substituent is fluoro, boronic acid, carboxylic acid, sulfonic acid. The most preferred substituent is fluoro or boronic acid. The most preferred Ar group is phenyl or p-fluorophenyl.

The 5:6 bicyclic group at P2 may have a partially saturated or aromatic five member ring and an aromatic six member ring. If one of D and E of the six member ring is nitrogen, the bond between them is a single bond but the lone pair of electrons of the nitrogen provides a conjugation pathway to render this six member ring “aromatic.” This bicyclic group may be all carbon or may contain 1, 2 or 3 nitrogens and/or oxygen or sulfur in the five member ring. Under these conditions, A, D and E can each independently be carbon or N with carbon being an sp² carbon. Also A may also independently be CR⁶ with C being an sp³ carbon. The variables Z and G may independently each be N, O or S or NR³, CR⁴ or C(R⁵)₂ with CR⁴ being an sp² carbon and C(R⁵)₂ being an sp³ carbon. However, Z and G cannot both be O, S, or O—S.

In the context of valence requirements and partial unsaturation to aromaticity for the P2 group, the corresponding five member ring cannot be formed as an all nitrogen ring or as four nitrogens and one carbon. These requirements for the ADEGZ ring provide the following skeleton embodiments for the 5:6 bicyclic P2 group. The substituents Rₙ and R¹, R³, R⁴, R⁵ and R⁶ are to be considered attached to each of these skeleton embodiments as provided in Formula I and the general, preferred, more preferred and especially more preferred definitions thereof.

With three nitrogens and two sp² carbons, the following structural embodiments are exemplified. This configuration would also include A, D and E as nitrogen and G and Z as sp² carbons as well as G, D and E as nitrogen and A and Z as sp² carbons.

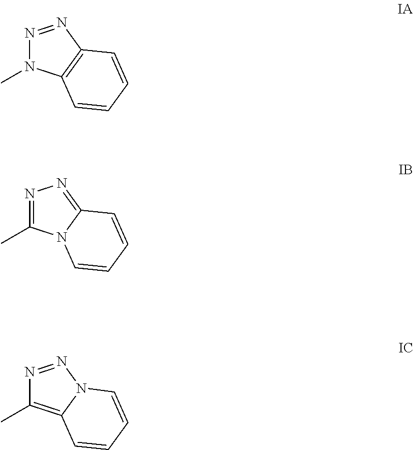

With two nitrogens and three sp² carbons, the following structural embodiments are exemplified.

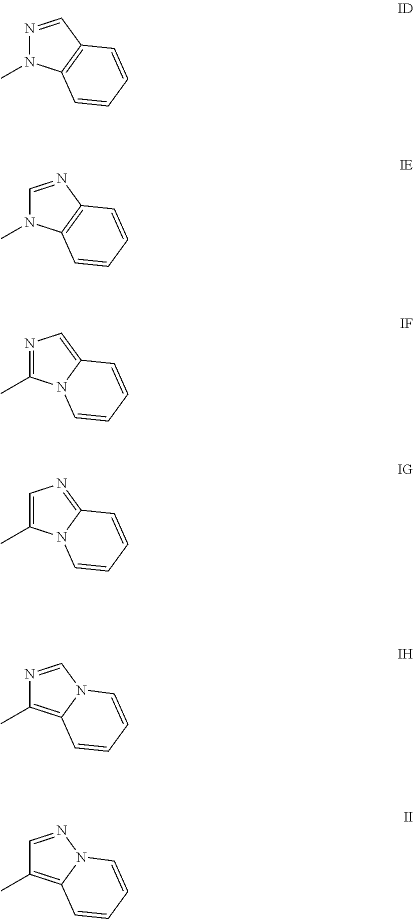

With one nitrogen and four sp² carbons or with two sp² carbons and two sp^{a} carbons, the following structural embodiments are exemplified.

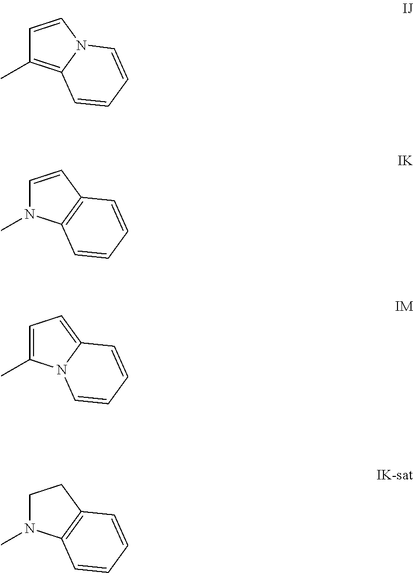

With one nitrogen as NR³, the following structural embodiments are exemplified.

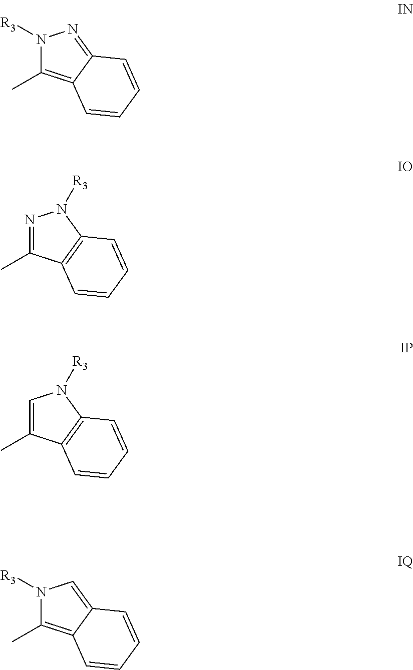

With O or S, the following structural embodiments are exemplified.

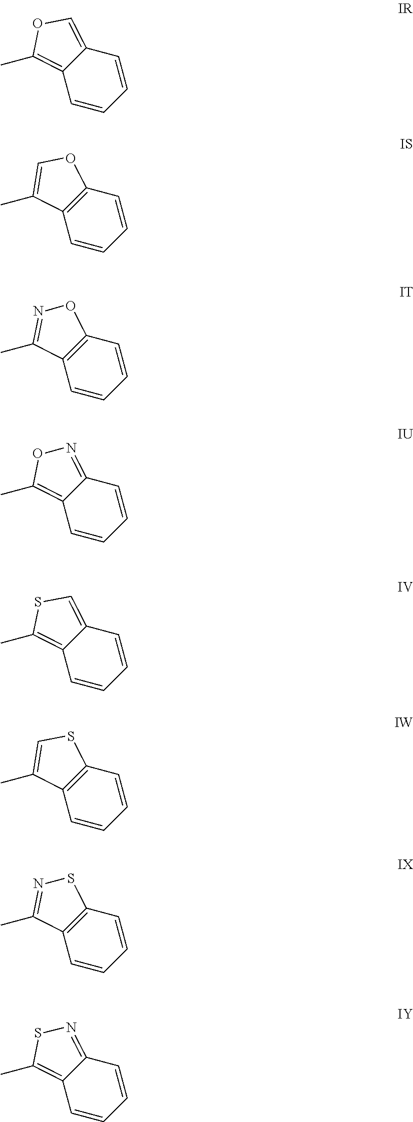

With all carbons of which three are sp³ and two are sp² or four are sp² and one is sp³, the following structural embodiments are exemplified (indan-1-yl and inden-1-yl respectively).

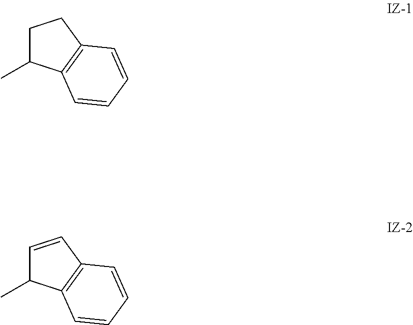

In addition to the foregoing descriptions of the R substituents for Formula I and the RT and I skeleton embodiments, substituents Ar, R¹, R², R^{2′} R³, R⁴ R⁵ and Rₙ for Formula I and the skeletons for the scaffold (RT's) and the P2 group (I's) have the following designations.

R¹ may be hydrogen or an aliphatic group as described in the foregoing Definitions sections. R1 may preferably be not hydrogen and may be selected from linear, branched or cyclic alkyl of 1 to 6 carbons, linear, branched or cyclic alkenyl of 2 to 6 carbons, linear, branched or cyclic alkoxyalkyl or alkoxyalkenyl of 2 to 6 carbons, the thia analog thereof, linear, branched or cyclic alkanoyl or alkenoyl of 2 to 6 carbons, linear, branched or cyclic alkanoylalkyl or alkenoylalkyl or 3 to 7 carbons, linear, branched or cyclic alkanoyloxy or alkanoyloxy of 2 to 6 carbons, or linar, branched or cyclic alkanoyloxyalkyl or alkenoyloxyalkyl of 3 to 7 carbons.

R¹ may be other than hydrogen or an aliphatic group and more preferably may be selected from the group consisting of B(OH)₂, B(OR)₂ wherein R is an alkyl group of 1 to 6 carbons, OR^{d}, (CH₂)ₙOR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d},—N(R^{d})C(O)N(R^{d})₂, N(R^{d})C(NR^{d})N(R^{d})₂, N(R^{d})S(O)ₜR^{d}, S(O)ₜOR^{d}, S(Oₜ)R^{d}, S(O)ₜN(R^{d})₂, N(R^{d})₂, (CH₂)ₙN(R^{d})₂, PO₃(R^{d})₂, C(O)R^{d} and CF₃. Each n is independently an integer of 1, 2 or 3, preferably 1. Each t is independently an integer of 1 or 2, preferably 2. Each R^{d} is independently hydrogen, alkyl of 1 to 6 carbons, fluoroalkyl of 1 to 6 carbons, carbocyclyl of 3 to 10 carbons, carbocyclylalkyl of 4 to 12 carbons, aryl of 6 to 10 carbons, aralkyl, heterocyclyl, heterocyclylalkyl, heteroaryl, heteroarylalkyl, alkenyl of 2 to 6 carbons, alkynyl or 2 to 6 carbons or any combination thereof. Preferably, R^{d} is hydrogen, phenyl or alkyl of 1 to 6 carbons, preferably 1 to 3 carbons, more preferably methyl or ethyl. More preferably, R^{d} is hydrogen, methyl or ethyl. Most preferably, R^{d} is hydrogen or methyl.

R¹ is even more preferably selected from the group consisting of boronic acid, boronic ester with the ester group being straight, branched or cyclic alkyl of 1 to 6 carbons, carboxylic acid, carboxyl ester with the ester group being straight, branched or cyclic alkyl of 1 to 6 carbons, carboxamide, N-alkyl carboxamide of 1 to 6 carbons in the straight, branched or cyclic alkyl group, sulfonic acid, sulfonyl ester with the ester group being straight, branched or cyclic alkyl of 1 to 6 carbons, sulfonamide, alkyl substituted sulfonamide with the alkyl group being straight, branched or cyclic of 1 to 6 carbons, amine (NH₂), mono or dialkyl amine with the alkyl being straight, branched or cyclic of 1 to 6 carbons, N-alkyl amino methyl with the alkyl group being straight, branched or cyclic of 1 to 6 carbons, nitrile, hydroxyl, straight, branched or cyclic hydroxy alkyl of 1 to 6 carbons in the alkyl group, or straight, branched or cyclic alkoxy of 1 to 6 carbons.

The position of R¹ as ortho to E, in other words at the 4 position of the 5:6 bicyclic P2/P3 heterocycle is an aspect for improved development of the inhibition activity of the nitrogen hexacycle compounds of Formula I against the p97 enzyme complex. Any polar or lipophilic (non-polar) group at this position confers higher activity than does hydrogen at this position. The presence of a polar, hydrogen bonding group such as an boronic acid, amide, carboxylic acid, sulfonamide, sulfonic acid, hydroxyl, alkylenyl alcohol (eg., CH₂OH and similar substituents), amine or alkylenyl amine (eg., CH₂NH₂) confers higher activity than does a lipophilic (non-polar) group such as methyl or ethyl.

R² and R^{2′} may each independently and preferably be selected from the group consisting of hydrogen, linear, branched or cyclic alkyl or alkenyl of 1 to 6 carbons (2 minimum for alkenyl), halogen, B(OH)₂, B(OR)₂ with 1 to 6 carbons in th R group, OR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d},—N(R^{d})C(O)N(R^{d})₂, N(R^{d})C(NR^{d})N(R^{d})₂, N(R^{d})S(O)ₜR^{d}, S(O)ₜOR^{d}, S(O)ₜN(R^{d})₂, N(R^{d})₂, (CH₂)_{q}N(R^{d})₂ and PO₃(R^{d})₂ wherein each R^{d} is independently hydrogen, alkyl, fluoroalkyl, carbocyclyl, carbocyclylalkyl, aryl, aralkyl, heterocyclyl, heterocyclylalkyl, heteroaryl, heteroarylalkyl alkenyl, alkynyl or any combination thereof. Each t is independently selected from the group of integers of 1 and 2. Each q is independently an integer of 0, 1, 2 or 3, n is an integer of 0, 1 or 2, preferably 1, more preferably 0. Preferably, R^{d} is hydrogen, phenyl or alkyl of 1 to 6 carbons, preferably 1 to 3 carbons, more preferably methyl or ethyl. More preferably, R^{d} is hydrogen, methyl or ethyl. Most preferably, R^{d} is hydrogen or methyl. R³ cannot be halogen.

R² and R^{2′} more preferably may each independently be selected from the group consisting of hydrogen, halogen (preferably fluoro, chloro or bromo, more preferably fluoro or chloro, most preferably fluoro), straight, branched or cyclic alkyl of 1 to 6 carbons, carboxylic acid, carboxyl ester with the ester group being straight, branched or cyclic alkyl of 1 to 6 carbons, N-alkyl amino methyl with the alkyl group being straight, branched or cyclic of 1 to 6 carbons, boronic acid, sulfonic acid, boronic ester with the ester groups each independently being straight, branched or cyclic alkyl of 1 to 6 carbons, sulfonyl ester with the ester group being straight, branched or cyclic alkyl of 1 to 6 carbons, sulfonamide, amine (NH₂), mono, di or trialkyl amine with the alkyl being straight, branched or cyclic of 1 to 6 carbons, nitrile, carboxamide, N-alkyl carboxamide of 1 to 6 carbons in the straight, branched or cyclic alkyl group, perfluoroalkyl of 1 to 3 carbons, straight, branched or cyclic alkoxy of 1 to 6 carbons.

R³ preferably is hydrogen, an alkyl or perfluoroalkyl group of 1 to 6 carbons or an alkylcarbonyl group of 2 to 6 carbons.

R⁴, the R⁵'s and Rₙ may preferably each be independently selected from the group consisting of hydrogen, linear, branched or cyclic alkyl or alkenyl of 1 to 6 carbons (2 minimum for alkenyl), halogen, B(OH)₂, B(OR)₂ with 1 to 6 carbons in th R group, OR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d},—N(R^{d})C(O)N(R^{d})₂, N(R^{d})C(NR^{d})N(R^{d})₂, N(R^{d})S(O)ₜR^{d}, S(O)ₜOR^{d}, S(O)ₜN(R^{d})₂, N(R^{d})₂, (CH₂)_{q}N(R^{d})₂ and PO₃(R^{d})₂ wherein each R^{d} is independently hydrogen, alkyl, fluoroalkyl, carbocyclyl, carbocyclylalkyl, aryl, aralkyl, heterocyclyl, heterocyclylalkyl, heteroaryl, heteroarylalkyl alkenyl, alkynyl or any combination thereof. Each t is independently selected from the group of integers of 1 and 2. Each q is independently an integer of 0, 1, 2 or 3, n is an integer of 0, 1 or 2, preferably 1, more preferably 0. Preferably, R^{d} is hydrogen, phenyl or alkyl of 1 to 6 carbons, preferably 1 to 3 carbons, more preferably methyl or ethyl. More preferably, R^{d} is hydrogen, methyl or ethyl. Most preferably, R^{d} is hydrogen or methyl. R³ cannot be halogen. Preferably one of the R⁵'s is hydrogen.

R⁴, the R⁵'s and IL more preferably may be each independently selected from the group consisting of hydrogen, halogen (preferably fluoro, chloro or bromo, more preferably fluoro or chloro, most preferably fluoro), straight, branched or cyclic alkyl of 1 to 6 carbons, carboxylic acid, carboxyl ester with the ester group being straight, branched or cyclic alkyl of 1 to 6 carbons, N-alkyl amino methyl with the alkyl group being straight, branched or cyclic of 1 to 6 carbons, boronic acid, sulfonic acid, boronic ester with each ester group independently being straight, branched or cyclic alkyl, sulfonyl ester with the ester group being straight, branched or cyclic alkyl of 1 to 6 carbons, sulfonamide, amine (NH₂), mono, di or trialkyl amine with the alkyl being straight, branched or cyclic of 1 to 6 carbons, nitrile, carboxamide, N-alkyl carboxamide of 1 to 6 carbons in the straight, branched or cyclic alkyl group, perfluoroalkyl of 1 to 3 carbons, straight, branched or cyclic alkoxy of 1 to 6 carbons. Preferably one of the R⁵'s is hydrogen.

R⁶ may be hydrogen or an alkyl group of 1 to 3 carbons.

More preferred embodiments of the invention include the nitrogen hexacycle compounds of Formula I where R¹ is selected from COOH, COOR with R being alkyl of 1 to 3 carbons, B(OH)₂, B(OR)₂ with R being alkyl of 1 to 3 carbons, OMe, OEt, CN, V(CH₂)ₘW, N(R^{a})₂, CO(NR^{a})₂ SO₂R^{a}, SO₂N(R^{a})₂. The preferred selections for R² and R^{2′} are the same as those listed for R¹ and also include hydrogen, methyl, ethyl, n-propyl, isopropyl, n-butyl, isobutyl. R³ is selected from H, Me, Et, Pr, iPr, Bu, iBu, COR^{a} and SO₂R^{a}. R⁴ and the R⁵'s are each independently selected from H, Me, Et, Pr, iPr, Bu, iBu, COOH, CONH(R^{a}), SO₂NH(R^{a}), B(OH)₂, B(OR)₂ with R being alkyl of 1 to 3 carbons. Rₙ is selected from H, halogen, CN, methyl or ethyl. The integer designator n is independently 0 or 1, preferably 0. The integer designator m is 0 or an integer of 1, 2 or 3, preferably 1 or 0. In each instance, R^{a} of these more preferred embodiments is H, Me, Et, Ph and when two R^{a}'s are present each is selected independently. Y for these preferred embodiments is O, S, NH, CO₂, CO, CONH and N-alkyl. W for these preferred embodiments is amine, alkylamine, alkoxy, alkonyloxy, carboxylic acid, carboxamide, carboxyl ester or N-alkyl carboxamide, sulfonic acid, sulfonamide, boronic acid or boronic alkyl ester.

Most preferred embodiments of the invention include the nitrogen hexacycle compounds of Formula I where R¹ is B(OH)₂, B(OMe or OEt)₂, CONH₂, CN, SO₂NH₂, COOH, COOMe, COOEt, CH₂NH₂, CH₂NHCOCH₃, CH₂NHSO₂CH₃, CH₂OH, CH₂CH₂OH or OH. R² and R^{2′} are each independently H, Me, Et, CONH₂, SO₂NH₂, B(OH)₂, B(OMe or OEt)₂, OMe, OEt, CN, F, Cl or Br, most especially, H or Me and of these two substituents, preferably H; R³ is H, Me, Et, COMe, COEt, SO₂Me or SO₂Et, most especially H or Me and of these two substituents, preferably H; R⁴ and the R⁵'s are each independently H, Me, Et, most especially H; R⁶ is H or Me, preferably H, and Rₙ is excluded by n as 0. Most preferably, only one B(OH)₂, B(OMe or OEt)₂ is present on Formula I when B(OH)₂, B(OMe or OEt)₂ is chosen.

When R¹ is not a polar hydrogen bonding group, it is preferred to have one of R², R^{2′} or the substituent of the Ar group be a polar hydrogen bonding group such as B(OH)₂, B(OMe or OEt)₂, CONH₂, CN, SO₂NH₂, COOH, COOMe, COOEt, CH₂NH₂, CH₂NHCOCH₃, CH₂NHSO₂CH₃, CH₂OH, CH₂CH₂OH or OH. Although it is not a requirement of the invention, it is believed that the presence of an accessible polar hydrogen bonding group at the R¹ position, the R² or R^{2′} position or on Ar facilitates competitive or non-competitive enzymatic site binding or allosteric binding with p97 and thereby promotes inhibition of enzymatic activity.

The number of boronic acid or boronic ester groups as substituents anywhere on Formula I is one.

### Preferences of Formulas IA-IZ

Among Formulas IA-IZ with the definitions and preferences for Arylalkyl amine, Rₙ and R¹-R⁶ given above, Formulas IA, ID, IE, IG, IH, IK, IO, IP, IS, IT, IU, IX, IY and IZ-2 are preferred. More preferred of these skeleton Formulas of P2 with the preferences for Arylalkyl amine, Rₙ and R¹-R⁶ given above are IA, ID, IE, IG, IH, IK, IP and IS. Most preferred of these skeleton embodiments of P2 with the preferences for Arylalkyl amine, Rₙ and R¹-R⁶ given above IA, ID, IE, IK, IP and IS.

### Preferred Nitrogen Hexacycle Compounds

Individual embodiments of the nitrogen hexacycle compounds of Formula I include the specific compounds named in the following Lists. These compounds are identified by their IUPAC names.

These lists of preferred and most preferred nitrogen hexacycle compounds are arranged according to the identity of the P2 group. Within each category of P2 group, the compounds are arranged according to the scaffold: a 1, 3, 5-Triazine or b) a 1, 2, 4-Triazine indicated by Formula I, the substituent R¹ is located at the S4 position of the P2 group. The sublists are also organized according to the identity of the substitutent at this S4 position. These lists are organized according to the arrangement of the scaffold and the P2 bicyclic substituent. The headings provide the designations of the P2 group. Because the individual compounds of each heading are also rearranged according to the identities of the scaffold and the group at S4 of P2, the compounds of each heading can be rearranged and separated also according to the scaffold and the group at the S4 position of P2. These individual subcategories based upon the identities of the scaffolds and the group at S4 of P2 are included herein as if explicitly set forth. Hence, these sub-lists based upon the identities of the scaffolds and the groups at S4 of P2 constitute a part of the embodiments of the invention.

The preferred list of compounds includes the more and most preferred compounds and the more preferred list includes the most preferred compounds also.

### Preferred Individual Compounds

### [1,2,3]triazolo[1,5-a]pyridine as P2

3-(5-((3-fluorobenzyl)amino)-6-carbamoyl-1,2,4-triazin-3-yl)-[1,2,3]triazolo[1,5-a]pyridine-7-carboxamide
3-(5-(benzylamino)-6-carbamoyl-1,2,4-triazin-3-yl)-[1,2,3]triazolo[1,5-a]pyridine-7-carboxamide
3-(5-((3-fluorobenzyl)amino)-6-ethyl-1,2,4-triazin-3-yl)-[1,2,3]triazolo[1,5-a]pyridine-7-carboxamide
3-(5-(benzylamino)-6-ethyl-1,2,4-triazin-3-yl)-[1,2,3]triazolo[1,5-a]pyridine-7-carboxamide
3-(5-((3-fluorobenzyl)amino)-1,2,4-triazin-3-yl)-[1,2,3]triazolo[1,5-a]pyridine-7-carboxamide
3-(5-(benzylamino)-1,2,4-triazin-3-yl)-[1,2,3]triazolo[1,5-a]pyridine-7-carboxamide
3-(5-((3-fluorobenzyl)amino)-6-methyl-1,2,4-triazin-3-yl)-[1,2,3]triazolo[1,5-a]pyridine-7-carboxamide
3-(5-(benzylamino)-6-methyl-1,2,4-triazin-3-yl)-[1,2,3]triazolo[1,5-a]pyridine-7-carboxamide
3-(5-((3-fluorobenzyl)amino)-6-(dimethylamino)-1,2,4-triazin-3-yl)-[1,2,3]triazolo[1,5-a]pyridine-7-carboxamide
3-(5-(benzylamino)-6-(dimethylamino)-1,2,4-triazin-3-yl)-[1,2,3]triazolo[1,5-a]pyridine-7-carboxamide
3-(5-((3-fluorobenzyl)amino)-6-methoxy-1,2,4-triazin-3-yl)-[1,2,3]triazolo[1,5-a]pyridine-7-carboxamide
3-(5-(benzylamino)-6-methoxy-1,2,4-triazin-3-yl)-[1,2,3]triazolo[1,5-a]pyridine-7-carboxamide
3-(4-((3-fluorobenzyl)amino)-6-carbamoyl-1,3,5-triazin-2-yl)-[1,2,3]triazolo[1,5-a]pyridine-7-carboxamide
3-(4-(benzylamino)-6-carbamoyl-1,3,5-triazin-2-yl)-[1,2,3]triazolo[1,5-a]pyridine-7-carboxamide
3-(4-((3-fluorobenzyl)amino)-6-ethyl-1,3,5-triazin-2-yl)-[1,2,3]triazolo[1,5-a]pyridine-7-carboxamide
3-(4-(benzylamino)-6-ethyl-1,3,5-triazin-2-yl)-[1,2,3]triazolo[1,5-a]pyridine-7-carboxamide
3-(4-((3-fluorobenzyl)amino)-1,3,5-triazin-2-yl)-[1,2,3]triazolo[1,5-a]pyridine-7-carboxamide
3-(4-(benzylamino)-1,3,5-triazin-2-yl)-[1,2,3]triazolo[1,5-a]pyridine-7-carboxamide
3-(4-((3-fluorobenzyl)amino)-6-methyl-1,3,5-triazin-2-yl)-[1,2,3]triazolo[1,5-a]pyridine-7-carboxamide
3-(4-(benzylamino)-6-methyl-1,3,5-triazin-2-yl)-[1,2,3]triazolo[1,5-a]pyridine-7-carboxamide
3-(4-((3-fluorobenzyl)amino)-6-(dimethylamino)-1,3,5-triazin-2-yl)-[1,2,3]triazolo[1,5-a]pyridine-7-carboxamide
3-(4-(benzylamino)-6-(dimethylamino)-1,3,5-triazin-2-yl)-[1,2,3]triazolo[1,5-a]pyridine-7-carboxamide
3-(4-((3-fluorobenzyl)amino)-6-methoxy-1,3,5-triazin-2-yl)-[1,2,3]triazolo[1,5-a]pyridine-7-carboxamide
3-(4-(benzylamino)-6-methoxy-1,3,5-triazin-2-yl)-[1,2,3]triazolo[1,5-a]pyridine-7-carboxamide

### [1,2,4]triazolo[4,3-a]pyridine as P2

3-(5-((3-fluorobenzyl)amino)-6-carbamoyl-1,2,4-triazin-3-yl)-[1,2,4]triazolo[4,3-a]pyridine-8-carboxamide
3-(5-(benzylamino)-6-carbamoyl-1,2,4-triazin-3-yl)-[1,2,4]triazolo[4,3-a]pyridine-8-carboxamide
3-(5-((3-fluorobenzyl)amino)-6-ethyl-1,2,4-triazin-3-yl)-[1,2,4]triazolo[4,3-a]pyridine-8-carboxamide
3-(5-(benzylamino)-6-ethyl-1,2,4-triazin-3-yl)-[1,2,4]triazolo[4,3-a]pyridine-8-carboxamide
3-(5-((3-fluorobenzyl)amino)-1,2,4-triazin-3-yl)-[1,2,4]triazolo[4,3-a]pyridine-8-carboxamide
3-(5-(benzylamino)-1,2,4-triazin-3-yl)-[1,2,4]triazolo[4,3-a]pyridine-8-carboxamide
3-(5-((3-fluorobenzyl)amino)-6-methyl-1,2,4-triazin-3-yl)-[1,2,4]triazolo[4,3-a]pyridine-8-carboxamide
3-(5-(benzylamino)-6-methyl-1,2,4-triazin-3-yl)-[1,2,4]triazolo[4,3-a]pyridine-8-carboxamide
3-(5-((3-fluorobenzyl)amino)-6-(dimethylamino)-1,2,4-triazin-3-yl)-[1,2,4]triazolo[4,3-a]pyridine-8-carboxamide
3-(5-(benzylamino)-6-(dimethylamino)-1,2,4-triazin-3-yl)-[1,2,4]triazolo[4,3-a]pyridine-8-carboxamide
3-(5-((3-fluorobenzyl)amino)-6-methoxy-1,2,4-triazin-3-yl)-[1,2,4]triazolo[4,3-a]pyridine-8-carboxamide
3-(5-(benzylamino)-6-methoxy-1,2,4-triazin-3-yl)-[1,2,4]triazolo[4,3-a]pyridine-8-carboxamide
3-(4-((3-fluorobenzyl)amino)-6-carbamoyl-1,3,5-triazin-2-yl)-[1,2,4]triazolo[4,3-a]pyridine-8-carboxamide
3-(4-(benzylamino)-6-carbamoyl-1,3,5-triazin-2-yl)-[1,2,4]triazolo[4,3-a]pyridine-8-carboxamide
3-(4-((3-fluorobenzyl)amino)-6-ethyl-1,3,5-triazin-2-yl)-[1,2,4]triazolo[4,3-a]pyridine-8-carboxamide
3-(4-(benzylamino)-6-ethyl-1,3,5-triazin-2-yl)-[1,2,4]triazolo[4,3-a]pyridine-8-carboxamide
3-(4-((3-fluorobenzyl)amino)-1,3,5-triazin-2-yl)-[1,2,4]triazolo[4,3-a]pyridine-8-carboxamide
3-(4-(benzylamino)-1,3,5-triazin-2-yl)-[1,2,4]triazolo[4,3-a]pyridine-8-carboxamide
3-(4-((3-fluorobenzyl)amino)-6-methyl-1,3,5-triazin-2-yl)-[1,2,4]triazolo[4,3-a]pyridine-8-carboxamide
3-(4-(benzylamino)-6-methyl-1,3,5-triazin-2-yl)-[1,2,4]triazolo[4,3-a]pyridine-8-carboxamide
3-(4-((3-fluorobenzyl)amino)-6-(dimethylamino)-1,3,5-triazin-2-yl)-[1,2,4]triazolo[4,3-a]pyridine-8-carboxamide
3-(4-(benzylamino)-6-(dimethylamino-1,3,5-triazin-2-yl)-[1,2,4]triazolo[4,3-a]pyridine-8-carboxamide
3-(4-((3-fluorobenzyl)amino)-6-methoxy-1,3,5-triazin-2-yl)-[1,2,4]triazolo[4,3-a]pyridine-8-carboxamide
3-(4-(benzylamino)-6-methoxy-1,3,5-triazin-2-yl)-[1,2,4]triazolo[4,3-a]pyridine-8-carboxamide

### 1H-benzo[d][1,2,3]triazole as P2

1-(5-((3-fluorobenzyl)amino)-6-carbamoyl-1,2,4-triazin-3-yl)-1H-benzo[d][1,2,3]triazole-4-carboxamide
1-(5-(benzylamino)-6-carbamoyl-1,2,4-triazin-3-yl)-1H-benzo[d][1,2,3]triazole-4-carboxamide
1-(5-((3-fluorobenzyl)amino)-6-ethyl-1,2,4-triazin-3-yl)-1H-benzo[d][1,2,3]triazole-4-carboxamide
1-(5-(benzylamino)-6-ethyl-1,2,4-triazin-3-yl)-1H-benzo[d][1,2,3]triazole-4-carboxamide
1-(5-((3-fluorobenzyl)amino)-1,2,4-triazin-3-yl)-1H-benzo[d][1,2,3]triazole-4-carboxamide
1-(5-(benzylamino)-1,2,4-triazin-3-yl)-1H-benzo[d][1,2,3]triazole-4-carboxamide
1-(5-((3-fluorobenzyl)amino)-6-methyl-1,2,4-triazin-3-yl)-1H-benzo[d][1,2,3]triazole-4-carboxamide
1-(5-(benzylamino)-6-methyl-1,2,4-triazin-3-yl)-1H-benzo[d][1,2,3]triazole-4-carboxamide
1-(5-((3-fluorobenzyl)amino)-6-(dimethylamino)-1,2,4-triazin-3-yl)-1H-benzo[d][1,2,3]triazole-4-carboxamide
1-(5-(benzylamino)-6-(dimethylamino)-1,2,4-triazin-3-yl)-1H-benzo[d][1,2,3]triazole-4-carboxamide
1-(5-((3-fluorobenzyl)amino)-6-methoxy-1,2,4-triazin-3-yl)-1H-benzo[d][1,2,3]triazole-4-carboxamide
1-(5-(benzylamino)-6-methoxy-1,2,4-triazin-3-yl)-1H-benzo[d][1,2,3]triazole-4-carboxamide
1-(4-((3-fluorobenzyl)amino)-6-carbamoyl-1,3,5-triazin-2-yl)-1H-benzo[d][1,2,3]triazole-4-carboxamide
1-(4-(benzylamino)-6-carbamoyl-1,3,5-triazin-2-yl)-1H-benzo[d][1,2,3]triazole-4-carboxamide
1-(4-((3-fluorobenzyl)amino)-6-ethyl-1,3,5-triazin-2-yl)-1H-benzo[d][1,2,3]triazole-4-carboxamide
1-(4-(benzylamino)-6-ethyl-1,3,5-triazin-2-yl)-1H-benzo[d][1,2,3]triazole-4-carboxamide
1-(4-((3-fluorobenzyl)amino)-1,3,5-triazin-2-yl)-1H-benzo[d][1,2,3]triazole-4-carboxamide
1-(4-(benzylamino)-1,3,5-triazin-2-yl)-1H-benzo[d][1,2,3]triazole-4-carboxamide
1-(4-((3-fluorobenzyl)amino)-6-methyl-1,3,5-triazin-2-yl)-1H-benzo[d][1,2,3]triazole-4-carboxamide
1-(4-(benzylamino)-6-methyl-1,3,5-triazin-2-yl)-1H-benzo[d][1,2,3]triazole-4-carboxamide
1-(4-((3-fluorobenzyl)amino)-6-(dimethylamino)-1,3,5-triazin-2-yl)-1H-benzo[d][1,2,3]triazole-4-carboxamide
1-(4-(benzylamino)-6-(dimethylamino)-1,3,5-triazin-2-yl)-1H-benzo[d][1,2,3]triazole-4-carboxamide
1-(4-((3-fluorobenzyl)amino)-6-methoxy-1,3,5-triazin-2-yl)-1H-benzo[d][1,2,3]triazole-4-carboxamide
1-(4-(benzylamino)-6-methoxy-1,3,5-triazin-2-yl)-1H-benzo[d][1,2,3]triazole-4-carboxamide

### 1H-indazole as P2

3-(5-((3-fluorobenzyl)amino)-6-carbamoyl-1,2,4-triazin-3-yl)-1H-indazole-7-carboxamide
3-(5-(benzylamino)-6-carbamoyl-1,2,4-triazin-3-yl)-1H-indazole-7-carboxamide
3-(5-((3-fluorobenzyl)amino)-6-ethyl-1,2,4-triazin-3-yl)-1H-indazole-7-carboxamide
3-(5-(benzylamino)-6-ethyl-1,2,4-triazin-3-yl)-1H-indazole-7-carboxamide
3-(5-((3-fluorobenzyl)amino)-1,2,4-triazin-3-yl)-1H-indazole-7-carboxamide
3-(5-(benzylamino)-1,2,4-triazin-3-yl)-1H-indazole-7-carboxamide
3-(5-((3-fluorobenzyl)amino)-6-methyl-1,2,4-triazin-3-yl)-1H-indazole-7-carboxamide
3-(5-(benzylamino)-6-methyl-1,2,4-triazin-3-yl)-1H-indazole-7-carboxamide
3-(5-((3-fluorobenzyl)amino)-6-(dimethylamino)-1,2,4-triazin-3-yl)-1H-indazole-7-carboxamide
3-(5-(benzylamino)-6-(dimethylamino)-1,2,4-triazin-3-yl)-1H-indazole-7-carboxamide
3-(5-((3-fluorobenzyl)amino)-6-methoxy-1,2,4-triazin-3-yl)-1H-indazole-7-carboxamide
3-(5-(benzylamino)-6-methoxy-1,2,4-triazin-3-yl)-1H-indazole-7-carboxamide
3-(4-((3-fluorobenzyl)amino)-6-carbamoyl-1,3,5-triazin-2-yl)-1H-indazole-7-carboxamide
3-(4-(benzylamino)-6-carbamoyl-1,3,5-triazin-2-yl)-1H-indazole-7-carboxamide
3-(4-((3-fluorobenzyl)amino)-6-ethyl-1,3,5-triazin-2-yl)-1H-indazole-7-carboxamide
3-(4-(benzylamino)-6-ethyl-1,3,5-triazin-2-yl)-1H-indazole-7-carboxamide
3-(4-((3-fluorobenzyl)amino)-1,3,5-triazin-2-yl)-1H-indazole-7-carboxamide
3-(4-(benzylamino)-1,3,5-triazin-2-yl)-1H-indazole-7-carboxamide
3-(4-((3-fluorobenzyl)amino)-6-methyl-1,3,5-triazin-2-yl)-1H-indazole-7-carboxamide
3-(4-(benzylamino)-6-methyl-1,3,5-triazin-2-yl)-1H-indazole-7-carboxamide
3-(4-((3-fluorobenzyl)amino)-6-(dimethylamino)-1,3,5-triazin-2-yl)-1H-indazole-7-carboxamide
3-(4-(benzylamino)-6-(dimethylamino)-1,3,5-triazin-2-yl)-1H-indazole-7-carboxamide
3-(4-((3-fluorobenzyl)amino)-6-methoxy-1,3,5-triazin-2-yl)-1H-indazole-7-carboxamide
3-(4-(benzylamino)-6-methoxy-1,3,5-triazin-2-yl)-1H-indazole-7-carboxamide
(3-(((3-(4-(aminomethyl)-1H-indazol-1-yl)-6-carbamoyl-1,2,4-triazin-5-yl)amino)methyl)phenyl)boronic acid
3-(4-(aminomethyl)-1H-indazol-1-yl)-5-((3-fluorobenzyl)amino)-1,2,4-triazine, pyridine, pyrimidine-6-carboxamide
3-(4-(aminomethyl)-1H-indazol-1-yl)-5-(benzylamino)-1,2,4-triazine, pyridine, pyrimidine-6-carboxamide
(3-(((3-(4-(methoxymethyl)-1H-indazol-1-yl)-6-carbamoyl-1,2,4-triazin-5-yl)amino)methyl)phenyl)boronic acid
3-(4-(methoxymethyl)-1H-indazol-1-yl)-5-((3-fluorobenzyl)amino)-1,2,4-triazine, pyridine, pyrimidine-6-carboxamide
3-(4-(methoxymethyl)-1H-indazol-1-yl)-5-(benzylamino)-1,2,4-triazine, pyridine, pyrimidine-6-carboxamide
(3-(((3-(4-carbamoyl-1H-indazol-1-yl)-6-mcarbamoyl-1,2,4-triazin-5-yl)amino)methyl)phenyl)boronic acid
1-(5-((3-fluorobenzyl)amino)-6-carbamoyl-1,2,4-triazin-3-yl)-1H-indazole-4-carboxamide
1-(5-(benzylamino)-6-carbamoyl-1,2,4-triazin-3-yl)-1H-indazole-4-carboxamide
(3-(((6-carbamoyl-3-(-1H-indazol-1-yl)-1,2,4-triazin-5-yl)amino)methyl)phenyl)boronic acid
5-((3-fluorobenzyl)amino)-3-(-1H-indazol-1-yl)-1,2,4-triazine, pyridine, pyrimidine-6-carboxamide
5-(benzylamino)-3-(-1H-indazol-1-yl)-1,2,4-triazine, pyridine, pyrimidine-6-carboxamide
(3-(((6-carbamoyl-3-(-4-sulfinamoyl-1H-indazol-1-yl)-1,2,4-triazin-5-yl)amino)methyl)phenyl)boronic acid
1-(5-((3-fluorobenzyl)amino)-6-carbamoyl-1,2,4-triazin-3-yl)-1H-indazole-4-sulfonamide
1-(5-(benzylamino)-6-carbamoyl-1,2,4-triazin-3-yl)-1H-indazole-4-sulfonamide
(3-(((3-(4-(aminomethyl)-1H-indazol-1-yl)-6-ethyl-1,2,4-triazin-5-yl)amino)methyl)phenyl)boronic acid
3-(4-(aminomethyl)-1H-indazol-1-yl)-N-(3-fluorobenzyl)-6-ethyl-1,2,4-triazin-5-amine
3-(4-(aminomethyl)-1H-indazol-1-yl)-N-benzyl-6-ethyl-1,2,4-triazin-5-amine
(3-(((3-(4-(methoxymethyl)-1H-indazol-1-yl)-6-ethyl-1,2,4-triazin-5-yl)amino)methyl)phenyl)boronic acid
3-(4-(methoxymethyl)-1H-indazol-1-yl)-N-(3-fluorobenzyl)-6-ethyl-1,2,4-triazin-5-amine
3-(4-(methoxymethyl)-1H-indazol-1-yl)-N-benzyl-6-ethyl-1,2,4-triazin-5-amine
(3-(((3-(4-carbamoyl-1H-indazol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-1H-indazole-4-carboxamide
1-(5-(benzylamino)-6-ethyl-1,2,4-triazin-3-yl)-1H-indazole-4-carboxamide
(3-(((6-ethyl-3-(-1H-indazol-1-yl)-1,2,4-triazin-5-yl)amino)methyl)phenyl)boronic acid
N-(3-fluorobenzyl)-6-ethyl-3-(-1H-indazol-1-yl)-1,2,4-triazin-5-amine
N-benzyl-6-ethyl-3-(-1H-indazol-1-yl)-1,2,4-triazin-5-amine
(3-(((6-ethyl-3-(-4-sulfinamoyl-1H-indazol-1-yl)-1,2,4-triazin-5-yl)amino)methyl)phenyl)boronic acid
1-(5-((3-fluorobenzyl)amino)-6-ethyl-1,2,4-triazin-3-yl)-1H-indazole-4-sulfonamide
1-(5-(benzylamino)-6-ethyl-1,2,4-triazin-3-yl)-1H-indazole-4-sulfonamide
(3-(((3-(4-(aminomethyl)-1H-indazol-1-yl)-6-1,2,4-triazin-5-yl)amino)methyl)phenyl)boronic acid
3-(4-(aminomethyl)-1H-indazol-1-yl)-N-(3-fluorobenzyl)-1,2,4-triazin-5-amine
3-(4-(aminomethyl)-1H-indazol-1-yl)-N-benzyl-1,2,4-triazin-5-amine
(3-(((3-(4-(methoxymethyl)-1H-indazol-1-yl)-6-1,2,4-triazin-5-yl)amino)methyl)phenyl)boronic acid
3-(4-(methoxymethyl)-1H-indazol-1-yl)-N-(3-fluorobenzyl)-1,2,4-triazin-5-amine
3-(4-(methoxymethyl)-1H-indazol-1-yl)-N-benzyl-1,2,4-triazin-5-amine
(3-(((3-(4-carbamoyl-1H-indazol-1-yl)-1,2,4-triazin-5-yl)amino)methyl)phenyl)boronic acid
1-(5-((3-fluorobenzyl)amino)-1,2,4-triazin-3-yl)-1H-indazole-4-carboxamide
1-(5-(benzylamino)-1,2,4-triazin-3-yl)-1H-indazole-4-carboxamide
(3-(((3-(-1H-indazol-1-yl)-1,2,4-triazin-5-yl)amino)methyl)phenyl)boronic acid
N-(3-fluorobenzyl)-3-(-1H-indazol-1-yl)-1,2,4-triazin-5-amine
N-benzyl-3-(-1H-indazol-1-yl)-1,2,4-triazin-5-amine
(3-(((3-(-4-sulfinamoyl-1H-indazol-1-yl)-1,2,4-triazin-5-yl)amino)methyl)phenyl)boronic acid
1-(5-((3-fluorobenzyl)amino)-1,2,4-triazin-3-yl)-1H-indazole-4-sulfonamide
1-(5-(benzylamino)-1,2,4-triazin-3-yl)-1H-indazole-4-sulfonamide
(3-(((3-(4-(aminomethyl)-1H-indazol-1-yl)-6-methyl-1,2,4-triazin-5-yl)amino)methyl)phenyl)boronic acid
3-(4-(aminomethyl)-1H-indazol-1-yl)-N-(3-fluorobenzyl)-6-methyl-1,2,4-triazin-5-amine
3-(4-(aminomethyl)-1H-indazol-1-yl)-N-benzyl-6-methyl-1,2,4-triazin-5-amine
(3-(((3-(4-(methoxymethyl)-1H-indazol-1-yl)-6-methyl-1,2,4-triazin-5-yl)amino)methyl)phenyl)boronic acid
3-(4-(methoxymethyl)-1H-indazol-1-yl)-N-(3-fluorobenzyl)-6-methyl-1,2,4-triazin-5-amine
3-(4-(methoxymethyl)-1H-indazol-1-yl)-N-benzyl-6-methyl-1,2,4-triazin-5-amine
(3-(((3-(4-carbamoyl-1H-indazol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-1H-indazole-4-carboxamide
1-(5-(benzylamino)-6-methyl-1,2,4-triazin-3-yl)-1H-indazole-4-carboxamide
(3-(((6-methyl-3-(-1H-indazol-1-yl)-1,2,4-triazin-5-yl)amino)methyl)phenyl)boronic acid
N-(3-fluorobenzyl)-6-methyl-3-(-1H-indazol-1-yl)-1,2,4-triazin-5-amine
N-benzyl-6-methyl-3-(-1H-indazol-1-yl)-1,2,4-triazin-5-amine
(3-(((6-methyl-3-(-4-sulfinamoyl-1H-indazol-1-yl)-1,2,4-triazin-5-yl)amino)methyl)phenyl)boronic acid
1-(5-((3-fluorobenzyl)amino)-6-methyl-1,2,4-triazin-3-yl)-1H-indazole-4-sulfonamide
1-(5-(benzylamino)-6-methyl-1,2,4-triazin-3-yl)-1H-indazole-4-sulfonamide
(3-(((3-(4-(aminomethyl)-1H-indazol-1-yl)-6-(dimethylamino)-1,2,4-triazin-5-yl)amino)methyl)phenyl)boronic acid
3-(4-(aminomethyl)-1H-indazol-1-yl)-N5-(3-fluorobenzyl)-N6,N6-dimethyl-1,2,4-triazine, pyridine, pyrimidine-5,6-diamine
3-(4-(aminomethyl)-1H-indazol-1-yl)-N5-benzyl-N6,N6-dimethyl-1,2,4-triazine, pyridine, pyrimidine-5,6-diamine
(3-(((3-(4-(methoxymethyl)-1H-indazol-1-yl)-6-(dimethylamino)-1,2,4-triazin-5-yl)amino)methyl)phenyl)boronic acid
3-(4-(methoxymethyl)-1H-indazol-1-yl)-N5-(3-fluorobenzyl)-N6,N6-dimethyl-1,2,4-triazine, pyridine, pyrimidine-5,6-diamine
3-(4-(methoxymethyl)-1H-indazol-1-yl)-N5-benzyl-N6,N6-dimethyl-1,2,4-triazine, pyridine, pyrimidine-5,6-diamine
1-(5-(benzylamino)-6-(dimethylamino)-1,2,4-triazin-3-yl)-1H-indazole-4-carboxylic acid
(3-(((3-(4-carbamoyl-1H-indazol-1-yl)-6-(dimethylamino)-1,2,4-triazin-5-yl)amino)methyl)phenyl)boronic acid
1-(5-((3-fluorobenzyl)amino)-6-(dimethylamino)-1,2,4-triazin-3-yl)-1H-indazole-4-carboxamide
1-(5-(benzylamino)-6-(dimethylamino)-1,2,4-triazin-3-yl)-1H-indazole-4-carboxamide
(3-(((6-(dimethylamino)-3-(-1H-indazol-1-yl)-1,2,4-triazin-5-yl)amino)methyl)phenyl)boronic acid
N5-(3-fluorobenzyl)-N6,N6-dimethyl-3-(-1H-indazol-1-yl)-1,2,4-triazine, pyridine, pyrimidine-5,6-diamine
N5-benzyl-N6,N6-dimethyl-3-(-1H-indazol-1-yl)-1,2,4-triazine, pyridine,
pyrimidine-5,6-diamine
(3-(((6-(dimethylamino)-3-(-4-sulfinamoyl-1H-indazol-1-yl)-1,2,4-triazin-5-yl)amino)methyl)phenyl)boronic acid
1-(5-((3-fluorobenzyl)amino)-6-(dimethylamino)-1,2,4-triazin-3-yl)-1H-indazole-4-sulfonamide
1-(5-(benzylamino)-6-(dimethylamino)-1,2,4-triazin-3-yl)-1H-indazole-4-sulfonamide
(3-(((3-(4-(aminomethyl)-1H-indazol-1-yl)-6-methoxy-1,2,4-triazin-5-yl)amino)methyl)phenyl)boronic acid
3-(4-(aminomethyl)-1H-indazol-1-yl)-N-(3-fluorobenzyl)-6-methoxy-1,2,4-triazin-5-amine
3-(4-(aminomethyl)-1H-indazol-1-yl)-N-benzyl-6-methoxy-1,2,4-triazin-5-amine
(3-(((3-(4-(methoxymethyl)-1H-indazol-1-yl)-6-methoxy-1,2,4-triazin-5-yl)amino)methyl)phenyl)boronic acid
3-(4-(methoxymethyl)-1H-indazol-1-yl)-N-(3-fluorobenzyl)-6-methoxy-1,2,4-triazin-5-amine
3-(4-(methoxymethyl)-1H-indazol-1-yl)-N-benzyl-6-methoxy-1,2,4-triazin-5-amine
(3-(((3-(4-carbamoyl-1H-indazol-1-yl)-6-methoxy-1,2,4-triazin-5-yl)amino)methyl)phenyl)boronic acid
1-(5-((3-fluorobenzyl)amino)-6-methoxy-1,2,4-triazin-3-yl)-1H-indazole-4-carboxamide
1-(5-(benzylamino)-6-methoxy-1,2,4-triazin-3-yl)-1H-indazole-4-carboxamide
(3-(((6-methoxy-3-(-1H-indazol-1-yl)-1,2,4-triazin-5-yl)amino)methyl)phenyl)boronic acid
N-(3-fluorobenzyl)-6-methoxy-3-(-1H-indazol-1-yl)-1,2,4-triazin-5-amine
N-benzyl-6-methoxy-3-(-1H-indazol-1-yl)-1,2,4-triazin-5-amine
(3-(((6-methoxy-3-(-4-sulfinamoyl-1H-indazol-1-yl)-1,2,4-triazin-5-yl)amino)methyl)phenyl)boronic acid
1-(5-((3-fluorobenzyl)amino)-6-methoxy-1,2,4-triazin-3-yl)-1H-indazole-4-sulfonamide
1-(5-(benzylamino)-6-methoxy-1,2,4-triazin-3-yl)-1H-indazole-4-sulfonamide
(3-(4-(3-fluorobenzylamino)-6-carbomyl-1,3,5-triazin-2-yl)-1H-indazol-7-yl)boronic acid
(3-(4-(benzylamino)-6-carbomyl-1,3,5-triazin-2-yl)-1H-indazol-7-yl)boronic acid
(3-(((4-(7-(aminomethyl)-1H-indazol-3-yl)-6-carbomyl-1,3,5-triazin-2-yl)amino)methyl)phenyl)boronic acid
4-(7-(aminomethyl)-1H-indazol-3-yl)-6-((3-fluorobenzyl)amino)-1,3,5-triazine, pyridine, pyrimidine-2-carboxamide
4-(7-(aminomethyl)-1H-indazol-3-yl)-6-(benzylamino)-1,3,5-triazine, pyridine,
pyrimidine-2-carboxamide
(3-(((4-(7-(methoxymethyl)-1H-indazol-3-yl)-6-carbomyl-1,3,5-triazin-2-yl)amino)methyl)phenyl)boronic acid
4-((3-fluorobenzylamino)-6-((7-(methoxymethyl)-1H-indazol-3-yl)-1,3,5-triazine, pyridine, pyrimidine-2-carboxamide
4-(benzylamino)-6-(7-(methoxymethyl)-1H-indazol-3-yl)-1,3,5-triazine, pyridine, pyrimidine-2-carboxamide
3-(4-((3-fluorobenzyl)amino)-6-carbomyl-1,3,5-triazin-2-yl)-1H-indazole-7-carboxylic acid
3-(4-(benzylamino)-6-carbomyl-1,3,5-triazin-2-yl)-1H-indazole-7-carboxylic acid
(3-(((4-(7-carbamoyl-1H-indazol-3-yl)-6-carbomyl-1,3,5-triazin-2-yl)amino)methyl)phenyl)boronic acid
3-(4-((3-fluorobenzyl)amino)-6-carbomyl-1,3,5-triazin-2-yl)-1H-indazole-7-carboxamide
3-(4-(benzylamino)-6-carbomyl-1,3,5-triazin-2-yl)-1H-indazole-7-carboxamide
(3-(((4-carbomyl-6-(1H-indazol-3-yl)-1,3,5-triazin-2-yl)amino)methyl)phenyl)boronic acid
4-((3-fluorobenzylamino)-6-(1H-indazol-3-yl)-1,3,5-triazine, pyridine,
pyrimidine-2-carboxamide
4-(benzylamino)-6-(1H-indazol-3-yl)-1,3,5-triazine, pyridine, pyrimidine-2-carboxamide
(3-(((4-(7-sulfinamoyl-1H-indazol-3-yl)-6-carbomyl-1,3,5-triazin-2-yl)amino)methyl)phenyl)boronic acid
3-(4-((3-fluorobenzyl)amino)-6-carbomyl-1,3,5-triazin-2-yl)-1H-indazole-7-sulfonamide
3-(4-(benzylamino)-6-carbomyl-1,3,5-triazin-2-yl)-1H-indazole-7-sulfonamide
(3-(4-(3-fluorobenzylamino)-6-ethyl-1,3,5-triazin-2-yl)-1H-indazol-7-yl)boronic acid
(3-(4-(benzylamino)-6-ethyl-1,3,5-triazin-2-yl)-1H-indazol-7-yl)boronic acid
(3-(((4-(7-(aminomethyl)-1H-indazol-3-yl)-6-ethyl-1,3,5-triazin-2-yl)amino)methyl)phenyl)boronic acid
4-(7-(aminomethyl)-1H-indazol-3-yl)-N-(3-fluorobenzyl)-6-ethyl-1,3,5-triazin-2-amine
4-(7-(aminomethyl)-1H-indazol-3-yl)-N-benzyl-6-ethyl-1,3,5-triazin-2-amine
(3-(((4-(7-(methoxymethyl)-1H-indazol-3-yl)-6-ethyl-1,3,5-triazin-2-yl)amino)methyl)phenyl)boronic acid
N-(3-fluorobenzyl)-4-methyl-6-((7-(methoxymethyl)-1H-indazol-3-yl)-1,3,5-triazin-2-amine
N-benzyl-4-ethyl-6-((7-(methoxymethyl)-1H-indazol-3-yl)-1,3,5-triazin-2-amine
3-(4-((3-fluorobenzyl)amino)-6-ethyl-1,3,5-triazin-2-yl)-1H-indazole-7-carboxylic acid
3-(4-(benzylamino)-6-ethyl-1,3,5-triazin-2-yl)-1H-indazole-7-carboxylic acid
(3-(((4-(7-carbamoyl-1H-indazol-3-yl)-6-ethyl-1,3,5-triazin-2-yl)amino)methyl)phenyl)boronic acid
3-(4-((3-fluorobenzyl)amino)-6-ethyl-1,3,5-triazin-2-yl)-1H-indazole-7-carboxamide
3-(4-(benzylamino)-6-ethyl-1,3,5-triazin-2-yl)-1H-indazole-7-carboxamide
(3-(((4-ethyl-6-(1H-indazol-3-yl)-1,3,5-triazin-2-yl)amino)methyl)phenyl)boronic acid
N-(3-fluorobenzyl)-4-methyl-6-(1H-indazol-3-yl)-1,3,5-triazin-2-amine
N-benzyl-4-ethyl-6-(1H-indazol-3-yl)-1,3,5-triazin-2-amine
(3-(((4-(7-sulfinamoyl-1H-indazol-3-yl)-6-ethyl-1,3,5-triazin-2-yl)amino)methyl)phenyl)boronic acid
3-(4-((3-fluorobenzyl)amino)-6-ethyl-1,3,5-triazin-2-yl)-1H-indazole-7-sulfonamide
3-(4-(benzylamino)-6-ethyl-1,3,5-triazin-2-yl)-1H-indazole-7-sulfonamide
(3-(4-(3-fluorobenzylamino)-1,3,5-triazin-2-yl)-1H-indazol-7-yl)boronic acid
(3-(4-(benzylamino)-1,3,5-triazin-2-yl)-1H-indazol-7-yl)boronic acid
(3-(((4-(7-(aminomethyl)-1H-indazol-3-yl)-6-1,3,5-triazin-2-yl)amino)methyl)phenyl)boronic acid
4-(7-(aminomethyl)-1H-indazol-3-yl)-N-(3-fluorobenzyl)-1,3,5-triazin-2-amine
4-(7-(aminomethyl)-1H-indazol-3-yl)-N-benzyl-1,3,5-triazin-2-amine
(3-(((4-(7-(methoxymethyl)-1H-indazol-3-yl)-6-1,3,5-triazin-2-yl)amino)methyl)phenyl)boronic acid
N-(3-fluorobenzyl)-4-methyl-6-((7-(methoxymethyl)-1H-indazol-3-yl)-1,3,5-triazin-2-amine
N-benzyl-4-((7-(methoxymethyl)-1H-indazol-3-yl)-1,3,5-triazin-2-amine
3-(4-((3-fluorobenzyl)amino)-1,3,5-triazin-2-yl)-1H-indazole-7-carboxylic acid
3-(4-(benzylamino)-1,3,5-triazin-2-yl)-1H-indazole-7-carboxylic acid
(3-(((4-(7-carbamoyl-1H-indazol-3-yl)-1,3,5-triazin-2-yl)amino)methyl)phenyl)boronic acid
3-(4-((3-fluorobenzyl)amino)-1,3,5-triazin-2-yl)-1H-indazole-7-carboxamide
3-(4-(benzylamino)-1,3,5-triazin-2-yl)-1H-indazole-7-carboxamide
(3-(((4-(1H-indazol-3-yl)-1,3,5-triazin-2-yl)amino)methyl)phenyl)boronic acid
N-(3-fluorobenzyl)-4-methyl-6-(1H-indazol-3-yl)-1,3,5-triazin-2-amine
N-benzyl-4-(1H-indazol-3-yl)-1,3,5-triazin-2-amine
(3-(((4-(7-sulfinamoyl-1H-indazol-3-yl)-1,3,5-triazin-2-yl)amino)methyl)phenyl)boronic acid
3-(4-((3-fluorobenzyl)amino)-1,3,5-triazin-2-yl)-1H-indazole-7-sulfonamide
3-(4-(benzylamino)-1,3,5-triazin-2-yl)-1H-indazole-7-sulfonamide
(3-(4-(3-fluorobenzylamino)-6-methyl-1,3,5-triazin-2-yl)-1H-indazol-7-yl)boronic acid
(3-(4-(benzylamino)-6-methyl-1,3,5-triazin-2-yl)-1H-indazol-7-yl)boronic acid
(3-(((4-(7-(aminomethyl)-1H-indazol-3-yl)-6-methyl-1,3,5-triazin-2-yl)amino)methyl)phenyl)boronic acid
4-(7-(aminomethyl)-1H-indazol-3-yl)-N-(3-fluorobenzyl)-6-methyl-1,3,5-triazin-2-amine
4-(7-(aminomethyl)-1H-indazol-3-yl)-N-benzyl-6-methyl-1,3,5-triazin-2-amine
(3-(((4-(7-(methoxymethyl)-1H-indazol-3-yl)-6-methyl-1,3,5-triazin-2-yl)amino)methyl)phenyl)boronic acid
N-(3-fluorobenzyl)-4-methyl-6-((7-(methoxymethyl)-1H-indazol-3-yl)-1,3,5-triazin-2-amine
N-benzyl-4-methyl-6-((7-(methoxymethyl)-1H-indazol-3-yl)-1,3,5-triazin-2-amine
3-(4-((3-fluorobenzyl)amino)-6-methyl-1,3,5-triazin-2-yl)-1H-indazole-7-carboxylic acid
3-(4-(benzylamino)-6-methyl-1,3,5-triazin-2-yl)-1H-indazole-7-carboxylic acid
(3-(((4-(7-carbamoyl-1H-indazol-3-yl)-6-methyl-1,3,5-triazin-2-yl)amino)methyl)phenyl)boronic acid
3-(4-((3-fluorobenzyl)amino)-6-methyl-1,3,5-triazin-2-yl)-1H-indazole-7-carboxamide
3-(4-(benzylamino)-6-methyl-1,3,5-triazin-2-yl)-1H-indazole-7-carboxamide
(3-(((4-methyl-6-(1H-indazol-3-yl)-1,3,5-triazin-2-yl)amino)methyl)phenyl)boronic acid
N-(3-fluorobenzyl)-4-methyl-6-(1H-indazol-3-yl)-1,3,5-triazin-2-amine
N-benzyl-4-methyl-6-(1H-indazol-3-yl)-1,3,5-triazin-2-amine
(3-(((4-(7-sulfinamoyl-1H-indazol-3-yl)-6-methyl-1,3,5-triazin-2-yl)amino)methyl)phenyl)boronic acid
3-(4-((3-fluorobenzyl)amino)-6-methyl-1,3,5-triazin-2-yl)-1H-indazole-7-sulfonamide
3-(4-(benzylamino)-6-methyl-1,3,5-triazin-2-yl)-1H-indazole-7-sulfonamide
(3-(4-(3-fluorobenzylamino)-6-(dimethylamino)-1,3,5-triazin-2-yl)-1H-indazol-7-yl)boronic acid
(3-(4-(benzylamino)-6-(dimethylamino)-1,3,5-triazin-2-yl)-1H-indazol-7-yl)boronic acid
(3-(((4-(7-(aminomethyl)-1H-indazol-3-yl)-6-(dimethylamino)-1,3,5-triazin-2-yl)amino)methyl)phenyl)boronic acid
6-(7-(aminomethyl)-1H-indazol-3-yl)-N2-(3-fluorobenzyl)-N4,N4-dimethyl-1,3,5-triazine, pyridine, pyrimidine-2,4-diamine
6-(7-(aminomethyl)-1H-indazol-3-yl)-N2-benzyl-N4,N4-dimethyl-1,3,5-triazine, pyridine, pyrimidine-2,4-diamine
(3-(((4-(7-(methoxymethyl)-1H-indazol-3-yl)-6-(dimethylamino)-1,3,5-triazin-2-yl)amino)methyl)phenyl)boronic acid
N2-(3-fluorobenzyl)-N4,N4-dimethyl-6-((7-(methoxymethyl)-1H-indazol-3-yl)-1,3,5-triazine, pyridine, pyrimidine-2,4-diamine
N2-benzyl-N4,N4-dimethyl-6-((7-(methoxymethyl)-1H-indazol-3-yl)-1,3,5-triazine, pyridine, pyrimidine-2,4-diamine
3-(4-((3-fluorobenzyl)amino)-6-(dimehtylamino)-1,3,5-triazin-2-yl)-1H-indazole-7-carboxylic acid
3-(4-(benzylamino)-6-(dimehtylamino)-1,3,5-triazin-2-yl)-1H-indazole-7-carboxylic acid
(3-(((4-(7-carbamoyl-1H-indazol-3-yl)-6-(dimethylamino)-1,3,5-triazin-2-yl)amino)methyl)phenyl)boronic acid
3-(4-((3-fluorobenzyl)amino)-6-(dimehtylamino)-1,3,5-triazin-2-yl)-1H-indazole-7-carboxamide
3-(4-(benzylamino)-6-(dimehtylamino)-1,3,5-triazin-2-yl)-1H-indazole-7-carboxamide
(3-(((4-(dimethylamino)-6-(1H-indazol-3-yl)-1,3,5-triazin-2-yl)amino)methyl)phenyl)boronic acid
N2-(3-fluorobenzyl)-N4,N4-dimethyl-6-(1H-indazol-3-yl)-1,3,5-triazine, pyridine, pyrimidine-2,4-diamine
N2-benzyl-N4,N4-dimethyl-6-(1H-indazol-3-yl)-1,3,5-triazine, pyridine, pyrimidine-2,4-diamine
(3-(((4-(7-sulfinamoyl-1H-indazol-3-yl)-6-(dimethylamino)-1,3,5-triazin-2-yl)amino)methyl)phenyl)boronic acid
3-(4-((3-fluorobenzyl)amino)-6-(dimehtylamino)-1,3,5-triazin-2-yl)-1H-indazole-7-sulfonamide
3-(4-(benzylamino)-6-(dimehtylamino)-1,3,5-triazin-2-yl)-1H-indazole-7-sulfonamide
(3-(4-(3-fluorobenzylamino)-6-methoxy-1,3,5-triazin-2-yl)-1H-indazol-7-yl)boronic acid
(3-(4-(benzylamino)-6-methoxy-1,3,5-triazin-2-yl)-1H-indazol-7-yl)boronic acid
(3-(((4-(7-(aminomethyl)-1H-indazol-3-yl)-6-methoxy-1,3,5-triazin-2-yl)amino)methyl)phenyl)boronic acid
4-(7-(aminomethyl)-1H-indazol-3-yl)-N-(3-fluorobenzyl)-6-methoxy-1,3,5-triazin-2-amine
4-(7-(aminomethyl)-1H-indazol-3-yl)-N-benzyl-6-methoxy-1,3,5-triazin-2-amine
(3-(((4-(7-(methoxymethyl)-1H-indazol-3-yl)-6-methoxy-1,3,5-triazin-2-yl)amino)methyl)phenyl)boronic acid
N-(3-fluorobenzyl)-4-methyl-6-((7-(methoxymethyl)-1H-indazol-3-yl)-1,3,5-triazin-2-amine
N-benzyl-4-methoxy-6-((7-(methoxymethyl)-1H-indazol-3-yl)-1,3,5-triazin-2-amine
3-(4-((3-fluorobenzyl)amino)-6-methoxy-1,3,5-triazin-2-yl)-1H-indazole-7-carboxylic acid
3-(4-(benzylamino)-6-methoxy-1,3,5-triazin-2-yl)-1H-indazole-7-carboxylic acid
(3-(((4-(7-carbamoyl-1H-indazol-3-yl)-6-methoxy-1,3,5-triazin-2-yl)amino)methyl)phenyl)boronic acid
3-(4-((3-fluorobenzyl)amino)-6-methoxy-1,3,5-triazin-2-yl)-1H-indazole-7-carboxamide
3-(4-(benzylamino)-6-methoxy-1,3,5-triazin-2-yl)-1H-indazole-7-carboxamide
(3-(((4-methoxy-6-(1H-indazol-3-yl)-1,3,5-triazin-2-yl)amino)methyl)phenyl)boronic acid
N-(3-fluorobenzyl)-4-methyl-6-(1H-indazol-3-yl)-1,3,5-triazin-2-amine
N-benzyl-4-methoxy-6-(1H-indazol-3-yl)-1,3,5-triazin-2-amine
(3-(((4-(7-sulfinamoyl-1H-indazol-3-yl)-6-methoxy-1,3,5-triazin-2-yl)amino)methyl)phenyl)boronic acid
3-(4-((3-fluorobenzyl)amino)-6-methoxy-1,3,5-triazin-2-yl)-1H-indazole-7-sulfonamide
3-(4-(benzylamino)-6-methoxy-1,3,5-triazin-2-yl)-1H-indazole-7-sulfonamide

### 2H-isoindole as P2

1-(5-((3-fluorobenzyl)amino)-6-carbamoyl-1,2,4-triazin-3-yl)-2H-isoindole-4-carboxamide
1-(5-(benzylamino)-6-carbamoyl-1,2,4-triazin-3-yl)-2H-isoindole-4-carboxamide
1-(5-((3-fluorobenzyl)amino)-6-ethyl-1,2,4-triazin-3-yl)-2H-isoindole-4-carboxamide
1-(5-(benzylamino)-6-ethyl-1,2,4-triazin-3-yl)-2H-isoindole-4-carboxamide
1-(5-((3-fluorobenzyl)amino)-1,2,4-triazin-3-yl)-2H-isoindole-4-carboxamide
1-(5-(benzylamino)-1,2,4-triazin-3-yl)-2H-isoindole-4-carboxamide
1-(5-((3-fluorobenzyl)amino)-6-methyl-1,2,4-triazin-3-yl)-2H-isoindole-4-carboxamide
1-(5-(benzylamino)-6-methyl-1,2,4-triazin-3-yl)-2H-isoindole-4-carboxamide
1-(5-((3-fluorobenzyl)amino)-6-(dimethylamino)-1,2,4-triazin-3-yl)-2H-isoindole-4-carboxamide
1-(5-(benzylamino)-6-(dimethylamino)-1,2,4-triazin-3-yl)-2H-isoindole-4-carboxamide
1-(5-((3-fluorobenzyl)amino)-6-methoxy-1,2,4-triazin-3-yl)-2H-isoindole-4-carboxamide
1-(5-(benzylamino)-6-methoxy-1,2,4-triazin-3-yl)-2H-isoindole-4-carboxamide
1-(4-((3-fluorobenzyl)amino)-6-carbamoyl-1,3,5-triazin-2-yl)-2H-isoindole-4-carboxamide
1-(4-(benzylamino)-6-carbamoyl-1,3,5-triazin-2-yl)-2H-isoindole-4-carboxamide
1-(4-((3-fluorobenzyl)amino)-6-ethyl-1,3,5-triazin-2-yl)-2H-isoindole-4-carboxamide
1-(4-(benzylamino)-6-ethyl-1,3,5-triazin-2-yl)-2H-isoindole-4-carboxamide
1-(4-((3-fluorobenzyl)amino)-1,3,5-triazin-2-yl)-2H-isoindole-4-carboxamide
1-(4-(benzylamino)-1,3,5-triazin-2-yl)-2H-isoindole-4-carboxamide
1-(4-((3-fluorobenzyl)amino)-6-methyl-1,3,5-triazin-2-yl)-2H-isoindole-4-carboxamide
1-(4-(benzylamino)-6-methyl-1,3,5-triazin-2-yl)-2H-isoindole-4-carboxamide
1-(4-((3-fluorobenzyl)amino)-6-(dimethylamino)-1,3,5-triazin-2-yl)-2H-isoindole-4-carboxamide
1-(4-(benzylamino)-6-(dimethylamino)-1,3,5-triazin-2-yl)-2H-isoindole-4-carboxamide
1-(4-((3-fluorobenzyl)amino)-6-methoxy-1,3,5-triazin-2-yl)-2H-isoindole-4-carboxamide
1-(4-(benzylamino)-6-methoxy-1,3,5-triazin-2-yl)-2H-isoindole-4-carboxamide

### 2-Me-1-indole as P2

(1-(5-((3-fluorobenzyl)amino)-6-carbamoyl-1,2,4-triazin-3-yl)-2-methyl-1H-indole-4-yl)boronic acid
(1-(5-(benzylamino)-6-carbamoyl-1,2,4-triazin-3-yl)-2-methyl-1H-indole-4-yl)boronic acid
(3-(((3-(4-(aminomethyl)-2-methyl-1H-indol-1-yl)-6-carbamoyl-1,2,4-triazin-5-yl)amino)methyl)phenyl)boronic acid
3-(4-(aminomethyl)-2-methyl-1H-indol-1-yl)-5-((3-fluorobenzyl)amino)-1,2,4-triazine, pyridine, pyrimidine-6-carboxamide
3-(4-(aminomethyl)-2-methyl-1H-indol-1-yl)-5-(benzylamino)-1,2,4-triazine, pyridine, pyrimidine-6-carboxamide
(3-(((3-(4-(methoxymethyl)-2-methyl-1H-indol-1-yl)-6-carbamoyl-1,2,4-triazin-5-yl)amino)methyl)phenyl)boronic acid
3-(4-(methoxymethyl)-2-methyl-1H-indol-1-yl)-5-((3-fluorobenzyl)amino)-1,2,4-triazine, pyridine, pyrimidine-6-carboxamide
3-(4-(methoxymethyl)-2-methyl-1H-indol-1-yl)-5-(benzylamino)-1,2,4-triazine, pyridine, pyrimidine-6-carboxamide
1-(5-((3-fluorobenzyl)amino)-6-carbamoyl-1,2,4-triazin-3-yl)-2-methyl-1H-indole-4-carboxylic acid
1-(5-(benzylamino)-6-carbamoyl-1,2,4-triazin-3-yl)-2-methyl-1H-indole-4-carboxylic acid
(3-(((3-(4-carbamoyl-2-methyl-1H-indol-1-yl)-6-mcarbamoyl-1,2,4-triazin-5-yl)amino)methyl)phenyl)boronic acid
1-(5-((3-fluorobenzyl)amino)-6-carbamoyl-1,2,4-triazin-3-yl)-2-methyl-1H-indole-4-carboxamide
1-(5-(benzylamino)-6-carbamoyl-1,2,4-triazin-3-yl)-2-methyl-1H-indole-4-carboxamide
(3-(((3-(4-carbamoyl-6-fluoro-2-methyl-1H-indol-1-yl)-6-mcarbamoyl-1,2,4-triazin-5-yl)amino)methyl)phenyl)boronic acid
1-(5-((3-fluorobenzyl)amino)-6-carbamoyl-1,2,4-triazin-3-yl)-6-fluoro-2-methyl-1H-indole-4-carboxamide
1-(5-(benzylamino)-6-carbamoyl-1,2,4-triazin-3-yl)-6-fluoro-2-methyl-1H-indole-4-carboxamide
(3-(((6-carbamoyl-3-(2-methyl-1H-indol-1-yl)-1,2,4-triazin-5-yl)amino)methyl)phenyl)boronic acid
5-((3-fluorobenzyl)amino)-3-(2-methyl-1H-indol-1-yl)-1,2,4-triazine, pyridine, pyrimidine-6-carboxamide
5-(benzylamino)-3-(2-methyl-1H-indol-1-yl)-1,2,4-triazine, pyridine, pyrimidine-6-carboxamide
(3-(((6-carbamoyl-3-(2-methyl-4-sulfinamoyl-1H-indol-1-yl)-1,2,4-triazin-5-yl)amino)methyl)phenyl)boronic acid
1-(5-((3-fluorobenzyl)amino)-6-carbamoyl-1,2,4-triazin-3-yl)-2-methyl-1H-indole-4-sulfonamide
1-(5-(benzylamino)-6-carbamoyl-1,2,4-triazin-3-yl)-2-methyl-1H-indole-4-sulfonamide
(1-(5-((3-fluorobenzyl)amino)-6-ethyl-1,2,4-triazin-3-yl)-2-methyl-1H-indole-4-yl)boronic acid
(1-(5-(benzylamino)-6-ethyl-1,2,4-triazin-3-yl)-2-methyl-1H-indole-4-yl)boronic acid
(3-(((3-(4-(aminomethyl)-2-methyl-1H-indol-1-yl)-6-ethyl-1,2,4-triazin-5-yl)amino)methyl)phenyl)boronic acid
3-(4-(aminomethyl)-2-methyl-1H-indol-1-yl)-N-(3-fluorobenzyl)-6-ethyl-1,2,4-triazin-5-amine
3-(4-(aminomethyl)-2-methyl-1H-indol-1-yl)-N-benzyl-6-ethyl-1,2,4-triazin-5-amine
(3-(((3-(4-(methoxymethyl)-2-methyl-1H-indol-1-yl)-6-ethyl-1,2,4-triazin-5-yl)amino)methyl)phenyl)boronic acid
3-(4-(methoxymethyl)-2-methyl-1H-indol-1-yl)-N-(3-fluorobenzyl)-6-ethyl-1,2,4-triazin-5-amine
3-(4-(methoxymethyl)-2-methyl-1H-indol-1-yl)-N-benzyl-6-ethyl-1,2,4-triazin-5-amine
1-(5-((3-fluorobenzyl)amino)-6-ethyl-1,2,4-triazin-3-yl)-2-methyl-1H-indole-4-carboxylic acid
1-(5-(benzylamino)-6-ethyl-1,2,4-triazin-3-yl)-2-methyl-1H-indole-4-carboxylic acid
(3-(((3-(4-carbamoyl-2-methyl-1H-indol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-2-methyl-1H-indole-4-carboxamide
1-(5-(benzylamino)-6-ethyl-1,2,4-triazin-3-yl)-2-methyl-1H-indole-4-carboxamide
(3-(((3-(4-carbamoyl-6-fluoro-2-methyl-1H-indol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-6-fluoro-2-methyl-1H-indole-4-carboxamide
1-(5-(benzylamino)-6-ethyl-1,2,4-triazin-3-yl)-6-fluoro-2-methyl-1H-indole-4-carboxamide
(3-(((6-ethyl-3-(2-methyl-1H-indol-1-yl)-1,2,4-triazin-5-yl)amino)methyl)phenyl)boronic acid
N-(3-fluorobenzyl)-6-ethyl-3-(2-methyl-1H-indol-1-yl)-1,2,4-triazin-5-amine
N-benzyl-6-ethyl-3-(2-methyl-1H-indol-1-yl)-1,2,4-triazin-5-amine
(3-(((6-ethyl-3-(2-methyl-4-sulfinamoyl-1H-indol-1-yl)-1,2,4-triazin-5-yl)amino)methyl)phenyl)boronic acid
1-(5-((3-fluorobenzyl)amino)-6-ethyl-1,2,4-triazin-3-yl)-2-methyl-1H-indole-4-sulfonamide
1-(5-(benzylamino)-6-ethyl-1,2,4-triazin-3-yl)-2-methyl-1H-indole-4-sulfonamide
(1-(5-((3-fluorobenzyl)amino)-1,2,4-triazin-3-yl)-2-methyl-1H-indole-4-yl)boronic acid
(1-(5-(benzylamino)-1,2,4-triazin-3-yl)-2-methyl-1H-indole-4-yl)boronic acid
(3-(((3-(4-(aminomethyl)-2-methyl-1H-indol-1-yl)-6-1,2,4-triazin-5-yl)amino)methyl)phenyl)boronic acid
3-(4-(aminomethyl)-2-methyl-1H-indol-1-yl)-N-(3-fluorobenzyl)-1,2,4-triazin-5-amine
3-(4-(aminomethyl)-2-methyl-1H-indol-1-yl)-N-benzyl-1,2,4-triazin-5-amine
(3-(((3-(4-(methoxymethyl)-2-methyl-1H-indol-1-yl)-6-1,2,4-triazin-5-yl)amino)methyl)phenyl)boronic acid
3-(4-(methoxymethyl)-2-methyl-1H-indol-1-yl)-N-(3-fluorobenzyl)-1,2,4-triazin-5-amine
3-(4-(methoxymethyl)-2-methyl-1H-indol-1-yl)-N-benzyl-1,2,4-triazin-5-amine
1-(5-((3-fluorobenzyl)amino)-1,2,4-triazin-3-yl)-2-methyl-1H-indole-4-carboxylic acid
1-(5-(benzylamino)-1,2,4-triazin-3-yl)-2-methyl-1H-indole-4-carboxylic acid
(3-(((3-(4-carbamoyl-2-methyl-1H-indol-1-yl)-1,2,4-triazin-5-yl)amino)methyl)phenyl)boronic acid
1-(5-((3-fluorobenzyl)amino)-1,2,4-triazin-3-yl)-2-methyl-1H-indole-4-carboxamide
1-(5-(benzylamino)-1,2,4-triazin-3-yl)-2-methyl-1H-indole-4-carboxamide
(3-(((3-(4-carbamoyl-6-fluoro-2-methyl-1H-indol-1-yl)-1,2,4-triazin-5-yl)amino)methyl)phenyl)boronic acid
1-(5-((3-fluorobenzyl)amino)-1,2,4-triazin-3-yl)-6-fluoro-2-methyl-1H-indole-4-carboxamide
1-(5-(benzylamino)-1,2,4-triazin-3-yl)-6-fluoro-2-methyl-1H-indole-4-carboxamide
(3-(((3-(2-methyl-1H-indol-1-yl)-1,2,4-triazin-5-yl)amino)methyl)phenyl)boronic acid
N-(3-fluorobenzyl)-3-(2-methyl-1H-indol-1-yl)-1,2,4-triazin-5-amine
N-benzyl-3-(2-methyl-1H-indol-1-yl)-1,2,4-triazin-5-amine
(3-(((3-(2-methyl-4-sulfinamoyl-1H-indol-1-yl)-1,2,4-triazin-5-yl)amino)methyl)phenyl)boronic acid
1-(5-((3-fluorobenzyl)amino)-1,2,4-triazin-3-yl)-2-methyl-1H-indole-4-sulfonamide
1-(5-(benzylamino)-1,2,4-triazin-3-yl)-2-methyl-1H-indole-4-sulfonamide
(1-(5-((3-fluorobenzyl)amino)-6-methyl-1,2,4-triazin-3-yl)-2-methyl-1H-indole-4-yl)boronic acid
(1-(5-(benzylamino)-6-methyl-1,2,4-triazin-3-yl)-2-methyl-1H-indole-4-yl)boronic acid
(3-(((3-(4-(aminomethyl)-2-methyl-1H-indol-1-yl)-6-methyl-1,2,4-triazin-5-yl)amino)methyl)phenyl)boronic acid
3-(4-(aminomethyl)-2-methyl-1H-indol-1-yl)-N-(3-fluorobenzyl)-6-methyl-1,2,4-triazin-5-amine
3-(4-(aminomethyl)-2-methyl-1H-indol-1-yl)-N-benzyl-6-methyl-1,2,4-triazin-5-amine
(3-(((3-(4-(methoxymethyl)-2-methyl-1H-indol-1-yl)-6-methyl-1,2,4-triazin-5-yl)amino)methyl)phenyl)boronic acid
3-(4-(methoxymethyl)-2-methyl-1H-indol-1-yl)-N-(3-fluorobenzyl)-6-methyl-1,2,4-triazin-5-amine
3-(4-(methoxymethyl)-2-methyl-1H-indol-1-yl)-N-benzyl-6-methyl-1,2,4-triazin-5-amine
1-(5-((3-fluorobenzyl)amino)-6-methyl-1,2,4-triazin-3-yl)-2-methyl-TH-indole-4-carboxylic acid
1-(5-(benzylamino)-6-methyl-1,2,4-triazin-3-yl)-2-methyl-1H-indole-4-carboxylic acid
(3-(((3-(4-carbamoyl-2-methyl-1H-indol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-2-methyl-TH-indole-4-carboxamide
1-(5-(benzylamino)-6-methyl-1,2,4-triazin-3-yl)-2-methyl-1H-indole-4-carboxamide
(3-(((3-(4-carbamoyl-6-fluoro-2-methyl-1H-indol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-6-fluoro-2-methyl-1H-indole-4-carboxamide
1-(5-(benzylamino)-6-methyl-1,2,4-triazin-3-yl)-6-fluoro-2-methyl-1H-indole-4-carboxamide
(3-(((6-methyl-3-(2-methyl-1H-indol-1-yl)-1,2,4-triazin-5-yl)amino)methyl)phenyl)boronic acid
N-(3-fluorobenzyl)-6-methyl-3-(2-methyl-1H-indol-1-yl)-1,2,4-triazin-5-amine
N-benzyl-6-methyl-3-(2-methyl-1H-indol-1-yl)-1,2,4-triazin-5-amine
(3-(((6-methyl-3-(2-methyl-4-sulfinamoyl-1H-indol-1-yl)-1,2,4-triazin-5-yl)amino)methyl)phenyl)boronic acid
1-(5-((3-fluorobenzyl)amino)-6-methyl-1,2,4-triazin-3-yl)-2-methyl-TH-indole-4-sulfonamide
1-(5-(benzylamino)-6-methyl-1,2,4-triazin-3-yl)-2-methyl-1H-indole-4-sulfonamide
(1-(5-((3-fluorobenzyl)amino)-6-(dimethylamino)-1,2,4-triazin-3-yl)-2-methyl-1H-indole-4-yl)boronic acid
(1-(5-(benzylamino)-6-(dimethylamino)-1,2,4-triazin-3-yl)-2-methyl-1H-indole-4-yl)boronic acid
(3-(((3-(4-(aminomethyl)-2-methyl-1H-indol-1-yl)-6-(dimethylamino)-1,2,4-triazin-5-yl)amino)methyl)phenyl)boronic acid
3-(4-(aminomethyl)-2-methyl-1H-indol-1-yl)-N5-(3-fluorobenzyl)-N6,N6-dimethyl-1,2,4-triazine, pyridine, pyrimidine-5,6-diamine
3-(4-(aminomethyl)-2-methyl-1H-indol-1-yl)-N5-benzyl-N6,N6-dimethyl-1,2,4-triazine, pyridine, pyrimidine-5,6-diamine
(3-(((3-(4-(methoxymethyl)-2-methyl-1H-indol-1-yl)-6-(dimethylamino)-1,2,4-triazin-5-yl)amino)methyl)phenyl)boronic acid
3-(4-(methoxymethyl)-2-methyl-1H-indol-1-yl)-N5-(3-fluorobenzyl)-N6,N6-dimethyl-1,2,4-triazine, pyridine, pyrimidine-5,6-diamine
3-(4-(methoxymethyl)-2-methyl-1H-indol-1-yl)-N5-benzyl-N6,N6-dimethyl-1,2,4-triazine, pyridine, pyrimidine-5,6-diamine
1-(5-((3-fluorobenzyl)amino)-6-(dimethylamino)-1,2,4-triazin-3-yl)-2-methyl-1H-indole-4-carboxylic acid
1-(5-(benzylamino)-6-(dimethylamino)-1,2,4-triazin-3-yl)-2-methyl-1H-indole-4-carboxylic acid
(3-(((3-(4-carbamoyl-2-methyl-1H-indol-1-yl)-6-(dimethylamino)-1,2,4-triazin-5-yl)amino)methyl)phenyl)boronic acid
1-(5-((3-fluorobenzyl)amino)-6-(dimethylamino)-1,2,4-triazin-3-yl)-2-methyl-1H-indole-4-carboxamide
1-(5-(benzylamino)-6-(dimethylamino)-1,2,4-triazin-3-yl)-2-methyl-1H-indole-4-carboxamide
(3-(((3-(4-carbamoyl-6-fluoro-2-methyl-1H-indol-1-yl)-6-(dimethylamino)-1,2,4-triazin-5-yl)amino)methyl)phenyl)boronic acid
1-(5-((3-fluorobenzyl)amino)-6-(dimethylamino)-1,2,4-triazin-3-yl)-6-fluoro-2-methyl-1H-indole-4-carboxamide
1-(5-(benzylamino)-6-(dimethylamino)-1,2,4-triazin-3-yl)-6-fluoro-2-methyl-1H-indole-4-carboxamide
(3-(((6-(dimethylamino)-3-(2-methyl-1H-indol-1-yl)-1,2,4-triazin-5-yl)amino)methyl)phenyl)boronic acid
N5-(3-fluorobenzyl)-N6,N6-dimethyl-3-(2-methyl-1H-indol-1-yl)-1,2,4-triazine, pyridine, pyrimidine-5,6-diamine
N5-benzyl-N6,N6-dimethyl-3-(2-methyl-1H-indol-1-yl)-1,2,4-triazine, pyridine, pyrimidine-5,6-diamine
(3-(((6-(dimethylamino)-3-(2-methyl-4-sulfinamoyl-1H-indol-1-yl)-1,2,4-triazin-5-yl)amino)methyl)phenyl)boronic acid
1-(5-((3-fluorobenzyl)amino)-6-(dimethylamino)-1,2,4-triazin-3-yl)-2-methyl-1H-indole-4-sulfonamide
1-(5-(benzylamino)-6-(dimethylamino)-1,2,4-triazin-3-yl)-2-methyl-1H-indole-4-sulfonamide
(1-(5-((3-fluorobenzyl)amino)-6-methoxy-1,2,4-triazin-3-yl)-2-methyl-1H-indole-4-yl)boronic acid
(1-(5-(benzylamino)-6-methoxy-1,2,4-triazin-3-yl)-2-methyl-1H-indole-4-yl)boronic acid
(3-(((3-(4-(aminomethyl)-2-methyl-1H-indol-1-yl)-6-methoxy-1,2,4-triazin-5-yl)amino)methyl)phenyl)boronic acid
3-(4-(aminomethyl)-2-methyl-1H-indol-1-yl)-N-(3-fluorobenzyl)-6-methoxy-1,2,4-triazin-5-amine
3-(4-(aminomethyl)-2-methyl-1H-indol-1-yl)-N-benzyl-6-methoxy-1,2,4-triazin-5-amine
(3-(((3-(4-(methoxymethyl)-2-methyl-1H-indol-1-yl)-6-methoxy-1,2,4-triazin-5-yl)amino)methyl)phenyl)boronic acid
3-(4-(methoxymethyl)-2-methyl-1H-indol-1-yl)-N-(3-fluorobenzyl)-6-methoxy-1,2,4-triazin-5-amine
3-(4-(methoxymethyl)-2-methyl-1H-indol-1-yl)-N-benzyl-6-methoxy-1,2,4-triazin-5-amine
1-(5-((3-fluorobenzyl)amino)-6-methoxy-1,2,4-triazin-3-yl)-2-methyl-1H-indole-4-carboxylic acid
1-(5-(benzylamino)-6-methoxy-1,2,4-triazin-3-yl)-2-methyl-1H-indole-4-carboxylic acid
(3-(((3-(4-carbamoyl-2-methyl-1H-indol-1-yl)-6-methoxy-1,2,4-triazin-5-yl)amino)methyl)phenyl)boronic acid
1-(5-((3-fluorobenzyl)amino)-6-methoxy-1,2,4-triazin-3-yl)-2-methyl-1H-indole-4-carboxamide
1-(5-(benzylamino)-6-methoxy-1,2,4-triazin-3-yl)-2-methyl-1H-indole-4-carboxamide
(3-(((3-(4-carbamoyl-6-fluoro-2-methyl-1H-indol-1-yl)-6-methoxy-1,2,4-triazin-5-yl)amino)methyl)phenyl)boronic acid
1-(5-((3-fluorobenzyl)amino)-6-methoxy-1,2,4-triazin-3-yl)-6-fluoro-2-methyl-1H-indole-4-carboxamide
1-(5-(benzylamino)-6-methoxy-1,2,4-triazin-3-yl)-6-fluoro-2-methyl-1H-indole-4-carboxamide
(3-(((6-methoxy-3-(2-methyl-1H-indol-1-yl)-1,2,4-triazin-5-yl)amino)methyl)phenyl)boronic acid
N-(3-fluorobenzyl)-6-methoxy-3-(2-methyl-1H-indol-1-yl)-1,2,4-triazin-5-amine
N-benzyl-6-methoxy-3-(2-methyl-1H-indol-1-yl)-1,2,4-triazin-5-amine
(3-(((6-methoxy-3-(2-methyl-4-sulfinamoyl-1H-indol-1-yl)-1,2,4-triazin-5-yl)amino)methyl)phenyl)boronic acid
1-(5-((3-fluorobenzyl)amino)-6-methoxy-1,2,4-triazin-3-yl)-2-methyl-H-indole-4-sulfonamide
1-(5-(benzylamino)-6-methoxy-1,2,4-triazin-3-yl)-2-methyl-1H-indole-4-sulfonamide
(1-(4-((3-fluorobenzyl)amino)-6-carbamoyl-1,3,5-triazin-2-yl)-2-methyl-1H-indol-4-yl)boronic acid
(1-(4-(benzylamino)-6-carbamoyl-1,3,5-triazin-2-yl)-2-methyl-1H-indol-4-yl)boronic acid
(3-(((4-(4-(aminomethyl)-2-methyl-1H-indol-1-yl)-6-carbamoyl-1,3,5-triazin-2-yl)amino)methyl)phenyl)boronic acid
4-(4-(aminomethyl)-2-methyl-1H-indol-1-yl)-6-((3-fluorobenzyl)amino)-1,3,5-triazine, pyridine, pyrimidine-2-carboxamide
4-(4-(aminomethyl)-2-methyl-1H-indol-1-yl)-6-(benzylamino)-1,3,5-triazine, pyridine, pyrimidine-2-carboxamide
(3-(((4-(4-(methoxymethy)-2-methyl-1H-indol-1-yl)-6-carbamoyl-1,3,5-triazin-2-yl)amino)methyl)phenyl)boronic acid
4-(4-(methoxymethyl)-2-methyl-1H-indol-1-yl)-6-((3-fluorobenzyl)amino)-1,3,5-triazine, pyridine, pyrimidine-2-carboxamide
4-(4-(methoxymethyl)-2-methyl-1H-indol-1-yl)-6-(benzylamino)-1,3,5-triazine, pyridine, pyrimidine-2-carboxamide
1-(4-((3-fluorobenzyl)amino)-6-carbamoyl-1,3,5-triazin-2-yl)-2-methyl-1H-indole-4-carboxylic acid
1-(4-(benzylamino)-6-carbamoyl-1,3,5-triazin-2-yl)-2-methyl-1H-indole-4-carboxylic acid
(3-(((4-(4-carbamoyl-2-methyl-1H-indol-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methyl-1H-indole-4-carboxamide
1-(4-(benzylamino)-6-carbamoyl-1,3,5-triazin-2-yl)-2-methyl-1H-indole-4-carboxamide
3-(4-(benzylamino)-6-carbamoyl-1,3,5-triazin-2-yl)imidazo[1,5-a]pyridine-8-carboxamide
(3-(((4-(4-carbamoyl-6-fluoro-2-methyl-1H-indol-1-yl)-6-carbamoyl-1,3,5-triazin-2-yl)amino)methyl)phenyl)boronic acid
1-(4-((3-flurorobenzyl)amino)-6-carbamoyl-1,3,5-triazin-2-yl)-6-fluoro-2-methyl-1H-indole-4-carboxamide
1-(4-(benzylamino)-6-carbamoyl-1,3,5-triazin-2-yl)-6-fluoro-2-methyl-1H-indole-4-carboxamide
(3-(((4-(2-methyl-1H-indol-1-yl)-6-carbamoyl-1,3,5-triazin-2-yl)amino)methyl)phenyl)boronic acid
4-((3-fluorobenzyl)amino)-6-(2-methyl-1H-indol-1-yl)-1,3,5-triazine, pyridine, pyrimidine-2-carboxamide
4-(benzylamino)-6-(2-methyl-1H-indol-1-yl)-1,3,5-triazine, pyridine, pyrimidine-2-carboxamide
(3-(((4-(4-sulfamoyll-2-methyl-1H-indol-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methyl-1H-indole-4-sulfonamide
1-(4-(benzylamino)-6-carbamoyl-1,3,5-triazin-2-yl)-2-methyl-1H-indole-4-sulfonamide
(1-(4-((3-fluorobenzyl)amino)-6-ethyl-1,3,5-triazin-2-yl)-2-methyl-1H-indol-4-yl)boronic acid
(1-(4-(benzylamino)-6-ethyl-1,3,5-triazin-2-yl)-2-methyl-1H-indol-4-yl)boronic acid
(3-(((4-(4-(aminomethyl)-2-methyl-1H-indol-1-yl)-6-ethyl-1,3,5-triazin-2-yl)amino)methyl)phenyl)boronic acid
4-(4-(aminomethyl)-2-methyl-1H-indol-1-yl)-N-(3-flurorobenzyl)-6-ethyl-1,3,5-triazin-2-amine
4-(4-(aminomethyl)-2-methyl-1H-indol-1-yl)-N-benzyl-6-ethyl-1,3,5-triazin-2-amine
(3-(((4-(4-(methoxymethy)-2-methyl-1H-indol-1-yl)-6-ethyl-1,3,5-triazin-2-yl)amino)methyl)phenyl)boronic acid
4-(4-(methoxymethyl)-2-methyl-1H-indol-1-yl)-N-(3-flurorobenzyl)-6-ethyl-1,3,5-triazin-2-amine
4-(4-(methoxymethyl)-2-methyl-1H-indol-1-yl)-N-benzyl-6-ethyl-1,3,5-triazin-2-amine
1-(4-((3-fluorobenzyl)amino)-6-ethyl-1,3,5-triazin-2-yl)-2-methyl-1H-indole-4-carboxylic acid
1-(4-(benzylamino)-6-ethyl-1,3,5-triazin-2-yl)-2-methyl-1H-indole-4-carboxylic acid
3-(4-(benzylamino)-6-ethyl-1,3,5-triazin-2-yl)imidazo[1,5-a]pyridine-8-carboxamide
(3-(((4-(4-carbamoyl-2-methyl-1H-indol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-1H-indole-4-carboxamide
1-(4-(benzylamino)-6-ethyl-1,3,5-triazin-2-yl)-2-methyl-1H-indole-4-carboxamide
(3-(((4-(4-carbamoyl-6-fluoro-2-methyl-1H-indol-1-yl)-6-ethyl-1,3,5-triazin-2-yl)amino)methyl)phenyl)boronic acid
1-(4-((3-flurorobenzyl)amino)-6-ethyl-1,3,5-triazin-2-yl)-6-fluoro-2-methyl-1H-indole-4-carboxamide
1-(4-(benzylamino)-6-ethyl-1,3,5-triazin-2-yl)-6-fluoro-2-methyl-1H-indole-4-carboxamide
(3-(((4-(2-methyl-1H-indol-1-yl)-6-ethyl-1,3,5-triazin-2-yl)amino)methyl)phenyl)boronic acid
N-(3-flurorobenzyl)-4-ethyl-6-(2-methyl-1H-indol-1-yl)-1,3,5-triazin-2-amine
N-benzyl-4-ethyl-6-(2-methyl-1H-indol-1-yl)-1,3,5-triazin-2-amine
(3-(((4-(4-sulfamoyll-2-methyl-1H-indol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-1H-indole-4-sulfonamide
1-(4-(benzylamino)-6-ethyl-1,3,5-triazin-2-yl)-2-methyl-1H-indole-4-sulfonamide
(1-(4-((3-fluorobenzyl)amino)-1,3,5-triazin-2-yl)-2-methyl-1H-indol-4-yl)boronic acid
(1-(4-(benzylamino)-1,3,5-triazin-2-yl)-2-methyl-1H-indol-4-yl)boronic acid
(3-(((4-(4-(aminomethyl)-2-methyl-1H-indol-1-yl)-1,3,5-triazin-2-yl)amino)methyl)phenyl)boronic acid
4-(4-(aminomethyl)-2-methyl-1H-indol-1-yl)-N-(3-flurorobenzyl)-1,3,5-triazin-2-amine
4-(4-(aminomethyl)-2-methyl-1H-indol-1-yl)-N-benzyl-1,3,5-triazin-2-amine
(3-(((4-(4-(methoxymethy)-2-methyl-1H-indol-1-yl)-1,3,5-triazin-2-yl)amino)methyl)phenyl)boronic acid
4-(4-(methoxymethyl)-2-methyl-1H-indol-1-yl)-N-(3-flurorobenzyl)-1,3,5-triazin-2-amine
4-(4-(methoxymethyl)-2-methyl-1H-indol-1-yl)-N-benzyl-1,3,5-triazin-2-amine
1-(4-((3-fluorobenzyl)amino)-1,3,5-triazin-2-yl)-2-methyl-1H-indole-4-carboxylic acid
1-(4-(benzylamino)-1,3,5-triazin-2-yl)-2-methyl-1H-indole-4-carboxylic acid
(3-(((4-(4-carbamoyl-6-fluoro-2-methyl-1H-indol-1-yl)-1,3,5-triazin-2-yl)amino)methyl)phenyl)boronic acid
1-(4-((3-fluorobenzyl)amino)-1,3,5-triazin-2-yl)-2-methyl-1H-indole-4-carboxamide
1-(4-(benzylamino)-1,3,5-triazin-2-yl)-2-methyl-1H-indole-4-carboxamide
3-(4-(benzylamino)-1,3,5-triazin-2-yl)imidazo[1,5-a]pyridine-8-carboxamide
(3-(((4-(4-carbamoyl-6-fluoro-2-methyl-1H-indol-1-yl)-1,3,5-triazin-2-yl)amino)methyl)phenyl)boronic acid
1-(4-((3-flurorobenzyl)amino)-1,3,5-triazin-2-yl)-6-fluoro-2-methyl-1H-indole-4-carboxamide
1-(4-(benzylamino)-1,3,5-triazin-2-yl)-6-fluoro-2-methyl-1H-indole-4-carboxamide
(3-(((4-(6-fluoro-2-methyl-1H-indol-1-yl)-1,3,5-triazin-2-yl)amino)methyl)phenyl)boronic acid
N-(3-flurorobenzyl)-4-(2-methyl-1H-indol-1-yl)-1,3,5-triazin-2-amine
N-benzyl-4-(2-methyl-1H-indol-1-yl)-1,3,5-triazin-2-amine
(3-(((4-(4-sulfamoyll-6-fluoro-2-methyl-1H-indol-1-yl)-1,3,5-triazin-2-yl)amino)methyl)phenyl)boronic acid
1-(4-((3-fluorobenzyl)amino)-1,3,5-triazin-2-yl)-2-methyl-1H-indole-4-sulfonamide
1-(4-(benzylamino)-1,3,5-triazin-2-yl)-2-methyl-1H-indole-4-sulfonamide
(1-(4-((3-fluorobenzyl)amino)-6-methyl-1,3,5-triazin-2-yl)-2-methyl-1H-indol-4-yl)boronic acid
(1-(4-(benzylamino)-6-methyl-1,3,5-triazin-2-yl)-2-methyl-1H-indol-4-yl)boronic acid
(3-(((4-(4-(aminomethyl)-2-methyl-1H-indol-1-yl)-6-methyl-1,3,5-triazin-2-yl)amino)methyl)phenyl)boronic acid
4-(4-(aminomethyl)-2-methyl-1H-indol-1-yl)-N-(3-flurorobenzyl)-6-methyl-1,3,5-triazin-2-amine
4-(4-(aminomethyl)-2-methyl-1H-indol-1-yl)-N-benzyl-6-methyl-1,3,5-triazin-2-amine
(3-(((4-(4-(methoxymethy)-2-methyl-1H-indol-1-yl)-6-methyl-1,3,5-triazin-2-yl)amino)methyl)phenyl)boronic acid
4-(4-(methoxymethyl)-2-methyl-1H-indol-1-yl)-N-(3-flurorobenzyl)-6-methyl-1,3,5-triazin-2-amine
4-(4-(methoxymethyl)-2-methyl-1H-indol-1-yl)-N-benzyl-6-methyl-1,3,5-triazin-2-amine
1-(4-((3-fluorobenzyl)amino)-6-methyl-1,3,5-triazin-2-yl)-2-methyl-1H-indole-4-carboxylic acid
1-(4-(benzylamino)-6-methyl-1,3,5-triazin-2-yl)-2-methyl-1H-indole-4-carboxylic acid
3-(4-(benzylamino)-6-methyl-1,3,5-triazin-2-yl)imidazo[1,5-a]pyridine-8-carboxamide
(3-(((4-(4-carbamoyl-2-methyl-1H-indol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-1H-indole-4-carboxamide
1-(4-(benzylamino)-6-methyl-1,3,5-triazin-2-yl)-2-methyl-1H-indole-4-carboxamide
(3-(((4-(4-carbamoyl-6-fluoro-2-methyl-1H-indol-1-yl)-6-methyl-1,3,5-triazin-2-yl)amino)methyl)phenyl)boronic acid
1-(4-((3-flurorobenzyl)amino)-6-methyl-1,3,5-triazin-2-yl)-6-fluoro-2-methyl-1H-indole-4-carboxamide
1-(4-(benzylamino)-6-methyl-1,3,5-triazin-2-yl)-6-fluoro-2-methyl-1H-indole-4-carboxamide
(3-(((4-(2-methyl-1H-indol-1-yl)-6-methyl-1,3,5-triazin-2-yl)amino)methyl)phenyl)boronic acid
N-(3-flurorobenzyl)-4-methyl-6-(2-methyl-1H-indol-1-yl)-1,3,5-triazin-2-amine
N-benzyl-4-methyl-6-(2-methyl-1H-indol-1-yl)-1,3,5-triazin-2-amine
(3-(((4-(4-sulfamoyll-2-methyl-1H-indol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-1H-indole-4-sulfonamide
1-(4-(benzylamino)-6-methyl-1,3,5-triazin-2-yl)-2-methyl-1H-indole-4-sulfonamide
(1-(4-((3-fluorobenzyl)amino)-6-(dimethylamino)-1,3,5-triazin-2-yl)-2-methyl-1H-indol-4-yl)boronic acid
(1-(4-(benzylamino)-6-(dimethylamino)-1,3,5-triazin-2-yl)-2-methyl-1H-indol-4-yl)boronic acid
(3-(((4-(4-(aminomethyl)-2-methyl-1H-indol-1-yl)-6-(dimethylamino)-1,3,5-triazin-2-yl)amino)methyl)phenyl)boronic acid
6-(4-(aminomethyl)-2-methyl-1H-indol-1-yl)-N2-(3-fluorobenzyl)-N4,N4-dimethyl-1,3,5-triazine, pyridine, pyrimidine-2,4-diamine
6-(4-(aminomethyl)-2-methyl-1H-indol-1-yl)-N2-benzyl-N4,N4-dimethyl-1,3,5-triazine, pyridine, pyrimidine-2,4-diamine
(3-(((4-(4-(methoxymethy)-2-methyl-1H-indol-1-yl)-6-(dimethylamino)-1,3,5-triazin-2-yl)amino)methyl)phenyl)boronic acid
N2-(3-fluorobenzyl)-6-(4-(methoxymethyl)-2-methyl-1H-indol-1-yl)-N4,N4-dimethyl-1,3,5-triazine, pyridine, pyrimidine-2,4-diamine
N2-benzyl-6-(4-(methoxymethyl)-2-methyl-1H-indol-1-yl)-N4,N4-dimethyl-1,3,5-triazine, pyridine, pyrimidine-2,4-diamine
1-(4-((3-fluorobenzyl)amino)-6-(dimethylamino)-1,3,5-triazin-2-yl)-2-methyl-1H-indole-4-carboxylic acid
1-(4-(benzylamino)-6-(dimethylamino)-1,3,5-triazin-2-yl)-2-methyl-1H-indole-4-carboxylic acid
(3-(((4-(4-carbamoyl-2-methyl-1H-indol-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methyl-1H-indole-4-carboxamide
1-(4-(benzylamino)-6-(dimethylamino)-1,3,5-triazin-2-yl)-2-methyl-1H-indole-4-carboxamide
3-(4-(benzylamino)-6-(dimethylamino)-1,3,5-triazin-2-yl)imidazo[1,5-a]pyridine-8-carboxamide
(3-(((4-(4-carbamoyl-6-fluoro-2-methyl-1H-indol-1-yl)-6-(dimethylamino)-1,3,5-triazin-2-yl)amino)methyl)phenyl)boronic acid
1-(4-((3-flurorobenzyl)amino)-6-(dimethylamino)-1,3,5-triazin-2-yl)-6-fluoro-2-methyl-1H-indole-4-carboxamide
1-(4-(benzylamino)-6-(dimethylamino)-1,3,5-triazin-2-yl)-6-fluoro-2-methyl-1H-indole-4-carboxamide
(3-(((4-(2-methyl-1H-indol-1-yl)-6-(dimethylamino)-1,3,5-triazin-2-yl)amino)methyl)phenyl)boronic acid
N2-(3-fluorobenzyl)-N4,N4-dimethyl-6-(2-methyl-1H-indol-1-yl)-1,3,5-triazine, pyridine, pyrimidine-2,4-diamine
N2-benzyl-N4,N4-dimethyl-6-(2-methyl-1H-indol-1-yl)-1,3,5-triazine, pyridine, pyrimidine-2,4-diamine
(3-(((4-(4-sulfamoyll-2-methyl-1H-indol-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methyl-1H-indole-4-sulfonamide
1-(4-(benzylamino)-6-(dimethylamino)-1,3,5-triazin-2-yl)-2-methyl-1H-indole-4-sulfonamide
(1-(4-((3-fluorobenzyl)amino)-6-methoxy-1,3,5-triazin-2-yl)-2-methyl-1H-indol-4-yl)boronic acid
(1-(4-(benzylamino)-6-methoxy-1,3,5-triazin-2-yl)-2-methyl-1H-indol-4-yl)boronic acid
(3-(((4-(4-(aminomethyl)-2-methyl-1H-indol-1-yl)-6-methoxy-1,3,5-triazin-2-yl)amino)methyl)phenyl)boronic acid
4-(4-(aminomethyl)-2-methyl-1H-indol-1-yl)-N-(3-flurorobenzyl)-6-methoxy-1,3,5-triazin-2-amine
4-(4-(aminomethyl)-2-methyl-1H-indol-1-yl)-N-benzyl-6-methoxy-1,3,5-triazin-2-amine
(3-(((4-(4-(methoxymethy)-2-methyl-1H-indol-1-yl)-6-methoxy-1,3,5-triazin-2-yl)amino)methyl)phenyl)boronic acid
4-(4-(methoxymethyl)-2-methyl-1H-indol-1-yl)-N-(3-flurorobenzyl)-6-methoxy-1,3,5-triazin-2-amine
4-(4-(methoxymethyl)-2-methyl-1H-indol-1-yl)-N-benzyl-6-methoxy-1,3,5-triazin-2-amine
1-(4-((3-fluorobenzyl)amino)-6-methoxy-1,3,5-triazin-2-yl)-2-methyl-1H-indole-4-carboxylic acid
1-(4-(benzylamino)-6-methoxy-1,3,5-triazin-2-yl)-2-methyl-1H-indole-4-carboxylic acid
(3-(((4-(4-carbamoyl-2-methyl-1H-indol-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methyl-1H-indole-4-carboxamide
1-(4-(benzylamino)-6-methoxy-1,3,5-triazin-2-yl)-2-methyl-1H-indole-4-carboxamide
3-(4-(benzylamino)-6-methoxy-1,3,5-triazin-2-yl)imidazo[1,5-a]pyridine-8-carboxamide
(3-(((4-(4-carbamoyl-6-fluoro-2-methyl-1H-indol-1-yl)-6-methoxy-1,3,5-triazin-2-yl)amino)methyl)phenyl)boronic acid
1-(4-((3-flurorobenzyl)amino)-6-methoxy-1,3,5-triazin-2-yl)-6-fluoro-2-methyl-1H-indole-4-carboxamide
1-(4-(benzylamino)-6-methoxy-1,3,5-triazin-2-yl)-6-fluoro-2-methyl-1H-indole-4-carboxamide
(3-(((4-(2-methyl-1H-indol-1-yl)-6-methoxy-1,3,5-triazin-2-yl)amino)methyl)phenyl)boronic acid
N-(3-flurorobenzyl)-4-methoxy-6-(2-methyl-1H-indol-1-yl)-1,3,5-triazin-2-amine
N-benzyl-4-methoxy-6-(2-methyl-1H-indol-1-yl)-1,3,5-triazin-2-amine
(3-(((4-(4-sulfamoyll-2-methyl-1H-indol-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methyl-1H-indole-4-sulfonamide
1-(4-(benzylamino)-6-methoxy-1,3,5-triazin-2-yl)-2-methyl-1H-indole-4-sulfonamide
(3-(5-((3-fluorobenzyl)amino)-6-carbamoyl-1,2,4-triazin-3-yl)-2-methyl-1H-indole-4-yl)boronic acid
(3-(5-(benzylamino)-6-carbamoyl-1,2,4-triazin-3-yl)-2-methyl-1H-indole-4-yl)boronic acid
(3-(((3-(4-(aminomethyl)-2-methyll-1H-indol-3-yl)-6-carbamoyl-1,2,4-triazin-5-yl)amino)methyl)phenyl)boronic acid
3-(7-(aminomethyl)-2-methyl-1H-indol-3-yl)-5-((3-fluorobenzyl)amino)-1,2,4-triazine, pyridine, pyrimidine-6-carboxamide
3-(7-(aminomethyl)-2-methyl-1H-indol-3-yl)-5-(benzylamino)-1,2,4-triazine, pyridine, pyrimidine-6-carboxamide
(3-(((3-(4-(methoxymethyl)-2-methyll-1H-indol-3-yl)-6-carbamoyl-1,2,4-triazin-5-yl)amino)methyl)phenyl)boronic acid
3-(7-(methoxymethyl)-2-methyl-1H-indol-3-yl)-5-((3-fluorobenzyl)amino)-1,2,4-triazine, pyridine, pyrimidine-6-carboxamide
3-(7-(methoxymethyl)-2-methyl-1H-indol-3-yl)-5-(benzylamino)-1,2,4-triazine, pyridine, pyrimidine-6-carboxamide
3-(5-((3-fluorobenzyl)amino)-6-carbamoyl-1,2,4-triazin-3-yl)-2-methyl-1H-indole-7-carboxylic acid
3-(5-(benzylamino)-6-carbamoyl-1,2,4-triazin-3-yl)-2-methyl-1H-indole-7-carboxylic acid
(3-(((3-(4-carbamoyl-2-methyll-1H-indol-3-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-2-methyl-1H-indole-7-carboxamide
3-(5-(benzylamino)-6-carbamoyl-1,2,4-triazin-3-yl)-2-methyl-1H-indole-7-carboxamide
(3-(((6-carbamoyl-3-(2-methyll-1H-indol-3-yl)-1,2,4-triazin-5-yl)amino)methyl)phenyl)boronic acid
5-((3-fluorobenzyl)amino)-3-(2-methyl-1H-indol-3-yl)-1,2,4-triazine, pyridine, pyrimidine-6-carboxamide
5-(benzylamino)-3-(2-methyl-1H-indol-3-yl)-1,2,4-triazine, pyridine, pyrimidine-6-carboxamide
(3-(((6-carbamoyl-3-(2-methyl-4-sulfinamoyll-1H-indol-3-yl)-1,2,4-triazin-5-yl)amino)methyl)phenyl)boronic acid
3-(5-((3-fluorobenzyl)amino)-6-carbamoyl-1,2,4-triazin-3-yl)-2-methyl-1H-indole-7-sulfonamide
3-(5-(benzylamino)-6-carbamoyl-1,2,4-triazin-3-yl)-2-methyl-1H-indole-7-sulfonamide
(3-(5-((3-fluorobenzyl)amino)-6-ethyl-1,2,4-triazin-3-yl)-2-methyl-1H-indole-4-yl)boronic acid
(3-(5-(benzylamino)-6-ethyl-1,2,4-triazin-3-yl)-2-methyl-1H-indole-4-yl)boronic acid
(3-(((3-(4-(aminomethyl)-2-methyll-1H-indol-3-yl)-6-ethyl-1,2,4-triazin-5-yl)amino)methyl)phenyl)boronic acid
3-(7-(aminomethyl)-2-methyl-1H-indol-3-yl)-N-(3-fluorobenzyl)-6-ethyl-1,2,4-triazin-5-amine
3-(7-(aminomethyl)-2-methyl-1H-indol-3-yl)-N-benzyl-6-ethyl-1,2,4-triazin-5-amine
(3-(((3-(4-(methoxymethyl)-2-methyll-1H-indol-3-yl)-6-ethyl-1,2,4-triazin-5-yl)amino)methyl)phenyl)boronic acid
3-(7-(methoxymethyl)-2-methyl-1H-indol-3-yl)-N-(3-fluorobenzyl)-6-ethyl-1,2,4-triazin-5-amine
3-(7-(methoxymethyl)-2-methyl-1H-indol-3-yl)-N-benzyl-6-ethyl-1,2,4-triazin-5-amine
3-(5-((3-fluorobenzyl)amino)-6-ethyl)-1,2,4-triazin-3-yl)-2-methyl-1H-indole-7-carboxylic acid
3-(5-(benzylamino)-6-ethyl)-1,2,4-triazin-3-yl)-2-methyl-1H-indole-7-carboxylic acid
(3-(((3-(4-carbamoyl-2-methyll-1H-indol-3-yl)-6-ethyl-1,2,4-triazin-5-yl)amino)methyl)phenyl)boronic acid
3-(5-((3-fluorobenzyl)amino)-6-ethyl-1,2,4-triazin-3-yl)-2-methyl-1H-indole-7-carboxamide
3-(5-(benzylamino)-6-ethyl-1,2,4-triazin-3-yl)-2-methyl-1H-indole-7-carboxamide
(3-(((6-ethyl-3-(2-methyll-1H-indol-3-yl)-1,2,4-triazin-5-yl)amino)methyl)phenyl)boronic acid
N-(3-fluorobenzyl)-6-ethyl-3-(2-methyl-1H-indol-3-yl)-1,2,4-triazin-5-amine
N-benzyl-6-ethyl-3-(2-methyl-1H-indol-3-yl)-1,2,4-triazin-5-amine
(3-(((6-ethyl-3-(2-methyl-4-sulfinamoyll-1H-indol-3-yl)-1,2,4-triazin-5-yl)amino)methyl)phenyl)boronic acid
3-(5-((3-fluorobenzyl)amino)-6-ethyl)-1,2,4-triazin-3-yl)-2-methyl-1H-indole-7-sulfonamide
3-(5-(benzylamino)-6-ethyl)-1,2,4-triazin-3-yl)-2-methyl-1H-indole-7-sulfonamide
(3-(5-((3-fluorobenzyl)amino)-1,2,4-triazin-3-yl)-2-methyl-1H-indole-4-yl)boronic acid
(3-(5-(benzylamino)-1,2,4-triazin-3-yl)-2-methyl-1H-indole-4-yl)boronic acid
(3-(((3-(4-(aminomethyl)-2-methyll-1H-indol-3-yl)-6-1,2,4-triazin-5-yl)amino)methyl)phenyl)boronic acid
3-(7-(aminomethyl)-2-methyl-1H-indol-3-yl)-N-(3-fluorobenzyl)-1,2,4-triazin-5-amine
3-(7-(aminomethyl)-2-methyl-1H-indol-3-yl)-N-benzyl-1,2,4-triazin-5-amine
(3-(((3-(4-(methoxymethyl)-2-methyll-1H-indol-3-yl)-6-1,2,4-triazin-5-yl)amino)methyl)phenyl)boronic acid
3-(7-(methoxymethyl)-2-methyl-1H-indol-3-yl)-N-(3-fluorobenzyl)-1,2,4-triazin-5-amine
3-(7-(methoxymethyl)-2-methyl-1H-indol-3-yl)-N-benzyl-1,2,4-triazin-5-amine
3-(5-((3-fluorobenzyl)amino)-1,2,4-triazin-3-yl)-2-methyl-1H-indole-7-carboxylic acid
3-(5-(benzylamino)-1,2,4-triazin-3-yl)-2-methyl-1H-indole-7-carboxylic acid
(3-(((3-(4-carbamoyl-2-methyll-1H-indol-3-yl)-1,2,4-triazin-5-yl)amino)methyl)phenyl)boronic acid
3-(5-((3-fluorobenzyl)amino)-1,2,4-triazin-3-yl)-2-methyl-1H-indole-7-carboxamide
3-(5-(benzylamino)-1,2,4-triazin-3-yl)-2-methyl-1H-indole-7-carboxamide
(3-(((3-(2-methyll-1H-indol-3-yl)-1,2,4-triazin-5-yl)amino)methyl)phenyl)boronic acid
N-(3-fluorobenzyl)-3-(2-methyl-1H-indol-3-yl)-1,2,4-triazin-5-amine
N-benzyl-3-(2-methyl-1H-indol-3-yl)-1,2,4-triazin-5-amine
(3-(((3-(2-methyl-4-sulfinamoyll-1H-indol-3-yl)-1,2,4-triazin-5-yl)amino)methyl)phenyl)boronic acid
3-(5-((3-fluorobenzyl)amino)-1,2,4-triazin-3-yl)-2-methyl-1H-indole-7-sulfonamide
3-(5-(benzylamino)-1,2,4-triazin-3-yl)-2-methyl-1H-indole-7-sulfonamide
(3-(5-((3-fluorobenzyl)amino)-6-methyl-1,2,4-triazin-3-yl)-2-methyl-1H-indol-7-yl)boronic acid
(3-(5-(benzylamino)-6-methyl-1,2,4-triazin-3-yl)-2-methyl-1H-indol-7-yl)boronic acid
(3-(((3-(4-(aminomethyl)-2-methyll-1H-indol-3-yl)-6-methyl-1,2,4-triazin-5-yl)amino)methyl)phenyl)boronic acid
3-(7-(aminomethyl)-2-methyl-1H-indol-3-yl)-N-(3-fluorobenzyl)-6-methyl-1,2,4-triazin-5-amine
3-(7-(aminomethyl)-2-methyl-1H-indol-3-yl)-N-benzyl-6-methyl-1,2,4-triazin-5-amine
(3-(((3-(4-(methoxymethyl)-2-methyll-1H-indol-3-yl)-6-methyl-1,2,4-triazin-5-yl)amino)methyl)phenyl)boronic acid
3-(7-(methoxymethyl)-2-methyl-1H-indol-3-yl)-N-(3-fluorobenzyl)-6-methyl-1,2,4-triazin-5-amine
3-(7-(methoxymethyl)-2-methyl-1H-indol-3-yl)-N-benzyl-6-methyl-1,2,4-triazin-5-amine
3-(5-((3-fluorobenzyl)amino)-6-methyl-1,2,4-triazin-3-yl)-2-methyl-1H-indole-7-carboxylic acid
3-(5-(benzylamino)-6-methyl-1,2,4-triazin-3-yl)-2-methyl-1H-indole-7-carboxylic acid
(3-(((3-(4-carbamoyl-2-methyll-1H-indol-3-yl)-6-methyl-1,2,4-triazin-5-yl)amino)methyl)phenyl)boronic acid
3-(5-((3-fluorobenzyl)amino)-6-methyl-1,2,4-triazin-3-yl)-2-methyl-1H-indole-7-carboxamide
3-(5-(benzylamino)-6-methyl-1,2,4-triazin-3-yl)-2-methyl-1H-indole-7-carboxamide
(3-(((6-methyl-3-(2-methyll-1H-indol-3-yl)-1,2,4-triazin-5-yl)amino)methyl)phenyl)boronic acid
N-(3-fluorobenzyl)-6-methyl-3-(2-methyl-1H-indol-3-yl)-1,2,4-triazin-5-amine
N-benzyl-6-methyl-3-(2-methyl-1H-indol-3-yl)-1,2,4-triazin-5-amine
(3-(((6-methyl-3-(2-methyl-4-sulfinamoyll-1H-indol-3-yl)-1,2,4-triazin-5-yl)amino)methyl)phenyl)boronic acid
3-(5-((3-fluorobenzyl)amino)-6-methyl-1,2,4-triazin-3-yl)-2-methyl-1H-indole-7-sulfonamide
3-(5-(benzylamino)-6-methyl-1,2,4-triazin-3-yl)-2-methyl-1H-indole-7-sulfonamide
(3-(5-((3-fluorobenzyl)amino)-6-(dimethylamino)-1,2,4-triazin-3-yl)-2-methyl-1H-indole-4-yl)boronic acid
(3-(5-(benzylamino)-6-(dimethylamino)-1,2,4-triazin-3-yl)-2-methyl-1H-indole-4-yl)boronic acid
(3-(((3-(4-(aminomethyl)-2-methyll-1H-indol-3-yl)-6-(dimethylamino)-1,2,4-triazin-5-yl)amino)methyl)phenyl)boronic acid
3-(7-(aminomethyl)-2-methyl-1H-indol-3-yl)-N5-(3-fluorobenzyl)-N6,N6-dimethyl-1,2,4-triazine, pyridine, pyrimidine-5,6-diamine
3-(7-(aminomethyl)-2-methyl-1H-indol-3-yl)-N5-benzyl-N6,N6-dimethyl-1,2,4-triazine, pyridine, pyrimidine-5,6-diamine
(3-(((3-(4-(methoxymethyl)-2-methyll-1H-indol-3-yl)-6-(dimethylamino)-1,2,4-triazin-5-yl)amino)methyl)phenyl)boronic acid
3-(7-(methoxymethyl)-2-methyl-1H-indol-3-yl)-N5-(3-fluorobenzyl)-N6,N6-dimethyl-1,2,4-triazine, pyridine, pyrimidine-5,6-diamine
3-(7-(methoxymethyl)-2-methyl-1H-indol-3-yl)-N5-benzyl-N6,N6-dimethyl-1,2,4-triazine, pyridine, pyrimidine-5,6-diamine
3-(5-((3-fluorobenzyl)amino)-6-(dimethylamino)-1,2,4-triazin-3-yl)-2-methyl-1H-indole-7-carboxylic acid
3-(5-(benzylamino)-6-(dimethylamino)-1,2,4-triazin-3-yl)-2-methyl-1H-indole-7-carboxylic acid
(3-(((3-(4-carbamoyl-2-methyll-1H-indol-3-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-2-methyl-1H-indole-7-carboxamide
3-(5-(benzylamino)-6-(dimethylamino)-1,2,4-triazin-3-yl)-2-methyl-1H-indole-7-carboxamide
(3-(((6-(dimethylamino)-3-(2-methyll-1H-indol-3-yl)-1,2,4-triazin-5-yl)amino)methyl)phenyl)boronic acid
N5-(3-fluorobenzyl)-N6,N6-dimethyl-3-(2-methyl-1H-indol-3-yl)-1,2,4-triazine, pyridine, pyrimidine-5,6-diamine
N5-benzyl-N6,N6-dimethyl-3-(2-methyl-1H-indol-3-yl)-1,2,4-triazine, pyridine, pyrimidine-5,6-diamine
(3-(((6-(dimethylamino)-3-(2-methyl-4-sulfinamoyll-1H-indol-3-yl)-1,2,4-triazin-5-yl)amino)methyl)phenyl)boronic acid
3-(5-((3-fluorobenzyl)amino)-6-(dimethylamino)-1,2,4-triazin-3-yl)-2-methyl-1H-indole-7-sulfonamide
3-(5-(benzylamino)-6-(dimethylamino)-1,2,4-triazin-3-yl)-2-methyl-1H-indole-7-sulfonamide
(3-(5-((3-fluorobenzyl)amino)-6-methoxy-1,2,4-triazin-3-yl)-2-methyl-1H-indole-4-yl)boronic acid
(3-(5-(benzylamino)-6-methoxy-1,2,4-triazin-3-yl)-2-methyl-1H-indole-4-yl)boronic acid
(3-(((3-(4-(aminomethyl)-2-methyll-1H-indol-3-yl)-6-methoxy-1,2,4-triazin-5-yl)amino)methyl)phenyl)boronic acid
3-(7-(aminomethyl)-2-methyl-1H-indol-3-yl)-N-(3-fluorobenzyl)-6-methoxy-1,2,4-triazin-5-amine
3-(7-(aminomethyl)-2-methyl-1H-indol-3-yl)-N-benzyl-6-methoxy-1,2,4-triazin-5-amine
(3-(((3-(4-(methoxymethyl)-2-methyll-1H-indol-3-yl)-6-methoxy-1,2,4-triazin-5-yl)amino)methyl)phenyl)boronic acid
3-(7-(methoxymethyl)-2-methyl-1H-indol-3-yl)-N-(3-fluorobenzyl)-6-methoxy-1,2,4-triazin-5-amine
3-(7-(methoxymethyl)-2-methyl-1H-indol-3-yl)-N-benzyl-6-methoxy-1,2,4-triazin-5-amine
3-(5-((3-fluorobenzyl)amino)-6-methoxy-1,2,4-triazin-3-yl)-2-methyl-1H-indole-7-carboxylic acid
3-(5-(benzylamino)-6-methoxy-1,2,4-triazin-3-yl)-2-methyl-1H-indole-7-carboxylic acid
(3-(((3-(4-carbamoyl-2-methyll-1H-indol-3-yl)-6-methoxy-1,2,4-triazin-5-yl)amino)methyl)phenyl)boronic acid
3-(5-((3-fluorobenzyl)amino)-6-methoxy-1,2,4-triazin-3-yl)-2-methyl-1H-indole-7-carboxamide
3-(5-(benzylamino)-6-methoxy-1,2,4-triazin-3-yl)-2-methyl-1H-indole-7-carboxamide
(3-(((6-methoxy-3-(2-methyll-1H-indol-3-yl)-1,2,4-triazin-5-yl)amino)methyl)phenyl)boronic acid
N-(3-fluorobenzyl)-6-methoxy-3-(2-methyl-1H-indol-3-yl)-1,2,4-triazin-5-amine
N-benzyl-6-methoxy-3-(2-methyl-1H-indol-3-yl)-1,2,4-triazin-5-amine
(3-(((6-methoxy-3-(2-methyl-4-sulfinamoyll-1H-indol-3-yl)-1,2,4-triazin-5-yl)amino)methyl)phenyl)boronic acid
3-(5-((3-fluorobenzyl)amino)-6-methoxy-1,2,4-triazin-3-yl)-2-methyl-1H-indole-7-sulfonamide
3-(5-(benzylamino)-6-methoxy-1,2,4-triazin-3-yl)-2-methyl-1H-indole-7-sulfonamide
(3-(4-(3-fluorobenzylamino)-6-carbomyl-1,3,5-triazin-2-yl)-2-methyl-1H-indol-7-yl)boronic acid
(3-(4-(benzylamino)-6-carbomyl-1,3,5-triazin-2-yl)-2-methyl-1H-indol-7-yl)boronic acid
(3-(((4-(7-(aminomethyl)-2-methyl-1H-indol-3-yl)-6-carbomyl-1,3,5-triazin-2-yl)amino)methyl)phenyl)boronic acid
4-(7-(aminomethyl)-2-methyl-1H-indol-3-yl)-6-((3-fluorobenzyl)amino)-1,3,5-triazine, pyridine, pyrimidine-2-carboxamide
4-(7-(aminomethyl)-2-methyl-1H-indol-3-yl)-6-(benzylamino)-1,3,5-triazine, pyridine, pyrimidine-2-carboxamide
(3-(((4-(7-(methoxymethyl)-2-methyl-1H-indol-3-yl)-6-carbomyl-1,3,5-triazin-2-yl)amino)methyl)phenyl)boronic acid
4-((3-fluorobenzylamino)-6-((7-(methoxymethyl)-2-methyl-1H-indol-3-yl)-1,3,5-triazine, pyridine, pyrimidine-2-carboxamide
4-(benzylamino)-6-(7-(methoxymethyl)-2-methyl-1H-indol-3-yl)-1,3,5-triazine, pyridine, pyrimidine-2-carboxamide
3-(4-((3-fluorobenzyl)amino)-6-carbomyl-1,3,5-triazin-2-yl)-2-methyl-1H-indole-7-carboxylic acid
3-(4-(benzylamino)-6-carbomyl-1,3,5-triazin-2-yl)-2-methyl-1H-indole-7-carboxylic acid
(3-(((4-(7-carbamoyl-2-methyl-1H-indol-3-yl)-6-carbomyl-1,3,5-triazin-2-yl)amino)methyl)phenyl)boronic acid
3-(4-((3-fluorobenzyl)amino)-6-carbomyl-1,3,5-triazin-2-yl)-2-methyl-1H-indole-7-carboxamide
3-(4-(benzylamino)-6-carbomyl-1,3,5-triazin-2-yl)-2-methyl-1H-indole-7-carboxamide
(3-(((4-carbomyl-6-(2-methyl-1H-indol-3-yl)-1,3,5-triazin-2-yl)amino)methyl)phenyl)boronic acid
4-((3-fluorobenzylamino)-6-(2-methyl-1H-indol-3-yl)-1,3,5-triazine, pyridine, pyrimidine-2-carboxamide
4-(benzylamino)-6-(2-methyl-1H-indol-3-yl)-1,3,5-triazine, pyridine, pyrimidine-2-carboxamide
(3-(((4-(7-sulfinamoyl-2-methyl-1H-indol-3-yl)-6-carbomyl-1,3,5-triazin-2-yl)amino)methyl)phenyl)boronic acid
3-(4-((3-fluorobenzyl)amino)-6-carbomyl-1,3,5-triazin-2-yl)-2-methyl-1H-indole-7-sulfonamide
3-(4-(benzylamino)-6-carbomyl-1,3,5-triazin-2-yl)-2-methyl-1H-indole-7-sulfonamide
(3-(4-(3-fluorobenzylamino)-6-ethyl-1,3,5-triazin-2-yl)-2-methyl-1H-indol-7-yl)boronic acid
(3-(4-(benzylamino)-6-ethyl-1,3,5-triazin-2-yl)-2-methyl-1H-indol-7-yl)boronic acid
(3-(((4-(7-(aminomethyl)-2-methyl-1H-indol-3-yl)-6-ethyl-1,3,5-triazin-2-yl)amino)methyl)phenyl)boronic acid
4-(7-(aminomethyl)-2-methyl-1H-indol-3-yl)-N-(3-fluorobenzyl)-6-ethyl-1,3,5-triazin-2-amine
4-(7-(aminomethyl)-2-methyl-1H-indol-3-yl)-N-benzyl-6-ethyl-1,3,5-triazin-2-amine
(3-(((4-(7-(methoxymethyl)-2-methyl-1,3,5-triazin-2-yl)amino)methyl)phenyl)boronic acid
N-(3-fluorobenzyl)-4-methyl-6-((7-(methoxymethyl)-2-methyl-1H-indol-3-yl)-1,3,5-triazin-2-amine
N-benzyl-4-ethyl-6-((7-(methoxymethyl)-2-methyl-1H-indol-3-yl)-1,3,5-triazin-2-amine
3-(4-((3-fluorobenzyl)amino)-6-ethyl-1,3,5-triazin-2-yl)-2-methyl-1H-indole-7-carboxylic acid
3-(4-(benzylamino)-6-ethyl-1,3,5-triazin-2-yl)-2-methyl-1H-indole-7-carboxylic acid
3-(4-((3-fluorobenzyl)amino)-6-ethyl-1,3,5-triazin-2-yl)-2-methyl-1H-indole-7-carboxamide
3-(4-(benzylamino)-6-ethyl-1,3,5-triazin-2-yl)-2-methyl-1H-indole-7-carboxamide
(3-(((4-(7-carbamoyl-2-methyl-1H-indol-3-yl)-6-ethyl-1,3,5-triazin-2-yl)amino)methyl)phenyl)boronic acid
(3-(((4-ethyl-6-(2-methyl-1H-indol-3-yl)-1,3,5-triazin-2-yl)amino)methyl)phenyl)boronic acid
N-(3-fluorobenzyl)-4-methyl-6-(2-methyl-1H-indol-3-yl)-1,3,5-triazin-2-amine
N-benzyl-4-ethyl-6-(2-methyl-1H-indol-3-yl)-1,3,5-triazin-2-amine
(3-(((4-(7-sulfinamoyl-2-methyl-1H-indol-3-yl)-6-ethyl-1,3,5-triazin-2-yl)amino)methyl)phenyl)boronic acid
3-(4-((3-fluorobenzyl)amino)-6-ethyl-1,3,5-triazin-2-yl)-2-methyl-1H-indole-7-sulfonamide
3-(4-(benzylamino)-6-ethyl-1,3,5-triazin-2-yl)-2-methyl-1H-indole-7-sulfonamide
(3-(4-(3-fluorobenzylamino)-1,3,5-triazin-2-yl)-2-methyl-1H-indol-7-yl)boronic acid
(3-(4-(benzylamino)-1,3,5-triazin-2-yl)-2-methyl-1H-indol-7-yl)boronic acid
(3-(((4-(7-(aminomethyl)-2-methyl-1H-indol-3-yl)-6-1,3,5-triazin-2-yl)amino)methyl)phenyl)boronic acid
4-(7-(aminomethyl)-2-methyl-1H-indol-3-yl)-N-(3-fluorobenzyl)-1,3,5-triazin-2-amine
4-(7-(aminomethyl)-2-methyl-1H-indol-3-yl)-N-benzyl-1,3,5-triazin-2-amine
(3-(((4-(7-(methoxymethyl)-2-methyl-1H-indol-3-yl)-6-1,3,5-triazin-2-yl)amino)methyl)phenyl)boronic acid
N-(3-fluorobenzyl)-4-methyl-6-((7-(methoxymethyl)-2-methyl-1H-indol-3-yl)-1,3,5-triazin-2-amine
N-benzyl-4-((7-(methoxymethyl)-2-methyl-1H-indol-3-yl)-1,3,5-triazin-2-amine
3-(4-((3-fluorobenzyl)amino)-1,3,5-triazin-2-yl)-2-methyl-1H-indole-7-carboxylic acid
3-(4-(benzylamino)-1,3,5-triazin-2-yl)-2-methyl-1H-indole-7-carboxylic acid
3-(4-((3-fluorobenzyl)amino)-1,3,5-triazin-2-yl)-2-methyl-1H-indole-7-carboxamide
3-(4-(benzylamino)-1,3,5-triazin-2-yl)-2-methyl-1H-indole-7-carboxamide
(3-(((4-(7-carbamoyl-2-methyl-1H-indol-3-yl)-1,3,5-triazin-2-yl)amino)methyl)phenyl)boronic acid
(3-(((4-(2-methyl-1H-indol-3-yl)-1,3,5-triazin-2-yl)amino)methyl)phenyl)boronic acid
N-(3-fluorobenzyl)-4-methyl-6-(2-methyl-1H-indol-3-yl)-1,3,5-triazin-2-amine
N-benzyl-4-(2-methyl-1H-indol-3-yl)-1,3,5-triazin-2-amine
(3-(((4-(7-sulfinamoyl-2-methyl-1H-indol-3-yl)-1,3,5-triazin-2-yl)amino)methyl)phenyl)boronic acid
3-(4-((3-fluorobenzyl)amino)-1,3,5-triazin-2-yl)-2-methyl-1H-indole-7-sulfonamide
3-(4-(benzylamino)-1,3,5-triazin-2-yl)-2-methyl-1H-indole-7-sulfonamide
(3-(4-(3-fluorobenzylamino)-6-methyl-1,3,5-triazin-2-yl)-2-methyl-1H-indol-7-yl)boronic acid
(3-(4-(benzylamino)-6-methyl-1,3,5-triazin-2-yl)-2-methyl-1H-indol-7-yl)boronic acid
(3-(((4-(7-(aminomethyl)-2-methyl-1H-indol-3-yl)-6-methyl-1,3,5-triazin-2-yl)amino)methyl)phenyl)boronic acid
4-(7-(aminomethyl)-2-methyl-1H-indol-3-yl)-N-(3-fluorobenzyl)-6-methyl-1,3,5-triazin-2-amine
4-(7-(aminomethyl)-2-methyl-1H-indol-3-yl)-N-benzyl-6-methyl-1,3,5-triazin-2-amine
(3-(((4-(7-(methoxymethyl)-2-methyl-1H-indol-3-yl)-6-methyl-1,3,5-triazin-2-yl)amino)methyl)phenyl)boronic acid
N-(3-fluorobenzyl)-4-methyl-6-((7-(methoxymethyl)-2-methyl-1H-indol-3-yl)-1,3,5-triazin-2-amine
N-benzyl-4-methyl-6-((7-(methoxymethyl)-2-methyl-1H-indol-3-yl)-1,3,5-triazin-2-amine
3-(4-((3-fluorobenzyl)amino)-6-methyl-1,3,5-triazin-2-yl)-2-methyl-1H-indole-7-carboxylic acid
3-(4-(benzylamino)-6-methyl-1,3,5-triazin-2-yl)-2-methyl-1H-indole-7-carboxylic acid
3-(4-((3-fluorobenzyl)amino)-6-methyl-1,3,5-triazin-2-yl)-2-methyl-1H-indole-7-carboxamide
3-(4-(benzylamino)-6-methyl-1,3,5-triazin-2-yl)-2-methyl-1H-indole-7-carboxamide
(3-(((4-(7-carbamoyl-2-methyl-1H-indol-3-yl)-6-methyl-1,3,5-triazin-2-yl)amino)methyl)phenyl)boronic acid
(3-(((4-methyl-6-(2-methyl-1H-indol-3-yl)-1,3,5-triazin-2-yl)amino)methyl)phenyl)boronic acid
N-(3-fluorobenzyl)-4-methyl-6-(2-methyl-1H-indol-3-yl)-1,3,5-triazin-2-amine
N-benzyl-4-methyl-6-(2-methyl-1H-indol-3-yl)-1,3,5-triazin-2-amine
(3-(((4-(7-sulfinamoyl-2-methyl-1H-indol-3-yl)-6-methyl-1,3,5-triazin-2-yl)amino)methyl)phenyl)boronic acid
3-(4-((3-fluorobenzyl)amino)-6-methyl-1,3,5-triazin-2-yl)-2-methyl-1H-indole-7-sulfonamide
3-(4-(benzylamino)-6-methyl-1,3,5-triazin-2-yl)-2-methyl-1H-indole-7-sulfonamide
(3-(4-(3-fluorobenzylamino)-6-(dimethylamino)-1,3,5-triazin-2-yl)-2-methyl-1H-indol-7-yl)boronic acid
(3-(4-(benzylamino)-6-(dimethylamino)-1,3,5-triazin-2-yl)-2-methyl-1H-indol-7-yl)boronic acid
(3-(((4-(7-(aminomethyl)-2-methyl-1H-indol-3-yl)-6-(dimethylamino)-1,3,5-triazin-2-yl)amino)methyl)phenyl)boronic acid
6-(7-(aminomethyl)-2-methyl-1H-indol-3-yl)-N2-(3-fluorobenzyl)-N4,N4-dimethyl-1,3,5-triazine, pyridine, pyrimidine-2,4-diamine
6-(7-(aminomethyl)-2-methyl-1H-indol-3-yl)-N2-benzyl-N4,N4-dimethyl-1,3,5-triazine, pyridine, pyrimidine-2,4-diamine
(3-(((4-(7-(methoxymethyl)-2-methyl-1H-indol-3-yl)-6-(dimethylamino)-1,3,5-triazin-2-yl)amino)methyl)phenyl)boronic acid
N2-(3-fluorobenzyl)-N4,N4-dimethyl-6-((7-(methoxymethyl)-2-methyl-1H-indol-3-yl)-1,3,5-triazine, pyridine, pyrimidine-2,4-diamine
N2-benzyl-N4,N4-dimethyl-6-((7-(methoxymethyl)-2-methyl-1H-indol-3-yl)-1,3,5-triazine, pyridine, pyrimidine-2,4-diamine
3-(4-((3-fluorobenzyl)amino)-6-(dimehtylamino)-1,3,5-triazin-2-yl)-2-methyl-1H-indole-7-carboxylic acid
3-(4-(benzylamino)-6-(dimehtylamino)-1,3,5-triazin-2-yl)-2-methyl-1H-indole-7-carboxylic acid
(3-(((4-(7-carbamoyl-2-methyl-1H-indol-3-yl)-6-(dimethylamino)-1,3,5-triazin-2-yl)amino)methyl)phenyl)boronic acid
3-(4-((3-fluorobenzyl)amino)-6-(dimehtylamino)-1,3,5-triazin-2-yl)-2-methyl-1H-indole-7-carboxamide
3-(4-(benzylamino)-6-(dimehtylamino)-1,3,5-triazin-2-yl)-2-methyl-1H-indole-7-carboxamide
(3-(((4-(dimethylamino)-6-(2-methyl-1H-indol-3-yl)-1,3,5-triazin-2-yl)amino)methyl)phenyl)boronic acid
N2-(3-fluorobenzyl)-N4,N4-dimethyl-6-(2-methyl-1H-indol-3-yl)-1,3,5-triazine, pyridine, pyrimidine-2,4-diamine
N2-benzyl-N4,N4-dimethyl-6-(2-methyl-1H-indol-3-yl)-1,3,5-triazine, pyridine, pyrimidine-2,4-diamine
(3-(((4-(7-sulfinamoyl-2-methyl-1H-indol-3-yl)-6-(dimethylamino)-1,3,5-triazin-2-yl)amino)methyl)phenyl)boronic acid
3-(4-((3-fluorobenzyl)amino)-6-(dimehtylamino)-1,3,5-triazin-2-yl)-2-methyl-1H-indole-7-sulfonamide
3-(4-(benzylamino)-6-(dimehtylamino)-1,3,5-triazin-2-yl)-2-methyl-1H-indole-7-sulfonamide
(3-(4-(3-fluorobenzylamino)-6-methoxy-1,3,5-triazin-2-yl)-2-methyl-1H-indol-7-yl)boronic acid
(3-(4-(benzylamino)-6-methoxy-1,3,5-triazin-2-yl)-2-methyl-1H-indol-7-yl)boronic acid
(3-(((4-(7-(aminomethyl)-2-methyl-1H-indol-3-yl)-6-methoxy-1,3,5-triazin-2-yl)amino)methyl)phenyl)boronic acid
4-(7-(aminomethyl)-2-methyl-1H-indol-3-yl)-N-(3-fluorobenzyl)-6-methoxy-1,3,5-triazin-2-amine
4-(7-(aminomethyl)-2-methyl-1H-indol-3-yl)-N-benzyl-6-methoxy-1,3,5-triazin-2-amine
(3-(((4-(7-(methoxymethyl)-2-methyl-1H-indol-3-yl)-6-methoxy-1,3,5-triazin-2-yl)amino)methyl)phenyl)boronic acid
N-(3-fluorobenzyl)-4-methyl-6-((7-(methoxymethyl)-2-methyl-1H-indol-3-yl)-1,3,5-triazin-2-amine
N-benzyl-4-methoxy-6-((7-(methoxymethyl)-2-methyl-1H-indol-3-yl)-1,3,5-triazin-2-amine
3-(4-((3-fluorobenzyl)amino)-6-methoxy-1,3,5-triazin-2-yl)-2-methyl-1H-indole-7-carboxylic acid
3-(4-(benzylamino)-6-methoxy-1,3,5-triazin-2-yl)-2-methyl-1H-indole-7-carboxylic acid
(3-(((4-(7-carbamoyl-2-methyl-1H-indol-3-yl)-6-methoxy-1,3,5-triazin-2-yl)amino)methyl)phenyl)boronic acid
3-(4-((3-fluorobenzyl)amino)-6-methoxy-1,3,5-triazin-2-yl)-2-methyl-1H-indole-7-carboxamide
3-(4-(benzylamino)-6-methoxy-1,3,5-triazin-2-yl)-2-methyl-1H-indole-7-carboxamide
(3-(((4-methoxy-6-(2-methyl-1H-indol-3-yl)-1,3,5-triazin-2-yl)amino)methyl)phenyl)boronic acid
N-(3-fluorobenzyl)-4-methyl-6-(2-methyl-1H-indol-3-yl)-1,3,5-triazin-2-amine
N-benzyl-4-methoxy-6-(2-methyl-1H-indol-3-yl)-1,3,5-triazin-2-amine
(3-(((4-(7-sulfinamoyl-2-methyl-1H-indol-3-yl)-6-methoxy-1,3,5-triazin-2-yl)amino)methyl)phenyl)boronic acid
3-(4-((3-fluorobenzyl)amino)-6-methoxy-1,3,5-triazin-2-yl)-2-methyl-1H-indole-7-sulfonamide
3-(4-(benzylamino)-6-methoxy-1,3,5-triazin-2-yl)-2-methyl-1H-indole-7-sulfonamide

### 2-Me-indoline as P2

(1-(5-(benzylamino)-6-carbamoyl-1,2,4-triazin-3-yl)-2-methyl-indoline-4-yl)boronic acid
(1-(5-((3-fluorobenzyl)amino)-6-carbamoyl-1,2,4-triazin-3-yl)-2-methyl-indoline-4-yl)boronic acid
3-(4-(aminomethyl)-2-methyl-indolin-1-yl)-5-(benzylamino)-1,2,4-triazine, pyridine, pyrimidine-6-carboxamide
3-(4-(aminomethyl)-2-methyl-indolin-1-yl)-5-((3-fluorobenzyl)amino)-1,2,4-triazine, pyridine, pyrimidine-6-carboxamide
(3-(((3-(4-(aminomethyl)-2-methyl-indolin-1-yl)-6-carbamoyl-1,2,4-triazin-5-yl)amino)methyl)phenyl)boronic acid
3-(4-(methoxymethyl)-2-methyl-indolin-1-yl)-5-(benzylamino)-1,2,4-triazine, pyridine, pyrimidine-6-carboxamide
3-(4-(methoxymethyl)-2-methyl-indolin-1-yl)-5-((3-fluorobenzyl)amino)-1,2,4-triazine, pyridine, pyrimidine-6-carboxamide
(3-(((3-(4-(methoxymethyl)-2-methyl-indolin-1-yl)-6-carbamoyl-1,2,4-triazin-5-yl)amino)methyl)phenyl)boronic acid
1-(5-(benzylamino)-6-carbamoyl-1,2,4-triazin-3-yl)-2-methyl-indoline-4-carboxylic acid
1-(5-((3-fluorobenzyl)amino)-6-carbamoyl-1,2,4-triazin-3-yl)-2-methyl-indoline-4-carboxylic acid
1-(5-(benzylamino)-6-carbamoyl-1,2,4-triazin-3-yl)-2-methyl-indoline-4-carboxamide
1-(5-((3-fluorobenzyl)amino)-6-carbamoyl-1,2,4-triazin-3-yl)-2-methyl-indoline-4-carboxamide
(3-(((3-(4-carbamoyl-2-methyl-indolin-1-yl)-6-mcarbamoyl-1,2,4-triazin-5-yl)amino)methyl)phenyl)boronic acid
1-(5-(benzylamino)-6-carbamoyl-1,2,4-triazin-3-yl)-6-fluoro-2-methyl-indoline-4-carboxamide
1-(5-((3-fluorobenzyl)amino)-6-carbamoyl-1,2,4-triazin-3-yl)-6-fluoro-2-methyl-indoline-4-carboxamide
(3-(((3-(4-carbamoyl-6-fluoro-2-methyl-indolin-1-yl)-6-mcarbamoyl-1,2,4-triazin-5-yl)amino)methyl)phenyl)boronic acid
5-(benzylamino)-3-(2-methyl-indolin-1-yl)-1,2,4-triazine, pyridine, pyrimidine-6-carboxamide
5-((3-fluorobenzyl)amino)-3-(2-methyl-indolin-1-yl)-1,2,4-triazine, pyridine, pyrimidine-6-carboxamide
(3-(((6-carbamoyl-3-(2-methyl-indolin-1-yl)-1,2,4-triazin-5-yl)amino)methyl)phenyl)boronic acid
1-(5-(benzylamino)-6-carbamoyl-1,2,4-triazin-3-yl)-2-methyl-indoline-4-sulfonamide
1-(5-((3-fluorobenzyl)amino)-6-carbamoyl-1,2,4-triazin-3-yl)-2-methyl-indoline-4-sulfonamide
(3-(((6-carbamoyl-3-(2-methyl-4-sulfinamoyl-indolin-1-yl)-1,2,4-triazin-5-yl)amino)methyl)phenyl)boronic acid
(1-(5-(benzylamino)-6-ethyl-1,2,4-triazin-3-yl)-2-methyl-indoline-4-yl)boronic acid
(1-(5-((3-fluorobenzyl)amino)-6-ethyl-1,2,4-triazin-3-yl)-2-methyl-indoline-4-yl)boronic acid
3-(4-(aminomethyl)-2-methyl-indolin-1-yl)-N-benzyl-6-ethyl-1,2,4-triazin-5-amine
3-(4-(aminomethyl)-2-methyl-indolin-1-yl)-N-(3-fluorobenzyl)-6-ethyl-1,2,4-triazin-5-amine
(3-(((3-(4-(aminomethyl)-2-methyl-indolin-1-yl)-6-ethyl-1,2,4-triazin-5-yl)amino)methyl)phenyl)boronic acid
3-(4-(methoxymethyl)-2-methyl-indolin-1-yl)-N-benzyl-6-ethyl-1,2,4-triazin-5-amine
3-(4-(methoxymethyl)-2-methyl-indolin-1-yl)-N-(3-fluorobenzyl)-6-ethyl-1,2,4-triazin-5-amine
(3-(((3-(4-(methoxymethyl)-2-methyl-indolin-1-yl)-6-ethyl-1,2,4-triazin-5-yl)amino)methyl)phenyl)boronic acid
1-(5-(benzylamino)-6-ethyl-1,2,4-triazin-3-yl)-2-methyl-indoline-4-carboxylic acid
1-(5-((3-fluorobenzyl)amino)-6-ethyl-1,2,4-triazin-3-yl)-2-methyl-indoline-4-carboxylic acid
1-(5-(benzylamino)-6-ethyl-1,2,4-triazin-3-yl)-2-methyl-indoline-4-carboxamide
1-(5-((3-fluorobenzyl)amino)-6-ethyl-1,2,4-triazin-3-yl)-2-methyl-indoline-4-carboxamide
(3-(((3-(4-carbamoyl-2-methyl-indolin-1-yl)-6-ethyl-1,2,4-triazin-5-yl)amino)methyl)phenyl)boronic acid
1-(5-(benzylamino)-6-ethyl-1,2,4-triazin-3-yl)-6-fluoro-2-methyl-indoline-4-carboxamide
1-(5-((3-fluorobenzyl)amino)-6-ethyl-1,2,4-triazin-3-yl)-6-fluoro-2-methyl-indoline-4-carboxamide
(3-(((3-(4-carbamoyl-6-fluoro-2-methyl-indolin-1-yl)-6-ethyl-1,2,4-triazin-5-yl)amino)methyl)phenyl)boronic acid
N-benzyl-6-ethyl-3-(2-methyl-indolin-1-yl)-1,2,4-triazin-5-amine
N-(3-fluorobenzyl)-6-ethyl-3-(2-methyl-indolin-1-yl)-1,2,4-triazin-5-amine
(3-(((6-ethyl-3-(2-methyl-indolin-1-yl)-1,2,4-triazin-5-yl)amino)methyl)phenyl)boronic acid
1-(5-(benzylamino)-6-ethyl-1,2,4-triazin-3-yl)-2-methyl-indoline-4-sulfonamide
1-(5-((3-fluorobenzyl)amino)-6-ethyl-1,2,4-triazin-3-yl)-2-methyl-indoline-4-sulfonamide
(3-(((6-ethyl-3-(2-methyl-4-sulfinamoyl-indolin-1-yl)-1,2,4-triazin-5-yl)amino)methyl)phenyl)boronic acid
(1-(5-(benzylamino)-1,2,4-triazin-3-yl)-2-methyl-indoline-4-yl)boronic acid
(1-(5-((3-fluorobenzyl)amino)-1,2,4-triazin-3-yl)-2-methyl-indoline-4-yl)boronic acid
3-(4-(aminomethyl)-2-methyl-indolin-1-yl)-N-benzyl-1,2,4-triazin-5-amine
3-(4-(aminomethyl)-2-methyl-indolin-1-yl)-N-(3-fluorobenzyl)-1,2,4-triazin-5-amine
(3-(((3-(4-(aminomethyl)-2-methyl-indolin-1-yl)-6-1,2,4-triazin-5-yl)amino)methyl)phenyl)boronic acid
3-(4-(methoxymethyl)-2-methyl-indolin-1-yl)-N-benzyl-1,2,4-triazin-5-amine
3-(4-(methoxymethyl)-2-methyl-indolin-1-yl)-N-(3-fluorobenzyl)-1,2,4-triazin-5-amine
(3-(((3-(4-(methoxymethyl)-2-methyl-indolin-1-yl)-6-1,2,4-triazin-5-yl)amino)methyl)phenyl)boronic acid
1-(5-(benzylamino)-1,2,4-triazin-3-yl)-2-methyl-indoline-4-carboxylic acid
1-(5-((3-fluorobenzyl)amino)-1,2,4-triazin-3-yl)-2-methyl-indoline-4-carboxylic acid
1-(5-(benzylamino)-1,2,4-triazin-3-yl)-2-methyl-indoline-4-carboxamide
1-(5-((3-fluorobenzyl)amino)-1,2,4-triazin-3-yl)-2-methyl-indoline-4-carboxamide
(3-(((3-(4-carbamoyl-2-methyl-indolin-1-yl)-1,2,4-triazin-5-yl)amino)methyl)phenyl)boronic acid
1-(5-(benzylamino)-1,2,4-triazin-3-yl)-6-fluoro-2-methyl-indoline-4-carboxamide
1-(5-((3-fluorobenzyl)amino)-1,2,4-triazin-3-yl)-6-fluoro-2-methyl-indoline-4-carboxamide
(3-(((3-(4-carbamoyl-6-fluoro-2-methyl-indolin-1-yl)-1,2,4-triazin-5-yl)amino)methyl)phenyl)boronic acid
N-benzyl-3-(2-methyl-indolin-1-yl)-1,2,4-triazin-5-amine
N-(3-fluorobenzyl)-3-(2-methyl-indolin-1-yl)-1,2,4-triazin-5-amine
(3-(((3-(2-methyl-indolin-1-yl)-1,2,4-triazin-5-yl)amino)methyl)phenyl)boronic acid
1-(5-(benzylamino)-1,2,4-triazin-3-yl)-2-methyl-indoline-4-sulfonamide
1-(5-((3-fluorobenzyl)amino)-1,2,4-triazin-3-yl)-2-methyl-indoline-4-sulfonamide
(3-(((3-(2-methyl-4-sulfinamoyl-indolin-1-yl)-1,2,4-triazin-5-yl)amino)methyl)phenyl)boronic acid
(1-(5-(benzylamino)-6-methyl-1,2,4-triazin-3-yl)-2-methyl-indoline-4-yl)boronic acid
(1-(5-((3-fluorobenzyl)amino)-6-methyl-1,2,4-triazin-3-yl)-2-methyl-indoline-4-yl)boronic acid
3-(4-(aminomethyl)-2-methyl-indolin-1-yl)-N-benzyl-6-methyl-1,2,4-triazin-5-amine
3-(4-(aminomethyl)-2-methyl-indolin-1-yl)-N-(3-fluorobenzyl)-6-methyl-1,2,4-triazin-5-amine
(3-(((3-(4-(aminomethyl)-2-methyl-indolin-1-yl)-6-methyl-1,2,4-triazin-5-yl)amino)methyl)phenyl)boronic acid
3-(4-(methoxymethyl)-2-methyl-indolin-1-yl)-N-benzyl-6-methyl-1,2,4-triazin-5-amine
3-(4-(methoxymethyl)-2-methyl-indolin-1-yl)-N-(3-fluorobenzyl)-6-methyl-1,2,4-triazin-5-amine
(3-(((3-(4-(methoxymethyl)-2-methyl-indolin-1-yl)-6-methyl-1,2,4-triazin-5-yl)amino)methyl)phenyl)boronic acid
1-(5-(benzylamino)-6-methyl-1,2,4-triazin-3-yl)-2-methyl-indoline-4-carboxylic acid
1-(5-((3-fluorobenzyl)amino)-6-methyl-1,2,4-triazin-3-yl)-2-methyl-indoline-4-carboxylic acid
1-(5-(benzylamino)-6-methyl-1,2,4-triazin-3-yl)-2-methyl-indoline-4-carboxamide
1-(5-((3-fluorobenzyl)amino)-6-methyl-1,2,4-triazin-3-yl)-2-methyl-indoline-4-carboxamide
(3-(((3-(4-carbamoyl-2-methyl-indolin-1-yl)-6-methyl-1,2,4-triazin-5-yl)amino)methyl)phenyl)boronic acid
1-(5-(benzylamino)-6-methyl-1,2,4-triazin-3-yl)-6-fluoro-2-methyl-indoline-4-carboxamide
1-(5-((3-fluorobenzyl)amino)-6-methyl-1,2,4-triazin-3-yl)-6-fluoro-2-methyl-indoline-4-carboxamide
(3-(((3-(4-carbamoyl-6-fluoro-2-methyl-indolin-1-yl)-6-methyl-1,2,4-triazin-5-yl)amino)methyl)phenyl)boronic acid
N-benzyl-6-methyl-3-(2-methyl-indolin-1-yl)-1,2,4-triazin-5-amine
N-(3-fluorobenzyl)-6-methyl-3-(2-methyl-indolin-1-yl)-1,2,4-triazin-5-amine
(3-(((6-methyl-3-(2-methyl-indolin-1-yl)-1,2,4-triazin-5-yl)amino)methyl)phenyl)boronic acid
1-(5-(benzylamino)-6-methyl-1,2,4-triazin-3-yl)-2-methyl-indoline-4-sulfonamide
1-(5-((3-fluorobenzyl)amino)-6-methyl-1,2,4-triazin-3-yl)-2-methyl-indoline-4-sulfonamide
(3-(((6-methyl-3-(2-methyl-4-sulfinamoyl-indolin-1-yl)-1,2,4-triazin-5-yl)amino)methyl)phenyl)boronic acid
(1-(5-(benzylamino)-6-(dimethylamino)-1,2,4-triazin-3-yl)-2-methyl-indoline-4-yl)boronic acid
(1-(5-((3-fluorobenzyl)amino)-6-(dimethylamino)-1,2,4-triazin-3-yl)-2-methyl-indoline-4-yl)boronic acid
3-(4-(aminomethyl)-2-methyl-indolin-1-yl)-N5-benzyl-N6,N6-dimethyl-1,2,4-triazine, pyridine, pyrimidine-5,6-diamine
3-(4-(aminomethyl)-2-methyl-indolin-1-yl)-N5-(3-fluorobenzyl)-N6,N6-dimethyl-1,2,4-triazine, pyridine, pyrimidine-5,6-diamine
(3-(((3-(4-(aminomethyl)-2-methyl-indolin-1-yl)-6-(dimethylamino)-1,2,4-triazin-5-yl)amino)methyl)phenyl)boronic acid
3-(4-(methoxymethyl)-2-methyl-indolin-1-yl)-N5-benzyl-N6,N6-dimethyl-1,2,4-triazine, pyridine, pyrimidine-5,6-diamine
3-(4-(methoxymethyl)-2-methyl-indolin-1-yl)-N5-(3-fluorobenzyl)-N6,N6-dimethyl-1,2,4-triazine, pyridine, pyrimidine-5,6-diamine
(3-(((3-(4-(methoxymethyl)-2-methyl-indolin-1-yl)-6-(dimethylamino)-1,2,4-triazin-5-yl)amino)methyl)phenyl)boronic acid
1-(5-(benzylamino)-6-(dimethylamino)-1,2,4-triazin-3-yl)-2-methyl-indoline-4-carboxylic acid
1-(5-((3-fluorobenzyl)amino)-6-(dimethylamino)-1,2,4-triazin-3-yl)-2-methyl-indoline-4-carboxylic acid
1-(5-(benzylamino)-6-(dimethylamino)-1,2,4-triazin-3-yl)-2-methyl-indoline-4-carboxamide
1-(5-((3-fluorobenzyl)amino)-6-(dimethylamino)-1,2,4-triazin-3-yl)-2-methyl-indoline-4-carboxamide
(3-(((3-(4-carbamoyl-2-methyl-indolin-1-yl)-6-(dimethylamino)-1,2,4-triazin-5-yl)amino)methyl)phenyl)boronic acid
1-(5-(benzylamino)-6-(dimethylamino)-1,2,4-triazin-3-yl)-6-fluoro-2-methyl-indoline-4-carboxamide
1-(5-((3-fluorobenzyl)amino)-6-(dimethylamino)-1,2,4-triazin-3-yl)-6-fluoro-2-methyl-indoline-4-carboxamide
(3-(((3-(4-carbamoyl-6-fluoro-2-methyl-indolin-1-yl)-6-(dimethylamino)-1,2,4-triazin-5-yl)amino)methyl)phenyl)boronic acid
N5-benzyl-N6,N6-dimethyl-3-(2-methyl-indolin-1-yl)-1,2,4-triazine, pyridine, pyrimidine-5,6-diamine
N5-(3-fluorobenzyl)-N6,N6-dimethyl-3-(2-methyl-indolin-1-yl)-1,2,4-triazine, pyridine, pyrimidine-5,6-diamine
(3-(((6-(dimethylamino)-3-(2-methyl-indolin-1-yl)-1,2,4-triazin-5-yl)amino)methyl)phenyl)boronic acid
1-(5-(benzylamino)-6-(dimethylamino)-1,2,4-triazin-3-yl)-2-methyl-indoline-4-sulfonamide
1-(5-((3-fluorobenzyl)amino)-6-(dimethylamino)-1,2,4-triazin-3-yl)-2-methyl-indoline-4-sulfonamide
(3-(((6-(dimethylamino)-3-(2-methyl-4-sulfinamoyl-indolin-1-yl)-1,2,4-triazin-5-yl)amino)methyl)phenyl)boronic acid
(1-(5-(benzylamino)-6-methoxy-1,2,4-triazin-3-yl)-2-methyl-indoline-4-yl)boronic acid
(1-(5-((3-fluorobenzyl)amino)-6-methoxy-1,2,4-triazin-3-yl)-2-methyl-indoline-4-yl)boronic acid
3-(4-(aminomethyl)-2-methyl-indolin-1-yl)-N-benzyl-6-methoxy-1,2,4-triazin-5-amine
3-(4-(aminomethyl)-2-methyl-indolin-1-yl)-N-(3-fluorobenzyl)-6-methoxy-1,2,4-triazin-5-amine
(3-(((3-(4-(aminomethyl)-2-methyl-indolin-1-yl)-6-methoxy-1,2,4-triazin-5-yl)amino)methyl)phenyl)boronic acid
3-(4-(methoxymethyl)-2-methyl-indolin-1-yl)-N-benzyl-6-methoxy-1,2,4-triazin-5-amine
3-(4-(methoxymethyl)-2-methyl-indolin-1-yl)-N-(3-fluorobenzyl)-6-methoxy-1,2,4-triazin-5-amine
(3-(((3-(4-(methoxymethyl)-2-methyl-indolin-1-yl)-6-methoxy-1,2,4-triazin-5-yl)amino)methyl)phenyl)boronic acid
1-(5-(benzylamino)-6-methoxy-1,2,4-triazin-3-yl)-2-methyl-indoline-4-carboxylic acid
1-(5-((3-fluorobenzyl)amino)-6-methoxy-1,2,4-triazin-3-yl)-2-methyl-indoline-4-carboxylic acid
1-(5-(benzylamino)-6-methoxy-1,2,4-triazin-3-yl)-2-methyl-indoline-4-carboxamide
1-(5-((3-fluorobenzyl)amino)-6-methoxy-1,2,4-triazin-3-yl)-2-methyl-indoline-4-carboxamide
(3-(((3-(4-carbamoyl-2-methyl-indolin-1-yl)-6-methoxy-1,2,4-triazin-5-yl)amino)methyl)phenyl)boronic acid
1-(5-(benzylamino)-6-methoxy-1,2,4-triazin-3-yl)-6-fluoro-2-methyl-indoline-4-carboxamide
1-(5-((3-fluorobenzyl)amino)-6-methoxy-1,2,4-triazin-3-yl)-6-fluoro-2-methyl-indoline-4-carboxamide
(3-(((3-(4-carbamoyl-6-fluoro-2-methyl-indolin-1-yl)-6-methoxy-1,2,4-triazin-5-yl)amino)methyl)phenyl)boronic acid
N-benzyl-6-methoxy-3-(2-methyl-indolin-1-yl)-1,2,4-triazin-5-amine
N-(3-fluorobenzyl)-6-methoxy-3-(2-methyl-indolin-1-yl)-1,2,4-triazin-5-amine
(3-(((6-methoxy-3-(2-methyl-indolin-1-yl)-1,2,4-triazin-5-yl)amino)methyl)phenyl)boronic acid
1-(5-(benzylamino)-6-methoxy-1,2,4-triazin-3-yl)-2-methyl-indoline-4-sulfonamide
1-(5-((3-fluorobenzyl)amino)-6-methoxy-1,2,4-triazin-3-yl)-2-methyl-indoline-4-sulfonamide
(3-(((6-methoxy-3-(2-methyl-4-sulfinamoyl-indolin-1-yl)-1,2,4-triazin-5-yl)amino)methyl)phenyl)boronic acid
(1-(4-((3-fluorobenzyl)amino)-6-carbamoyl-1,3,5-triazin-2-yl)-2-methyl-indolin-4-yl)boronic acid
(1-(4-(benzylamino)-6-carbamoyl-1,3,5-triazin-2-yl)-2-methyl-indolin-4-yl)boronic acid
(3-(((4-(4-(aminomethyl)-2-methyl-indolin-1-yl)-6-carbamoyl-1,3,5-triazin-2-yl)amino)methyl)phenyl)boronic acid
4-(4-(aminomethyl)-2-methyl-indolin-1-yl)-6-((3-fluorobenzyl)amino)-1,3,5-triazine, pyridine, pyrimidine-2-carboxamide
4-(4-(aminomethyl)-2-methyl-indolin-1-yl)-6-(benzylamino)-1,3,5-triazine, pyridine, pyrimidine-2-carboxamide
(3-(((4-(4-(methoxymethy)-2-methyl-indolin-1-yl)-6-carbamoyl-1,3,5-triazin-2-yl)amino)methyl)phenyl)boronic acid
4-(4-(methoxymethyl)-2-methyl-indolin-1-yl)-6-((3-fluorobenzyl)amino)-1,3,5-triazine, pyridine, pyrimidine-2-carboxamide
4-(4-(methoxymethyl)-2-methyl-indolin-1-yl)-6-(benzylamino)-1,3,5-triazine, pyridine, pyrimidine-2-carboxamide
1-(4-((3-fluorobenzyl)amino)-6-carbamoyl-1,3,5-triazin-2-yl)-2-methyl-indoline-4-carboxylic acid
1-(4-(benzylamino)-6-carbamoyl-1,3,5-triazin-2-yl)-2-methyl-indoline-4-carboxylic acid
(3-(((4-(4-carbamoyl-2-methyl-indolin-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methyl-indoline-4-carboxamide
1-(4-(benzylamino)-6-carbamoyl-1,3,5-triazin-2-yl)-2-methyl-indoline-4-carboxamide
(3-(((4-(4-carbamoyl-6-fluoro-2-methyl-indolin-1-yl)-6-carbamoyl-1,3,5-triazin-2-yl)amino)methyl)phenyl)boronic acid
1-(4-((3-flurorobenzyl)amino)-6-carbamoyl-1,3,5-triazin-2-yl)-6-fluoro-2-methyl-indoline-4-carboxamide
1-(4-(benzylamino)-6-carbamoyl-1,3,5-triazin-2-yl)-6-fluoro-2-methyl-indoline-4-carboxamide
(3-(((4-(2-methyl-indolin-1-yl)-6-carbamoyl-1,3,5-triazin-2-yl)amino)methyl)phenyl)boronic acid
4-((3-fluorobenzyl)amino)-6-(2-methyl-indolin-1-yl)-1,3,5-triazine, pyridine,
pyrimidine-2-carboxamide
4-(benzylamino)-6-(2-methyl-indolin-1-yl)-1,3,5-triazine, pyridine, pyrimidine-2-carboxamide
(3-(((4-(4-sulfamoyll-2-methyl-indolin-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methyl-indoline-4-sulfonamide
1-(4-(benzylamino)-6-carbamoyl-1,3,5-triazin-2-yl)-2-methyl-indoline-4-sulfonamide
(1-(4-((3-fluorobenzyl)amino)-6-ethyl-1,3,5-triazin-2-yl)-2-methyl-indolin-4-yl)boronic acid
(1-(4-(benzylamino)-6-ethyl-1,3,5-triazin-2-yl)-2-methyl-indolin-4-yl)boronic acid
(3-(((4-(4-(aminomethyl)-2-methyl-indolin-1-yl)-6-ethyl-1,3,5-triazin-2-yl)amino)methyl)phenyl)boronic acid
4-(4-(aminomethyl)-2-methyl-indolin-1-yl)-N-(3-flurorobenzyl)-6-ethyl-1,3,5-triazin-2-amine
4-(4-(aminomethyl)-2-methyl-indolin-1-yl)-N-benzyl-6-ethyl-1,3,5-triazin-2-amine
(3-(((4-(4-(methoxymethy)-2-methyl-indolin-1-yl)-6-ethyl-1,3,5-triazin-2-yl)amino)methyl)phenyl)boronic acid
4-(4-(methoxymethyl)-2-methyl-indolin-1-yl)-N-(3-flurorobenzyl)-6-ethyl-1,3,5-triazin-2-amine
4-(4-(methoxymethyl)-2-methyl-indolin-1-yl)-N-benzyl-6-ethyl-1,3,5-triazin-2-amine
1-(4-((3-fluorobenzyl)amino)-6-ethyl-1,3,5-triazin-2-yl)-2-methyl-indoline-4-carboxylic acid
1-(4-(benzylamino)-6-ethyl-1,3,5-triazin-2-yl)-2-methyl-indoline-4-carboxylic acid
(3-(((4-(4-carbamoyl-2-methyl-indolin-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-indoline-4-carboxamide
1-(4-(benzylamino)-6-ethyl-1,3,5-triazin-2-yl)-2-methyl-indoline-4-carboxamide
(3-(((4-(4-carbamoyl-6-fluoro-2-methyl-indolin-1-yl)-6-ethyl-1,3,5-triazin-2-yl)amino)methyl)phenyl)boronic acid
1-(4-((3-flurorobenzyl)amino)-6-ethyl-1,3,5-triazin-2-yl)-6-fluoro-2-methyl-indoline-4-carboxamide
1-(4-(benzylamino)-6-ethyl-1,3,5-triazin-2-yl)-6-fluoro-2-methyl-indoline-4-carboxamide
(3-(((4-(2-methyl-indolin-1-yl)-6-ethyl-1,3,5-triazin-2-yl)amino)methyl)phenyl)boronic acid
N-(3-flurorobenzyl)-4-ethyl-6-(2-methyl-indolin-1-yl)-1,3,5-triazin-2-amine
N-benzyl-4-ethyl-6-(2-methyl-indolin-1-yl)-1,3,5-triazin-2-amine
(3-(((4-(4-sulfamoyll-2-methyl-indolin-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-indoline-4-sulfonamide
1-(4-(benzylamino)-6-ethyl-1,3,5-triazin-2-yl)-2-methyl-indoline-4-sulfonamide
(1-(4-((3-fluorobenzyl)amino)-1,3,5-triazin-2-yl)-2-methyl-indolin-4-yl)boronic acid
(1-(4-(benzylamino)-1,3,5-triazin-2-yl)-2-methyl-indolin-4-yl)boronic acid
(3-(((4-(4-(aminomethyl)-2-methyl-indolin-1-yl)-1,3,5-triazin-2-yl)amino)methyl)phenyl)boronic acid
4-(4-(aminomethyl)-2-methyl-indolin-1-yl)-N-(3-flurorobenzyl)-1,3,5-triazin-2-amine
4-(4-(aminomethyl)-2-methyl-indolin-1-yl)-N-benzyl-1,3,5-triazin-2-amine
(3-(((4-(4-(methoxymethy)-2-methyl-indolin-1-yl)-1,3,5-triazin-2-yl)amino)methyl)phenyl)boronic acid
4-(4-(methoxymethyl)-2-methyl-indolin-1-yl)-N-(3-flurorobenzyl)-1,3,5-triazin-2-amine
4-(4-(methoxymethyl)-2-methyl-indolin-1-yl)-N-benzyl-1,3,5-triazin-2-amine
1-(4-((3-fluorobenzyl)amino)-1,3,5-triazin-2-yl)-2-methyl-indoline-4-carboxylic acid
1-(4-(benzylamino)-1,3,5-triazin-2-yl)-2-methyl-indoline-4-carboxylic acid
(3-(((4-(4-carbamoyl-6-fluoro-2-methyl-indolin-1-yl)-1,3,5-triazin-2-yl)amino)methyl)phenyl)boronic acid
1-(4-((3-fluorobenzyl)amino)-1,3,5-triazin-2-yl)-2-methyl-indoline-4-carboxamide
1-(4-(benzylamino)-1,3,5-triazin-2-yl)-2-methyl-indoline-4-carboxamide
(3-(((4-(4-carbamoyl-6-fluoro-2-methyl-indolin-1-yl)-1,3,5-triazin-2-yl)amino)methyl)phenyl)boronic acid
1-(4-((3-flurorobenzyl)amino)-1,3,5-triazin-2-yl)-6-fluoro-2-methyl-indoline-4-carboxamide
1-(4-(benzylamino)-1,3,5-triazin-2-yl)-6-fluoro-2-methyl-indoline-4-carboxamide
(3-(((4-(6-fluoro-2-methyl-indolin-1-yl)-1,3,5-triazin-2-yl)amino)methyl)phenyl)boronic acid
N-(3-flurorobenzyl)-4-(2-methyl-indolin-1-yl)-1,3,5-triazin-2-amine
N-benzyl-4-(2-methyl-indolin-1-yl)-1,3,5-triazin-2-amine
(3-(((4-(4-sulfamoyll-6-fluoro-2-methyl-indolin-1-yl)-1,3,5-triazin-2-yl)amino)methyl)phenyl)boronic acid
1-(4-((3-fluorobenzyl)amino)-1,3,5-triazin-2-yl)-2-methyl-indoline-4-sulfonamide
1-(4-(benzylamino)-1,3,5-triazin-2-yl)-2-methyl-indoline-4-sulfonamide
(1-(4-((3-fluorobenzyl)amino)-6-methyl-1,3,5-triazin-2-yl)-2-methyl-indolin-4-yl)boronic acid
(1-(4-(benzylamino)-6-methyl-1,3,5-triazin-2-yl)-2-methyl-indolin-4-yl)boronic acid
(3-(((4-(4-(aminomethyl)-2-methyl-indolin-1-yl)-6-methyl-1,3,5-triazin-2-yl)amino)methyl)phenyl)boronic acid
4-(4-(aminomethyl)-2-methyl-indolin-1-yl)-N-(3-flurorobenzyl)-6-methyl-1,3,5-triazin-2-amine
4-(4-(aminomethyl)-2-methyl-indolin-1-yl)-N-benzyl-6-methyl-1,3,5-triazin-2-amine
(3-(((4-(4-(methoxymethy)-2-methyl-indolin-1-yl)-6-methyl-1,3,5-triazin-2-yl)amino)methyl)phenyl)boronic acid
4-(4-(methoxymethyl)-2-methyl-indolin-1-yl)-N-(3-flurorobenzyl)-6-methyl-1,3,5-triazin-2-amine
4-(4-(methoxymethyl)-2-methyl-indolin-1-yl)-N-benzyl-6-methyl-1,3,5-triazin-2-amine
1-(4-((3-fluorobenzyl)amino)-6-methyl-1,3,5-triazin-2-yl)-2-methyl-indoline-4-carboxylic acid
1-(4-(benzylamino)-6-methyl-1,3,5-triazin-2-yl)-2-methyl-indoline-4-carboxylic acid
(3-(((4-(4-carbamoyl-2-methyl-indolin-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-indoline-4-carboxamide
1-(4-(benzylamino)-6-methyl-1,3,5-triazin-2-yl)-2-methyl-indoline-4-carboxamide
(3-(((4-(4-carbamoyl-6-fluoro-2-methyl-indolin-1-yl)-6-methyl-1,3,5-triazin-2-yl)amino)methyl)phenyl)boronic acid
1-(4-((3-flurorobenzyl)amino)-6-methyl-1,3,5-triazin-2-yl)-6-fluoro-2-methyl-indoline-4-carboxamide
1-(4-(benzylamino)-6-methyl-1,3,5-triazin-2-yl)-6-fluoro-2-methyl-indoline-4-carboxamide
(3-(((4-(2-methyl-indolin-1-yl)-6-methyl-1,3,5-triazin-2-yl)amino)methyl)phenyl)boronic acid
N-(3-flurorobenzyl)-4-methyl-6-(2-methyl-indolin-1-yl)-1,3,5-triazin-2-amine
N-benzyl-4-methyl-6-(2-methyl-indolin-1-yl)-1,3,5-triazin-2-amine
(3-(((4-(4-sulfamoyll-2-methyl-indolin-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-indoline-4-sulfonamide
1-(4-(benzylamino)-6-methyl-1,3,5-triazin-2-yl)-2-methyl-indoline-4-sulfonamide
(1-(4-((3-fluorobenzyl)amino)-6-(dimethylamino)-1,3,5-triazin-2-yl)-2-methyl-indolin-4-yl)boronic acid
(1-(4-(benzylamino)-6-(dimethylamino)-1,3,5-triazin-2-yl)-2-methyl-indolin-4-yl)boronic acid
(3-(((4-(4-(aminomethyl)-2-methyl-indolin-1-yl)-6-(dimethylamino)-1,3,5-triazin-2-yl)amino)methyl)phenyl)boronic acid
6-(4-(aminomethyl)-2-methyl-indolin-1-yl)-N2-(3-fluorobenzyl)-N4,N4-dimethyl-1,3,5-triazine, pyridine, pyrimidine-2,4-diamine
6-(4-(aminomethyl)-2-methyl-indolin-1-yl)-N2-benzyl-N4,N4-dimethyl-1,3,5-triazine, pyridine, pyrimidine-2,4-diamine
(3-(((4-(4-(methoxymethy)-2-methyl-indolin-1-yl)-6-(dimethylamino)-1,3,5-triazin-2-yl)amino)methyl)phenyl)boronic acid
N2-(3-fluorobenzyl)-6-(4-(methoxymethyl)-2-methyl-indolin-1-yl)-N4,N4-dimethyl-1,3,5-triazine, pyridine, pyrimidine-2,4-diamine
N2-benzyl-6-(4-(methoxymethyl)-2-methyl-indolin-1-yl)-N4,N4-dimethyl-1,3,5-triazine, pyridine, pyrimidine-2,4-diamine
1-(4-((3-fluorobenzyl)amino)-6-(dimethylamino)-1,3,5-triazin-2-yl)-2-methyl-indoline-4-carboxylic acid
1-(4-(benzylamino)-6-(dimethylamino)-1,3,5-triazin-2-yl)-2-methyl-indoline-4-carboxylic acid
(3-(((4-(4-carbamoyl-2-methyl-indolin-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methyl-indoline-4-carboxamide
1-(4-(benzylamino)-6-(dimethylamino)-1,3,5-triazin-2-yl)-2-methyl-indoline-4-carboxamide
(3-(((4-(4-carbamoyl-6-fluoro-2-methyl-indolin-1-yl)-6-(dimethylamino)-1,3,5-triazin-2-yl)amino)methyl)phenyl)boronic acid
1-(4-((3-flurorobenzyl)amino)-6-(dimethylamino)-1,3,5-triazin-2-yl)-6-fluoro-2-methyl-indoline-4-carboxamide
1-(4-(benzylamino)-6-(dimethylamino)-1,3,5-triazin-2-yl)-6-fluoro-2-methyl-indoline-4-carboxamide
(3-(((4-(2-methyl-indolin-1-yl)-6-(dimethylamino)-1,3,5-triazin-2-yl)amino)methyl)phenyl)boronic acid
N2-(3-fluorobenzyl)-N4,N4-dimethyl-6-(2-methyl-indolin-1-yl)-1,3,5-triazine, pyridine, pyrimidine-2,4-diamine
N2-benzyl-N4,N4-dimethyl-6-(2-methyl-indolin-1-yl)-1,3,5-triazine, pyridine,
pyrimidine-2,4-diamine
(3-(((4-(4-sulfamoyll-2-methyl-indolin-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methyl-indoline-4-sulfonamide
1-(4-(benzylamino)-6-(dimethylamino)-1,3,5-triazin-2-yl)-2-methyl-indoline-4-sulfonamide
(1-(4-((3-fluorobenzyl)amino)-6-methoxy-1,3,5-triazin-2-yl)-2-methyl-indolin-4-yl)boronic acid
(1-(4-(benzylamino)-6-methoxy-1,3,5-triazin-2-yl)-2-methyl-indolin-4-yl)boronic acid
(3-(((4-(4-(aminomethyl)-2-methyl-indolin-1-yl)-6-methoxy-1,3,5-triazin-2-yl)amino)methyl)phenyl)boronic acid
4-(4-(aminomethyl)-2-methyl-indolin-1-yl)-N-(3-flurorobenzyl)-6-methoxy-1,3,5-triazin-2-amine
4-(4-(aminomethyl)-2-methyl-indolin-1-yl)-N-benzyl-6-methoxy-1,3,5-triazin-2-amine
(3-(((4-(4-(methoxymethy)-2-methyl-indolin-1-yl)-6-methoxy-1,3,5-triazin-2-yl)amino)methyl)phenyl)boronic acid
4-(4-(methoxymethyl)-2-methyl-indolin-1-yl)-N-(3-flurorobenzyl)-6-methoxy-1,3,5-triazin-2-amine
4-(4-(methoxymethyl)-2-methyl-indolin-1-yl)-N-benzyl-6-methoxy-1,3,5-triazin-2-amine
1-(4-((3-fluorobenzyl)amino)-6-methoxy-1,3,5-triazin-2-yl)-2-methyl-indoline-4-carboxylic acid
1-(4-(benzylamino)-6-methoxy-1,3,5-triazin-2-yl)-2-methyl-indoline-4-carboxylic acid
(3-(((4-(4-carbamoyl-2-methyl-indolin-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methyl-indoline-4-carboxamide
1-(4-(benzylamino)-6-methoxy-1,3,5-triazin-2-yl)-2-methyl-indoline-4-carboxamide
(3-(((4-(4-carbamoyl-6-fluoro-2-methyl-indolin-1-yl)-6-methoxy-1,3,5-triazin-2-yl)amino)methyl)phenyl)boronic acid
1-(4-((3-flurorobenzyl)amino)-6-methoxy-1,3,5-triazin-2-yl)-6-fluoro-2-methyl-indoline-4-carboxamide
1-(4-(benzylamino)-6-methoxy-1,3,5-triazin-2-yl)-6-fluoro-2-methyl-indoline-4-carboxamide
(3-(((4-(2-methyl-indolin-1-yl)-6-methoxy-1,3,5-triazin-2-yl)amino)methyl)phenyl)boronic acid
N-(3-flurorobenzyl)-4-methoxy-6-(2-methyl-indolin-1-yl)-1,3,5-triazin-2-amine
N-benzyl-4-methoxy-6-(2-methyl-indolin-1-yl)-1,3,5-triazin-2-amine
(3-(((4-(4-sulfamoyll-2-methyl-indolin-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methyl-indoline-4-sulfonamide
1-(4-(benzylamino)-6-methoxy-1,3,5-triazin-2-yl)-2-methyl-indoline-4-sulfonamide

### 2-Me-indolizine

(3-(((3-(8-carbamoyl-2-methylindolizin-3-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-2-methylindolizine-8-carboxamide
3-(5-(benzylamino)-6-carbamoyl-1,2,4-triazin-3-yl)-2-methylindolizine-8-carboxamide
(3-(((4-(8-carbamoyl-2-methylindolizin-3-yl)-6-carbamoyl-1,3,5-triazin-2-yl)amino)methyl)phenyl)boronic acid
3-(4-((3-fluorobenzyl)amino))-6-carbamoyl-1,3,5-triazin-2-yl)-2-methylindolizine-8-carboxamide
3-(4-(benzylamino)-6-carbamoyl-1,3,5-triazin-2-yl)-2-methylindolizine-8-carboxamide
3-(5-((3-fluorobenzyl)amino)-6-ethyl-1,2,4-triazin-3-yl)-2-methylindolizine-8-carboxamide
3-(5-(benzylamino)-6-ethyl-1,2,4-triazin-3-yl)-2-methylindolizine-8-carboxamide
(3-(((3-(8-carbamoyl-2-methylindolizin-3-yl)-6-ethyl-1,2,4-triazin-5-yl)amino)methyl)phenyl)boronic acid
3-(4-((3-fluorobenzyl)amino))-6-ethyl-1,3,5-triazin-2-yl)-2-methylindolizine-8-carboxamide
3-(4-(benzylamino)-6-ethyl-1,3,5-triazin-2-yl)-2-methylindolizine-8-carboxamide
(3-(((4-(8-carbamoyl-2-methylindolizin-3-yl)-6-ethyl-1,3,5-triazin-2-yl)amino)methyl)phenyl)boronic acid
(3-(((3-(8-carbamoyl-2-methylindolizin-3-yl)-1,2,4-triazin-5-yl)amino)methyl)phenyl)boronic acid
3-(5-((3-fluorobenzyl)amino)-1,2,4-triazin-3-yl)-2-methylindolizine-8-carboxamide
3-(5-(benzylamino)-1,2,4-triazin-3-yl)-2-methylindolizine-8-carboxamide
(3-(((4-(8-carbamoyl-2-methylindolizin-3-yl)-1,3,5-triazin-2-yl)amino)methyl)phenyl)boronic acid
3-(4-((3-fluorobenzyl)amino))-1,3,5-triazin-2-yl)-2-methylindolizine-8-carboxamide
3-(4-(benzylamino)-1,3,5-triazin-2-yl)-2-methylindolizine-8-carboxamide
3-(5-((3-fluorobenzyl)amino)-6-methyl-1,2,4-triazin-3-yl)-2-methylindolizine-8-carboxamide
3-(5-(benzylamino)-6-methyl-1,2,4-triazin-3-yl)-2-methylindolizine-8-carboxamide
(3-(((3-(8-carbamoyl-2-methylindolizin-3-yl)-6-methyl-1,2,4-triazin-5-yl)amino)methyl)phenyl)boronic acid
3-(4-((3-fluorobenzyl)amino))-6-methyl-1,3,5-triazin-2-yl)-2-methylindolizine-8-carboxamide
3-(4-(benzylamino)-6-methyl-1,3,5-triazin-2-yl)-2-methylindolizine-8-carboxamide
(3-(((4-(8-carbamoyl-2-methylindolizin-3-yl)-6-methyl-1,3,5-triazin-2-yl)amino)methyl)phenyl)boronic acid
(3-(((3-(8-carbamoyl-2-methylindolizin-3-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-2-methylindolizine-8-carboxamide
3-(5-(benzylamino)-6-(dimethylamino)-1,2,4-triazin-3-yl)-2-methylindolizine-8-carboxamide
(3-(((4-(8-carbamoyl-2-methylindolizin-3-yl)-6-(dimethylamino)-1,3,5-triazin-2-yl)amino)methyl)phenyl)boronic acid
3-(4-((3-fluorobenzyl)amino))-6-(dimethylamino)-1,3,5-triazin-2-yl)-2-methylindolizine-8-carboxamide
3-(4-(benzylamino)-6-(dimethylamino)-1,3,5-triazin-2-yl)-2-methylindolizine-8-carboxamide
(3-(((3-(8-carbamoyl-2-methylindolizin-3-yl)-6-methoxy-1,2,4-triazin-5-yl)amino)methyl)phenyl)boronic acid
3-(5-((3-fluorobenzyl)amino)-6-methoxy-1,2,4-triazin-3-yl)-2-methylindolizine-8-carboxamide
3-(5-(benzylamino)-6-methoxy-1,2,4-triazin-3-yl)-2-methylindolizine-8-carboxamide
(3-(((4-(8-carbamoyl-2-methylindolizin-3-yl)-6-methoxy-1,3,5-triazin-2-yl)amino)methyl)phenyl)boronic acid
3-(4-((3-fluorobenzyl)amino))-6-methoxy-1,3,5-triazin-2-yl)-2-methylindolizine-8-carboxamide
3-(4-(benzylamino)-6-methoxy-1,3,5-triazin-2-yl)-2-methylindolizine-8-carboxamide
(3-(((3-(5-carbamoyl-2-methylindolizin-1-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-2-methylindolizine-5-carboxamide
3-(5-(benzylamino)-6-carbamoyl-1,2,4-triazin-3-yl)-2-methylindolizine-5-carboxamide
3-(5-((3-fluorobenzyl)amino)-6-ethyl-1,2,4-triazin-3-yl)-2-methylindolizine-5-carboxamide
3-(5-(benzylamino)-6-ethyl-1,2,4-triazin-3-yl)-2-methylindolizine-5-carboxamide
(3-(((3-(5-carbamoyl-2-methylindolizin-1-yl)-6-ethyl-1,2,4-triazin-5-yl)amino)methyl)phenyl)boronic acid
(3-(((3-(5-carbamoyl-2-methylindolizin-1-yl)-1,2,4-triazin-5-yl)amino)methyl)phenyl)boronic acid
3-(5-((3-fluorobenzyl)amino)-1,2,4-triazin-3-yl)-2-methylindolizine-5-carboxamide
3-(5-(benzylamino)-1,2,4-triazin-3-yl)-2-methylindolizine-5-carboxamide
3-(5-((3-fluorobenzyl)amino)-6-methyl-1,2,4-triazin-3-yl)-2-methylindolizine-5-carboxamide
3-(5-(benzylamino)-6-methyl-1,2,4-triazin-3-yl)-2-methylindolizine-5-carboxamide
(3-(((3-(5-carbamoyl-2-methylindolizin-1-yl)-6-methyl-1,2,4-triazin-5-yl)amino)methyl)phenyl)boronic acid
(3-(((3-(5-carbamoyl-2-methylindolizin-1-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-2-methylindolizine-5-carboxamide
3-(5-(benzylamino)-6-(dimethylamino)-1,2,4-triazin-3-yl)-2-methylindolizine-5-carboxamide
(3-(((3-(5-carbamoyl-2-methylindolizin-1-yl)-6-methoxy-1,2,4-triazin-5-yl)amino)methyl)phenyl)boronic acid
3-(5-((3-fluorobenzyl)amino)-6-methoxy-1,2,4-triazin-3-yl)-2-methylindolizine-5-carboxamide
3-(5-(benzylamino)-6-methoxy-1,2,4-triazin-3-yl)-2-methylindolizine-5-carboxamide
(3-(((4-(8-carbamoyl-2-methylindolizin-1-yl)-6-mcarbamoyl-1,3,5-triazin-2-yl)amino)methyl)phenyl)boronic acid
3-(4-((3-fluorobenzyl)amino)-6-carbamoyl-1,3,5-triazin-2-yl)-2-methylindolizine-8-carboxamide
3-(4-(benzylamino)-6-carbamoyl-1,3,5-triazin-2-yl)-2-methylindolizine-8-carboxamide
3-(4-((3-fluorobenzyl)amino)-6-ethyl-1,3,5-triazin-2-yl)-2-methylindolizine-8-carboxamide
3-(4-(benzylamino)-6-ethyl-1,3,5-triazin-2-yl)-2-methylindolizine-8-carboxamide
(3-(((4-(8-carbamoyl-2-methylindolizin-1-yl)-6-ethyl-1,3,5-triazin-2-yl)amino)methyl)phenyl)boronic acid
(3-(((4-(8-carbamoyl-2-methylindolizin-1-yl)-1,3,5-triazin-2-yl)amino)methyl)phenyl)boronic acid
3-(4-((3-fluorobenzyl)amino)-1,3,5-triazin-2-yl)-2-methylindolizine-8-carboxamide
3-(4-(benzylamino)-1,3,5-triazin-2-yl)-2-methylindolizine-8-carboxamide
3-(4-((3-fluorobenzyl)amino)-6-methyl-1,3,5-triazin-2-yl)-2-methylindolizine-8-carboxamide
3-(4-(benzylamino)-6-methyl-1,3,5-triazin-2-yl)-2-methylindolizine-8-carboxamide
(3-(((4-(8-carbamoylindolizin-3-yl)-6-methyl-1,3,5-triazin-2-yl)amino)methyl)phenyl)boronic acid
(3-(((4-(8-carbamoyl-2-methylindolizin-1-yl)-6-(dimethylamino)-1,3,5-triazin-2-yl)amino)methyl)phenyl)boronic acid
3-(4-((3-fluorobenzyl)amino)-6-(dimethylamino)-1,3,5-triazin-2-yl)-2-methylindolizine-8-carboxamide
3-(4-(benzylamino)-6-(dimethylamino)-1,3,5-triazin-2-yl)-2-methylindolizine-8-carboxamide
(3-(((4-(8-carbamoyl-2-methylindolizin-1-yl)-6-methoxy-1,3,5-triazin-2-yl)amino)methyl)phenyl)boronic acid
3-(4-((3-fluorobenzyl)amino)-6-methoxy-1,3,5-triazin-2-yl)-2-methylindolizine-8-carboxamide
3-(4-(benzylamino)-6-methoxy-1,3,5-triazin-2-yl)-2-methylindolizine-8-carboxamide

### 2-Me-pyrazolo[1,5-a]pyridine

(3-(((3-(7-carbamoyl-2-methylpyrazolo[1,5-a]pyridin-3-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-2-methylpyrazolo[1,5-a]pyridine-7-carboxamide
3-(5-(benzylamino)-6-carbamoyl-1,2,4-triazin-3-yl)-2-methylpyrazolo[1,5-a]pyridine-7-carboxamide
3-(5-((3-fluorobenzyl)amino)-6-ethyl-1,2,4-triazin-3-yl)-2-methylpyrazolo[1,5-a]pyridine-7-carboxamide
3-(5-(benzylamino)-6-ethyl-1,2,4-triazin-3-yl)-2-methylpyrazolo[1,5-a]pyridine-7-carboxamide
(3-(((3-(7-carbamoyl-2-methylpyrazolo[1,5-a]pyridin-3-yl)-6-ethyl-1,2,4-triazin-5-yl)amino)methyl)phenyl)boronic acid
(3-(((3-(7-carbamoyl-2-methylpyrazolo[1,5-a]pyridin-3-yl)-1,2,4-triazin-5-yl)amino)methyl)phenyl)boronic acid
3-(5-((3-fluorobenzyl)amino)-1,2,4-triazin-3-yl)-2-methylpyrazolo[1,5-a]pyridine-7-carboxamide
3-(5-(benzylamino)-1,2,4-triazin-3-yl)-2-methylpyrazolo[1,5-a]pyridine-7-carboxamide
3-(5-((3-fluorobenzyl)amino)-6-methyl-1,2,4-triazin-3-yl)-2-methylpyrazolo[1,5-a]pyridine-7-carboxamide
3-(5-(benzylamino)-6-methyl-1,2,4-triazin-3-yl)-2-methylpyrazolo[1,5-a]pyridine-7-carboxamide
(3-(((3-(7-carbamoyl-2-methylpyrazolo[1,5-a]pyridin-3-yl)-6-methyl-1,2,4-triazin-5-yl)amino)methyl)phenyl)boronic acid
(3-(((3-(7-carbamoyl-2-methylpyrazolo[1,5-a]pyridin-3-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-2-methylpyrazolo[1,5-a]pyridine-7-carboxamide
3-(5-(benzylamino)-6-(dimethylamino)-1,2,4-triazin-3-yl)-2-methylpyrazolo[1,5-a]pyridine-7-carboxamide
(3-(((3-(7-carbamoyl-2-methylpyrazolo[1,5-a]pyridin-3-yl)-6-methoxy-1,2,4-triazin-5-yl)amino)methyl)phenyl)boronic acid
3-(5-((3-fluorobenzyl)amino)-6-methoxy-1,2,4-triazin-3-yl)-2-methylpyrazolo[1,5-a]pyridine-7-carboxamide
3-(5-(benzylamino)-6-methoxy-1,2,4-triazin-3-yl)-2-methylpyrazolo[1,5-a]pyridine-7-carboxamide
(3-(((4-(7-carbamoylpyrazolo[1,5-a]pyridin-3-yl)-6-carbamoyl-1,3,5-triazin-2-yl)amino)methyl)phenyl)boronic acid
3-(4-(3-fluorobenzylamino)-6-carbamoyl-1,3,5-triazin-2-yl)pyrazolo[1,5-a]pyridine-7-carboxamide
3-(4-(benzylamino)-6-carbamoyl-1,3,5-triazin-2-yl)pyrazolo[1,5-a]pyridine-7-carboxamide
3-(4-(3-fluorobenzylamino)-6-ethyl-1,3,5-triazin-2-yl)pyrazolo[1,5-a]pyridine-7-carboxamide
3-(4-(benzylamino)-6-ethyl-1,3,5-triazin-2-yl)pyrazolo[1,5-a]pyridine-7-carboxamide
(3-(((4-(7-carbamoylpyrazolo[1,5-a]pyridin-3-yl)-6-ethyl-1,3,5-triazin-2-yl)amino)methyl)phenyl)boronic acid
(3-(((4-(7-carbamoylpyrazolo[1,5-a]pyridin-3-yl)-1,3,5-triazin-2-yl)amino)methyl)phenyl)boronic acid
3-(4-(3-fluorobenzylamino)-1,3,5-triazin-2-yl)pyrazolo[1,5-a]pyridine-7-carboxamide
3-(4-(benzylamino)-1,3,5-triazin-2-yl)pyrazolo[1,5-a]pyridine-7-carboxamide
3-(4-(3-fluorobenzylamino)-6-methyl-1,3,5-triazin-2-yl)pyrazolo[1,5-a]pyridine-7-carboxamide
3-(4-(benzylamino)-6-methyl-1,3,5-triazin-2-yl)pyrazolo[1,5-a]pyridine-7-carboxamide
(3-(((4-(7-carbamoylpyrazolo[1,5-a]pyridin-3-yl)-6-methyl-1,3,5-triazin-2-yl)amino)methyl)phenyl)boronic acid
(3-(((4-(7-carbamoylpyrazolo[1,5-a]pyridin-3-yl)-6-(dimethylamino)-1,3,5-triazin-2-yl)amino)methyl)phenyl)boronic acid
3-(4-(3-fluorobenzylamino)-6-(dimethylamino)-1,3,5-triazin-2-yl)pyrazolo[1,5-a]pyridine-7-carboxamide
3-(4-(benzylamino)-6-(dimethylamino)-1,3,5-triazin-2-yl)pyrazolo[1,5-a]pyridine-7-carboxamide
(3-(((4-(7-carbamoylpyrazolo[1,5-a]pyridin-3-yl)-6-methoxy-1,3,5-triazin-2-yl)amino)methyl)phenyl)boronic acid
3-(4-(3-fluorobenzylamino)-6-methoxy-1,3,5-triazin-2-yl)pyrazolo[1,5-a]pyridine-7-carboxamide
3-(4-(benzylamino)-6-methoxy-1,3,5-triazin-2-yl)pyrazolo[1,5-a]pyridine-7-carboxamide

### 2-OMe-1-benzimidazole

(1-(5-((3-fluorobenzyl)amino)-6-carbamoyl-1,2,4-triazin-3-yl)-2-methoxy-1H-benzo[d]imidazole-4-yl)boronic acid
(1-(5-(benzylamino)-6-carbamoyl-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-carbamoyl-1,2,4-triazin-5-yl)amino)methyl)phenyl)boronic acid
3-(4-(aminomethyl)-2-methoxy-1H-benzo[d]imidazol-1-yl)-5-((3-fluorobenzyl)amino)-1,2,4-triazine, pyridine, pyrimidine-6-carboxamide
3-(4-(aminomethyl)-2-methoxy-1H-benzo[d]imidazol-1-yl)-5-(benzylamino)-1,2,4-triazine, pyridine, pyrimidine-6-carboxamide
(3-(((3-(4-(methoxymethyl)-2-methoxy-1H-benzo[d]imidazol-1-yl)-6-carbamoyl-1,2,4-triazin-5-yl)amino)methyl)phenyl)boronic acid
3-(4-(methoxymethyl)-2-methoxy-1H-benzo[d]imidazol-yl)-5-((3-fluorobenzyl)amino)-1,2,4-triazine, pyridine, pyrimidine-6-carboxamide
3-(4-(methoxymethyl)-2-methoxy-1H-benzo[d]imidazol-1-yl)-5-(benzylamino)-1,2,4-triazine, pyridine, pyrimidine-6-carboxamide
1-(5-((3-fluorobenzyl)amino)-6-carbamoyl-1,2,4-triazin-3-yl)-2-methoxy-1H-benzo[d]imidazole-4-carboxylic acid
1-(5-(benzylamino)-6-carbamoyl-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-6-mcarbamoyl-1,2,4-triazin-5-yl)amino)methyl)phenyl)boronic acid
1-(5-((3-fluorobenzyl)amino)-6-carbamoyl-1,2,4-triazin-3-yl)-2-methoxy-1H-benzo[d]imidazole-4-carboxamide
1-(5-(benzylamino)-6-carbamoyl-1,2,4-triazin-3-yl)-2-methoxy-1H-benzo[d]imidazole-4-carboxamide
(3-(((6-carbamoyl-3-(2-methoxy-1H-benzo[d]imidazol-1-yl)-1,2,4-triazin-5-yl)amino)methyl)phenyl)boronic acid
5-((3-fluorobenzyl)amino)-3-(2-methoxy-1H-benzo[d]imidazol-1-yl)-1,2,4-triazine, pyridine, pyrimidine-6-carboxamide
5-(benzylamino)-3-(2-methoxy-1H-benzo[d]imidazol-1-yl)-1,2,4-triazine, pyridine, pyrimidine-6-carboxamide
(3-(((6-carbamoyl-3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-6-carbamoyl-1,2,4-triazin-3-yl)-2-methoxy-1H-benzo[d]imidazole-4-sulfonamide
1-(5-(benzylamino)-6-carbamoyl-1,2,4-triazin-3-yl)-2-methoxy-1H-benzo[d]imidazole-4-sulfonamide
(1-(5-((3-fluorobenzyl)amino)-6-ethyl-1,2,4-triazin-3-yl)-2-methoxy-1H-benzo[d]imidazole-4-yl)boronic acid
(1-(5-(benzylamino)-6-ethyl-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-ethyl-1,2,4-triazin-5-yl)amino)methyl)phenyl)boronic acid
3-(4-(aminomethyl)-2-methoxy-1H-benzo[d]imidazol-1-yl)-N-(3-fluorobenzyl)-6-ethyl-1,2,4-triazin-5-amine
3-(4-(aminomethyl)-2-methoxy-1H-benzo[d]imidazol-1-yl)-N-benzyl-6-ethyl-1,2,4-triazin-5-amine
(3-(((3-(4-(methoxymethyl)-2-methoxy-1H-benzo[d]imidazol-1-yl)-6-ethyl-1,2,4-triazin-5-yl)amino)methyl)phenyl)boronic acid
3-(4-(methoxymethyl)-2-methoxy-1H-benzo[d]imidazol-1-yl)-N-(3-fluorobenzyl)-6-ethyl-1,2,4-triazin-5-amine
3-(4-(methoxymethyl)-2-methoxy-1H-benzo[d]imidazol-1-yl)-N-benzyl-6-ethyl-1,2,4-triazin-5-amine
1-(5-((3-fluorobenzyl)amino)-6-ethyl-1,2,4-triazin-3-yl)-2-methoxy-1H-benzo[d]imidazole-4-carboxylic acid
1-(5-(benzylamino)-6-ethyl-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-2-methoxy-1H-benzo[d]imidazole-4-carboxamide
1-(5-(benzylamino)-6-ethyl-1,2,4-triazin-3-yl)-2-methoxy-1H-benzo[d]imidazole-4-carboxamide
(3-(((6-ethyl-3-(2-methoxy-1H-benzo[d]imidazol-1-yl)-1,2,4-triazin-5-yl)amino)methyl)phenyl)boronic acid
N-(3-fluorobenzyl)-6-ethyl-3-(2-methoxy-1H-benzo[d]imidazol-1-yl)-1,2,4-triazin-5-amine
N-benzyl-6-ethyl-3-(2-methoxy-1H-benzo[d]imidazol-1-yl)-1,2,4-triazin-5-amine
(3-(((6-ethyl-3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-6-ethyl-1,2,4-triazin-3-yl)-2-methoxy-1H-benzo[d]imidazole-4-sulfonamide
1-(5-(benzylamino)-6-ethyl-1,2,4-triazin-3-yl)-2-methoxy-1H-benzo[d]imidazole-4-sulfonamide
(1-(5-((3-fluorobenzyl)amino)-1,2,4-triazin-3-yl)-2-methoxy-1H-benzo[d]imidazole-4-yl)boronic acid
(1-(5-(benzylamino)-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-1,2,4-triazin-5-yl)amino)methyl)phenyl)boronic acid
3-(4-(aminomethyl)-2-methoxy-1H-benzo[d]imidazol-1-yl)-N-(3-fluorobenzyl)-1,2,4-triazin-5-amine
3-(4-(aminomethyl)-2-methoxy-1H-benzo[d]imidazol-1-yl)-N-benzyl-1,2,4-triazin-5-amine
(3-(((3-(4-(methoxymethyl)-2-methoxy-1H-benzo[d]imidazol-1-yl)-6-1,2,4-triazin-5-yl)amino)methyl)phenyl)boronic acid
3-(4-(methoxymethyl)-2-methoxy-1H-benzo[d]imidazol-1-yl)-N-(3-fluorobenzyl)-1,2,4-triazin-5-amine
3-(4-(methoxymethyl)-2-methoxy-1H-benzo[d]imidazol-1-yl)-N-benzyl-1,2,4-triazin-5-amine
1-(5-((3-fluorobenzyl)amino)-1,2,4-triazin-3-yl)-2-methoxy-1H-benzo[d]imidazole-4-carboxylic acid
1-(5-(benzylamino)-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-1,2,4-triazin-5-yl)amino)methyl)phenyl)boronic acid
1-(5-((3-fluorobenzyl)amino)-1,2,4-triazin-3-yl)-2-methoxy-1H-benzo[d]imidazole-4-carboxamide
1-(5-(benzylamino)-1,2,4-triazin-3-yl)-2-methoxy-1H-benzo[d]imidazole-4-carboxamide
(3-(((3-(2-methoxy-1H-benzo[d]imidazol-1-yl)-1,2,4-triazin-5-yl)amino)methyl)phenyl)boronic acid
N-(3-fluorobenzyl)-3-(2-methoxy-1H-benzo[d]imidazol-1-yl)-1,2,4-triazin-5-amine
N-benzyl-3-(2-methoxy-1H-benzo[d]imidazol-1-yl)-1,2,4-triazin-5-amine
(3-(((3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-1,2,4-triazin-3-yl)-2-methoxy-1H-benzo[d]imidazole-4-sulfonamide
1-(5-(benzylamino)-1,2,4-triazin-3-yl)-2-methoxy-1H-benzo[d]imidazole-4-sulfonamide
(1-(5-((3-fluorobenzyl)amino)-6-methyl-1,2,4-triazin-3-yl)-2-methoxy-1H-benzo[d]imidazole-4-yl)boronic acid
(1-(5-(benzylamino)-6-methyl-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-methyl-1,2,4-triazin-5-yl)amino)methyl)phenyl)boronic acid
3-(4-(aminomethyl)-2-methoxy-1H-benzo[d]imidazol-1-yl)-N-(3-fluorobenzyl)-6-methyl-1,2,4-triazin-5-amine
3-(4-(aminomethyl)-2-methoxy-1H-benzo[d]imidazol-1-yl)-N-benzyl-6-methyl-1,2,4-triazin-5-amine
(3-(((3-(4-(methoxymethyl)-2-methoxy-1H-benzo[d]imidazol-1-yl)-6-methyl-1,2,4-triazin-5-yl)amino)methyl)phenyl)boronic acid
3-(4-(methoxymethyl)-2-methoxy-1H-benzo[d]imidazol-1-yl)-N-(3-fluorobenzyl)-6-methyl-1,2,4-triazin-5-amine
3-(4-(methoxymethyl)-2-methoxy-1H-benzo[d]imidazol-1-yl)-N-benzyl-6-methyl-1,2,4-triazin-5-amine
1-(5-((3-fluorobenzyl)amino)-6-methyl-1,2,4-triazin-3-yl)-2-methoxy-1H-benzo[d]imidazole-4-carboxylic acid
1-(5-(benzylamino)-6-methyl-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-2-methoxy-1H-benzo[d]imidazole-4-carboxamide
1-(5-(benzylamino)-6-methyl-1,2,4-triazin-3-yl)-2-methoxy-1H-benzo[d]imidazole-4-carboxamide
(3-(((6-methyl-3-(2-methoxy-1H-benzo[d]imidazol-1-yl)-1,2,4-triazin-5-yl)amino)methyl)phenyl)boronic acid
N-(3-fluorobenzyl)-6-methyl-3-(2-methoxy-1H-benzo[d]imidazol-1-yl)-1,2,4-triazin-5-amine
N-benzyl-6-methyl-3-(2-methoxy-1H-benzo[d]imidazol-1-yl)-1,2,4-triazin-5-amine
(3-(((6-methyl-3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-6-methyl-1,2,4-triazin-3-yl)-2-methoxy-1H-benzo[d]imidazole-4-sulfonamide
1-(5-(benzylamino)-6-methyl-1,2,4-triazin-3-yl)-2-methoxy-1H-benzo[d]imidazole-4-sulfonamide
(1-(5-((3-fluorobenzyl)amino)-6-(dimethylamino)-1,2,4-triazin-3-yl)-2-methoxy-1H-benzo[d]imidazole-4-yl)boronic acid
(1-(5-(benzylamino)-6-(dimethylamino)-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-(dimethylamino)-1,2,4-triazin-5-yl)amino)methyl)phenyl)boronic acid
3-(4-(aminomethyl)-2-methoxy-1H-benzo[d]imidazol-1-yl)-N5-(3-fluorobenzyl)-N6,N6-dimethyl-1,2,4-triazine, pyridine, pyrimidine-5,6-diamine
3-(4-(aminomethyl)-2-methoxy-1H-benzo[d]imidazol-1-yl)-N5-benzyl-N6,N6-dimethyl-1,2,4-triazine, pyridine, pyrimidine-5,6-diamine
(3-(((3-(4-(methoxymethyl)-2-methoxy-H-benzo[d]imidazol-1-yl)-6-(dimethylamino)-1,2,4-triazin-5-yl)amino)methyl)phenyl)boronic acid
3-(4-(methoxymethyl)-2-methoxy-1H-benzo[d]imidazol-1-yl)-N5-(3-fluorobenzyl)-N6,N6-dimethyl-1,2,4-triazine, pyridine, pyrimidine-5,6-diamine
3-(4-(methoxymethyl)-2-methoxy-1H-benzo[d]imidazol-1-yl)-N5-benzyl-N6,N6-dimethyl-1,2,4-triazine, pyridine, pyrimidine-5,6-diamine
1-(5-((3-fluorobenzyl)amino)-6-(dimethylamino)-1,2,4-triazin-3-yl)-2-methoxy-1H-benzo[d]imidazole-4-carboxylic acid
1-(5-(benzylamino)-6-(dimethylamino)-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-6-(dimethylamino)-1,2,4-triazin-5-yl)amino)methyl)phenyl)boronic acid
1-(5-((3-fluorobenzyl)amino)-6-(dimethylamino)-1,2,4-triazin-3-yl)-2-methoxy-1H-benzo[d]imidazole-4-carboxamide
1-(5-(benzylamino)-6-(dimethylamino)-1,2,4-triazin-3-yl)-2-methoxy-1H-benzo[d]imidazole-4-carboxamide
(3-(((6-(dimethylamino)-3-(2-methoxy-1H-benzo[d]imidazol-1-yl)-1,2,4-triazin-5-yl)amino)methyl)phenyl)boronic acid
N5-(3-fluorobenzyl)-N6,N6-dimethyl-3-(2-methoxy-1H-benzo[d]imidazol-1-yl)-1,2,4-triazine, pyridine, pyrimidine-5,6-diamine
N5-benzyl-N6,N6-dimethyl-3-(2-methoxy-1H-benzo[d]imidazol-1-yl)-1,2,4-triazine, pyridine, pyrimidine-5,6-diamine
(3-(((6-(dimethylamino)-3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-6-(dimethylamino)-1,2,4-triazin-3-yl)-2-methoxy-1H-benzo[d]imidazole-4-sulfonamide
1-(5-(benzylamino)-6-(dimethylamino)-1,2,4-triazin-3-yl)-2-methoxy-1H-benzo[d]imidazole-4-sulfonamide
(1-(5-((3-fluorobenzyl)amino)-6-methoxy-1,2,4-triazin-3-yl)-2-methoxy-1H-benzo[d]imidazole-4-yl)boronic acid
(1-(5-(benzylamino)-6-methoxy-1,2,4-triazin-3-yl)-2-methoxy-1H-benzo[d]imidazole-4-yl)boronic acid
(3-(((3-(4-(aminomethyl)-2-methoxy-1H-benzo[d]imidazol-1-yl)-6-methoxy-1,2,4-triazin-5-yl)amino)methyl)phenyl)boronic acid
3-(4-(aminomethyl)-2-methoxy-1H-benzo[d]imidazol-1-yl)-N-(3-fluorobenzyl)-6-methoxy-1,2,4-triazin-5-amine
3-(4-(aminomethyl)-2-methoxy-1H-benzo[d]imidazol-1-yl)-N-benzyl-6-methoxy-1,2,4-triazin-5-amine
(3-(((3-(4-(methoxymethyl)-2-methoxy-1H-benzo[d]imidazol-1-yl)-6-methoxy-1,2,4-triazin-5-yl)amino)methyl)phenyl)boronic acid
3-(4-(methoxymethyl)-2-methoxy-1H-benzo[d]imidazol-1-yl)-N-(3-fluorobenzyl)-6-methoxy-1,2,4-triazin-5-amine
3-(4-(methoxymethyl)-2-methoxy-1H-benzo[d]imidazol-1-yl)-N-benzyl-6-methoxy-1,2,4-triazin-5-amine
1-(5-((3-fluorobenzyl)amino)-6-methoxy-1,2,4-triazin-3-yl)-2-methoxy-H-benzo[d]imidazole-4-carboxylic acid
1-(5-(benzylamino)-6-methoxy-1,2,4-triazin-3-yl)-2-methoxy-1H-benzo[d]imidazole-4-carboxylic acid
(3-(((3-(4-carbamoyl-2-methoxy-1H-benzo[d]imidazol-1-yl)-6-methoxy-1,2,4-triazin-5-yl)amino)methyl)phenyl)boronic acid
1-(5-((3-fluorobenzyl)amino)-6-methoxy-1,2,4-triazin-3-yl)-2-methoxy-H-benzo[d]imidazole-4-carboxamide
1-(5-(benzylamino)-6-methoxy-1,2,4-triazin-3-yl)-2-methoxy-1H-benzo[d]imidazole-4-carboxamide
(3-(((6-methoxy-3-(2-methoxy-1H-benzo[d]imidazol-1-yl)-1,2,4-triazin-5-yl)amino)methyl)phenyl)boronic acid
N-(3-fluorobenzyl)-6-methoxy-3-(2-methoxy-1H-benzo[d]imidazol-1-yl)-1,2,4-triazin-5-amine
N-benzyl-6-methoxy-3-(2-methoxy-1H-benzo[d]imidazol-1-yl)-1,2,4-triazin-5-amine
(3-(((6-methoxy-3-(2-methoxy-4-sulfinamoyl-1H-benzo[d]imidazol-1-yl)-1,2,4-triazin-5-yl)amino)methyl)phenyl)boronic acid
1-(5-((3-fluorobenzyl)amino)-6-methoxy-1,2,4-triazin-3-yl)-2-methoxy-H-benzo[d]imidazole-4-sulfonamide
1-(5-(benzylamino)-6-methoxy-1,2,4-triazin-3-yl)-2-methoxy-1H-benzo[d]imidazole-4-sulfonamide
(1-(4-((3-fluorobenzyl)amino)-6-carbamoyl-1,3,5-triazin-2-yl)-2-methoxy-1H-benzo[d]imidazol-4-yl)boronic acid
(1-(4-(benzylamino)-6-carbamoyl-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-carbomyl-1,3,5-triazin-2-yl)amino)methyl)phenyl)boronic acid
4-(4-(aminomethyl)-2-methoxy-1H-benzo[d]imidazol-1-yl)-6-((3-fluorobenzyl)amino)-1,3,5-triazine, pyridine, pyrimidine-2-carboxamide
4-(4-(aminomethyl)-2-methoxy-1H-benzo[d]imidazol-1-yl)-6-(benzylamino)-1,3,5-triazine, pyridine, pyrimidine-2-carboxamide
(3-(((4-carbamoyl-6-(2-methoxy-4-(methoxymethyl)-1H-benzo[d]imidazol-1-yl)-1,3,5-triazin-2-yl)amino)methyl)phenyl)boronic acid
4-((3-fluorobenzyl)amino)-6-(2-methoxy-4-(methoxymethyl)-1H-benzo[d]imidazol-1-yl)-1,3,5-triazine, pyridine, pyrimidine-2-carboxamide
4-(benzylamino)-6-(2-methoxy-4-(methoxymethyl)-1H-benzo[d]imidazol-1-yl)-1,3,5-triazine, pyridine, pyrimidine-2-carboxamide
1-(4-((3-fluorobenzyl)amino)-6-carbamoyl-1,3,5-triazin-2-yl)-2-methoxy-1H-benzo[d]imidazole-4-carboxylic acid
1-(4-(benzylamino)-6-carbamoyl-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methoxy-1H-benzo[d]imidazole-4-carboxamide
1-(4-(benzylamino)-6-carbamoyl-1,3,5-triazin-2-yl)-2-methoxy-1H-benzo[d]imidazole-4-carboxamide
(3-(((4-carbamoyl-6-(2-methoxy-1H-benzo[d]imidazol-1-yl)-1,3,5-triazin-2-yl)amino)methyl)phenyl)boronic acid
4-((3-fluorobenzyl)amino)-6-(2-methoxy-1H-benzo[d]imidazol-1-yl)-1,3,5-triazine, pyridine, pyrimidine-2-carboxamide
4-(benzylamino)-6-(2-methoxy-1H-benzo[d]imidazol-1-yl)-1,3,5-triazine, pyridine, pyrimidine-2-carboxamide
(3-(((4-(4-sulfinamoyl-2-methoxy-1H-benzo[d]imidazol-1-yl)-6-carbamoyl-1,3,5-triazin-2-yl)amino)methyl)phenyl)boronic acid
1-(4-((3-fluorobenzyl)amino)-6-carbamoyl-1,3,5-triazin-2-yl)-2-methoxy-1H-benzo[d]imidazole-4-sulfonamide
1-(4-(benzylamino)-6-carbamoyl-1,3,5-triazin-2-yl)-2-methoxy-1H-benzo[d]imidazole-4-sulfonamide
((1-(4-((3-fluorobenzyl)amino)-6-ethyl-1,3,5-triazin-2-yl)-2-methoxy-1H-benzo[d]imidazol-4-yl)boronic acid
(1-(4-(benzylamino)-6-ethyl-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-ethyl-1,3,5-triazin-2-yl)amino)methyl)phenyl)boronic acid
4-(4-(aminomethyl)-2-methoxy-1H-benzo[d]imidazol-1-yl)-N-(3-fluorobenzyl)-6-ethyl-1,3,5-triazin-2-amine
4-(4-(aminomethyl)-2-methoxy-1H-benzo[d]imidazol-1-yl)-N-benzyl-6-ethyl-1,3,5-triazin-2-amine
(3-(((4-(2-methoxy-4-(methoxymethyl)-1H-benzo[d]imidazol-1-yl)-6-ethyl-1,3,5-triazin-2-yl)amino)methyl)phenyl)boronic acid
N-(3-fluorobenzyl)-4-(2-methoxy-4-(methoxymethyl)-1H-benzo[d]imidazol-1-yl)-6-ethyl-1,3,5-triazin-2-amine
N-benzyl-4-(2-methoxy-4-(methoxymethyl)-1H-benzo[d]imidazol-1-yl)-6-ethyl-1,3,5-triazin-2-amine
1-(4-((3-fluorobenzyl)amino)-6-ethyl-1,3,5-triazin-2-yl)-2-methoxy-1H-benzo[d]imidazole-4-carboxylic acid
1-(4-(benzylamino)-6-ethyl-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methoxy-1H-benzo[d]imidazole-4-carboxamide
1-(4-(benzylamino)-6-ethyl-1,3,5-triazin-2-yl)-2-methoxy-1H-benzo[d]imidazole-4-carboxamide
(3-(((4-(2-methoxy-1H-benzo[d]imidazol-1-yl)-6-ethyl-1,3,5-triazin-2-yl)amino)methyl)phenyl)boronic acid
N-(3-fluorobenzyl)-4-(2-methoxy-1H-benzo[d]imidazol-1-yl)-6-ethyl-1,3,5-triazin-2-amine
N-benzyl-4-(2-methoxy-1H-benzo[d]imidazol-1-yl)-6-ethyl-1,3,5-triazin-2-amine
(3-(((4-(4-sulfinamoyl-2-methoxy-1H-benzo[d]imidazol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methoxy-1H-benzo[d]imidazole-4-sulfonamide
1-(4-(benzylamino)-6-ethyl-1,3,5-triazin-2-yl)-2-methoxy-1H-benzo[d]imidazole-4-sulfonamide
((1-(4-((3-fluorobenzyl)amino)-1,3,5-triazin-2-yl)-2-methoxy-1H-benzo[d]imidazol-4-yl)boronic acid
(1-(4-(benzylamino)-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-1,3,5-triazin-2-yl)amino)methyl)phenyl)boronic acid
4-(4-(aminomethyl)-2-methoxy-1H-benzo[d]imidazol-1-yl)-N-(3-fluorobenzyl)-1,3,5-triazin-2-amine
4-(4-(aminomethyl)-2-methoxy-1H-benzo[d]imidazol-1-yl)-N-benzyl-1,3,5-triazin-2-amine
(3-(((4-(2-methoxy-4-(methoxymethyl)-1H-benzo[d]imidazol-1-yl)-1,3,5-triazin-2-yl)amino)methyl)phenyl)boronic acid
N-(3-fluorobenzyl)-4-(2-methoxy-4-(methoxymethyl)-1H-benzo[d]imidazol-1-yl)-1,3,5-triazin-2-amine
N-benzyl-4-(2-methoxy-4-(methoxymethyl)-1H-benzo[d]imidazol-1-yl)-1,3,5-triazin-2-amine
1-(4-((3-fluorobenzyl)amino)-1,3,5-triazin-2-yl)-2-methoxy-1H-benzo[d]imidazole-4-carboxylic acid
1-(4-(benzylamino)-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-1,3,5-triazin-2-yl)amino)methyl)phenyl)boronic acid
1-(4-((3-fluorobenzyl)amino)-1,3,5-triazin-2-yl)-2-methoxy-1H-benzo[d]imidazole-4-carboxamide
1-(4-(benzylamino)-1,3,5-triazin-2-yl)-2-methoxy-1H-benzo[d]imidazole-4-carboxamide
(3-(((4-(2-methoxy-1H-benzo[d]imidazol-1-yl)-1,3,5-triazin-2-yl)amino)methyl)phenyl)boronic acid
N-(3-fluorobenzyl)-4-(2-methoxy-1H-benzo[d]imidazol-1-yl)-1,3,5-triazin-2-amine
N-benzyl-4-(2-methoxy-1H-benzo[d]imidazol-1-yl)-1,3,5-triazin-2-amine
(3-(((4-(4-sulfinamoyl-2-methoxy-1H-benzo[d]imidazol-1-yl)-1,3,5-triazin-2-yl)amino)methyl)phenyl)boronic acid
1-(4-((3-fluorobenzyl)amino)-1,3,5-triazin-2-yl)-2-methoxy-1H-benzo[d]imidazole-4-sulfonamide
1-(4-(benzylamino)-1,3,5-triazin-2-yl)-2-methoxy-1H-benzo[d]imidazole-4-sulfonamide
((1-(4-((3-fluorobenzyl)amino)-6-methyl-1,3,5-triazin-2-yl)-2-methoxy-1H-benzo[d]imidazol-4-yl)boronic acid
(1-(4-(benzylamino)-6-methyl-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-methyl-1,3,5-triazin-2-yl)amino)methyl)phenyl)boronic acid
4-(4-(aminomethyl)-2-methoxy-1H-benzo[d]imidazol-1-yl)-N-(3-fluorobenzyl)-6-methyl-1,3,5-triazin-2-amine
4-(4-(aminomethyl)-2-methoxy-1H-benzo[d]imidazol-1-yl)-N-benzyl-6-methyl-1,3,5-triazin-2-amine
(3-(((4-(2-methoxy-4-(methoxymethyl)-1H-benzo[d]imidazol-1-yl)-6-methyl-1,3,5-triazin-2-yl)amino)methyl)phenyl)boronic acid
N-(3-fluorobenzyl)-4-(2-methoxy-4-(methoxymethyl)-1H-benzo[d]imidazol-1-yl)-6-methyl-1,3,5-triazin-2-amine
N-benzyl-4-(2-methoxy-4-(methoxymethyl)-1H-benzo[d]imidazol-1-yl)-6-methyl-1,3,5-triazin-2-amine
1-(4-((3-fluorobenzyl)amino)-6-methyl-1,3,5-triazin-2-yl)-2-methoxy-1H-benzo[d]imidazole-4-carboxylic acid
1-(4-(benzylamino)-6-methyl-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methoxy-1H-benzo[d]imidazole-4-carboxamide
1-(4-(benzylamino)-6-methyl-1,3,5-triazin-2-yl)-2-methoxy-1H-benzo[d]imidazole-4-carboxamide
(3-(((4-(2-methoxy-1H-benzo[d]imidazol-1-yl)-6-methyl-1,3,5-triazin-2-yl)amino)methyl)phenyl)boronic acid
N-(3-fluorobenzyl)-4-(2-methoxy-1H-benzo[d]imidazol-1-yl)-6-methyl-1,3,5-triazin-2-amine
N-benzyl-4-(2-methoxy-1H-benzo[d]imidazol-1-yl)-6-methyl-1,3,5-triazin-2-amine
(3-(((4-(4-sulfinamoyl-2-methoxy-1H-benzo[d]imidazol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methoxy-1H-benzo[d]imidazole-4-sulfonamide
1-(4-(benzylamino)-6-methyl-1,3,5-triazin-2-yl)-2-methoxy-1H-benzo[d]imidazole-4-sulfonamide
(1-(4-((3-fluorobenzyl)amino)-6-(dimethylamino)-1,3,5-triazin-2-yl)-2-methoxy-1H-benzo[d]imidazol-4-yl)boronic acid
(1-(4-(benzylamino)-6-(dimethylamino)-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-(dimethylamino)-1,3,5-triazin-2-yl)amino)methyl)phenyl)boronic acid
6-(4-(aminomethyl)-2-methoxy-1H-benzo[d]imidazol-1-yl)-N2-(3-fluorobenzyl)-N4,N4-dimethyl-1,3,5-triazine, pyridine, pyrimidine-2,4-diamine
6-(4-(aminomethyl)-2-methoxy-1H-benzo[d]imidazol-1-yl)-N2-benzyl-N4,N4-dimethyl-1,3,5-triazine, pyridine, pyrimidine-2,4-diamine
(3-(((4-(2-methoxy-4-(methoxymethyl)-1H-benzo[d]imidazol-1-yl)-6-(dimethylamino)-1,3,5-triazin-2-yl)amino)methyl)phenyl)boronic acid
N2-(3-fluorobenzyl)-6-(2-methoxy-4-(methoxymethyl)-1H-benzo[d]imidazol-1-yl)-N4,N4-dimethyl-1,3,5-triazine, pyridine, pyrimidine-2,4-diamine
N2-benzyl-6-(2-methoxy-4-(methoxymethyl)-1H-benzo[d]imidazol-1-yl)-N4,N4-dimethyl-1,3,5-triazine, pyridine, pyrimidine-2,4-diamine
1-(4-((3-fluorobenzyl)amino)-6-(dimethylamino)-1,3,5-triazin-2-yl)-2-methoxy-1H-benzo[d]imidazole-4-carboxylic acid
1-(4-(benzylamino)-6-(dimethylamino)-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methoxy-1H-benzo[d]imidazole-4-carboxamide
1-(4-(benzylamino)-6-(dimethylamino)-1,3,5-triazin-2-yl)-2-methoxy-1H-benzo[d]imidazole-4-carboxamide
(3-(((4-(dimethylamino)-6-(2-methoxy-1H-benzo[d]imidazol-1-yl)-1,3,5-triazin-2-yl)amino)methyl)phenyl)boronic acid
N2-(3-fluorobenzyl)-6-(2-methoxy-1H-benzo[d]imidazol-1-yl)-N4,N4-dimethyl-1,3,5-triazine, pyridine, pyrimidine-2,4-diamine
N2-benzyl-6-(2-methoxy-1H-benzo[d]imidazol-1-yl)-N4,N4-dimethyl-1,3,5-triazine, pyridine, pyrimidine-2,4-diamine
(3-(((4-(4-sulfinamoyl-2-methoxy-1H-benzo[d]imidazol-1-yl)-6-(dimethylamino)-1,3,5-triazin-2-yl)amino)methyl)phenyl)boronic acid
1-(4-((3-fluorobenzyl)amino)-6-(dimethylamino)-1,3,5-triazin-2-yl)-2-methoxy-1H-benzo[d]imidazole-4-sulfonamide
1-(4-(benzylamino)-6-(dimethylamino)-1,3,5-triazin-2-yl)-2-methoxy-1H-benzo[d]imidazole-4-sulfonamide
(1-(4-((3-fluorobenzyl)amino)-6-methoxy-1,3,5-triazin-2-yl)-2-methoxy-1H-benzo[d]imidazol-4-yl)boronic acid
(1-(4-(benzylamino)-6-methoxy-1,3,5-triazin-2-yl)-2-methoxy-1H-benzo[d]imidazol-4-yl)boronic acid
(3-(((4-(4-(aminomethyl)-2-methoxy-1H-benzo[d]imidazol-1-yl)-6-methox)-1,3,5-triazin-2-yl)amino)methyl)phenyl)boronic acid
4-(4-(aminomethyl)-2-methoxy-1H-benzo[d]imidazol-1-yl)-N-(3-fluorobenzyl)-6-methoxy-1,3,5-triazin-2-amine
4-(4-(aminomethyl)-2-methoxy-1H-benzo[d]imidazol-1-yl)-N-benzyl-6-methoxy-1,3,5-triazin-2-amine
(3-(((4-(2-methoxy-4-(methoxymethyl)-1H-benzo[d]imidazol-1-yl)-6-methoxy-1,3,5-triazin-2-yl)amino)methyl)phenyl)boronic acid
N-(3-fluorobenzyl)-4-(2-methoxy-4-(methoxymethyl)-1H-benzo[d]imidazol-1-yl)-6-methoxy-1,3,5-triazin-2-amine
N-benzyl-4-(2-methoxy-4-(methoxymethyl)-1H-benzo[d]imidazol-1-yl)-6-methoxy-1,3,5-triazin-2-amine
1-(4-((3-fluorobenzyl)amino)-6-methoxy-1,3,5-triazin-2-yl)-2-methoxy-1H-benzo[d]imidazole-4-carboxylic acid
1-(4-(benzylamino)-6-methoxy-1,3,5-triazin-2-yl)-2-methoxy-1H-benzo[d]imidazole-4-carboxylic acid
(3-(((4-(4-carbamoyl-2-methoxy-1H-benzo[d]imidazol-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methoxy-1H-benzo[d]imidazole-4-carboxamide
1-(4-(benzylamino)-6-methoxy-1,3,5-triazin-2-yl)-2-methoxy-1H-benzo[d]imidazole-4-carboxamide
(3-(((4-methoxy-6-(2-methoxy-1H-benzo[d]imidazol-1-yl)-1,3,5-triazin-2-yl)amino)methyl)phenyl)boronic acid
N-(3-fluorobenzyl)-4-(2-methoxy-1H-benzo[d]imidazol-1-yl)-6-methoxy-1,3,5-triazin-2-amine
N-benzyl-4-(2-methoxy-1H-benzo[d]imidazol-1-yl)-6-methoxy-1,3,5-triazin-2-amine
(3-(((4-(4-sulfinamoyl-2-methoxy-1H-benzo[d]imidazol-1-yl)-6-methoxy-1,3,5-triazin-2-yl)amino)methyl)phenyl)boronic acid
1-(4-((3-fluorobenzyl)amino)-6-methoxy-1,3,5-triazin-2-yl)-2-methoxy-1H-benzo[d]imidazole-4-sulfonamide
1-(4-(benzylamino)-6-methoxy-1,3,5-triazin-2-yl)-2-methoxy-1H-benzo[d]imidazole-4-sulfonamide

### 3a,7a-dihydrobenzo[b]thiophene

3-(5-((3-fluorobenzyl)amino)-6-carbamoyl-1,2,4-triazin-3-yl)-3a,7a-dihydrobenzo[b]thiophene-7-carboxamide
3-(5-(benzylamino)-6-carbamoyl-1,2,4-triazin-3-yl)-3a,7a-dihydrobenzo[b]thiophene-7-carboxamide
3-(5-((3-fluorobenzyl)amino)-6-ethyl-1,2,4-triazin-3-yl)-3a,7a-dihydrobenzo[b]thiophene-7-carboxamide
3-(5-(benzylamino)-6-ethyl-1,2,4-triazin-3-yl)-3a,7a-dihydrobenzo[b]thiophene-7-carboxamide
3-(5-((3-fluorobenzyl)amino)-1,2,4-triazin-3-yl)-3a,7a-dihydrobenzo[b]thiophene-7-carboxamide
3-(5-(benzylamino)-1,2,4-triazin-3-yl)-3a,7a-dihydrobenzo[b]thiophene-7-carboxamide
3-(5-((3-fluorobenzyl)amino)-6-methyl-1,2,4-triazin-3-yl)-3a,7a-dihydrobenzo[b]thiophene-7-carboxamide
3-(5-(benzylamino)-6-methyl-1,2,4-triazin-3-yl)-3a,7a-dihydrobenzo[b]thiophene-7-carboxamide
3-(5-((3-fluorobenzyl)amino)-6-(dimethylamino)-1,2,4-triazin-3-yl)-3a,7a-dihydrobenzo[b]thiophene-7-carboxamide
3-(5-(benzylamino)-6-(dimethylamino)-1,2,4-triazin-3-yl)-3a,7a-dihydrobenzo[b]thiophene-7-carboxamide
3-(5-((3-fluorobenzyl)amino)-6-methoxy-1,2,4-triazin-3-yl)-3a,7a-dihydrobenzo[b]thiophene-7-carboxamide
3-(5-(benzylamino)-6-methoxy-1,2,4-triazin-3-yl)-3a,7a-dihydrobenzo[b]thiophene-7-carboxamide
3-(4-((3-fluorobenzyl)amino)-6-carbamoyl-1,3,5-triazin-2-yl)-3a,7a-dihydrobenzo[b]thiophene-7-carboxamide
3-(4-(benzylamino)-6-carbamoyl-1,3,5-triazin-2-yl)-3a,7a-dihydrobenzo[b]thiophene-7-carboxamide
3-(4-((3-fluorobenzyl)amino)-6-methyl-1,3,5-triazin-2-yl)-3a,7a-dihydrobenzo[b]thiophene-7-carboxamide
3-(4-(benzylamino)-6-methyl-1,3,5-triazin-2-yl)-3a,7a-dihydrobenzo[b]thiophene-7-carboxamide
3-(4-((3-fluorobenzyl)amino)-6-methyl-1,3,5-triazin-2-yl)-3a,7a-dihydrobenzo[b]thiophene-7-carboxamide
3-(4-(benzylamino)-6-methyl-1,3,5-triazin-2-yl)-3a,7a-dihydrobenzo[b]thiophene-7-carboxamide
3-(4-((3-fluorobenzyl)amino)-6-methyl-1,3,5-triazin-2-yl)-3a,7a-dihydrobenzo[b]thiophene-7-carboxamide
3-(4-(benzylamino)-6-methyl-1,3,5-triazin-2-yl)-3a,7a-dihydrobenzo[b]thiophene-7-carboxamide
3-(4-((3-fluorobenzyl)amino)-6-(dimethylamino)-1,3,5-triazin-2-yl)-3a,7a-dihydrobenzo[b]thiophene-7-carboxamide
3-(4-(benzylamino)-6-(dimethylamino)1-1,3,5-triazin-2-yl)-3a,7a-dihydrobenzo[b]thiophene-7-carboxamide
3-(4-((3-fluorobenzyl)amino)-6-methyl-1,3,5-triazin-2-yl)-3a,7a-dihydrobenzo[b]thiophene-7-carboxamide
3-(4-(benzylamino)-6-methyl-1,3,5-triazin-2-yl)-3a,7a-dihydrobenzo[b]thiophene-7-carboxamide

### 3a,7a-dihydrobenzo[d]isothiazole

3-(5-((3-fluorobenzyl)amino)-6-carbamoyl-1,2,4-triazin-3-yl)-3a,7a-dihydrobenzo[d]isothiazole-7-carboxamide
3-(5-(benzylamino)-6-carbamoyl-1,2,4-triazin-3-yl)-3a,7a-dihydrobenzo[d]isothiazole-7-carboxamide
3-(5-((3-fluorobenzyl)amino)-6-ethyl-1,2,4-triazin-3-yl)-3a,7a-dihydrobenzo[d]isothiazole-7-carboxamide
3-(5-(benzylamino)-6-ethyl-1,2,4-triazin-3-yl)-3a,7a-dihydrobenzo[d]isothiazole-7-carboxamide
3-(5-((3-fluorobenzyl)amino)-1,2,4-triazin-3-yl)-3a,7a-dihydrobenzo[d]isothiazole-7-carboxamide
3-(5-(benzylamino)-1,2,4-triazin-3-yl)-3a,7a-dihydrobenzo[d]isothiazole-7-carboxamide
3-(5-((3-fluorobenzyl)amino)-6-methyl-1,2,4-triazin-3-yl)-3a,7a-dihydrobenzo[d]isothiazole-7-carboxamide
3-(5-(benzylamino)-6-methyl-1,2,4-triazin-3-yl)-3a,7a-dihydrobenzo[d]isothiazole-7-carboxamide
3-(5-((3-fluorobenzyl)amino)-6-(dimethylamino)-1,2,4-triazin-3-yl)-3a,7a-dihydrobenzo[d]isothiazole-7-carboxamide
3-(5-(benzylamino)-6-(dimethylamino)-1,2,4-triazin-3-yl)-3a,7a-dihydrobenzo[d]isothiazole-7-carboxamide
3-(5-((3-fluorobenzyl)amino)-6-methoxy-1,2,4-triazin-3-yl)-3a,7a-dihydrobenzo[d]isothiazole-7-carboxamide
3-(5-(benzylamino)-6-methoxy-1,2,4-triazin-3-yl)-3a,7a-dihydrobenzo[d]isothiazole-7-carboxamide
3-(4-((3-fluorobenzyl)amino)-6-carbamoyl-1,3,5-triazin-2-yl)-3a,7a-dihydrobenzo[d]isothiazole-7-carboxamide
3-(4-(benzylamino)-6-carbamoyl-1,3,5-triazin-2-yl)-3a,7a-dihydrobenzo[d]isothiazole-7-carboxamide
3-(4-((3-fluorobenzyl)amino)-6-methyl-1,3,5-triazin-2-yl)-3a,7a-dihydrobenzo[d]isothiazole-7-carboxamide
3-(4-(benzylamino)-6-methyl-1,3,5-triazin-2-yl)-3a,7a-dihydrobenzo[d]isothiazole-7-carboxamide
3-(4-((3-fluorobenzyl)amino)-6-methyl-1,3,5-triazin-2-yl)-3a,7a-dihydrobenzo[d]isothiazole-7-carboxamide
3-(4-(benzylamino)-6-methyl-1,3,5-triazin-2-yl)-3a,7a-dihydrobenzo[d]isothiazole-7-carboxamide
3-(4-((3-fluorobenzyl)amino)-6-methyl-1,3,5-triazin-2-yl)-3a,7a-dihydrobenzo[d]isothiazole-7-carboxamide
3-(4-(benzylamino)-6-methyl-1,3,5-triazin-2-yl)-3a,7a-dihydrobenzo[d]isothiazole-7-carboxamide
3-(4-((3-fluorobenzyl)amino)-6-(dimethylamino)-1,3,5-triazin-2-yl)-3a,7a-dihydrobenzo[d]isothiazole-7-carboxamide
3-(4-(benzylamino)-6-(dimethylamino)1-1,3,5-triazin-2-yl)-3a,7a-dihydrobenzo[d]isothiazole-7-carboxamide
3-(4-((3-fluorobenzyl)amino)-6-methyl-1,3,5-triazin-2-yl)-3a,7a-dihydrobenzo[d]isothiazole-7-carboxamide
3-(4-(benzylamino)-6-methyl-1,3,5-triazin-2-yl)-3a,7a-dihydrobenzo[d]isothiazole-7-carboxamide
3-(5-((3-fluorobenzyl)amino)-6-carbamoyl-1,2,4-triazin-3-yl)-3a,7a-dihydrobenzo[d]isoxazole-7-carboxamide
3-(5-(benzylamino)-6-carbamoyl-1,2,4-triazin-3-yl)-3a,7a-dihydrobenzo[d]isoxazole-7-carboxamide
3-(5-((3-fluorobenzyl)amino)-6-ethyl-1,2,4-triazin-3-yl)-3a,7a-dihydrobenzo[d]isoxazole-7-carboxamide
3-(5-(benzylamino)-6-ethyl-1,2,4-triazin-3-yl)-3a,7a-dihydrobenzo[d]isoxazole-7-carboxamide
3-(5-((3-fluorobenzyl)amino)-1,2,4-triazin-3-yl)-3a,7a-dihydrobenzo[d]isoxazole-7-carboxamide
3-(5-(benzylamino)-1,2,4-triazin-3-yl)-3a,7a-dihydrobenzo[d]isoxazole-7-carboxamide
3-(5-((3-fluorobenzyl)amino)-6-methyl-1,2,4-triazin-3-yl)-3a,7a-dihydrobenzo[d]isoxazole-7-carboxamide
3-(5-(benzylamino)-6-methyl-1,2,4-triazin-3-yl)-3a,7a-dihydrobenzo[d]isoxazole-7-carboxamide
3-(5-((3-fluorobenzyl)amino)-6-(dimethylamino)-1,2,4-triazin-3-yl)-3a,7a-dihydrobenzo[d]isoxazole-7-carboxamide
3-(5-(benzylamino)-6-(dimethylamino)-1,2,4-triazin-3-yl)-3a,7a-dihydrobenzo[d]isoxazole-7-carboxamide
3-(5-((3-fluorobenzyl)amino)-6-methoxy-1,2,4-triazin-3-yl)-3a,7a-dihydrobenzo[d]isoxazole-7-carboxamide
3-(5-(benzylamino)-6-methoxy-1,2,4-triazin-3-yl)-3a,7a-dihydrobenzo[d]isoxazole-7-carboxamide
3-(4-((3-fluorobenzyl)amino)-6-carbamoyl-1,3,5-triazin-2-yl)-3a,7a-dihydrobenzo[d]isoxazole-7-carboxamide
3-(4-(benzylamino)-6-carbamoyl-1,3,5-triazin-2-yl)-3a,7a-dihydrobenzo[d]isoxazole-7-carboxamide
3-(4-((3-fluorobenzyl)amino)-6-methyl-1,3,5-triazin-2-yl)-3a,7a-dihydrobenzo[d]isoxazole-7-carboxamide
3-(4-(benzylamino)-6-methyl-1,3,5-triazin-2-yl)-3a,7a-dihydrobenzo[d]isoxazole-7-carboxamide
3-(4-((3-fluorobenzyl)amino)-6-methyl-1,3,5-triazin-2-yl)-3a,7a-dihydrobenzo[d]isoxazole-7-carboxamide
3-(4-(benzylamino)-6-methyl-1,3,5-triazin-2-yl)-3a,7a-dihydrobenzo[d]isoxazole-7-carboxamide
3-(4-((3-fluorobenzyl)amino)-6-methyl-1,3,5-triazin-2-yl)-3a,7a-dihydrobenzo[d]isoxazole-7-carboxamide
3-(4-(benzylamino)-6-methyl-1,3,5-triazin-2-yl)-3a,7a-dihydrobenzo[d]isoxazole-7-carboxamide
3-(4-((3-fluorobenzyl)amino)-6-(dimethylamino)-1,3,5-triazin-2-yl)-3a,7a-dihydrobenzo[d]isoxazole-7-carboxamide
3-(4-(benzylamino)-6-(dimethylamino)1-1,3,5-triazin-2-yl)-3a,7a-dihydrobenzo[d]isoxazole-7-carboxamide
3-(4-((3-fluorobenzyl)amino)-6-methyl-1,3,5-triazin-2-yl)-3a,7a-dihydrobenzo[d]isoxazole-7-carboxamide
3-(4-(benzylamino)-6-methyl-1,3,5-triazin-2-yl)-3a,7a-dihydrobenzo[d]isoxazole-7-carboxamide
3a,7a-dihydrobenzofuran
3-(5-((3-fluorobenzyl)amino)-6-carbamoyl-1,2,4-triazin-3-yl)-3a,7a-dihydrobenzofuran-7-carboxamide
3-(5-(benzylamino)-6-carbamoyl-1,2,4-triazin-3-yl)-3a,7a-dihydrobenzofuran-7-carboxamide
3-(5-((3-fluorobenzyl)amino)-6-ethyl-1,2,4-triazin-3-yl)-3a,7a-dihydrobenzofuran-7-carboxamide
3-(5-(benzylamino)-6-ethyl-1,2,4-triazin-3-yl)-3a,7a-dihydrobenzofuran-7-carboxamide
3-(5-((3-fluorobenzyl)amino)-1,2,4-triazin-3-yl)-3a,7a-dihydrobenzofuran-7-carboxamide
3-(5-(benzylamino)-1,2,4-triazin-3-yl)-3a,7a-dihydrobenzofuran-7-carboxamide
3-(5-((3-fluorobenzyl)amino)-6-methyl-1,2,4-triazin-3-yl)-3a,7a-dihydrobenzofuran-7-carboxamide
3-(5-(benzylamino)-6-methyl-1,2,4-triazin-3-yl)-3a,7a-dihydrobenzofuran-7-carboxamide
3-(5-((3-fluorobenzyl)amino)-6-(dimethylamino)-1,2,4-triazin-3-yl)-3a,7a-dihydrobenzofuran-7-carboxamide
3-(5-(benzylamino)-6-(dimethylamino)-1,2,4-triazin-3-yl)-3a,7a-dihydrobenzofuran-7-carboxamide
3-(5-((3-fluorobenzyl)amino)-6-methoxy-1,2,4-triazin-3-yl)-3a,7a-dihydrobenzofuran-7-carboxamide
3-(5-(benzylamino)-6-methoxy-1,2,4-triazin-3-yl)-3a,7a-dihydrobenzofuran-7-carboxamide
3-(4-((3-fluorobenzyl)amino)-6-carbamoyl-1,3,5-triazin-2-yl)-3a,7a-dihydrobenzofuran-7-carboxamide
3-(4-(benzylamino)-6-carbamoyl-1,3,5-triazin-2-yl)-3a,7a-dihydrobenzofuran-7-carboxamide
3-(4-((3-fluorobenzyl)amino)-6-methyl-1,3,5-triazin-2-yl)-3a,7a-dihydrobenzofuran-7-carboxamide
3-(4-(benzylamino)-6-methyl-1,3,5-triazin-2-yl)-3a,7a-dihydrobenzofuran-7-carboxamide
3-(4-((3-fluorobenzyl)amino)-6-methyl-1,3,5-triazin-2-yl)-3a,7a-dihydrobenzofuran-7-carboxamide
3-(4-(benzylamino)-6-methyl-1,3,5-triazin-2-yl)-3a,7a-dihydrobenzofuran-7-carboxamide
3-(4-((3-fluorobenzyl)amino)-6-methyl-1,3,5-triazin-2-yl)-3a,7a-dihydrobenzofuran-7-carboxamide
3-(4-(benzylamino)-6-methyl-1,3,5-triazin-2-yl)-3a,7a-dihydrobenzofuran-7-carboxamide
3-(4-((3-fluorobenzyl)amino)-6-(dimethylamino)-1,3,5-triazin-2-yl)-3a,7a-dihydrobenzofuran-7-carboxamide
3-(4-(benzylamino)-6-(dimethylamino)-1,3,5-triazin-2-yl)-3a,7a-dihydrobenzofuran-7-carboxamide
3-(4-((3-fluorobenzyl)amino)-6-methyl-1,3,5-triazin-2-yl)-3a,7a-dihydrobenzofuran-7-carboxamide
3-(4-(benzylamino)-6-methyl-1,3,5-triazin-2-yl)-3a,7a-dihydrobenzofuran-7-carboxamide

### Benzo[c]isothiazole

3-(5-((3-fluorobenzyl)amino)-6-carbamoyl-1,2,4-triazin-3-yl)-benzo[c]isothiazole-7-carboxamide
3-(5-(benzylamino)-6-carbamoyl-1,2,4-triazin-3-yl)-benzo[c]isothiazole-7-carboxamide
3-(5-((3-fluorobenzyl)amino)-6-ethyl-1,2,4-triazin-3-yl)-benzo[c]isothiazole-7-carboxamide
3-(5-(benzylamino)-6-ethyl-1,2,4-triazin-3-yl)-benzo[c]isothiazole-7-carboxamide
3-(5-((3-fluorobenzyl)amino)-1,2,4-triazin-3-yl)-benzo[c]isothiazole-7-carboxamide
3-(5-(benzylamino)-1,2,4-triazin-3-yl)-benzo[c]isothiazole-7-carboxamide
3-(5-((3-fluorobenzyl)amino)-6-methyl-1,2,4-triazin-3-yl)-benzo[c]isothiazole-7-carboxamide
3-(5-(benzylamino)-6-methyl-1,2,4-triazin-3-yl)-benzo[c]isothiazole-7-carboxamide
3-(5-((3-fluorobenzyl)amino)-6-(dimethylamino)-1,2,4-triazin-3-yl)-benzo[c]isothiazole-7-carboxamide
3-(5-(benzylamino)-6-(dimethylamino)-1,2,4-triazin-3-yl)-benzo[c]isothiazole-7-carboxamide
3-(5-((3-fluorobenzyl)amino)-6-methoxy-1,2,4-triazin-3-yl)-benzo[c]isothiazole-7-carboxamide
3-(5-(benzylamino)-6-methoxy-1,2,4-triazin-3-yl)-benzo[c]isothiazole-7-carboxamide
3-(4-((3-fluorobenzyl)amino)-6-carbamoyll-1,3,5-triazin-2-yl)benzo[c]isothiazole-7-carboxamide
3-(4-(benzylamino)-6-carbamoyll-1,3,5-triazin-2-yl)benzo[c]isothiazole-7-carboxamide
3-(4-((3-fluorobenzyl)amino)-6-ethyl-1,3,5-triazin-2-yl)benzo[c]isothiazole-7-carboxamide
3-(4-(benzylamino)-6-ethyl-1,3,5-triazin-2-yl)benzo[c]isothiazole-7-carboxamide
3-(4-((3-fluorobenzyl)amino)-1,3,5-triazin-2-yl)benzo[c]isothiazole-7-carboxamide
3-(4-(benzylamino)-1,3,5-triazin-2-yl)benzo[c]isothiazole-7-carboxamide
3-(4-((3-fluorobenzyl)amino)-6-methyl-1,3,5-triazin-2-yl)benzo[c]isothiazole-7-carboxamide
3-(4-(benzylamino)-6-methyl-1,3,5-triazin-2-yl)benzo[c]isothiazole-7-carboxamide
3-(4-((3-fluorobenzyl)amino)-6-(dimethylamino)-1,3,5-triazin-2-yl)benzo[c]isothiazole-7-carboxamide
3-(4-(benzylamino)-6-(dimethylamino)-1,3,5-triazin-2-yl)benzo[c]isothiazole-7-carboxamide
3-(4-((3-fluorobenzyl)amino)-6-methoxy-1,3,5-triazin-2-yl)benzo[c]isothiazole-7-carboxamide
3-(4-(benzylamino)-6-methoxy-1,3,5-triazin-2-yl)benzo[c]isothiazole-7-carboxamide
3-(5-((3-fluorobenzyl)amino)-6-carbamoyl-1,2,4-triazin-3-yl)-benzo[c]isoxazole-7-carboxamide
3-(5-(benzylamino)-6-carbamoyl-1,2,4-triazin-3-yl)-benzo[c]isoxazole-7-carboxamide
3-(5-((3-fluorobenzyl)amino)-6-ethyl-1,2,4-triazin-3-yl)-benzo[c]isoxazole-7-carboxamide
3-(5-(benzylamino)-6-ethyl-1,2,4-triazin-3-yl)-benzo[c]isoxazole-7-carboxamide
3-(5-((3-fluorobenzyl)amino)-1,2,4-triazin-3-yl)-benzo[c]isoxazole-7-carboxamide
3-(5-(benzylamino)-1,2,4-triazin-3-yl)-benzo[c]isoxazole-7-carboxamide
3-(5-((3-fluorobenzyl)amino)-6-methyl-1,2,4-triazin-3-yl)-benzo[c]isoxazole-7-carboxamide
3-(5-(benzylamino)-6-methyl-1,2,4-triazin-3-yl)-benzo[c]isoxazole-7-carboxamide
3-(5-((3-fluorobenzyl)amino)-6-(dimethylamino)-1,2,4-triazin-3-yl)-benzo[c]isoxazole-7-carboxamide
3-(5-(benzylamino)-6-(dimethylamino)-1,2,4-triazin-3-yl)-benzo[c]isoxazole-7-carboxamide
3-(5-((3-fluorobenzyl)amino)-6-methoxy-1,2,4-triazin-3-yl)-benzo[c]isoxazole-7-carboxamide
3-(5-(benzylamino)-6-methoxy-1,2,4-triazin-3-yl)-benzo[c]isoxazole-7-carboxamide
3-(4-((3-fluorobenzyl)amino)-6-carbamoyll-1,3,5-triazin-2-yl)benzo[c]isoxazole-7-carboxamide
3-(4-(benzylamino)-6-carbamoyll-1,3,5-triazin-2-yl)benzo[c]isoxazole-7-carboxamide
3-(4-((3-fluorobenzyl)amino)-6-ethyl-1,3,5-triazin-2-yl)benzo[c]isoxazole-7-carboxamide
3-(4-(benzylamino)-6-ethyl-1,3,5-triazin-2-yl)benzo[c]isoxazole-7-carboxamide
3-(4-((3-fluorobenzyl)amino)-1,3,5-triazin-2-yl)benzo[c]isoxazole-7-carboxamide
3-(4-(benzylamino)-1,3,5-triazin-2-yl)benzo[c]isoxazole-7-carboxamide
3-(4-((3-fluorobenzyl)amino)-6-methyl-1,3,5-triazin-2-yl)benzo[c]isoxazole-7-carboxamide
3-(4-(benzylamino)-6-methyl-1,3,5-triazin-2-yl)benzo[c]isoxazole-7-carboxamide
3-(4-((3-fluorobenzyl)amino)-6-(dimethylamino)-1,3,5-triazin-2-yl)benzo[c]isoxazole-7-carboxamide
3-(4-(benzylamino)-6-(dimethylamino)-1,3,5-triazin-2-yl)benzo[c]isoxazole-7-carboxamide
3-(4-((3-fluorobenzyl)amino)-6-methoxy-1,3,5-triazin-2-yl)benzo[c]isoxazole-7-carboxamide
3-(4-(benzylamino)-6-methoxy-1,3,5-triazin-2-yl)benzo[c]isoxazole-7-carboxamide

### Benzo[c]thiophene

1-(5-((3-fluorobenzyl)amino)-6-carbamoyl-1,2,4-triazin-3-yl)-benzo[c]thiophene-4-carboxamide
1-(5-(benzylamino)-6-carbamoyl-1,2,4-triazin-3-yl)-benzo[c]thiophene-4-carboxamide
1-(5-((3-fluorobenzyl)amino)-6-ethyl-1,2,4-triazin-3-yl)-benzo[c]thiophene-4-carboxamide
1-(5-(benzylamino)-6-ethyl-1,2,4-triazin-3-yl)-benzo[c]thiophene-4-carboxamide
1-(5-((3-fluorobenzyl)amino)-1,2,4-triazin-3-yl)-benzo[c]thiophene-4-carboxamide
1-(5-(benzylamino)-1,2,4-triazin-3-yl)-benzo[c]thiophene-4-carboxamide
1-(5-((3-fluorobenzyl)amino)-6-methyl-1,2,4-triazin-3-yl)-benzo[c]thiophene-4-carboxamide
1-(5-(benzylamino)-6-methyl-1,2,4-triazin-3-yl)-benzo[c]thiophene-4-carboxamide
1-(5-((3-fluorobenzyl)amino)-6-(dimethylamino)-1,2,4-triazin-3-yl)-benzo[c]thiophene-4-carboxamide
1-(5-(benzylamino)-6-(dimethylamino)-1,2,4-triazin-3-yl)-benzo[c]thiophene-4-carboxamide
1-(5-((3-fluorobenzyl)amino)-6-methoxy-1,2,4-triazin-3-yl)-benzo[c]thiophene-4-carboxamide
1-(5-(benzylamino)-6-methoxy-1,2,4-triazin-3-yl)-benzo[c]thiophene-4-carboxamide
1-(4-((3-fluorobenzyl)amino)-6-carbamoyl-1,3,5-triazin-2-yl)-benzo[c]thiophene-4-carboxamide
1-(4-(benzylamino)-6-carbamoyl-1,3,5-triazin-2-yl)-benzo[c]thiophene-4-carboxamide
1-(4-((3-fluorobenzyl)amino)-6-ethyl-1,3,5-triazin-2-yl)-benzo[c]thiophene-4-carboxamide
1-(4-(benzylamino)-6-ethyl-1,3,5-triazin-2-yl)-benzo[c]thiophene-4-carboxamide
1-(4-((3-fluorobenzyl)amino)-1,3,5-triazin-2-yl)-benzo[c]thiophene-4-carboxamide
1-(4-(benzylamino)-1,3,5-triazin-2-yl)-benzo[c]thiophene-4-carboxamide
1-(4-((3-fluorobenzyl)amino)-6-methyl-1,3,5-triazin-2-yl)-benzo[c]thiophene-4-carboxamide
1-(4-(benzylamino)-6-methyl-1,3,5-triazin-2-yl)-benzo[c]thiophene-4-carboxamide
1-(4-((3-fluorobenzyl)amino)-6-(dimethylamino)-1,3,5-triazin-2-yl)-benzo[c]thiophene-4-carboxamide
1-(4-(benzylamino)-6-(dimethylamino)-1,3,5-triazin-2-yl)-benzo[c]thiophene-4-carboxamide
1-(4-((3-fluorobenzyl)amino)-6-methoxy-1,3,5-triazin-2-yl)-benzo[c]thiophene-4-carboxamide
1-(4-(benzylamino)-6-methoxy-1,3,5-triazin-2-yl)-benzo[c]thiophene-4-carboxamide
Imidazo[1,2-a]pyridine
(3-(((3-(4-carbamoyl-2-methylimidazo[1,2-a]pyridin-3-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-2-methylimidazo[1,2-a]pyridine-8-carboxamide
3-(5-(benzylamino)-6-carbamoyl-1,2,4-triazin-3-yl)-2-methylimidazo[1,2-a]pyridine-8-carboxamide
3-(5-((3-fluorobenzyl)amino)-6-ethyl-1,2,4-triazin-3-yl)-2-methylimidazo[1,2-a]pyridine-8-carboxamide
3-(5-(benzylamino)-6-ethyl-1,2,4-triazin-3-yl)-2-methylimidazo[1,2-a]pyridine-8-carboxamide
(3-(((3-(4-carbamoyl-2-methylimidazo[1,2-a]pyridin-3-yl)-6-ethyl-1,2,4-triazin-5-yl)amino)methyl)phenyl)boronic acid
(3-(((3-(4-carbamoyl-2-methylimidazo[1,2-a]pyridin-3-yl)-1,2,4-triazin-5-yl)amino)methyl)phenyl)boronic acid
3-(5-((3-fluorobenzyl)amino)-1,2,4-triazin-3-yl)-2-methylimidazo[1,2-a]pyridine-8-carboxamide
3-(5-(benzylamino)-1,2,4-triazin-3-yl)-2-methylimidazo[1,2-a]pyridine-8-carboxamide
3-(5-((3-fluorobenzyl)amino)-6-methyl-1,2,4-triazin-3-yl)-2-methylimidazo[1,2-a]pyridine-8-carboxamide
3-(5-(benzylamino)-6-methyl-1,2,4-triazin-3-yl)-2-methylimidazo[1,2-a]pyridine-8-carboxamide
(3-(((3-(4-carbamoyl-2-methylimidazo[1,2-a]pyridin-3-yl)-6-methyl-1,2,4-triazin-5-yl)amino)methyl)phenyl)boronic acid
(3-(((3-(4-carbamoyl-2-methylimidazo[1,2-a]pyridin-3-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-2-methylimidazo[1,2-a]pyridine-8-carboxamide
3-(5-(benzylamino)-6-(dimethylamino)-1,2,4-triazin-3-yl)-2-methylimidazo[1,2-a]pyridine-8-carboxamide
(3-(((3-(4-carbamoyl-2-methylimidazo[1,2-a]pyridin-3-yl)-6-methoxy-1,2,4-triazin-5-yl)amino)methyl)phenyl)boronic acid
3-(5-((3-fluorobenzyl)amino)-6-methoxy-1,2,4-triazin-3-yl)-2-methylimidazo[1,2-a]pyridine-8-carboxamide
3-(5-(benzylamino)-6-methoxy-1,2,4-triazin-3-yl)-2-methylimidazo[1,2-a]pyridine-8-carboxamide
(3-(((4-(8-carbamoyl-2-methylimidazo[1,2-a]pyridin-3-yl)-6-carbamoyl-1,3,5-triazin-2-yl)amino)methyl)phenyl)boronic acid
3-(4-((3-fluorobenzyl)amino))-6-carbamoyl-1,3,5-triazin-2-yl)-2-methylimidazo[1,2-a]pyridine-8-carboxamide
3-(4-(benzylamino)-6-mcarbamoyl-1,3,5-triazin-2-yl)-2-methylimidazo[1,2-a]pyridine-8-carboxamide
3-(4-((3-fluorobenzyl)amino))-6-ethyl-1,3,5-triazin-2-yl)-2-methylimidazo[1,2-a]pyridine-8-carboxamide
3-(4-(benzylamino)-6-ethyl-1,3,5-triazin-2-yl)-2-methylimidazo[1,2-a]pyridine-8-carboxamide
(3-(((4-(8-carbamoyl-2-methylimidazo[1,2-a]pyridin-3-yl)-6-ethyl-1,3,5-triazin-2-yl)amino)methyl)phenyl)boronic acid
(3-(((4-(8-carbamoyl-2-methylimidazo[1,2-a]pyridin-3-yl)-6-1,3,5-triazin-2-yl)amino)methyl)phenyl)boronic acid
3-(4-((3-fluorobenzyl)amino))-1,3,5-triazin-2-yl)-2-methylimidazo[1,2-a]pyridine-8-carboxamide
3-(4-(benzylamino)-1,3,5-triazin-2-yl)-2-methylimidazo[1,2-a]pyridine-8-carboxamide
3-(4-((3-fluorobenzyl)amino))-6-methyl-1,3,5-triazin-2-yl)-2-methylimidazo[1,2-a]pyridine-8-carboxamide
3-(4-(benzylamino)-6-methyl-1,3,5-triazin-2-yl)-2-methylimidazo[1,2-a]pyridine-8-carboxamide
(3-(((4-(8-carbamoyl-2-methylimidazo[1,2-a]pyridin-3-yl)-6-methyl-1,3,5-triazin-2-yl)amino)methyl)phenyl)boronic acid
(3-(((4-(8-carbamoyl-2-methylimidazo[1,2-a]pyridin-3-yl)-6-(dimethylamino)-1,3,5-triazin-2-yl)amino)methyl)phenyl)boronic acid
3-(4-((3-fluorobenzyl)amino))-6-(dimethylamino)-1,3,5-triazin-2-yl)-2-methylimidazo[1,2-a]pyridine-8-carboxamide
3-(4-(benzylamino)-6-(dimethylamino)-1,3,5-triazin-2-yl)-2-methylimidazo[1,2-a]pyridine-8-carboxamide
(3-(((4-(8-carbamoyl-2-methylimidazo[1,2-a]pyridin-3-yl)-6-methoxy-1,3,5-triazin-2-yl)amino)methyl)phenyl)boronic acid
3-(4-((3-fluorobenzyl)amino))-6-methoxy-1,3,5-triazin-2-yl)-2-methylimidazo[1,2-a]pyridine-8-carboxamide
3-(4-(benzylamino)-6-methoxy-1,3,5-triazin-2-yl)-2-methylimidazo[1,2-a]pyridine-8-carboxamide

### Imidazo[1,5-a]pyridine

(3-(((3-(4-carbamoyl-imidazo[1,5-a]pyridin-3-yl)-6-mcarbamoyl-1,2,4-triazin-5-yl)amino)methyl)phenyl)boronic acid
(3-(((3-(5-carbamoyl-imidazo[1,5-a]pyridin-1-yl)-6-mcarbamoyl-1,2,4-triazin-5-yl)amino)methyl)phenyl)boronic acid
3-(5-((3-fluorobenzyl)amino)-6-carbamoyl-1,2,4-triazin-3-yl)-imidazo[1,5-a]pyridine-8-carboxamide
1-(5-((3-fluorobenzyl)amino)-6-carbamoyl-1,2,4-triazin-3-yl)-imidazo[1,5-a]pyridine-5-carboxamide
1-(5-(benzylamino)-6-carbamoyl-1,2,4-triazin-3-yl)-imidazo[1,5-a]pyridine-5-carboxamide
3-(5-((3-fluorobenzyl)amino)-6-ethyl-1,2,4-triazin-3-yl)-imidazo[1,5-a]pyridine-8-carboxamide
1-(5-((3-fluorobenzyl)amino)-6-ethyl-1,2,4-triazin-3-yl)-imidazo[1,5-a]pyridine-5-carboxamide
1-(5-(benzylamino)-6-ethyl-1,2,4-triazin-3-yl)-imidazo[1,5-a]pyridine-5-carboxamide
(3-(((3-(4-carbamoyl-imidazo[1,5-a]pyridin-3-yl)-6-ethyl-1,2,4-triazin-5-yl)amino)methyl)phenyl)boronic acid
(3-(((3-(5-carbamoyl-imidazo[1,5-a]pyridin-1-yl)-6-ethyl-1,2,4-triazin-5-yl)amino)methyl)phenyl)boronic acid
(3-(((3-(4-carbamoyl-imidazo[1,5-a]pyridin-3-yl)-1,2,4-triazin-5-yl)amino)methyl)phenyl)boronic acid
(3-(((3-(5-carbamoyl-imidazo[1,5-a]pyridin-1-yl)-1,2,4-triazin-5-yl)amino)methyl)phenyl)boronic acid
3-(5-((3-fluorobenzyl)amino)-1,2,4-triazin-3-yl)-imidazo[1,5-a]pyridine-8-carboxamide
1-(5-((3-fluorobenzyl)amino)-1,2,4-triazin-3-yl)-imidazo[1,5-a]pyridine-5-carboxamide
1-(5-(benzylamino)-1,2,4-triazin-3-yl)-imidazo[1,5-a]pyridine-5-carboxamide
3-(5-((3-fluorobenzyl)amino)-6-methyl-1,2,4-triazin-3-yl)-imidazo[1,5-a]pyridine-8-carboxamide
1-(5-((3-fluorobenzyl)amino)-6-methyl-1,2,4-triazin-3-yl)-imidazo[1,5-a]pyridine-5-carboxamide
1-(5-(benzylamino)-6-methyl-1,2,4-triazin-3-yl)-imidazo[1,5-a]pyridine-5-carboxamide
(3-(((3-(4-carbamoyl-imidazo[1,5-a]pyridin-3-yl)-6-methyl-1,2,4-triazin-5-yl)amino)methyl)phenyl)boronic acid
(3-(((3-(5-carbamoyl-imidazo[1,5-a]pyridin-1-yl)-6-methyl-1,2,4-triazin-5-yl)amino)methyl)phenyl)boronic acid
(3-(((3-(4-carbamoyl-imidazo[1,5-a]pyridin-3-yl)-6-(dimethylamino)-1,2,4-triazin-5-yl)amino)methyl)phenyl)boronic acid
(3-(((3-(5-carbamoyl-imidazo[1,5-a]pyridin-1-yl)-6-(dimethylamino)-1,2,4-triazin-5-yl)amino)methyl)phenyl)boronic acid
3-(5-((3-fluorobenzyl)amino)-6-(dimethylamino)-1,2,4-triazin-3-yl)-imidazo[1,5-a]pyridine-8-carboxamide
1-(5-((3-fluorobenzyl)amino)-6-(dimethylamino)-1,2,4-triazin-3-yl)-imidazo[1,5-a]pyridine-5-carboxamide
1-(5-(benzylamino)-6-(dimethylamino)-1,2,4-triazin-3-yl)-imidazo[1,5-a]pyridine-5-carboxamide
(3-(((3-(4-carbamoyl-imidazo[1,5-a]pyridin-3-yl)-6-methoxy-1,2,4-triazin-5-yl)amino)methyl)phenyl)boronic acid
(3-(((3-(5-carbamoyl-imidazo[1,5-a]pyridin-1-yl)-6-methoxy-1,2,4-triazin-5-yl)amino)methyl)phenyl)boronic acid
3-(5-((3-fluorobenzyl)amino)-6-methoxy-1,2,4-triazin-3-yl)-imidazo[1,5-a]pyridine-8-carboxamide
1-(5-((3-fluorobenzyl)amino)-6-methoxy-1,2,4-triazin-3-yl)-imidazo[1,5-a]pyridine-5-carboxamide
1-(5-(benzylamino)-6-methoxy-1,2,4-triazin-3-yl)-imidazo[1,5-a]pyridine-5-carboxamide
(3-(((4-(8-carbamoylimidazo[1,5-a]pyridin-3-yl)-6-carbamoyl-1,3,5-triazin-2-yl)amino)methyl)phenyl)boronic acid
(3-(((4-(5-carbamoyl-imidazo[1,5-a]pyridin-1-yl)-6-mcarbamoyl-1,3,5-triazin-2-yl)amino)methyl)phenyl)boronic acid
3-(4-((3-fluorobenzyl)amino)-6-carbamoyl-1,3,5-triazin-2-yl)imidazo[1,5-a]pyridine-8-carboxamide
1-(4-((3-fluorobenzyl)amino)-6-carbamoyl-1,3,5-triazin-2-yl)-imidazo[1,5-a]pyridine-5-carboxamide
1-(4-(benzylamino)-6-carbamoyl-1,3,5-triazin-2-yl)-imidazo[1,5-a]pyridine-5-carboxamide
3-(4-((3-fluorobenzyl)amino)-6-ethyl-1,3,5-triazin-2-yl)imidazo[1,5-a]pyridine-8-carboxamide
1-(4-((3-fluorobenzyl)amino)-6-ethyl-1,3,5-triazin-2-yl)-imidazo[1,5-a]pyridine-5-carboxamide
1-(4-(benzylamino)-6-ethyl-1,3,5-triazin-2-yl)-imidazo[1,5-a]pyridine-5-carboxamide
(3-(((4-(8-carbamoylimidazo[1,5-a]pyridin-3-yl)-6-ethyl-1,3,5-triazin-2-yl)amino)methyl)phenyl)boronic acid
(3-(((4-(5-carbamoyl-imidazo[1,5-a]pyridin-1-yl)-6-ethyl-1,3,5-triazin-2-yl)amino)methyl)phenyl)boronic acid
(3-(((4-(8-carbamoylimidazo[1,5-a]pyridin-3-yl)-1,3,5-triazin-2-yl)amino)methyl)phenyl)boronic acid
(3-(((4-(5-carbamoyl-imidazo[1,5-a]pyridin-1-yl)-1,3,5-triazin-2-yl)amino)methyl)phenyl)boronic acid
3-(4-((3-fluorobenzyl)amino)-1,3,5-triazin-2-yl)imidazo[1,5-a]pyridine-8-carboxamide
1-(4-((3-fluorobenzyl)amino)-1,3,5-triazin-2-yl)-imidazo[1,5-a]pyridine-5-carboxamide
1-(4-(benzylamino)-1,3,5-triazin-2-yl)-imidazo[1,5-a]pyridine-5-carboxamide
3-(4-((3-fluorobenzyl)amino)-6-methyl-1,3,5-triazin-2-yl)imidazo[1,5-a]pyridine-8-carboxamide
1-(4-((3-fluorobenzyl)amino)-6-methyl-1,3,5-triazin-2-yl)-imidazo[1,5-a]pyridine-5-carboxamide
1-(4-(benzylamino)-6-methyl-1,3,5-triazin-2-yl)-imidazo[1,5-a]pyridine-5-carboxamide
(3-(((4-(8-carbamoylimidazo[1,5-a]pyridin-3-yl)-6-methyl-1,3,5-triazin-2-yl)amino)methyl)phenyl)boronic acid
(3-(((4-(5-carbamoyl-imidazo[1,5-a]pyridin-1-yl)-6-methyl-1,3,5-triazin-2-yl)amino)methyl)phenyl)boronic acid
(3-(((4-(8-carbamoylimidazo[1,5-a]pyridin-3-yl)-6-(dimethylamino)-1,3,5-triazin-2-yl)amino)methyl)phenyl)boronic acid
(3-(((4-(5-carbamoyl-imidazo[1,5-a]pyridin-1-yl)-6-(dimethylamino)-1,3,5-triazin-2-yl)amino)methyl)phenyl)boronic acid
3-(4-((3-fluorobenzyl)amino)-6-(dimethylamino)-1,3,5-triazin-2-yl)imidazo[1,5-a]pyridine-8-carboxamide
1-(4-((3-fluorobenzyl)amino)-6-(dimethylamino)-1,3,5-triazin-2-yl)-imidazo[1,5-a]pyridine-5-carboxamide
1-(4-(benzylamino)-6-(dimethylamino)-1,3,5-triazin-2-yl)-imidazo[1,5-a]pyridine-5-carboxamide
(3-(((4-(8-carbamoylimidazo[1,5-a]pyridin-3-yl)-6-methoxy-1,3,5-triazin-2-yl)amino)methyl)phenyl)boronic acid
(3-(((4-(5-carbamoyl-imidazo[1,5-a]pyridin-1-yl)-6-methoxy-1,3,5-triazin-2-yl)amino)methyl)phenyl)boronic acid
3-(4-((3-fluorobenzyl)amino)-6-methoxy-1,3,5-triazin-2-yl)imidazo[1,5-a]pyridine-8-carboxamide
1-(4-((3-fluorobenzyl)amino)-6-methoxy-1,3,5-triazin-2-yl)-imidazo[1,5-a]pyridine-5-carboxamide
1-(4-(benzylamino)-6-methoxy-1,3,5-triazin-2-yl)-imidazo[1,5-a]pyridine-5-carboxamide
Isobenzofuran
1-(5-((3-fluorobenzyl)amino)-6-carbamoyl-1,2,4-triazin-3-yl)-isobenzofuran-4-carboxamide
1-(5-(benzylamino)-6-carbamoyl-1,2,4-triazin-3-yl)-isobenzofuran-4-carboxamide
1-(5-((3-fluorobenzyl)amino)-6-ethyl-1,2,4-triazin-3-yl)-isobenzofuran-4-carboxamide
1-(5-(benzylamino)-6-ethyl-1,2,4-triazin-3-yl)-isobenzofuran-4-carboxamide
1-(5-((3-fluorobenzyl)amino)-1,2,4-triazin-3-yl)-isobenzofuran-4-carboxamide
1-(5-(benzylamino)-1,2,4-triazin-3-yl)-isobenzofuran-4-carboxamide
1-(5-((3-fluorobenzyl)amino)-6-methyl-1,2,4-triazin-3-yl)-isobenzofuran-4-carboxamide
1-(5-(benzylamino)-6-methyl-1,2,4-triazin-3-yl)-isobenzofuran-4-carboxamide
1-(5-((3-fluorobenzyl)amino)-6-(dimethylamino)-1,2,4-triazin-3-yl)-isobenzofuran-4-carboxamide
1-(5-(benzylamino)-6-(dimethylamino)-1,2,4-triazin-3-yl)-isobenzofuran-4-carboxamide
1-(5-((3-fluorobenzyl)amino)-6-methoxy-1,2,4-triazin-3-yl)-isobenzofuran-4-carboxamide
1-(5-(benzylamino)-6-methoxy-1,2,4-triazin-3-yl)-isobenzofuran-4-carboxamide
1-(4-((3-fluorobenzyl)amino)-6-carbamoyl-1,3,5-triazin-2-yl)isobenzofuran-4-carboxamide
1-(4-(benzylamino)-6-carbamoyl-1,3,5-triazin-2-yl)isobenzofuran-4-carboxamide
1-(4-((3-fluorobenzyl)amino)-6-ethyl-1,3,5-triazin-2-yl)isobenzofuran-4-carboxamide
1-(4-(benzylamino)-6-ethyl-1,3,5-triazin-2-yl)isobenzofuran-4-carboxamide
1-(4-((3-fluorobenzyl)amino)-1,3,5-triazin-2-yl)isobenzofuran-4-carboxamide
1-(4-(benzylamino)-1,3,5-triazin-2-yl)isobenzofuran-4-carboxamide
1-(4-((3-fluorobenzyl)amino)-6-methyl-1,3,5-triazin-2-yl)isobenzofuran-4-carboxamide
1-(4-(benzylamino)-6-methyl-1,3,5-triazin-2-yl)isobenzofuran-4-carboxamide
1-(4-((3-fluorobenzyl)amino)-6-(dimethylamino)-1,3,5-triazin-2-yl)isobenzofuran-4-carboxamide
1-(4-(benzylamino)-6-(dimethylamino)-1,3,5-triazin-2-yl)isobenzofuran-4-carboxamide
1-(4-((3-fluorobenzyl)amino)-6-methoxy-1,3,5-triazin-2-yl)isobenzofuran-4-carboxamide
1-(4-(benzylamino)-6-methoxy-1,3,5-triazin-2-yl)isobenzofuran-4-carboxamide

### Most Preferred

1-(5-(benzylamino)-6-ethyl-1,2,4-triazin-3-yl)-1H-indazole-4-carboxamide
1-(5-(benzylamino)-6-methyl-1,2,4-triazin-3-yl)-1H-indazole-4-carboxamide
3-(4-((3-fluorobenzyl)amino)-6-ethyl-1,3,5-triazin-2-yl)-1H-indazole-7-carboxamide
3-(4-(benzylamino)-6-ethyl-1,3,5-triazin-2-yl)-1H-indazole-7-carboxamide
3-(4-((3-fluorobenzyl)amino)-6-methyl-1,3,5-triazin-2-yl)-1H-indazole-7-carboxamide
3-(4-(benzylamino)-6-methyl-1,3,5-triazin-2-yl)-1H-indazole-7-carboxamide
(1-(5-((3-fluorobenzyl)amino)-6-ethyl-1,2,4-triazin-3-yl)-2-methyl-1H-indole-4-yl)boronic acid
(1-(5-(benzylamino)-6-ethyl-1,2,4-triazin-3-yl)-2-methyl-1H-indole-4-yl)boronic acid
(3-(((3-(4-carbamoyl-2-methyl-1H-indol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-2-methyl-1H-indole-4-carboxamide
1-(5-(benzylamino)-6-ethyl-1,2,4-triazin-3-yl)-2-methyl-1H-indole-4-carboxamide
(1-(5-((3-fluorobenzyl)amino)-6-methyl-1,2,4-triazin-3-yl)-2-methyl-1H-indole-4-yl)boronic acid
(1-(5-(benzylamino)-6-methyl-1,2,4-triazin-3-yl)-2-methyl-1H-indole-4-yl)boronic acid
(3-(((3-(4-carbamoyl-2-methyl-1H-indol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-2-methyl-1H-indole-4-carboxamide
1-(5-(benzylamino)-6-methyl-1,2,4-triazin-3-yl)-2-methyl-1H-indole-4-carboxamide
(1-(4-((3-fluorobenzyl)amino)-6-ethyl-1,3,5-triazin-2-yl)-2-methyl-1H-indol-4-yl)boronic acid
(1-(4-(benzylamino)-6-ethyl-1,3,5-triazin-2-yl)-2-methyl-1H-indol-4-yl)boronic acid
(3-(((4-(4-carbamoyl-2-methyl-1H-indol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-1H-indole-4-carboxamide
1-(4-(benzylamino)-6-ethyl-1,3,5-triazin-2-yl)-2-methyl-1H-indole-4-carboxamide
1-(4-((3-fluorobenzyl)amino)-6-ethyl-1,3,5-triazin-2-yl)-2-methyl-1H-indole-4-sulfonamide
1-(4-(benzylamino)-6-ethyl-1,3,5-triazin-2-yl)-2-methyl-1H-indole-4-sulfonamide
(1-(4-((3-fluorobenzyl)amino)-6-methyl-1,3,5-triazin-2-yl)-2-methyl-1H-indol-4-yl)boronic acid
(1-(4-(benzylamino)-6-methyl-1,3,5-triazin-2-yl)-2-methyl-1H-indol-4-yl)boronic acid
(3-(((4-(4-carbamoyl-2-methyl-1H-indol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-1H-indole-4-carboxamide
1-(4-(benzylamino)-6-methyl-1,3,5-triazin-2-yl)-2-methyl-1H-indole-4-carboxamide
1-(4-((3-fluorobenzyl)amino)-6-methyl-1,3,5-triazin-2-yl)-2-methyl-1H-indole-4-sulfonamide
1-(4-(benzylamino)-6-methyl-1,3,5-triazin-2-yl)-2-methyl-1H-indole-4-sulfonamide
3-(5-(benzylamino)-6-ethyl-1,2,4-triazin-3-yl)-2-methyl-1H-indole-7-carboxamide
3-(5-(benzylamino)-6-methyl-1,2,4-triazin-3-yl)-2-methyl-1H-indole-7-carboxamide
3-(4-((3-fluorobenzyl)amino)-6-ethyl-1,3,5-triazin-2-yl)-2-methyl-1H-indole-7-carboxamide
3-(4-(benzylamino)-6-ethyl-1,3,5-triazin-2-yl)-2-methyl-1H-indole-7-carboxamide
3-(4-((3-fluorobenzyl)amino)-6-methyl-1,3,5-triazin-2-yl)-2-methyl-1H-indole-7-carboxamide
3-(4-(benzylamino)-6-methyl-1,3,5-triazin-2-yl)-2-methyl-1H-indole-7-carboxamide
1-(5-(benzylamino)-6-ethyl-1,2,4-triazin-3-yl)-2-methoxy-1H-benzo[d]imidazole-4-carboxamide
1-(5-(benzylamino)-6-methyl-1,2,4-triazin-3-yl)-2-methoxy-1H-benzo[d]imidazole-4-carboxamide
1-(4-((3-fluorobenzyl)amino)-6-ethyl-1,3,5-triazin-2-yl)-2-methoxy-1H-benzo[d]imidazole-4-carboxamide
1-(4-(benzylamino)-6-ethyl-1,3,5-triazin-2-yl)-2-methoxy-1H-benzo[d]imidazole-4-carboxamide
1-(4-((3-fluorobenzyl)amino)-6-methyl-1,3,5-triazin-2-yl)-2-methoxy-1H-benzo[d]imidazole-4-carboxamide
1-(4-(benzylamino)-6-methyl-1,3,5-triazin-2-yl)-2-methoxy-1H-benzo[d]imidazole-4-carboxamide

### Synthetic Preparation

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

The nitrogen hexacycle compounds can be prepared by the literature methods cited in the following text. The following schemes depict established, known syntheses of these scaffolds.

The substituents of the nitrogen hexacycle compounds can be synthesized and attached to these scaffolds by the literature methods cited in the following text. The following schemes depict the known techniques for accomplishing this joinder.

### General Synthetic Schemes for Triazines:

Compounds of the present invention can be synthesized using the following methods. General reaction conditions are given and reaction products can be purified by general known methods including crystallization, silica gel chromatography using various organic solvents such as hexane, cyclohexane, ethyl acetate, methanol and the like, preparative high pressure liquid chromatography or preparative reverse phase high pressure liquid chromatography.

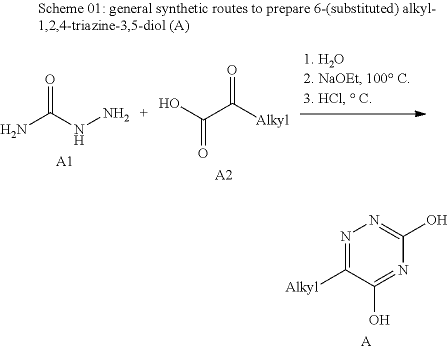

Multiple synthetic routes to prepare 6-(substituted)alkyl-1,2,4-triazine-3,5-diols A have been reported and a common approach is to treat hydrazinecarboxamide and alfa-ketocarboxylic acids as descripted in the literatures such as Hannah L. Maslen, J. Med. Chem. 47, 5482, 2004.

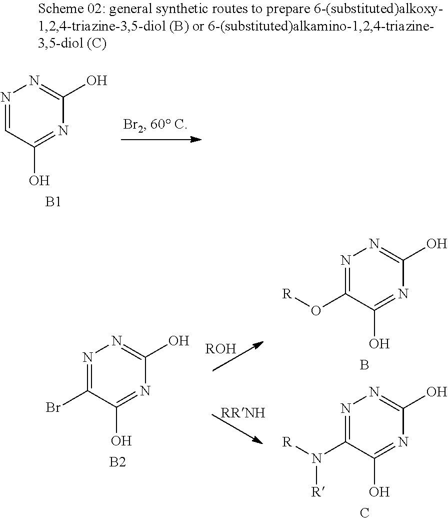

1,2,4-triazine-3,5-diol B1 can be easily converted into 6-bromo-1,2,4-triazine-3,5-diol B2, then the latter can react with alcohols or amines to insert ether or amine groups into its 6-position.

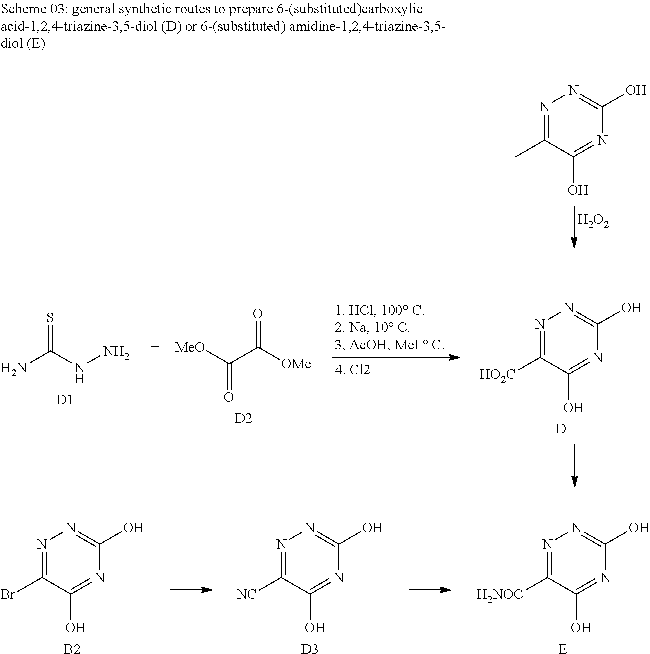

Multiple synthetic routes to prepare 6-carboxylic-1,2,4-triazine-3,5-diol D have been reported. Simple oxidation of 6-methyl-1,2,4-triazine-3,5-diol offered a good yield of the acid, alternatively, hydrazinecarbothioamide reacted with dimethyl oxalate followed by treatment with chlorine as descripted in Shieru Furukubo et al, PCT Int. Appl. 2007063934 yielded the same product. The amides E can be prepared from acid D or nitrile D3, which was prepared from substitution of Br within the intermediate B2.

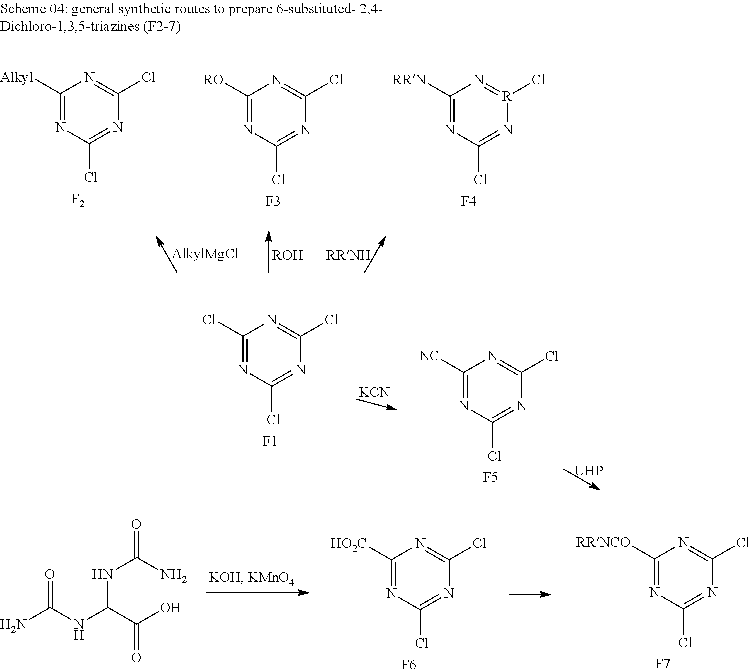

General approaches to prepare intermediates of 6-substituted-2,4-dichloro-1,3,5-triazines (F2-7) was summarized herein. 2,4,6-Trichloro-1,3,5-nitrogen hexacycle F1 reacted with Grignard reagents to yield 6-alkyl intermediates F2. F1 reacted with alcohols or amines to achieve 6-substituted ethers F3 or amines F4, respectively. One of chlorine of F1 can be converted into cyano group F5 and then the latter can be transferred into amide F7. Alternatively, 2,2-diureidoacetic acid was coverted to acid F6 by oxidation as descripted in M. Poje, Tetrahedron, 44, 6723, 1988, the latter can be used for preparation of amides as well.

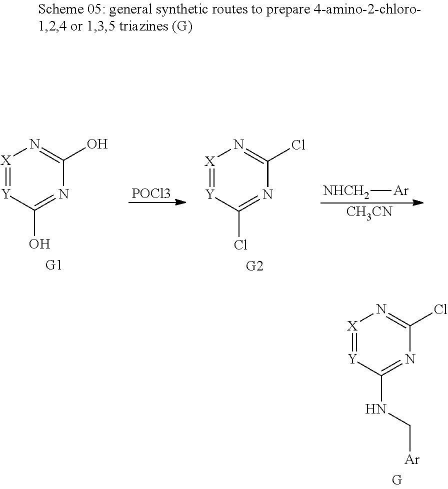

Triazinediones can be reacted with an excess of POCl₃ at reflux for 3-12 hours optionally in the presence of a tertiary amine such as triethyl amine, diisopropyl ethyl amine or dimethyl analine to give the fused dicholortriazines of the general structure. Other chlorinating agents such as thionyl chloride or PCl₅ can be substituted for POCl₃.

Dicholortriazines of the general structure can be reacted with excess amounts of various substituted amines at temperatures ranging from room temperature to reflux in a solvent such as acetonitrile or dimethylformamide to give amino-substituted-2-chloro triazines of the general structure G.

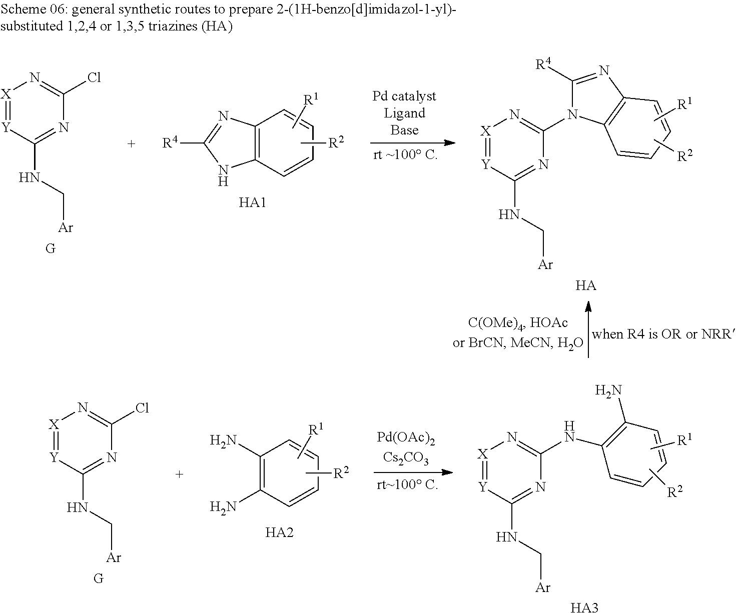

A general synthetic approach to install benzo[d]imidazole HA through its 1-position into the 2-position of 1,3,5-triazines or 3-position of 1,2,4-triazines to yield the desired molecules HA is Pd-based coupling reaction. A common condition is Pd(dba)₂ as a transition metal catalyst and X-phos as a ligand and cesium carbonate as a base and dioxane an organic solvent. The reaction temperature varies from the room temperature to reflux. For example, if amino is Boc-protected nitrogen, an extra step to deBoc can be achieved to the desired final products. For example if R¹ is a nitrile (CN) it can be converted to an amide in the presence of urea hydrogen peroxide (UHP). Alternatively, in some cases of R⁴ is alkoxy or amino groups, coupling reaction can be take place between the 2 or 3-position of triazines and benzene-1,2-diamines HA2 suing Pd(OAc)₂ as the catalyst and CsCO₃ as the base, then cyclization can occur with either bromocyanide or tetramethoxymethane.

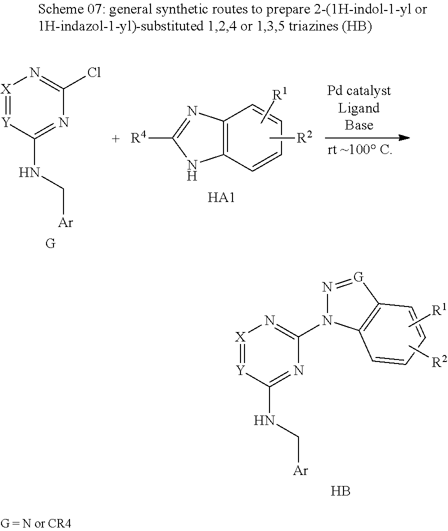

Coupling to a solution of the substituted triazines with an indole or indazole HB1 can be effective to achieve the desired molecules HB using methods similar to that described in Zhou, H.-J. et. al. WO 2014015291. To a solution of substituted triazine such as G is added a indole or a substituted ones such as HB1 and a base such as sodium carbonate or cesium carbonate, in the presence of a palladium catalyst such as Pd(OAc)₂ and a ligand such as triphenylphosphine in a solvent such as dioxane and the reaction can optionally be heated to reflux for up to 48 hours.

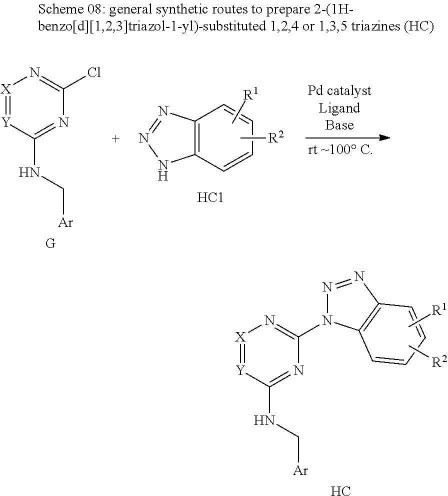

Compounds containing the structure of triazines-2 or 3-yl-1,2,3-benzotriazoles HC can be produced using methods similar to those described in Zhou, H.-J. et. al. WO 2014015291 and Ohlmeyer, Michael J. et al WO 2008060301 and as described above by using a substituted 1,2,3-benzotriazole HCl.

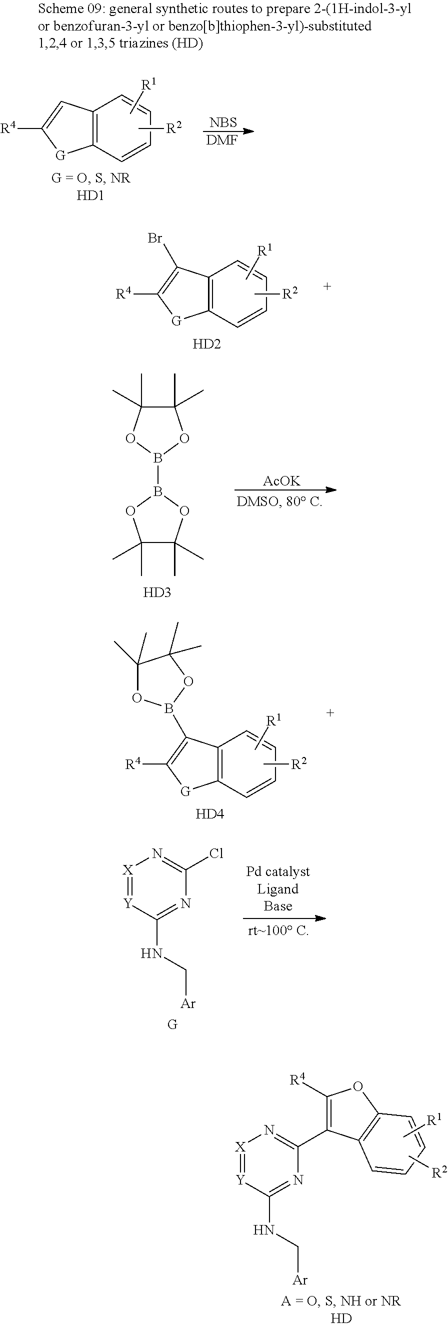

Under various conditions such as NBS in DMF, bromination of 3-unsubstituted intermediates HD1 (X═O, S, either substituted or properly protected nitrogen) would take place region-selectively on their 3-position to yield 3-Br-substituted intermediates HD2. They then can be converted into boronic esters HD4 by treatment with boronic ester HD3 under various conditions. Then Pd-based coupling reaction similar to those described in Zhou, H.-J. et. al. WO 2014015291 between intermediates HD4 and G provided the desired molecules. For example if A is protected nitrogen, an extra step to deprotection can be achieved using reported conditions.

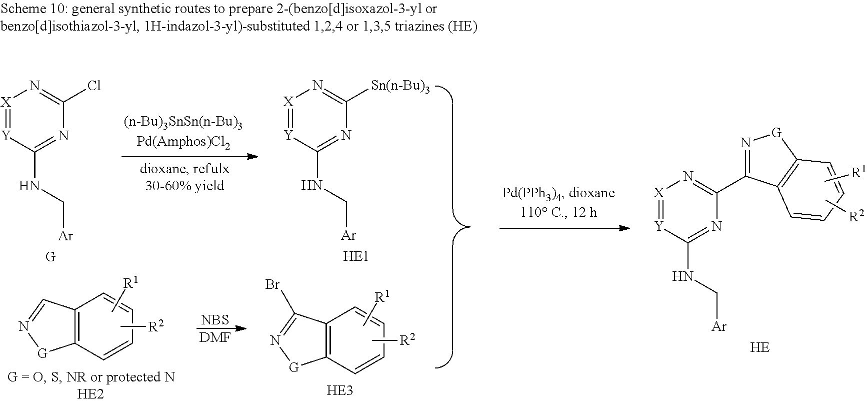

The key intermediate HE1, tributyl-nitrogen hexacycle-2 or -3-tin can be prepared from the intermediates G following the similar procedure in the reference (Castanedo, Georgette et al, PCT Int. Appl., 2010138589). Bromonation can occur selectively into the 3-position of these 5,6-bicycloaromatic rings HE2. Then Pd-based coupling reaction similar to those described in Zhou, H.-J. et. al. WO 2014015291 between intermediates HE1 and HE3 provided the desired molecules HE. For example if A is protected nitrogen, an extra step to deprotection can be achieved using reported conditions.

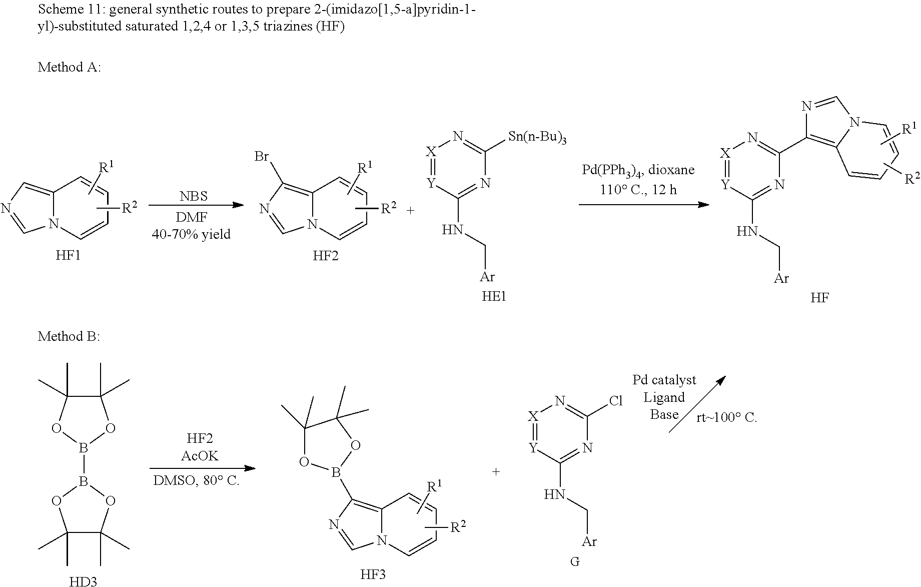

Bromonation can occur selectively into the 3-position of imidazo[1,5-a]pyridine HF1. Then Pd-based coupling reaction similar to those described above between intermediates HE1 and HF2 provided the desired molecules HF. Alternatively, bromides HF2 can be converted into boronic esters HF3 by treatment with boronic ester HD3 under various conditions. Then Pd-based coupling reaction similar to those described above between intermediates HF3 and G provided the desired molecules.

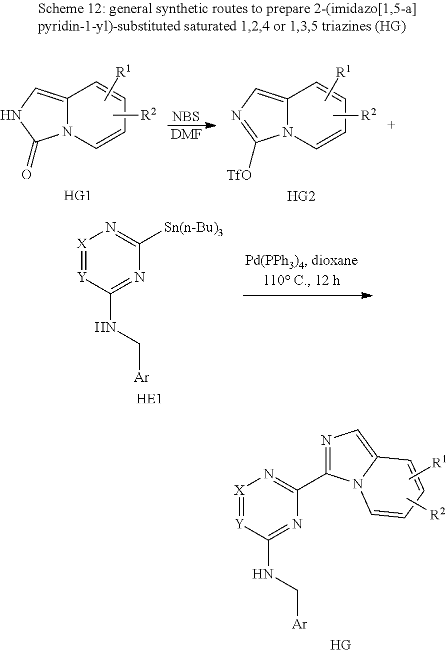

Imidazo[1,5-a]pyridin-3(2H)-one HG1 can be converted into its triflate HG2. Then Pd-based coupling reaction similar to those described above between intermediates HE1 and HG2 provided the desired molecules HG.

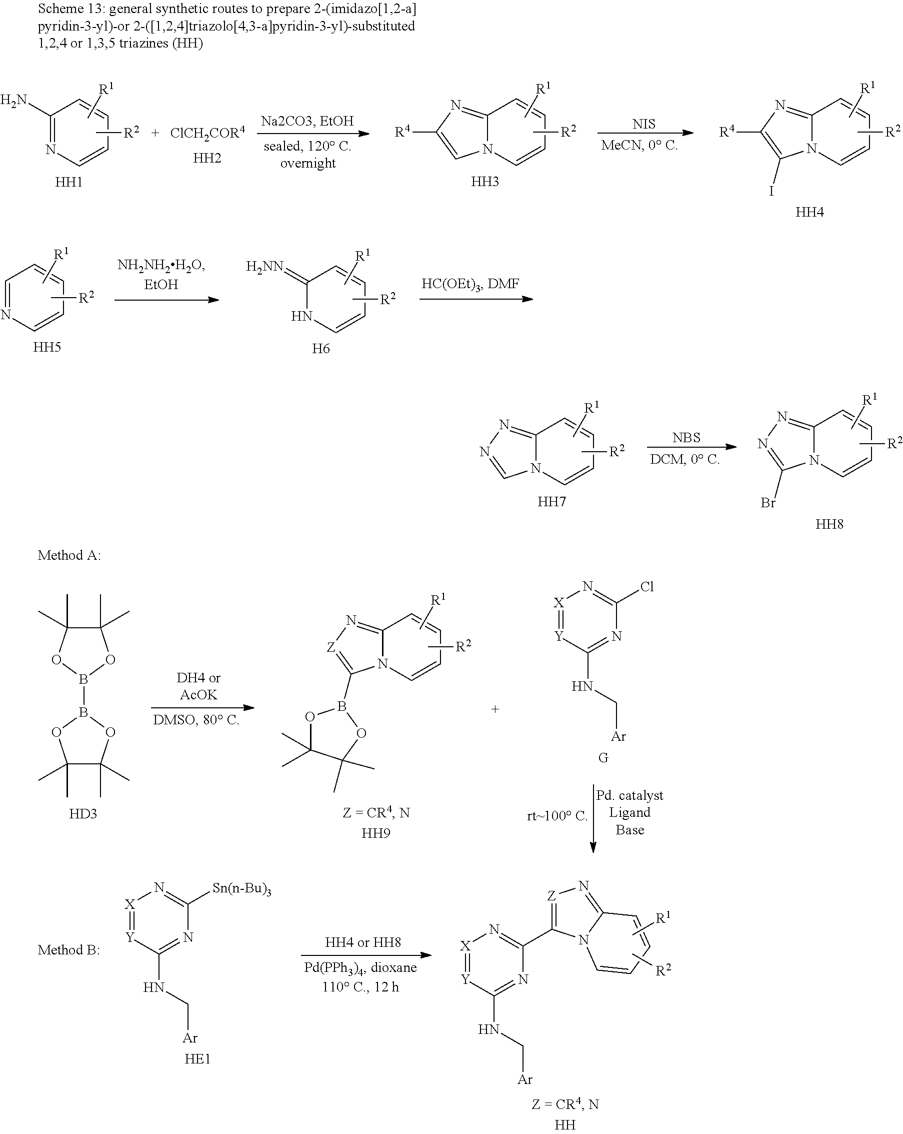

Imidazo[1,2-a]pyridine HH3 can be prepared by treatment 2-aminopyridine HH1 with aldehydes or ketones HH2 by a method similar to those described in the references such as Ebetino, Frank Hallock et al. PCT Int. Appl., 2010033978. Iodination with NIS can yield 3-I-Imidazo[1,2-a]pyridine HH4 by a method similar to those described in the references such as Bifulco, Neil, Jr. et al. PCT Int. Appl., 2014011900. [1,2,4]triazolo[4,3-a]pyridine-8-carbonitrile HH7 can be prepared by substituted pyridine HH5 in a two-step procedures, reaction with hydrazine followed by treatment with triethoxymethane by a method similar to those described in the references such as such as Allen, Shelley et al, PCT Int. Appl., 2010022076; Potts, K. T. and Burton, H. R., Journal of Organic Chemistry, 31(1), 251-60; 1966. Then bromination with NBS can yield the intermediate HH8. HH4 or HH8 can be converted into boronic esters HH9 then by treatment with boronic ester HD3 under various conditions. Then Pd-based coupling reaction similar to those described above between intermediates HH9 and G provided the desired molecules HH. Alternatively, Pd-based coupling reaction similar to those described above between intermediates HE1 and HH4 or HH8 provided the desired molecules HH as well.

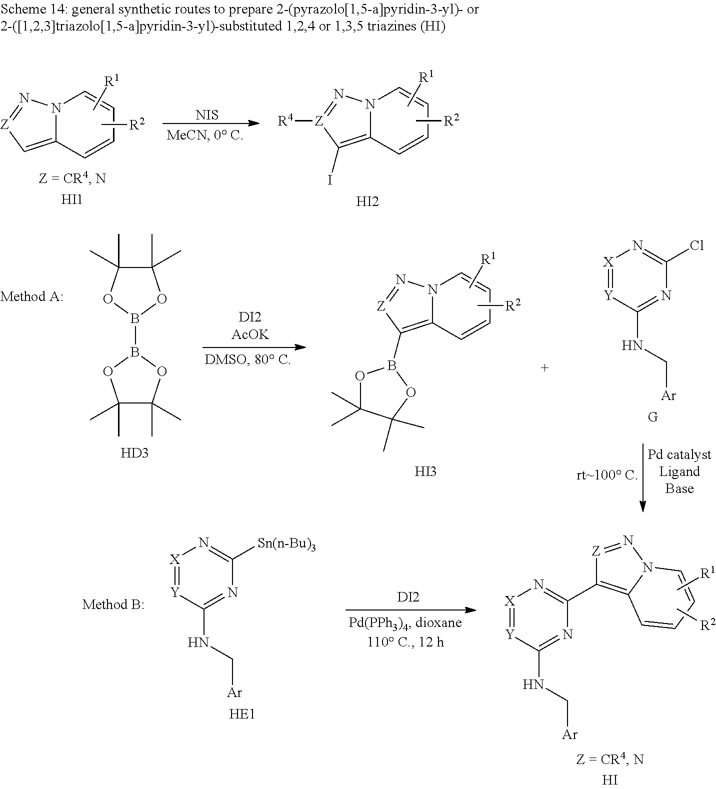

Various methods can be used to prepare substituted 3-pyrimidin-2-ylpyrazolo[1,5-a]pyridines HI1 (Z═CR⁴) as outlined in Tsuchiya, et. al. Chemical & Pharmaceutical Bulletin 1983, 31, 4568; Hajos, G. and Riedl, Z. Science of Synthesis, 2002, 12, 613 and Aboul-Fadl, T. et al. Synthesis, 2000, 12, 1727-1732. Various methods can be used to prepare substituted [1,2,3]triazolo[1,5-a]pyridine DI1 (Z═N) as outlined in Latham, Elliot J. and Stanforth, Stephen P. Journal of Heterocyclic Chemistry, 32(3), 787-9; 199; Sheng et al, Organic Letters, 14(14), 3744-3747; 2012 and Prakash, Om et al, Synthetic Communications, 30(3), 417-425; 2000. Iodination with NIS can yield 3-iodo-pyrazolo[1,5-a]pyridine HI2 by a method similar to those described in the references such as Bifulco, Neil, Jr. et al. PCT Int. Appl., 2014011900; Wan, Huixin et al, PCT Int. Appl., 2013170774, 21 Nov. 2013. HI2 can be then converted into boronic esters HI3 then by treatment with boronic ester HD3 under various conditions. Then Pd-based coupling reaction similar to those described above between intermediates HI3 and G provided the desired molecules HI. Alternatively, Pd-based coupling reaction similar to those described above between intermediates HE1 and HI2 provided the desired molecules HI as well.

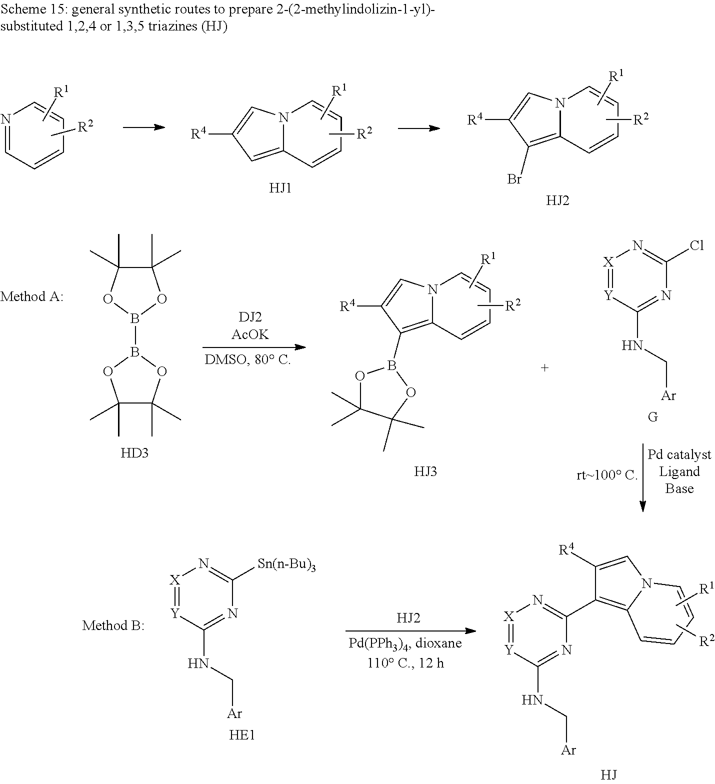

1-Bromo-lindolizine HJ2 can be prepare from substituted pyridine using methods as outlined in Iizuka, Masato and Shimizu, Kazuo, PCT Int. Appl., 2012043638. HJ2 can be then converted into boronic esters HJ3 then by treatment with boronic ester HD3 under various conditions. Then Pd-based coupling reaction similar to those described above between intermediates HJ3 and G provided the desired molecules HJ. Alternatively, Pd-based coupling reaction similar to those described above between intermediates HE1 and HJ2 provided the desired molecules HI as well.

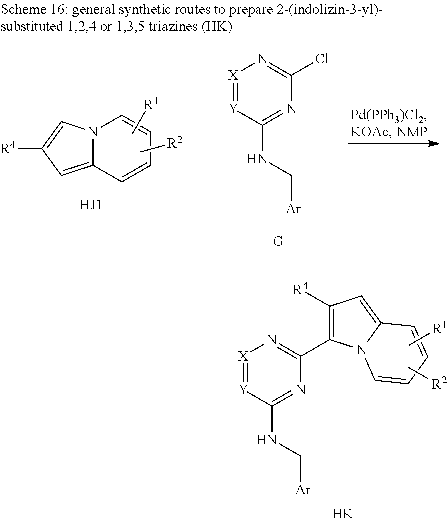

Pd-based coupling reaction between HJ1 and G using approaches similar to those described above provided the desired molecules HK.

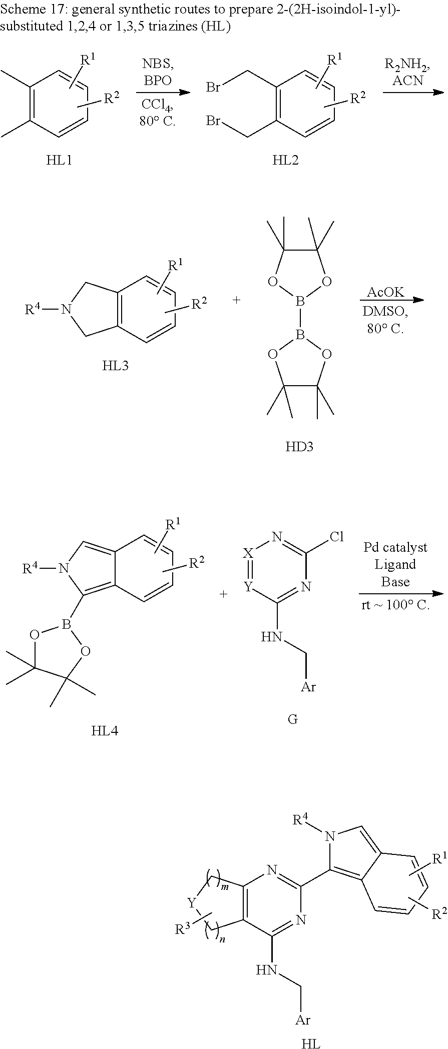

The boronic ester of 2H-isoindole HL4 can be prepared from substituted 1,2-dimethylbenzene HL1 following the procedure in the reference (Ohmura, Toshimichi et al Journal of the American Chemical Society, 131(17), 6070-6071; 2009). Then Pd-based coupling reaction similar to those described above between intermediates HL4 and G provided the desired molecules HL.

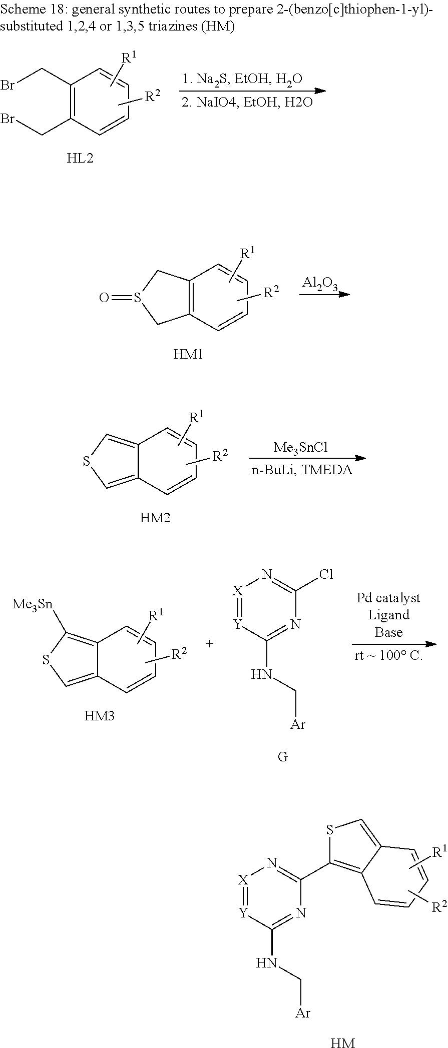

The key intermediates HM3, benzo[c]thiophen-1-yltrimethylstannane can be prepared from the intermediates HL2 following the similar procedure in the reference (Kawabata, Kohsuke and Goto, Hiromasa, Journal of Materials Chemistry, 22(44), 23514-23524; 2012). Then Pd-based coupling reaction similar to those described above between intermediates HM3 and G provided the desired molecules HM.

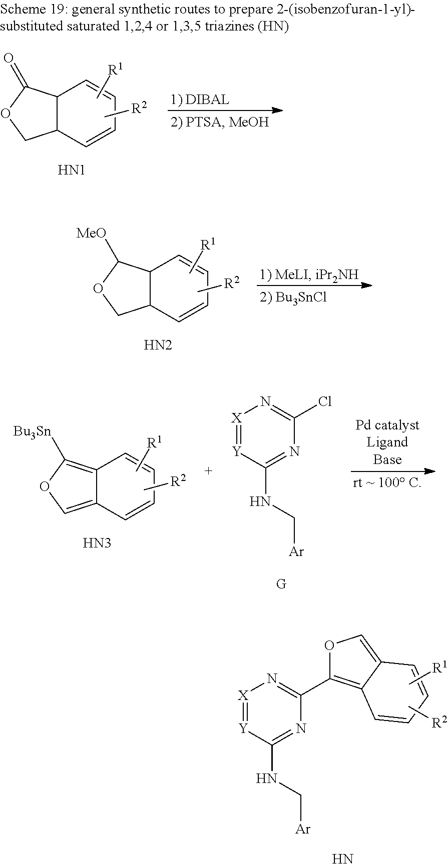

The key intermediates HN3, tributyl(isobenzofuran-1-yl)stannane can be prepared from the intermediates HN2, which can be prepared from lactone HN1. Then Pd-based coupling reaction similar to those described above between intermediates HN3 and G provided the desired molecules HN.

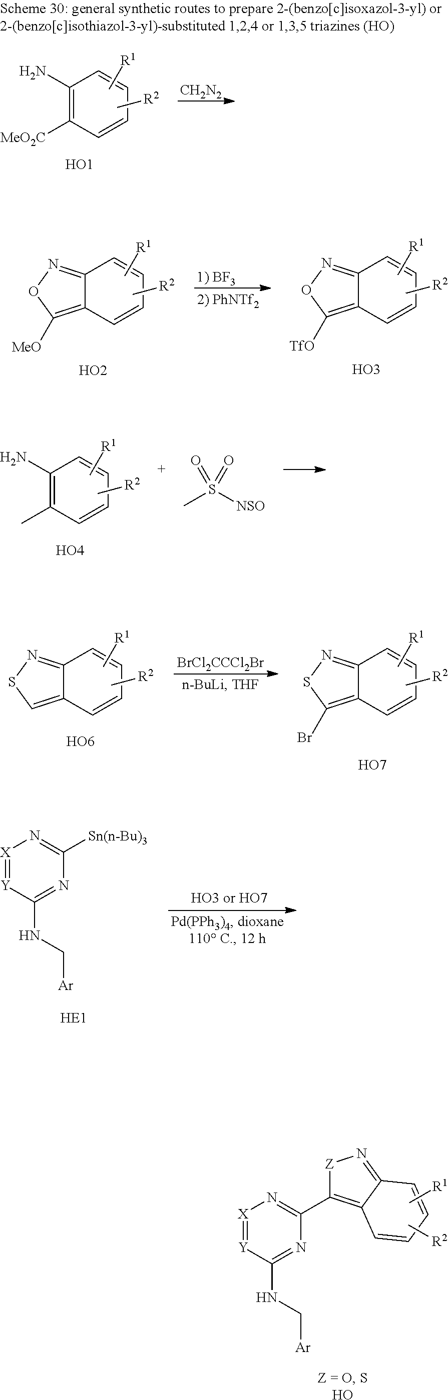

Substituted 3-methoxybenzo[c]isoxazoles HO2 can be prepared from 2-aminobenzoates HO1 following the similar procedure in the references such as Chauhan, Mohinder S. and McKinnon, David M., Canadian Journal of Chemistry, 53(9), 1336-42; Smalley, R. K., Science of Synthesis, 11, 337-382; 2002. Then demethylation and conversion hydroxyl into triflate group can yield the intermediate HO3. Substituted benzo[c]isothiazole HO6 can be prepared from substituted o-toluidine DO4 following the similar procedure in the references such as Puetz, Claudia et al, Eur. Pat. Appl., 1352910. Then bromination with BNS can selectively into Br into its position HO7. Then Pd-based coupling reaction similar to those described above between intermediates HO3 or HO7 and G provided the desired molecules HO.

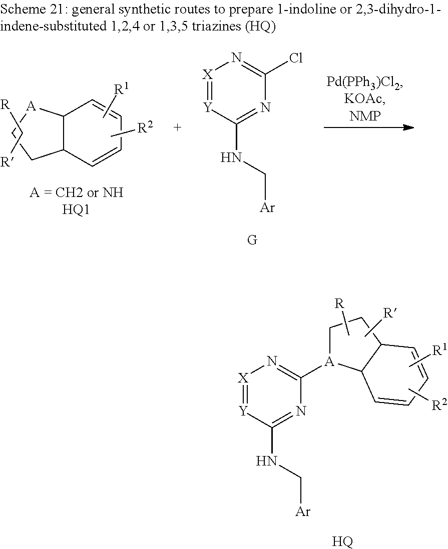

Pd-based coupling reaction between HQ1 and G using approaches similar to those described above provided the desired molecules HQ.

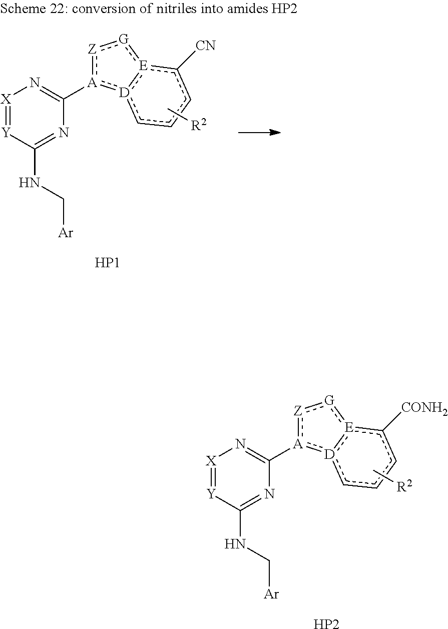

In some cases the desired compounds HP1 prepared in Schemes 6-20 above can have a nitrile substitution at the position indicated in Scheme 21. This substituent can be converted to the corresponding carboxamide HP2. Nitriles HP1 are dissolved in a 1/10 ratio of water/DMSO and treated with urea-hydrogen peroxide (UHP) and a base such as potassium carbonate. Reaction mixture is stirred at room temperature for up to 18 hours and then is poured into ice water and stirred for two hours. The resulting solid is filtered, dried and if necessary purified by column chromatography to give the desired amides HP2.

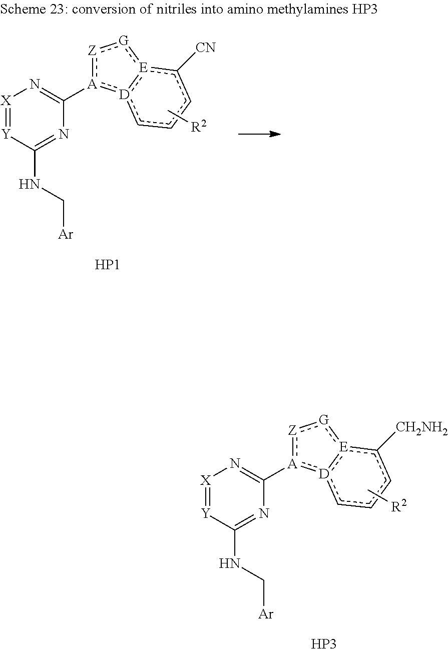

In some cases the desired compounds HP1 prepared in Schemes 6-20 above can have a nitrile substitution at the position indicated in Scheme 22. This substituent can be converted to the corresponding methylamines HP3. A solution of nitrile HP1 in an aprotic organic solvent such as THF is treated with LAH and the resulting mixture is stirred for up to 18 hours. The reaction mixture is treated with 15% NaOH in water and the reaction is stirred for one hour and is then filtered. The THF is removed under reduced pressure to give the product HP3 which can be further purified by column chromatography.

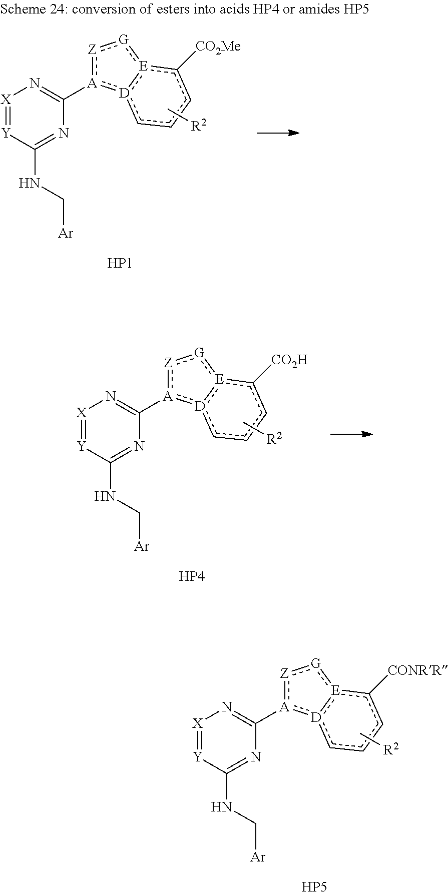

In some cases the desired compounds HP1 prepared in Schemes 6-20 above can have a carboxylate ester at the position indicated in Scheme 23. This functionality can be readily converted to the corresponding acids HP4 or substituted amides HP5 using standard methodology.

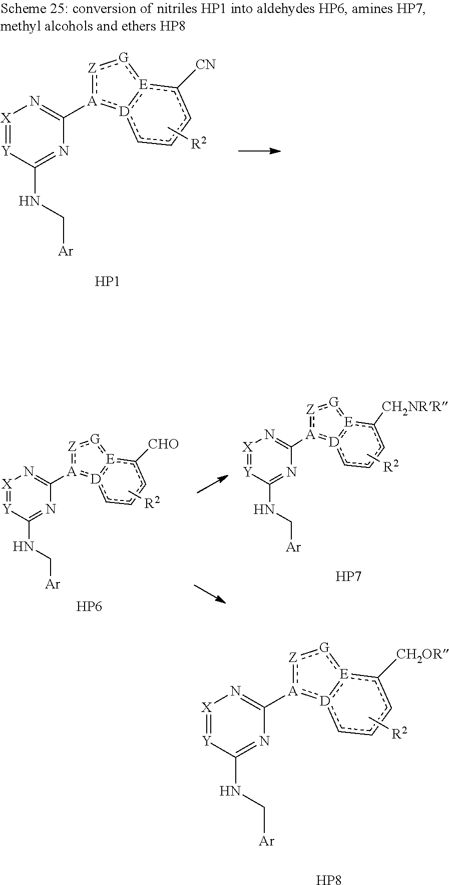

In some cases the desired compounds HP1 prepared in Schemes 6-20 above can have a nitrile at the position indicated in Scheme 22. This functionality can be readily converted to aldehydes HP6 and the corresponding amines HP7 or alcohols or ethers HP8.

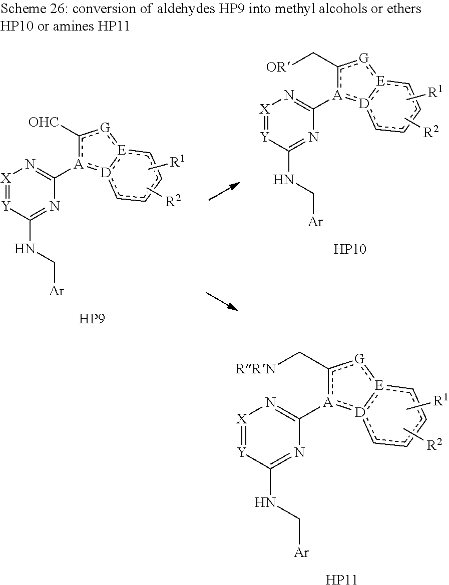

In some cases the desired compounds HP9 prepared in Schemes 6-20 above can have an aldehyde at the position indicated in Scheme 25. This functionality can be readily converted to the corresponding alcohols or ethers HP10 or amines HP11 or using standard methodology.

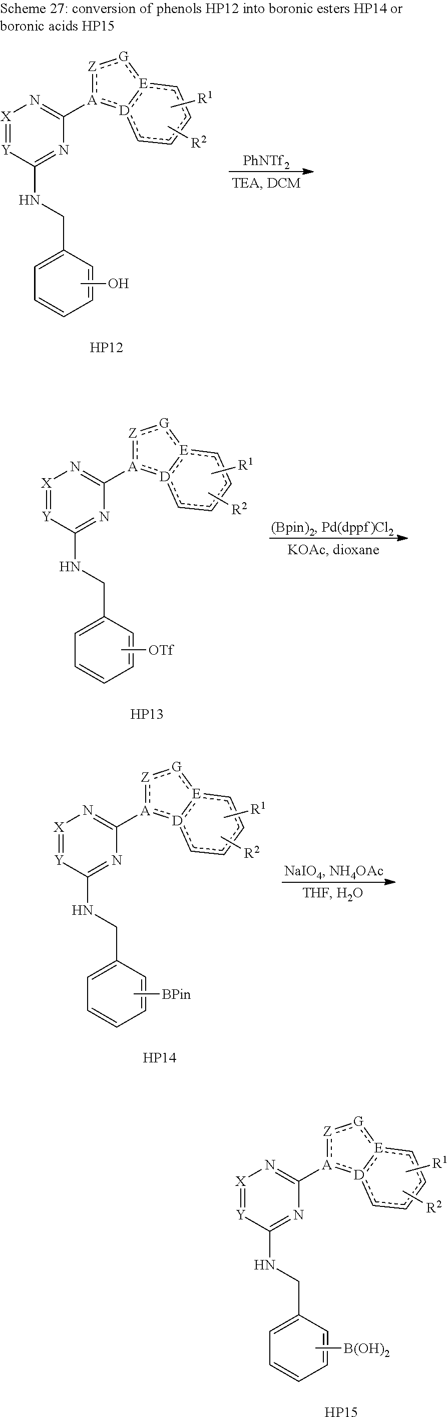

In some cases the desired phenols HP12 prepared in Schemes 6-20 above can have a hydoxy group substituted on the aromatic ring indicated in Scheme 26. This functionality can be readily converted to the corresponding triflates HP13, the latter can be converted into boronic esters HP14 and the corresponding boronic acids HP15.

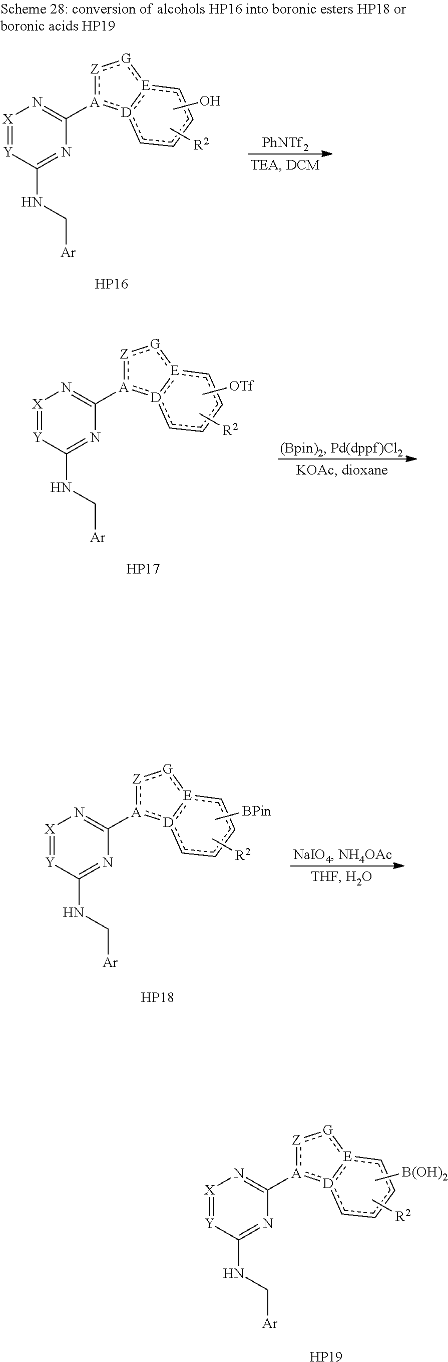

In some cases the desired compounds containing hydroxyl group as R1 or R4 HP16 prepared in Schemes 6-20 above can have a hydoxy group substituted on the aromatic ring indicated in Scheme 27. Similarly, this functionality can be readily converted to the corresponding triflates HP17, the latter can be converted into boronic esters HP18 and the corresponding boronic acids HP19.

### Biological Assays

The biological activities of the nitrogen hexacycle compounds of the invention can be determined by their examination in in vitro and cellular assays using protocols well established to identify and select compounds that will exhibit anti-cancer activity. The present invention focuses upon the ability of the nitrogen hexacycle compounds to intersect with the p97 proteosome complex. As described in the Background, the function of the p97 complex is essential for continued cellular viability. Inhibition of the activity of the complex will cause protein build-up in the cell and consequent apoptosis. The biological assays allow an assessment of the biological activities of the nitrogen hexacycle compounds of the invention.

The primary biological analyses are in vitro assays and cellular based assays for determining the inhibitory capability of the triazine, pyridine, pyrimidine compounds of the invention of the invention against Valosin-containing protein, i.e., p97. The assays also provide a primary indication of bioavailability of the nitrogen hexacycle compounds of the invention.

The ability to inhibit the p97 complex is studied through use of a p97 in vitro assay using a tagged p97 substrate pursuant to the method of Christianson in Nat Cell Biol. (2011) 14:93 for a p97 cell-based assay. A cell based assay is used to test the anti-tumor effects of inhibitors on cultured cancer cells. This anti-tumor assay is based upon cultured cancer cells using the commercially available cell titer glo assay provided by Promega. Additional assays enable assessment of bioavailability through art recognized model studies designed to demonstrate the ability of the compounds of the invention to reach target cells in vivo. While all compounds tested displayed a degree of anti-tumor activity, the assays also allowed identification of triazine, pyridine, pyrimidine compounds as candidates that may be selected for further examined by in vivo anti-tumor testing in mouse, guinea pig and dog models. The selected candidates were shown to have highly desirable pharmacokinetic properties in these in vitro assays.

### P97 ATPase Biochemical Assay

The ATPase assay is performed according the following protocol: Purified enzyme (20 nM p97), substrate (20 μM ATP) and a dose titration of compounds are mixed in buffer (50 mM TRIS pH 7.5, 20 mM MgCl₂, 0.02% TX-100, 1 mM DTT, 0.2% (v/v) glycerol) and incubated at 37° C. for 15 minutes. The reaction is terminated and the level of product generated is measured using the ADP Glo Assay Kit (Promega, Madison Wis.). Plotting product generated versus compound concentration and using a four-parameter fit model generates an IC50 value for each compounds.

### P97 Cell-Based Assay

On target cell-based effects of compounds of the invention are monitored using the reporter cell line HEK-293 TCRα-GFP as described in Christianson et al. Nat. Cell Biol. (2011) 14:93. Inhibition of turnover of the TCRα-GFP reporter is a hallmark of p97 inhibition. The protocol for TCRα-GFP monitoring reporter turnover is as follows: Reporter cells are seeded and incubated with proteasome inhibitor MG132 to accumulate TCRα-GFP. Subsequently, MG132-containing media is removed and a dose titration of compound plus cycloheximide is incubated with the cells. At the end of the incubation, compound and media are removed, cells are fixed and GFP fluorescence is measured by standard epifluorescent microscopy techniques. Plotting fluorescence versus compound concentration and using a four-parameter fit model generates an IC50 value for each compound.

Image-analysis is used to generate quantitative data from these assays that can be fit to a four-parameter sigmoid curve to derive IC50 values. Substrates of the ubiquitin-proteasome system, such as p53, are monitored after tumor cell lines are incubated with compounds for several hours. Accumulation of these proteins indicates an inhibition of proteasome-mediated degradation. Accumulation of lysine-48 chain linkage of poly-ubiquitin is also monitored by immunofluorescence as an indicator of ubiquitin-proteasome system inhibition. Both LC3 and SQSTM1 are mediators of autophagy. The localization and amounts of these proteins are monitored by immunofluorescence and report on the activity and inhibition of autophagy in response to p97 inhibition.

### Cultured Cancer Cell Assay

Anti-tumor effects are monitored in cultured cancer cells after several days of compound treatment. The cell titer glo assay (Promega) measures the amount of ATP present as a proxy for cellular viability. Cellular counting is done using high-content microscopy followed by image analysis. A hanging drop 3D-culture system (3D Biomatrix) is used followed by cell titer glo to measure growth in a tumor-like environment.

### Absorption Assay

The ability of compounds to be absorbed from the lumen of the gastrointestinal tract after oral administration was assessed by measuring their permability through Caco-2 cell monolayers. SunD, et al., Curr. Opin. Drug Discov. Develop[(2004) 75. The in vitro permeability of compound (2 μM in Kreb's buffer or HBSS buffer with n=2) was determined using 21-day old Caco-2 cell monolayers. The permeation coefficient was determined for both Apical to Basolateral (A to B) and Basolateral to Apical (B to A) after 120 min at 37° C. The efflux ratio was calculated based on the ratio of permeation coefficient of B to A vs. A to B to determine the potential of compound as substrate for efflux pump (e.g. Pgp). The protocol for this Caco-2 assay and the corresponding detailed description are provided in the following experimental section.

### Metabolic Stability Assay

Metabolic stability of compounds can be assessed by measuring their half lives in liver microsomal preparations. Roserts, Sa, et al., Xenobiotica (2001) 37:557. Compounds are applied to a preparation of mouse liver microsomes in the presence of NADPH and their half lives are determined by measuring the rate of disappearance of the compounds from the preparation by determining the concentration at 0, 15, 30 and 60 minutes using LCMS/MS. The protocol for determining metabolic stability in a mouse liver assay and the corresponding detailed description are provided in the following experimental section.

### Nonspecific Binding Assay

Many compounds are known to bind nonspecifically to proteins found in high abundance in the plasma. The fraction of unbound drug (free fraction) is available for interaction with targets found in tissues. Banker, M. J. et al., Curr. Drug Metab. (2008) 9:854. The ability of compounds to escape a chamber containing blood plasma to a chamber containing only buffer can be assessed by measuring the concentration that appears in the buffer chamber and the concentration that remains in the plasma chamber. These measurements can be used to determine the fraction of compound bound to plasma proteins and its free fraction (100-percent bound to plasma proteins). The protocol for determining non-specific protein binding in a plasma protein binding assay and the corresponding detailed description are provided in the following experimental section.

The results of the primary assay conducted with selected triazine, pyridine, pyrimidine compounds and substituted quinazoline compounds of the invention show that the triazine, pyridine, pyrimidine compounds of the invention display significant inhibitory activity (IC₅₀) against the enzymatic action of p97 toward its natural substrate. Some of these compounds also have greater potency in cell based assays and have in vitro pharmacokinetic properties consistent with good oral bioavailability.

Table I presents the results of several of these assays conducted upon the triazine, pyridine, pyrimidine compounds of the invention.

TABLE I

| | p97 IC50 **** <30 nM | A549 CTG | A549 K48 |
|---|---|---|---|
| | *** <100 nM | IC50 | IC50 |
| | ** <1000 nM | *** <1 uM ** | *** <1 uM |
| IUPAC NAME | * <10 uM | <3 uM * <10 uM | ** <3 uM * <10 uM |
| 1-(5-(benzylamino)-6-methyl-1,2,4-triazin-3-yl)-2- | * | | |
| methoxy-1H-benzo[d]imidazole-4-carbonitrile | | | |
| 1-(5-((3-fluorobenzyl)amino)-6-methyl-1,2,4- | ** | * | ** |
| triazin-3-yl)-2-methyl-1H-indole-4-carboxamide | | | |
| 2-(aminomethyl)-1-(5-(benzylamino)-6-methyl- | * | | |
| 1,2,4-triazin-3-yl)-1H-indole-4-carboxamide | | | |
| 1-(5-(benzylamino)-6-ethyl-1,2,4-triazin-3-yl)-2- | **** | *** | *** |
| methyl-1H-indole-4-carboxamide | | | |
| 1-(4-(benzylamino)-6-methyl-1,3,5-triazin-2-yl)-2- | *** | * | ** |
| methyl-1H-indole-4-carboxamide | | | |
| 1-(5-(benzylamino)-6-methyl-1,2,4-triazin-3-yl)-2- | **** | ** | ** |
| methyl-1H-indole-4-carboxamide | | | |

### Mechanism of Action and Medical Treatment

In certain embodiments, the invention is directed to methods of inhibiting p97. The nitrogen hexacycle compounds for use in the methods disclosed herein bind to the active site of p97, e.g., noncovalently or covalently. In certain such embodiments, the covalent binding may be reversible or irreversible.

The compounds of the invention and their pharmaceutical compositions are capable of acting as “inhibitors” of p97 which means that they are capable of blocking or reducing the activity of an enzyme, for example, inhibition of various activities of p97. An inhibitor can act with competitive, uncompetitive, or noncompetitive inhibition. An inhibitor can bind reversibly or irreversibly, and therefore the term includes compounds that are suicide the enzyme, or it can cause a conformational change elsewhere on the enzyme.

The compounds of the invention and their pharmaceutical compositions function as therapeutic agents in that they are capable of preventing, ameliorating, modifying and/or affecting a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The ability to prevent, ameliorate, modify and/or affect in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The compounds of the invention and their pharmaceutical compositions are capable of functioning prophylacticly and/or therapeutically and include administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

### Administration

Compounds prepared as described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
(3) humectants, such as glycerol;
(4) disintegrating agents, such as agar-agar, calcium carbonate, potato or tapioca starch, alginic acid, certain silicates, and sodium carbonate;
(5) solution retarding agents, such as paraffin;
(6) absorption accelerators, such as quaternary ammonium compounds;
(7) wetting agents, such as, for example, acetyl alcohol and glycerol monostearate;
(8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents.
   In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound of the invention in a pharmaceutically acceptable mixture will vary depending on several factors, including the dosage of the compound to be administered, the pharmacokinetic characteristics of the compound(s) employed, and the route of administration.

In general, the compositions of this invention may be provided in an aqueous solution containing about 0.1-10% w/v of a compound disclosed herein, among other substances, for parenteral administration. Typical dose ranges are from about 0.01 to about 50 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

Another aspect of the invention provides a conjoint therapy wherein one or more other therapeutic agents are administered with the compounds and compositions of the invention. Such conjoint treatment will achieve the same or similar treatment accounting for the additive effects of the conjoined therapeutic agents other than the compounds of the invention.

In certain embodiments, a compound of the invention is conjointly administered with one or more proteasome inhibitor(s). In certain embodiments, a compound of the invention is conjointly administered with a chemotherapeutic. Suitable chemotherapeutics may include, natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates (busulfan), nitrosoureas (carmustine (BCNU) and analogs, streptozocin), trazenes-dacarbazinine (DTIC); antiproliferative/antimitotic antimetabolites such as folic acid analogs (methotrexate), triazine, pyridine, pyrimidineanalogs (fluorouracil, floxuridine, and cytarabine), purine analogs and related inhibitors (mercaptopurine, thioguanine, pentostatin and 2-chlorodeoxyadenosine); aromatase inhibitors carboplatin), procarbazine, hydroxyurea, mitotane, aminoglutethimide; hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, a compound of the invention is conjointly administered with a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, paramethasone, prednicarbate, prednisolone, prednisolone 25-diethylaminoacetate, prednisolone, sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof.

In certain embodiments, a compound of the invention is conjointly administered with an immunotherapeutic agent. Suitable immunotherapeutic agents may include, but are not limited to, cyclosporine, thalidomide, and monoclonal antibodies. The monoclonal antibodies can be either naked or conjugated such as rituximab, tositumomab, alemtuzumab, epratuzumab, ibritumomab tiuxetan, gemtuzumab ozogamicin, bevacizumab, cetuximab, erlotinib and trastuzumab.

### Treatment of Cancer

Exemplary forms of cancer which may be treated by the methods of the invention include, but are not limited to, prostate cancer, bladder cancer, lung cancer (including either small cell or non-small cell cancer), colon cancer, kidney cancer, liver cancer, breast cancer, cervical cancer, endometrial or other uterine cancer, ovarian cancer, testicular cancer, cancer of the penis, cancer of the vagina, cancer of the urethra, gall bladder cancer, esophageal cancer, or pancreatic cancer.

Additional exemplary forms of cancer which may be treated by the methods of the invention include, but are not limited to, cancer of skeletal or smooth muscle, stomach cancer, cancer of the small intestine, cancer of the salivary gland, anal cancer, rectal cancer, tyroid cancer, parathyroid cancer, pituitary cancer, and nasopharyngeal cancer.

The compounds of the present invention and their salts and solvates, thereof, may be employed alone or in combination with other therapeutic agents for the treatment of the diseases or conditions associated with inappropriate P97 activity.

Additional diseases that can be treated according to the methods of the invention include in addition to cancer, auto-immune disorders, metabolic diseases, infection diseases, neurological diseases, graft versus host disease and other hereditary diseases outlined here: abeta-lipoproteinema, acerulopasminemia, alpha-1-antichymotrypsin (ACT) deficiency, aspartylglucosaminuria, autosomal dominant retinitis pigmentosa, brugada syndrome, Charcot-Marie-Tooth syndrome, congenital adrenal hyperplasia, congenital chloride diarrhea, congenital hypothyroidism, congenital long QT syndrome, congenital nephritic syndrome, congenital sucrase-isomaltase deficiency, Crigler-Najjar type II, cystic fibrosis, diabetes mellitus, diastrophic displasia, DubinJohnson syndrome, Fabri disease, familial chylomicronemia, familial glucocorticoid deficiency, familial hypercholesterolemia, Gaucher disease, heavy chain disease, hereditary emphysema, hereditary emphysema with liver injury, hereditary hemochromatosis, hereditary hypofibrinogenemia, hereditary myeloperoxidase, hereditary spherocytosis, hirschprung disease, hypogonadotropic hypogonadism, infantile systemic hyalinosis, infentile neuronal ceroid lipofuscinosis, laron syndrome, liver failure, lupus erythematosus, marfan syndrome, medullary cystic kidney disease, familial juvenile hyperuricemic nephropathy, Menkes disease, nephrogenic diabetes, neurohypophyseal diabetes insipidus, oculocutaneous albinism, osteogenesis imperfect, PelizaeusMerzbacher disease, Pendred syndrome, persistent hyperinsulinemic hypoglycemia of infancy, primary hypothyroidism, Protein C deficiency, pseudoachondropla with multiple epiphyseal dysplasia, severe congenital neutropenia, Stargardt-like macular dystrophy, steroid-resistant nephrotic syndrome, TaySachs, Type I hereditary angioedema, tyroxine binding globulin deficiency, von Willebrand disease type IIA, X-linked Charot-Marie-Tooth disease, X-linked hypophosphatemia, Alzheimer disease autosomal recessive juvenile parkinsonism, combined factors V and VIII deficiency, cranio-lenticulo-sutural dysplasia, hypotonia and dysmorphism, inclusion body myopathy Paget's disease of the bone and fronto-temporal dementia (IBMPFD), lipid absorption disorders, Marinesco-Sjoegren syndrome, Parkinson, polycystic liver disease, spondylo-epiphyseal dysplasia tarda, WalcottRallison syndrome and Lou Gehrig's disease (ALS).

In various embodiments, compounds of the invention may be used to treat neoplastic growth, angiogenesis, infection, inflammation, immune-related diseases, ischemia and reperfusion injury, multiple sclerosis, rheumatoid arthritis, neurodegenerative conditions, or psoriasis.

Neoplastic growth may include cancer. Suitably, the present invention relates to a method for treating or lessening the severity of a cancer selected from: brain (gliomas), glioblastomas, breast, Wilm's tumor, Ewing's sarcoma, rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, chronic myelogenous leukemia, chronic lymphocytic leukemia, Hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

In various embodiments, the cancer is selected from brain cancer (gliomas), glioblastomas, breast cancer, colon cancer, head and neck cancer, kidney cancer, lung cancer, liver cancer, melanoma, ovarian cancer, pancreatic cancer, prostate cancer, sarcoma and thyroid cancer.

In various embodiments, the cancer to be treated is associated with the proteasome. See Voorhees et al., The Proteasome as a Target for Cancer Therapy, Clinical Cancer Research, vol. 9, 6316-6325, December 2003, incorporated by reference in its entirety. In various embodiments, the cancer is associated with a particular target, such as NFkB, p44/42 MAPK, P-gp, TopI, TopIIalpha.

In various embodiments, the cancer is a solid tumor. In various embodiments, the cancer is selected from multiple myeloma, metastatic breast cancer, non-small cell lung cancer, prostate cancer, advanced colorectal cancer, ovarian or primary peritoneal carcinoma, hormone refractory prostate cancer, squamous cell carcinoma of the head and neck, metastatic pancreatic adenocarcinoma, gastroesophageal junction or stomach, or non-Hodgkin's lymphoma.

A method of using the compounds described herein for treating a disorder characterized by an inappropriate level of proteasome activity, or in which a reduction of the normal level of proteasome activity yields a clinical benefit. This disorder can include cancer or immune disorders characterized by excessive cell proliferation or cellular signaling. Among cancers, this includes human cancers that overexpress c-Myc or express an oncogenic form of the K-Ras protein.

Neurodegenerative diseases and conditions may include without limitation stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, ALS, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis). Compounds of the invention may be used to treat Alzheimer's disease, including administering to a subject an effective amount of an agent or composition (e.g., pharmaceutical composition) disclosed herein.

Compounds of the invention may be used to treat cachexia and muscle-wasting diseases. Compounds of the invention may be used to treat such conditions wherein the condition is related to cancer, chronic infectious diseases, fever, muscle disuse (atrophy) and denervation, nerve injury, fasting, renal failure associated with acidosis, diabetes, and hepatic failure.

Compounds of the invention can be used to treat hyperproliferative conditions such as diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders). The treatment of burn victims is often hampered by fibrosis, thus, an additional embodiment of the application is the topical or systemic administration of the inhibitors to treat burns. Wound closure following surgery is often associated with disfiguring scars, which may be prevented by inhibition of fibrosis. Thus, in certain embodiments, the application relates to a method for the prevention or reduction of scarring.

Compounds of the invention can be used to treat ischemic conditions or reperfusion injury for example acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

Compounds of the invention can be used for the inhibition of TNFalpha to prevent and/or treat septic shock.

Compounds of the invention can be used for inhibiting antigen presentation in a cell, including exposing the cell to an agent described herein. A compound of the invention may be used to treat immune-related conditions such as allergy, asthma, organ/tissue rejection (graft-versus-host disease), and auto-immune diseases, including, but not limited to, lupus, rheumatoid arthritis, psoriasis, multiple sclerosis, and inflammatory bowel diseases (such as ulcerative colitis and Crohn's disease). Thus, a further embodiment is a method for moedulating the immune system of a subject (e.g., inhibiting transplant rejection, allergies, auto-immune diseases, and asthma), including administering to the subject an effective amount of a compound of the invention.

Compounds of the invention can be used in methods for altering the repertoire of antigenic peptides produced by the proteasome or other protein assembly with multicatalytic activity.

Compounds of the invention can be used in methods for inhibiting IKB-alpha degradation, including contacting the cell with an agent identified herein. A further embodiment is a method for reducing the cellular content of NF-KB in a cell, muscle, organ, or subject, including contacting the cell, muscle, organ, or subject with a compound of the invention.

Compounds of the invention can be used in methods for affecting cyclin-dependent eukaryotic cell cycles. Compounds of the invention can be used in methods for treating a proliferative disease in a subject (e.g., cancer, psoriasis, or restenosis). Compounds of the invention can be used for treating cyclin-related inflammation in a subject.

One embodiment is a method for treating p53-related apoptosis, including administering to a subject an effective amount of a compound of the invention.

In another embodiment, the agents of the present application are useful for the treatment of a parasitic infection, such as infections caused by protozoan parasites. In certain such embodiments, the agents are useful for the treatment of parasitic infections in humans caused by a protozoan parasite selected from Plasmodium sps., Trypanosoma sps., Leishmania sps., Pneumocystis carinii, Toxoplasma gondii, Entamoeba histolytica, Entamoeba invadens, and Giardia lamblia. In certain embodiments, the agents are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds useful as proteasome inhibitors in the treatment of parasitic diseases are described in WO 98/10779, which is incorporated herein in its entirety.

In particular, the methods of treatment include inhibiting, arresting, ameliorating, minimizing and/or eliminating malconditions associated with the inability of cells to metabolize, degrade or otherwise remove ubiquitin tagged proteins and peptides because the tag has been cleaved, degraded, removed or otherwise rendered disfunctional as a result of P97 metalloprotease domain activity. Included are methods in which a human disorder characterized by abnormal regulatory peptide degradation resulting in excessive cell proliferation or cell signaling. The methods are directed to administration of an effective amount of a compound or pharmaceutical formulation disclosed above so that the abnormal regulatory peptide degradation is ameliorated, reduced or inhibited. In particular, the human disorders include a cancer or immune disorder, a cancer resulting from overexpression of c-Myc or expression of an oncogenic form of the K-Ras protein. The methods also include inhibition or amelioration of P97 metalloprotease domain activity in a human patient suffering from abnormal P97 metalloprotease domain activity on ubiquitin modified proteins. As described above, these methods involve administering to the patient an effective amount of a compound or pharmaceutical formulation disclosed above so that the abnormal P97 metalloprotease domain activity is ameliorated, reduced or inhibited.

### Additional Embodiments of the Compounds of the Invention

Additional embodiments of the compounds of the invention include the following variations of the substituents R¹ to R⁶ and Rₙ. Each of these variations can be combined with any other variation as is appropriate for the final structure of the nitrogen hexacycle scaffold desired to form a full nitrogen hexacycle compound of the invention.

The number designations for the carbons include all integers between the lowest and highest number. Individual numbers of carbon atoms separate and distinct from other numbers of the same group are also included. For example for an alkyl of 1 to 6 carbons, an alkyl group of 1, 2, 3, 4, 5 or 6 carbons is included as well as each individual number designation separate and distinct from other number designations so that an alkyl of 1 to 6 carbons includes separately, methyl, ethyl, propyl, butyl, pentyl and hexyl.
1) Linear, branched or cyclic alkyl of 1 to 6 carbons,
2) Linear, branched or cyclic alkoxy of 1 to 6 carbons,
3) Amine and aminoalkyl (eg, —NHR and —NR₂)
4) Carboxylic acid,
5) Carboxylic ester wherein the alkoxy group of the ester is from 1 to 6 branched or straight carbons or the alcohol esterifying group is phenoxy,
6) Linear, branched or cyclic alkylenyl carboxylic acid or ester of 2 to 7 carbons in the alkylenyl group and 1 to 6 branched or straight carbons in the ester group, for example a linear alkylenyl carboxylic acid of 2 carbons in the alkylenyl group is —CH₂CH₂COOH,
7) Branched or straight alkylenyl amine of 1 to 6 carbons (eg, —R—NH₂),
8) Linear, branched or cyclic perfluoroalkyl of 1 to 6 carbons,
9) Linear, branched or cyclic trifluoroalkyl of 1 to 6 carbons wherein the trifluoro group is on the terminating or end carbon,
10) Hydroxyl,
11) Linear, branched or cyclic alkylenyl hydroxyl of 1 to 6 carbons,
12) Carboxamide eg., —CONH₂,
13) Linear, branched or cyclic alkylenylcarboxamide of 1 to 6 carbons in the alkylenyl group,
14) N-substituted carboxamide, wherein the N substituent is an aryl group, heteroaryl group or heterocycle group as defined in the DEFINITIONS section, eg., —CONHAr or —CONHHet,
15) N-substituted carboxamide wherein the N substituent is an alkaryl group, an alkheteroaryl group or an alkheterocycle group as defined in the DEFINITIONS section, and wherein the “alk” group is a linear, branched or cyclic alkylenyl group of 1 to 6 carbons, eg., —CONH—R—Ar or CONH—R-Het,
16) N-substituted carboxamide wherein the N substituent is a branched or straight alkyl group of 1 to 10 carbons, the polyfluorinated version thereof, or a substituted version thereof, eg., —CONH—R, wherein the substituent of the alkyl group is halogen, cyano, carboxyl, ester of 1 to 6 branched or straight chain carbons in the alkoxy or phenoxy portion, carboxamide, sulfoxamide, alkoxy of 1 to 6 carbons, urea, carbamate of 1 to 10 carbons, amine, mono or dialkyl amine having from 1 to 6 carbons in the alkyl group with the alkyl group being straight or branched, hydroxyalkyl of 1 to 10 branched or straight chain carbons or a cycloalkyl group as defined in the DEFINITIONS section,
17) Aminocarbonylalkyl, eg., —NHCOR, wherein R is a linear, branched or cyclic alkyl of 1 to 6 carbons,
18) Alkyleneaminocarbonylalkyl, eg., —RNHCOR, wherein the alkylenyl is linear, branched or cyclic and is 1 to 6 carbons and the alkyl is linear, branched or cyclic and is 1 to 6 carbons,
19) A heterocyclic system comprised of one or more of the following: an azetidine or substituted azetidine attached as any of the R groups, pyrrolidine or substituted pyrrolidine attached as any of the R groups, piperidine or substituted piperidine attached as any of the R groups, a piperazine or substituted piperazine attached as any of the R groups, a morphorpline or substituted morpholine attached as any of the R groups, with the proviso that when the R group is attached to a nitrogen, the resulting heterocyclic system results in a stable R—N configuration,
20) Preferred aryl, heteroaryl and heterocycle groups for 14 and 15 include phenyl, halogen substituted phenyl, aminophenyl, benzoic acid, tolyl, xylyl, anisolyl, trifluoromethylphenyl, benzyl, tetrahydrofuran, pyrrolidinyl, tetrahydronaphthalene, cyclohexyl or alkyl substituted cyclohexyl with the alkyl group having 1 to 6 carbons, cyclohexyl or alkyl substituted cyclohexyl with the alkyl group having 1 to 6 carbons, cyclopentyl or alkyl substituted cyclopentyl with the alkyl group having 1 to 6 carbons, pyrazolyl, imidazolyl, piperidinyl, piperazinyl, pyrimidinyl, morpholinyl, pyrrolyl, thiophenyl, substituted versions of any of the foregoing aryl, heteroaryl or heterocycle groups wherein the chemical substituent is halogen, cyano, carboxyl, ester of 1 to 10 branched or straight chain carbons in the alkoxy or phenoxy portion, amine, carboxamide, sulfoxamide, urea, carbamate of 1 to 10 carbons, hydroxyl, thiol, alkoxy, anisolyl, phenyl, benzyl or a cycloalkyl group as defined in the DEFINITIONS section,
21) Derivatives of 14-17 wherein the N of the carboxamide or aminocarbonyl has a second substituent and the second substituent is a branched or straight chain alkyl of 1 to 6 carbons,
22) N-substituted carboxamide wherein the N substituent is a mono, di, tri or tetra amino acid and the amino acid moieties include glycinyl, alaninyl, leucinyl, valinyl, phenylalaninyl, lysinyl, argininyl, histidinyl, serinyl, aspariginyl, glutaminyl, aspartic, glutamic such that the amino acid moieties may be combined in any combination of two, three or four moieties including but not limited to a tetramer of four different moieties, a tetramer of two and two different moieties, a tetramer of three of one moiety and one of a different moiety, a trimer of two of one moiety and one of another moiety or a trimer of three different moieties, a dimer of two different moieties of the same moiety, and a monomer of any of the designated moieties. The nitrogen of an amino acid moiety may serve as the nitrogen of the carboxyamide group. The C-terminus of the amino acid monomer, dimer or trimer may be a carboxylic acid or a carboxamide. The order of amino acid moieties in the tetramer, trimer or dimer may be any order.
23) Any of the substituents designated by items 1-3, 5-9, 11, 13-22 which additionally includes any functional group selected from F, Cl, Br, I, OR′, B(OH)₂, B(OMe or Et)₂, OC(O)N(R′)₂, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl.
24) In addition to the groups of substituents set forth in 1 through 23 above, each individual substituent and individual combination is included separately and individually as if it were individually recited.
25) Additional embodiments of the compounds of the invention further include each individual compound listed on the compound List above.
26) Hydrogen.

### Examples

The following describes the preparation of representative compounds of the invention in greater detail. The following examples are offered for illustrative purposes, and are not intended to limit the invention in any manner. Those of skill in the art will readily recognize a variety of noncritical parameters which can be changed or modified to yield essentially the same results.

Those skilled in the art will recognize, or be able to ascertain using no more than routine experimentation, numerous equivalents to the syntheses of the compounds and methods of use thereof described herein. Although certain exemplary embodiments are depicted and described herein, it will be appreciated that compound of the invention can be prepared according to the methods generally available to one of ordinary skill in the art. All of the above-cited references and publications are hereby incorporated by reference.

Unless otherwise noted, all solvents, chemicals, and reagents were obtained commercially and used without purification. The ¹H NMR spectra were obtained in CDCl₃, d₆-DMSO, CD₃OD, or d₆-acetone at 25° C. at 300 MHz on an OXFORD (Varian) spectrometer with chemical shift (δ, ppm) reported relative to TMS as an internal standard. HPLC-MS chromatograms and mass spectra were obtained with Shimadzu LC-MS-2020 system. The prep-HPLC instruments used to purify some compounds were either a Gilson GX-281(Gilson) or a P230 Preparative Gradient System (Elite). Preparative chira HPLC seperations were performed using an Elite P230 Preparative Gradient System, a Thar Prep-80 or Thar SFC X-5. Reactions using microwave irriadation were performed on a CEM Discover SP instrument.

### Synthetic Examples

### Synthesis of Compound A

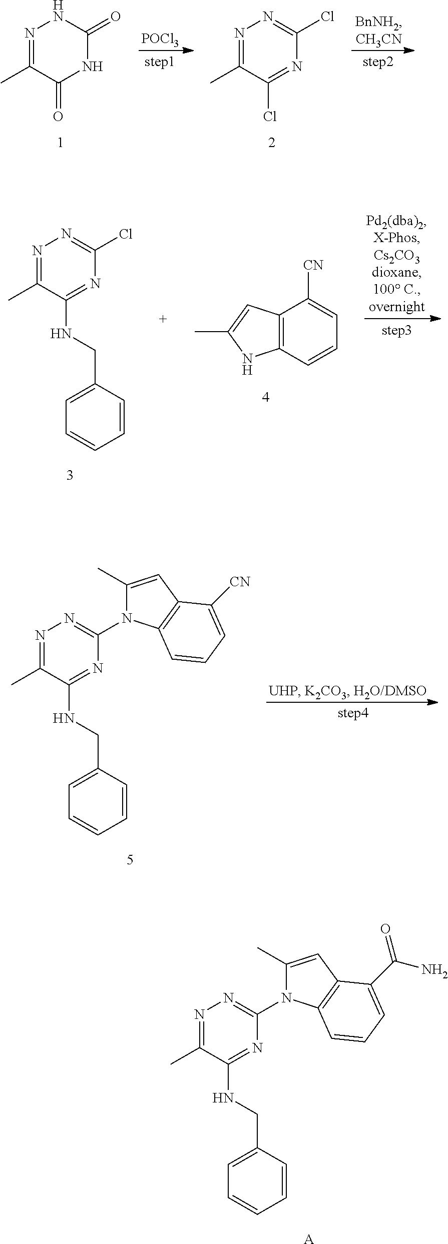

To a solution of 6-methyl-1,2,4-triazine-3,5(2H,4H)-dione 1 (600 mg, 4.7 mmol) in POCl₃ (50 mL). The reaction mixture was stirred at 110° C. for 4 hours. The reaction mixture was concentrated under vacuum to get crude 3,5-dichloro-6-methyl-1,2,4-triazine2 (650 mg, 84%), which was used for the next step directly. LRMS (M+H+) m/z: calcd 163.97; found 164.

To a solution of 3,5-dichloro-6-methyl-1,2,4-triazine2 (650 mg, 3.9 mmol) in CH₃CN (30 mL) was added benzylamine (640 mg, 5.9 mmol). The reaction was stirred overnight at room temperature. The resulting mixture was concentrated under vacuum, and the residue was purified by flash chromatography using a mixture of hexane and ethyl acetate (1:1) to afford N-benzyl-3-chloro-6-methyl-1,2,4-triazin-5-amine 3 (420 mg, 45%). LRMS (M+H⁺) m/z: calcd 235.07; found 235.

A mixture of the N-benzyl-3-chloro-6-methyl-1,2,4-triazin-5-amine 3 (420 mg, 1.7 mmol), 2-methyl-1H-indole-4-carbonitrile 4 (248 mg, 1.5 mmol), tris(dibenzylideneacetone) dipalladium(O) (240 mg, 0.3 mmol), X-phos (240 mg, 0.5 mmol) and Cₛ₂CO₃ (1048 mg, 3.2 mmol) in dioxane (30 mL) was heated at 100° C. for 16 hours under nitrogen atmosphere. The reaction mixture was cooled down to room temperature and concentrated under vacuum, and the residue was purified by flash chromatography (DCM:MeOH=20:1) to afford 1-(5-(benzylamino)-6-methyl-1,2,4-triazin-3-yl)-2-methyl-1H-indole-4-carbonitrile 5 (520 mg, 80%). LRMS (M+H⁺) m/z: calcd 355.16; found 355.

To a solution of the 1-(5-(benzylamino)-6-methyl-1,2,4-triazin-3-yl)-2-methyl-1H-indole-4-carbonitrile 5 (120 mg, 0.31 mmol) in DMSO (20 mL) were added UHP (297 mg, 3.1 mmol), K₂CO₃ (19 mg, 0.14 mmol) and water (1 mL), the reaction was stirred at room temperature overnight followed by dilution with water (50 mL). The resulting solid was collected and purified by flash chromatography (DCM:MeOH=20:1) to afford 1-(5-(benzylamino)-6-methyl-1,2,4-triazin-3-yl)-2-methyl-1H-indole-4-carboxamide A as a yellow solid (90 mg, 73%). LRMS (M+H⁺)m/z: calcd 373.17; found 373. HPLC purity (214 nm): 99%. ¹HNMR (300 MHz, CD₃OD): δ 8.67-8.64 (m, 1H), 7.77-7.76 (m, 1H), 7.76-7.75 (m, 1H), 7.69-7.66 (m, 1H), 7.48-7.45 (m, 5H), 7.44-7.38 (m, 1H), 6.97-6.95 (m, 1H), 6.92-6.90 (m, 1H), 4.67-4.65 (m, 2H), 2.54-2.50 (m, 3H), 2.49-2.45 (m, 3H).

### Synthesis of Compound B

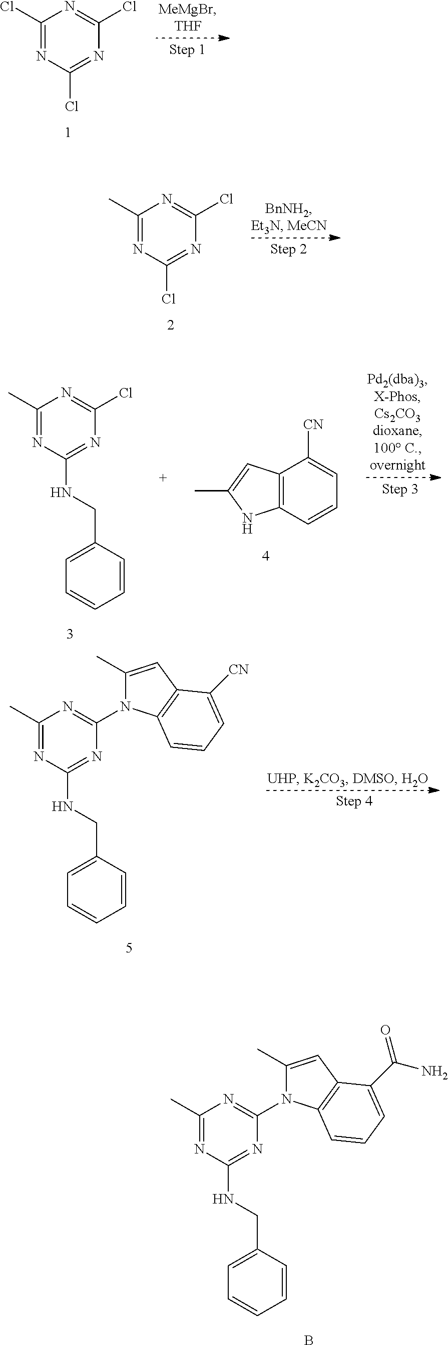

A 3.0 M solution of MeMgBr in Et₂O (10.0 mL, 30 mmol) was added slowly to a white suspension of 2,4,6-trichloro-1,3,5-triazine1 (3.68 g, 20 mmol) in CH₂Cl₂ (25 mL) at 0° C., and the resulting yellow suspension was allowed to warm to 22° C. and was stirred for 3 h. The reaction was carefully quenched with saturated aqueous NH₄Cl at 0° C. and then diluted with H₂O and CH₂Cl₂ (25 mL). The organic layer was separated, dried, filtered, and concentrated to give 2,4-Dichloro-6-methyl-1,3,5-triazine2 which was used without further purification. Yield (2.94 g, 90%), yellow solid. ¹H NMR (400 MHz, CDCl₃): δ 2.74 (s, 3H).

To a solution of 2,4-Dichloro-6-methyl-1,3,5-triazine2 (320 mg, 2 mmol) in CH₃CN (20 mL) was added phenylmethanamine (235 mg, 2.2 mmol) and TEA (0.8 mL, 6 mmol). Then the reaction solution was stirred at room temperature overnight. The resulting mixture was concentrated and purified by combiflash (petroleum ether/ethyl acetate=3:1) to give N-benzyl-4-chloro-6-methyl-1,3,5-triazin-2-amine 3 (0.29 g, 60%). LRMS (M+H+) m/z: calcd 235.07; found 235.

To a solution of N-benzyl-4-chloro-6-methyl-1,3,5-triazin-2-amine 3 (290 mg, 1.2 mmol) and 2-methyl-1H-indole-4-carbonitrile 4 (187 mg, 1.2 mmol) in dioxane (10 mL) was added Pd₂(dba)₃ (183 mg, 0.2 mmol), X-Phos (190 mg, 0.4 mmol) and Cs₂CO₃ (650 mg, 2 mmol). The mixture was degassed for 3 times, and then stirred at 100° C. for 12 hours. The resulting mixture was concentrated in vacuo and the residue was purified by Combiflash (petroleum ether/ethyl acetate=3:1) to give the 1-(4-(benzylamino)-6-methyl-1,3,5-triazin-2-yl)-2-methyl-1H-indole-4-carbonitrile 5 (150 mg, 35%). LRMS (M+H+) m/z: calcd 355.16; found 355.

To a solution of 1-(4-(benzylamino)-6-methyl-1,3,5-triazin-2-yl)-2-methyl-1H-indole-4-carbonitrile 5 (150 mg, 0.42 mmol) in DMSO (1.6 mL) was added UHP (320 mg, 3.4 mmol) and K₂CO₃ (29 mg, 0.21 mmol). Then water (0.07 mL) was added to the mixture and stirred at room temperature for 2 hours. Water was added to the mixture, filtrated and the cake was dried to give the crude product, which was purified by Combiflash (dichloromethane/methanol=20:1) to give 1-(6-(benzylamino)-2-methylpyrimidin-4-yl)-2-methyl-1H-indole-4-carboxamide B (60 mg, 38%). LRMS (M+H+) m/z: calcd 373.17; found 373. HPLC purity (214 nm): 97%. ¹HNMR (300 MHz, DMSO): δ 8.80-8.65 (m, 1H), 8.38 (d, J=7.5 Hz, 1H), 7.85-7.80 (br, 1H), 7.68-6.97 (m, 9H), 4.62-4.59 (m, 2H), 2.74 (s, 1H), 2.61 (s, 2H), 2.42 (s, 3H).

### Biological Protocols

The in vitro and in vivo biological assays to determine the anti-cancer properties of the triazine compounds of the invention are summarized above. The details of these protocols show how the assays are carried out.

### P97 Biochemical Assay Protocol

The p97 assay is an initial screening assay used to determine inhibitory activity of the triazine compounds of the invention against the p97 complex. As discussed above, inhibition of activity of the p97 proteosome complex can enable apoptosis and cause elimination of neoplastic cells (cancer cells). The method follows that of Christianson in Nat. Cell Biol., (2011) 14:93.

The Reagents Used for the p97 Assay Include:

Assay Buffer is a mixture of 50 mM TRIS pH 7.5, 20 mM MgCl₂, 0.02% TX-100, 1 mM DTT and 0.2% (v/v) Glycerol. The well plate is Platetype: Corning 3674, 384w plate. The identification kit is an ADP glo kit (Promega): stop buffer, detection reagent.

The Assay Protocol is Conducted as Follows:

Serial dilute compound in DMSO in a 1:3.33-fold 10 point serial dilution.

in each well of 384w plate add the following reagents:

0.5 μL compound serial diluted in DMSO (Final Conc. 10%)

2 μL ATP (Final Conc.=20 uM, diluted in assay buffer)

2.5 μL p97 (Final Conc.=20 nM, diluted in assay buffer)

Incubate at 37 deg C. for 15 min.

Add 5 μL of stop buffer, incubate at RT for 40 min.

Add 10 μL of detection reagent, incubate at RT for 30 min.

Read luminescence on Envision plate reader.

Upon obtaining the data from the luminescence reading, the data may be analyzed as follows:

Normalize luminescence data using no enzyme (full inhibition) and no compound (no inhibition) controls. Plot normalized luminescence data against log-transformed concentration values and fit to a sigmoidal curve to determine IC50 values (done in Collaborative Drug Discovery software).

Caco-2 Permeability Assay

This assay is designed as a model to indicate the permeability of a triazine compound of this invention through the gut-blood barrier. The result will yield indications of whether or not the triazine compound may be efficiently absorbed into the blood stream of a patient. Efficient, effective absorption of an orally administered drug determines in part its bioavailability. For the triazine compounds of the invention, this assay is a model to evaluate the bioavailability of the compounds as a result of their ability to pass through biological barriers to entry into the physiological system of the patient.

The experimental goal of the Caco-2 assay is to measure directional Caco-2 permeability of test compounds in cultured Caco-2 monolayer.

The test compounds are the triazine compounds of the invention.

### Set-Up

Instruments
Tissue culture CO₂ incubator with humidity control
Liquid handler
Orbital shaker
EVOM Epithelial Volt-ohmmeter fitted with planar electrodes (World Precision Instruments, Sarasota, Fla.) required for measuring transepithelial electrical resistance (TEER)
Bench top centrifuge with 96-well plate adaptor
Caco-2 cells (Human colorectal adenocarcinoma, ATCC #37-HTB, passage 30-45)
Cells seeded onto PET membranes (1 nm pore size, 0.31 cm² surface area) inside Falcon HTS multiwell Insert system using 24-well plates (Becton Dickinson plates, Part #351181, Fisher Scientific, Inc.) at a density of 23,000 cells/well. Cells grown 20-23 days with medium changed every 2-3 days

### Reagents

Ringers buffer solution (pH 7.4 at 25° C.)
Ringers buffer with 1% Methanol
   Blk solution: Ringers buffer: Methanol=2:1 (v/v); 100% Methanol including internal standard (IS); 10 mM stock dosing solution in DMSO; 100 μM dosing solution in buffer.

### Protocol Summary

Caco-2 permeability: 20-23 day/Passage 30-45
24-well format transwell: 0.31 cm2 surface area
   Donor conc: 100 μM including 1% DMSO
   A: 300 μL pH 7.4/B: 1200 μL pH 7.4 Ringers buffer
   Directionality: A B and B A (N=4)
   Donor side sampling: 20 μL at beginning and end (90 min)
   Receiver side sampling: 100 μL at 30, 50, 70, and 90 min
      Incubation at 50 oscillations per minute, 37° C., 5% CO₂, 95% humidity
Analysis: LC-UV, LC-MS, or LSC
   Output: Peff (cm/sec)=(dX/dt)/(A*Co*60), dX/dt: transported amount (nmole) versus time (minute) profile in the receiver chamber; A: surface area (cm²); and Co: initial donor concentration (mM)
Positive control: Atenolol and propranolol
Membrane integrity: TEER>200 Ocm²
   Amount required: Approximately 1 mg or 100 μl of 10 mM test compound in DMSO
   Instruments: CO₂ incubator with humidity control, liquid handler, epithelial volt-ohmmeter for TEER, Caco-2 cells (ATCC #37-HTB), and 24-well insert plates (PET membranes, 1 μm pore size, 0.31 cm² plates, Part #351181) surface area, Becton Dickinson
Throughput: 6 compounds/2 Caco-2 plates/1 FTE/day

TABLE 24
Preparation of Ringers with Glucose (Isotonic = 290 mOsm/kg), pH 7.4

| | | | | | Mass | Mass |
|---|---|---|---|---|---|---|
| | Molecular | Con- | | Mass (g) | (g) | (g) |
| Chemical | Wt | centration | | for 1 L | for 2 L | for 4 L |
| CaSO4 2H2O | 172.2 | 1.25 | mM | 0.2152 | 0.4305 | 0.861 |
| MgSO4 7H2O | 246.5 | 1.1 | mM | 0.2712 | 0.5423 | 1.0846 |
| KCl | 74.55 | 5 | mM | 0.3728 | 0.7455 | 1.491 |
| Na2HPO4 | 142 | 1.15 | mM | 0.1633 | 0.3266 | 0.6532 |
| NaH2PO4 H2O | 138 | 0.3 | mM | 0.0414 | 0.0828 | 0.1656 |
| NaHCO3 | 84.01 | 25 | mM | 2.1 | 4.2 | 8.401 |
| Glucose | 180.2 | 25 | mM | 4.505 | 9.01 | 18.02 |
| (C6H12O6) | | | | | | |
| NaCl | 58.44 | 110 | mM | 6.428 | 12.86 | 25.71 |

### Preparation of 4 L Solution

1. To 3.5 L distilled water, add Calcium Sulfate and Magnesium Sulfate.
Note: Add Calcium Sulfate and Magnesium Sulfate first due to low solubility and add the remaining ingredients in the order listed in Table 1.

2. Adjust the final volume of the solution to 4 L with distilled water, with continuous stirring.

3. Adjust final solution to a pH of 7.4 using 1N HCl or 1N NaOH.

4. Make the buffer iso-osmotic using NaCl. Measure tonicity of the solution using a tonometer. Given that an isotonic solution is equivalent to 0.9% NaCl (290 mOsm/L),
Y={(290-x)/290}×9 mg×4000 mL, where y=NaCl required (in mg) to make the solution isotonic and x=observed tonicity of solution (reported as mOsm/L).

### Preparation of Dosing Solution in 15 ML PP Tube

1. 100 μM dosing solution in RG: 140 μL 10 mM stock+(14 mL 140 μL) RG

### Preparation of Calibration in 96 Shallow Well

1. Prepare 10 μM standard: 100 μl of 100 μM dosing solution+0.9 mL Ringers with 1% Methanol.
2. Prepare analytical standard solutions 10, 5, 2, 1, 0.5, 0.2, 0.1, 0.05, 0.02, 0.01, and 0 μM. (See Table 26)

TABLE 25
Preparation of analytical calibration in 96 shallow well

| | 1 | 2 | 3 | 4 | 5 | 6 | 7 | 8 | 9 | 10 | 11 | 12 |
|---|---|---|---|---|---|---|---|---|---|---|---|---|
| | 0 | 20 μL of | 20 μL of | 20 μL of | 20 μL of | 20 μL of | 20 μL of | 20 μL of | 40 μL of | 100 μL of | 200 μL of | Source |
| | | 0.1 μM | 0.2 μM | 0.5 μM | 1 μM | 2 μM | 5 μM | 10 μM | 10 μM | 10 μM | 10 μM | solution |
| | 180 μL | 180 μL | 180 μL | 180 μL | 180 μL | 180 μL | 180 μL | 180 μL | 160 μL | 100 μL | 0 | 1% MeOH |
| | | | | | | | | | | | | in buffer |
| Comp 1 | Blk | 0.01 μM | 0.02 μM | 0.05 μM | 0.1 μM | 0.2 μM | 0.5 μM | 1 μM | 2 μM | 5 μM | 10 μM | |
| Comp 2 | | | | | | | | | | | | |
| Comp 3 | | | | | | | | | | | | |

### Transport Studies

### Dosing and Sampling

1. Equilibrate both sides of the monolayers for 10 minutes with prewarmed (37° C.) drug-free Ringers buffer (300 μL apical side, 1,200 μL basolateral side) supplemented with glucose (25 mM).

2. Measure TEER under 37° C. water bath conditions.

Note: The TEER value serves as a quality control check for monolayer integrity. At 21 days post-seeding, each Caco-2 cell monolayer should have a TEER value of greater than or equal to 2000×cm² and those not meeting this criteria are not suitable for permeability evaluations.

3. When studying A to B transport: Fill basolateral side with 1,200 μL of Ringers buffer. Initiate transport experiments by transferring test drug dosing solution (3204) to apical side.

4. When studying B to A transport: Fill apical side with 300 μl of Ringers buffer. Initiate transport experiments by transferring test drug dosing solution (1,220 μL) to basolateral side. Transport studies for each direction (A to B, B to A) are performed in quadruplicate for each test drug.

5. Start timer after dosing last donor well.

6. Remove 20 μL aliquots from the donor wells at 0 minutes (Dₒ) and transfer these aliquots to the donor site of the 96-well plate containing 180 μL buffer with 1% Methanol. This step effectively dilutes the D₀ ten times.

7. Initiate transport studies by placing plate on orbital shaker maintained inside a prewarmed (37° C.) and humidified (5% CO₂) incubator. Studies are performed under stirring conditions at 50 oscillations per minute.

8. Remove 100 μL aliquots from the receiver side of the monolayer at 30, 50, 70, and 90 minutes postdosing and transfer these aliquots to the corresponding 96-well sample plate (See Table 26). Replace with an equivalent volume of prewarmed buffer.

9. Remove 20 μL aliquots from the donor side of the monolayer at 90 minutes postdosing (D_{f}) and transfer these aliquots to a donor site of a 96-well plate containing 180 μL Ringers buffer with 1% Methanol. This step effectively dilutes the D_{f} ten times.

10. Replace both sides of monolayer with fresh, drug-free, prewarmed Ringers buffer (300 μL apical side, 1,200 μL basolateral side) and equilibrate for 10 minutes.

11. Measure TEER under 37° C. water bath conditions.

### Sample Handling

The following steps refer to 96-well analytical plate for Caco-2, Table 26.

1. Transfer 20 μL of diluted D₀ and D_{f} to corresponding 96-well sample plate with each well containing 80 μL buffer with 1% Methanol. This step effectively dilutes the samples five times further. Therefore, donor samples are diluted 50 times from their initial concentration.

2. Transfer 100 μl of analytical calibration (from 0 to 10 μM) to the sample plate row 1.

3. Add 50 μL Methanol including IS to all sample wells and mix (standards, samples, and D₀ and D_{f}).

4. Transfer 150 μL of Blk solution to the analytical plate row 2.

5. Seal the analytical plate with adhesive sealing film and store samples with label at −80° C. for LC-UV or LC-MS analysis.

6. Analyze 20 μL aliquots of the individual permeability samples and the standards using a suitable analytical instrument.

7. Peff=(dX/dt)/(A×C₀×60), where P_{eff} is the effective permeability in cm/sec, X=mass transported, A is the surface area (cm)² available for transport, C₀ is the initial donor drug concentration (μM), and dX/dt is the slope of the best fit line through the transported amount (nmole) versus time (min) profile in the receiver chamber.

TABLE 26
Analytical Plate for Caco-2 (96-well plate)

| 0 | 0.01 μM | 0.02 μM | 0.05 μM | 0.1 μM | 0.2 μM | 0.5 μM | 1 μM | 2 μM | 5 μM | 10 μM | |
|---|---|---|---|---|---|---|---|---|---|---|---|
| Blk | Blk | Blk | Blk | A to B | | | B to A | Blk | Blk | Blk | Blk |
| 30-Jan | Feb-30 | 30-Mar | 30-Apr | | | | | 30-May | 30-Jun | 30-Jul | 30-Aug |
| Jan-50 | Feb-50 | Mar-50 | Apr-50 | | | | | May-50 | Jun-50 | Jul-50 | Aug-50 |
| Jan-70 | Feb-70 | Mar-70 | Apr-70 | | | | | May-70 | Jun-70 | Jul-70 | Aug-70 |
| Jan-90 | Feb-90 | Mar-90 | Apr-90 | | | | | May-90 | Jun-90 | Jul-90 | Aug-90 |
| 1-Do | 2-Do | 3-Do | 4-Do | | | | | 5-Do | 6-Do | 7-Do | 8-Do |
| 1-Df | 2-Df | 3-Df | 4-Df | | | | | 5-Df | 6-Df | 7-Df | 8-Df |

### Positive Control Data

Mean data in Table 27 represent the mean value from 12 separate inter-day experiments.

TABLE 27
P_{eff} (×E−6 cm/sec) in pH 7.4 Caco-2

| | A B | B A |
|---|---|---|
| Atenolol | | |
| Mean | 1.08 | 2.29 |
| Range | 0.69-1.80 | 1.69-2.68 |
| Propranolol | | |
| Mean | 28.53 | 20.91 |
| Range | 18.50-36.80 | 16.30-31.40 |

### Mouse Liver Microsome Assay

The liver microsome assay is a model for studying the metabolic stability of the triazine compounds of the invention. Metabolic stability is another aspect determining bioavailability. The facility of a compound to be bioabsorbed into the blood stream as shown by the Caco-2 model indicates the degree to which an oral dose of the compound will reach the blood stream. The body efficiently metabolizes substances to rid them from the body and/or to utilize them as nutrients. This aspect of bioavailability can be determined by such model studies as liver microsomal metabolism. Whether by oxidation, conjugation or any other biological pathway, metabolism of a drug determines at least in part the lifetime of the drug in the body.

The mouse liver microsome assay is a model designed to establish drug half-life in vivo. The liver enzymes are responsible to conversion of substances to materials that can be readily excreted by the body. Other routes for such metabolism include kidney metabolism, cellular metabolism and the like.

In this protocol, the compound is combined with a liver microsomal preparation (protein) and NADPH. The mixture is incubated and the rate of disappearance of the compound from the test solution is measured. Measurement is made by screening for the compound concentration at specified times using liquid chromatography in combination with mass spectroscopy.

Concentrations of reactants ready for formulation as the test solution:

Protein: 1.0 mg/ml

Compound: 1 um

Organic solvent: 0.4% DMSO

Medium: 0.1 M Potassium Phosphate (KB)

1 mM NADPH (sigma N1630, FW 833.3, make freshly)

Prepare test article (TA, i.e., a compound of the invention) by dissolving solid TA in DMSO to make a 0.25 mM solution

Amounts of Reactant Solutions to be Combined to Form the Test Solution:

423 ul KB (potassium phosphate)

+25 ul MLM (20 mg/ml) (mouse liver microsomal preparation)

448 ul

+2 ul Test compound (a triazine compound at 0.25 mM DMSO)

+50 ul NADPH stock (10 mM, 10×)

500 ul

Test Protocol for Conducting the Assay
1. Add 423 ul KB to an 8-strip deep well tubes
2. Add 25 ul of MLM for condition 1
3. Place on ice, add 2 ul cmpds (250×stock in DMSO, stock at 0.25 mM)
4. Preincubate the reaction mixture at 37 C for 3 to 5 minutes (shaking at 150 rpm)
5. Initiate reaction by adding 50 ul NADPH for condition 1
6. Add 50 ul KB for condition 2
7. An aliquot of samples of 100 ul were collected at 0, 15, 30, and 60 min time point, and 200 ul of acetonitrile mixture containing IS was added to quench the reaction.
8. Centrifuge for 10 min at 4000 rpm
9. The supernatant were injected for liquid chromatographic tandem mass spectrometry (LC-MS/MS) analysis

Procedure of Protein Binding Using 96-Well Equilibrium Dialyzer

Non-specific protein binding is another facet affecting bioavailability and effectiveness of a drug. To assay a compound for non-specific binding, the compound is combined with human blood plasma and the solution dialyzed against a membrane constructed to prevent passage of larger molecules such as human plasma proteins but allow passage of small molecules such as the compounds of the invention. Typically, such membranes allow passage of such compounds irrespective of their salt or neutral form. The dialysate (solution passing through the membrane) is examined by liquid chromatography mass spectrometric techniques to determine the identity and concentration of the compound present. The concentration of compound in the dialysate compared with the concentration of compound combined with blood plasma indicates whether or not non-specific protein binding has occurred.

Equipment and Reagent:

96-Well Equilibrium Dialyzer (made by: Harvard Apparatus)

Plate Rotator with DIALYZER plates secured in clamp fixture

Buffer: DPBS (gibco, 1×)

Compound Concentration: 1 μM (˜0.5 in μg/mL) in Human Plasma

Procedure:
1. Seal the empty Sample Side well on the colored side with cap strips.
2. Invert the plate and carefully pipet a volume of buffer, 200 μL equal to the sample volume into the wells on the Buffer Side (clear frame) without touching the membranes by allowing the liquid to flow along the inner side wall of each well.
3. Gently seal the filled buffer wells with cap strips.
4. Invert the plate and carefully remove the cap strips from the sample side wells. Pipet desired samples, without touching the membranes.
5. Reseal the sample wells with the cap strips.
6. Slide the assembled DIALYZER Plate into a Plate Rotator and hand tighten the snobs. Turn on and allow rotating until equilibrium has been reached (24 hours at 37 C), remove the DIALYZER Plate from the Rotator.
7. After equilibrium has been reached, remove the DIALYZER Plate from the rotator.
8. Carefully remove the cap strips from the Buffer Side of the Plated (clear frame) and slowly pipet out the analysis samples from the wells taking care not to touch or puncture the membranes. Samples will include control at 4 C and stability at 37 C samples in PBS and plasma.

MS Analysis:
Prepare standard range 5, 10, 50, 100, 500 and 1000 ng/mL in Plasma
Pipet 10 μL each of standard and sample into 40 μL of blank buffer/blank plasma them (ratio: 1 plasma/4 DPBS), mix them.
Add 200 μL of Is (internal standard) in ACN, mix well.
Centrifuge the samples and transfer supernatant solution for LC/MS analysis.

### The Cell Assay Protocol

The cellular assay provides information about the anti-neoplastic activity of the compounds of the invention. The compounds are tested against cultured cancer cells to determine whether or not the compounds of the invention are capable of intersecting with cancer cells to minimize or eliminate such cells. The assay involves establishing colonies of such cells and then treating them with the test compound under specified conditions and analysis regima to determine results.

Day 1, Cell Plating to Establish Colonies of Cancer Cells

Cell Plating:
Seed cells ˜16 hrs prior to compound treatment
Plate 25 μL of A549 cells in every well of 384-well plate using multidrop.
   Two (2) black plates for IF at 2500 cells/well
   Let plate sit at room temp for 10-15 minutes prior to putting in incubator to allow cells to stick in middle of plate.
   One (1) white plate for viability at 500 cells/well.

Day 2 Treatment of Cultured Cells with Test Compounds

Treat Cells:
Serial dilute compounds with a 10 point 2-fold serial dilution in DMSO to make 250× stock compound solution
Dilute compounds 1:125 in cell culture media to make a 2× solution
Add 25 μl of dilution compounds to cell plates in well duplicates
Put cells back in incubator (6 hr incubation for black plates, 72 hr incubation for white plates).

Fix/Stain Black Plates:
Incubate cells in black plates with compound at 37 deg C. for 6 hrs.
add 15 μL of 16% Paraformaldehyde (PFA) directly into media of each well,
   incubate at room temp for 5 min, flick plate and wash in 50 μL of PBS
block in 50 μL of Blocking Buffer for 30 minutes (can go up to several hours)
   Blocking buffer: 1×PBS, 1% BSA, 0.3% Triton-X100, Hoechst (1:10,000)
incubate in 25 μL of primary antibody in blocking buffer at 4 deg C. over night

Primary Antibodies:
Plate A K48-Ub 1:20,000 (millipore 05-1307 Lot 2049282) Rabbit
   CHOP/Gadd153 1:2,000 (SC-7351) Mouse
Plate B P53 1:2,000 (SC-6243) Rabbit
   p62/SQSTM1 1:2,000 (SC-28359) Mouse
   overnight at 4 deg C.

Secondary Antibodies:
AlexaFluor488 Goat anti-Rabbit 1:2,000 (Life Tech A11008)
AlexaFluor555 Goat anti-Mouse 1:2,000 (Life Tech A21422)

Day 3/4

Black Plate Staining (Cont):
wash black plates 3× in 50 μL PBS (˜5 min each)
   incubate in 25 μl of secondary antibody (1:2,000) in blocking buffer for 1-2 hrs at room temp (alexafluor488-anti-Rabbit/alexafluor555-anti-Mouse)
wash 4× in 50 μL PBS (˜5 min each)
leave plates in PBS for imaging
clean bottom of plates with 70% EtOH

Imaging:
Image plates in high content microscope with 405 nm, 488 nm and 555 nm filters

Data Analysis:
Nuclear counts and cellular intensities of each markers are measured using Hoechst as a nuclear marker with an automated image analysis protocol using Matlab software (Math Works)

Day 5

Viability Assay:
Thaw an aliquot of frozen cell titer glo (Promega G7572) at room temperature.
Add 45 mL of NaCl/PBS solution to 5 ml of cell titer glo (10×).
Remove white plates from incubator, leave at room temp for 30 minutes.
Add 25 μl of diluted cell titer glo to each well.
Shake plate for >1 minute.
Incubate plate for >5 minutes to stabilize luminescence.
   Luminescence is stable for up to 3 hours.
Read luminescence on plate reader

### Statements of the Invention

1. A nitrogen hexacycle compound of Formula I

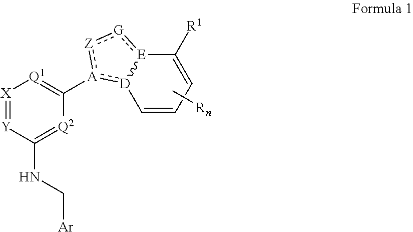

Wherein:
   One of X and Y is nitrogen and the other is CR²;
   One of Q¹ and Q² is nitrogen and the other is CR^{2′}, or both of Q¹ and Q² are nitrogen;
   The bicyclic ADEGZ group is a P2 group;
   The five member ring of the P2 group is partially saturated or aromatic;
   The six member ring of the P2 group is aromatic;
   A, D and E each are independently C or nitrogen, the carbon of C being an sp² carbon and A additionally may be CR⁶, the carbon of CR⁶ being an sp³ carbon;
   The squiggle bond between D and E is a single bond if one of either of D and E is nitrogen, and the squiggle bond between D and E is a double bond if both of D and E are carbon;
   The dotted bonds of the five member ring may be single or double bonds depending upon the valence and electon configuration of the bonded atom(s).
   G and Z are each independently nitrogen, NR³, CR⁴, C(R⁵)₂, oxygen or sulfur, the carbon of CR⁴ being an sp² carbon and the carbon of C(R⁵)₂ being an sp³ carbon, provided that:
   when one of G and Z is oxygen or sulfur, each of A, D and E is an sp² carbon;
   G and Z are not together a combination of oxygen and sulfur and are not both oxygen or both sulfur;
   when G and Z are both C(R⁵)₂, A is CR⁶ or N and D and E are both sp² carbons;
   when G and Z are both CR⁴, A is CR⁶ or N and D and E are both sp² carbons;
   R¹ is selected from hydrogen, an aliphatic group optionally substituted by a functional group or a functional group;
   R², R^{2′}, Rₙ, R⁴ and each of R⁵ are each independently selected from hydrogen, an aliphatic group optionally substituted by a functional group, an optionally substituted aromatic group and a functional group with one of R⁵ preferably being hydrogen;
   R³ is hydrogen, an aliphatic group optionally substituted by a functional group and a functional group;
   R⁶ is hydrogen or an alkyl group of 1 to 3 carbons;
   n is 0 or an integer of 1 or 2;
   Ar is an aryl group optionally substituted by a functional group or an aliphatic group;
   The number of boronic acid or boronic ester groups bonded anywhere to Formula I is one, the boronic acid and boronic ester groups being specific groups of a functional group.
2. A nitrogen hexacycle compound according to statement 1 wherein A is nitrogen, G is CR⁴, D and E are sp² carbon, and Z is nitrogen or CR⁴.
3. A nitrogen hexacycle compound according to statement 1 wherein A and G both are nitrogen, D and E both are sp² carbon, and Z is nitrogen or CR⁴.
4. A nitrogen hexacycle compound according to statement 1 wherein A, D and E all are sp² carbon, G is NR³ or oxygen and Z is CR⁴
5. A nitrogen hexacycle compound according to statement 1 wherein A, D and E all are sp² carbon, G is NR³ or oxygen and Z is nitrogen.
6. A nitrogen hexacycle compound of statement 1 wherein G and E both are nitrogen and A and D are both sp² carbon.
7. A nitrogen hexacycle compound of statement 1 wherein G and D are nitrogen and A and E are both sp² carbon.
8. A nitrogen hexacycle compound of statement 1 wherein Z and G are both CR⁴ or C(R⁵)₂, A is CR⁶ or N and D and E are both sp² carbon.
9. A compound according to statement 1 wherein A is CR⁶, Z is nitrogen, G, D and E are all sp² carbon.
10. A nitrogen hexacycle compound of any one of statements 1-9 wherein R¹ is not hydrogen but is a functional group or an aliphatic group optionally substituted by a functional group.
11. A nitrogen hexacycle compound of any one of statements 1-10 wherein R¹ is an aliphatic group selected from linear, branched or cyclic alkyl of 1 to 6 carbons, linear, branched or cyclic alkenyl of 2 to 6 carbons, linear, branched or cyclic alkoxyalkyl or alkoxyalkenyl of 2 to 6 carbons, the thia analog thereof, linear, branched or cyclic alkanoyl or alkenoyl of 2 to 6 carbons, linear, branched or cyclic alkanoylalkyl or alkenoylalkyl or 3 to 7 carbons, linear, branched or cyclic alkanoyloxy or alkanoyloxy of 2 to 6 carbons, or linear, branched or cyclic alkanoyloxyalkyl or alkenoyloxyalkyl of 3 to 7 carbons.
12. A nitrogen hexacycle compound of any one of statements 1-9 wherein R¹ is not hydrogen or an aliphatic group, but is a functional group that is polar and preferably is hydrogen bonding.
13. A nitrogen hexacycle compound of statement 12 wherein R¹ is a polar, hydrogen bonding functional group.
14. A nitrogen hexacycle compound of statement 13 wherein R¹ is selected from the group consisting of B(OH)₂, B(OR)₂ wherein R is an alkyl group of 1 to 6 carbons, OR^{d}, (CH₂)ₙOR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, —N(R^{d})C(O)N(R^{d})₂, N(R^{d})C(NR^{d})N(R^{d})₂, N(R^{d})S(O)ₜR^{d}, S(O)ₜOR^{d}, S(Oₜ)R^{d}, S(O)ₜN(R^{d})₂, N(R^{d})₂, (CH₂)ₙN(R^{d})₂, PO₃(R^{d})₂, C(O)R^{d} and CF₃; each n is independently an integer of 1, 2 or 3, preferably 1; each t is independently an integer of 1 or 2, preferably 2; each R^{d} is independently hydrogen, alkyl of 1 to 6 carbons, fluoroalkyl of 1 to 6 carbons, carbocyclyl of 3 to 10 carbons, carbocyclylalkyl of 4 to 12 carbons, aryl of 6 to 10 carbons, aralkyl, heterocyclyl, heterocyclylalkyl, heteroaryl, heteroarylalkyl, alkenyl of 2 to 6 carbons, alkynyl or 2 to 6 carbons or any combination thereof, and R^{d} preferably is hydrogen or alkyl of 1 to 4 carbons, more preferably hydrogen or methyl, most preferably hydrogen.
15. A nitrogen hexacycle compound of statement 14 wherein R^{d} is hydrogen, phenyl or alkyl of 1 to 6 carbons, preferably 1 to 3 carbons, more preferably methyl or ethyl.
16. A nitrogen hexacycle compound of statement 15 wherein R^{d} is hydrogen.
17. A nitrogen hexacycle compound of statement 15 wherein R^{d} is methyl.
18. A nitrogen hexacycle compound of statement 14 wherein R¹ is selected from the group consisting of boronic acid, boronic ester with the ester group being straight, branched or cyclic alkyl of 1 to 6 carbons, carboxylic acid, carboxyl ester with the ester group being straight, branched or cyclic alkyl of 1 to 6 carbons, carboxamide, N-alkyl carboxamide of 1 to 6 carbons in the straight, branched or cyclic alkyl group, sulfonic acid, sulfonyl ester with the ester group being straight, branched or cyclic alkyl of 1 to 6 carbons, sulfonamide, alkyl substituted sulfonamide with the alkyl group being straight, branched or cyclic of 1 to 6 carbons, amine (NH₂), mono or dialkyl amine with the alkyl being straight, branched or cyclic of 1 to 6 carbons, N-alkyl amino methyl with the alkyl group being straight, branched or cyclic of 1 to 6 carbons, nitrile, or straight, branched or cyclic alkoxy of 1 to 6 carbons.
19. A nitrogen hexacycle compound of statement 14 wherein R¹ is selected from the group consisting of boronic acid, boronic alkyl ester of 1 to 3 carbons in the alkyl group, carboxyl, sulfonoxy, carboxamide, sulfonamide, aminocarbonyl alkyl, aminosulfonylalkyl, amine, monoalkyl amine, hydroxyl, hydroxyalkyl, alkoxy, cyano, nitro, carboxylic ester, sulfonic ester, methylenyl or ethylenyl carboxylic acid or sulfonic acid, methylenyl or ethylenyl carboxylic or sulfonic ester, methylenyl or ethylenyl carboxamide or sulfonamide.
20. A nitrogen hexacycle compound of statement 14 wherein R¹ is selected from the group consisting of from OMe, OEt, CN, V(CH₂)ₘW, N(R^{a})₂, CO(NR^{a})₂, B(OH)₂, B(OR)₂ wherein R is an alkyl group of 1 to 6 carbons, SO₂R^{a} and SO₂N(R^{a})₂; wherein each R^{a} independently is H, Me, Et; n is independently 0 or 1; m is 0, 1, 2 or 3; W is amino, alkylamino, alkoxy, carboxy, carboxylic acid, carboxamide, carboxyl ester or N-alkyl carboxamide, sulfonic acid, sulfonamide, boronic acid, boronic Me or Et ester; and V is O, S, NH, CO₂, CONH and NH.
21. A nitrogen hexacycle compound of any one of statements 1-20 wherein R² and R^{2′} are each independently selected from the group consisting of hydrogen, linear, branched or cyclic alkyl or alkenyl of 1 to 6 carbons (2 minimum for alkenyl), halogen, B(OH)₂, B(OR^{g}) wherein R^{g} is an alkyl of 1 to 3 carbons, OR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, N(R^{d})C(O)N(R^{d})₂, N(R^{d})C(NR^{d})N(R^{d})₂, N(R^{d})S(O)ₜR^{d}, S(O)ₜOR^{d}, S(O)ₜN(R^{d})₂, N(R^{d})₂, (CH₂)ₜN(R^{d})₂ and PO₃(R^{d})₂ wherein each R^{d} is independently hydrogen, alkyl, fluoroalkyl, carbocyclyl, carbocyclylalkyl, aryl, aralkyl, heterocyclyl, heterocyclylalkyl, heteroaryl, heteroarylalkyl alkenyl, alkynyl or any combination thereof; each t is independently selected from the group of integers of 1 and 2; each q is independently an integer of 0, 1, 2 or 3; n is an integer of 0, 1 or 2, preferably 1, more preferably 0, and R^{d} preferably is hydrogen or alkyl of 1 to 4 carbons, more preferably hydrogen or methyl, most preferably hydrogen.
22. A nitrogen hexacycle compound of statement 21 wherein R^{d} is hydrogen, phenyl or alkyl of 1 to 6 carbons, preferably 1 to 3 carbons, more preferably methyl or ethyl.
23. A nitrogen hexacycle compound of statement 21 wherein R^{d} is hydrogen.
24. A nitrogen hexacycle compound of statement 21 wherein R^{d} is methyl.
25. A nitrogen hexacycle compound of statement 21 wherein R² and R^{2′} are each independently selected from the group consisting of hydrogen, halogen, straight, branched or cyclic alkyl of 1 to 6 carbons, B(OH)₂, B(OR)₂ wherein R is an alkyl group of 1 to 6 carbons, carboxylic acid, carboxyl ester with the ester group being straight, branched or cyclic alkyl of 1 to 6 carbons, N-alkyl amino methyl with the alkyl group being straight, branched or cyclic of 1 to 6 carbons, sulfonic acid, sulfonyl ester with the ester group being straight, branched or cyclic alkyl of 1 to 6 carbons, sulfonamide, amine (NH₂), mono, di or trialkyl amine with the alkyl being straight, branched or cyclic of 1 to 6 carbons, nitrile, carboxamide, N-alkyl carboxamide of 1 to 6 carbons in the straight, branched or cyclic alkyl group, perfluoroalkyl of 1 to 3 carbons, and straight, branched or cyclic alkoxy of 1 to 6 carbons.
26. A nitrogen hexacycle compound of any one of statements 1-25 wherein Rₙ, R⁴ and each of R⁵ are each independently selected from hydrogen, halogen, straight or branched alkyl of 1 to 6 carbons, carboxylic acid, carboxamide, substituted aminomethyl, sulfonic acid, sulfonamide, amine, mono, di or trialkyl amine of 1 to 6 carbons, nitrile, N-alkyl carboxamide of 1 to 6 carbons in the alkyl group, perfluoroalkyl of 1 to 3 carbons, alkoxy of 1 to 6 carbons, boronic acid or boronic ester having 1 to 3 carbons in the ester group.
27. A nitrogen hexacycle compound of statement 26 wherein n of Rₙ is 0, R³ is hydrogen or methyl, R⁴ and R⁵ are each independently hydrogen or methyl, and R² and R^{2′} are each independently selected from H, Me, Et, propyl (Pr), isopropyl (iPr), butyl (Bu), isobutyl (iBu), OMe, OEt, CN, OCH₂CH₂X, N(R^{a})₂, CO(NR^{a})₂, B(OH)₂, B(OR)₂ wherein R is an alkyl group of 1 to 6 carbons, SO₂R^{a}, SO₂N(R^{a})₂; R^{a} is H, Me, Et, Ph and when two R^{a}'s are present, each is selected independently; and X is amino, alkylamino, alkoxy, carboxy, carboxamide, carboxyl ester or N-alkyl carboxyamide.
28. A nitrogen hexacycle compound of statement 27 wherein:
   R¹ is B(OH)₂, B(OMe or Et)₂, CONH₂, CN, SO₂NH₂, COOH, COOMe, COOEt, CH₂NH₂, CH₂NHCOCH₃, CH₂NHSO₂CH₃ or CH₂OH; R² and R^{2′} are each independently H, Me, Et, COOH, CONH₂, SO₃H, SO₂NH₂, B(OH)₂, B(OMe or Et)₂, OMe, OEt, CN, F, Cl or Br, most especially, H or Me and of these two substituents, preferably H; R³ is H, Me, Et, COMe, COEt, SO₂Me or SO₂Et, most especially H or Me and of these two substituents, preferably H; R⁴ and each of R⁵ are each independently H, Me or Et, most especially H; Rₙ is excluded by n as 0; R⁶ is hydrogen.
29. A nitrogen hexacycle compound of any one of statements 1-28 wherein Ar is phenyl or substituted phenyl with the substituent being is fluoro, trifluoromethyl, boronic acid, boronic alkyl ester with a C1 to C6 alkyl, carboxylic acid, carboxylic alkyl ester with a C1 to C6 alkyl, carboxyamide, sulfonic acid, sulfonamide; preferably fluoro, boronic acid, boronic ester, carboxylic acid, carboxamid, sulfonic acid, sulfonic ester; more preferably fluoro, boronic acid, carboxylic acid, sulfonic acid; most preferred fluoro or boronic acid.
30. A nitrogen hexacycle compound of statement 29 wherein Ar is phenyl, phenyl-4-boronic acid or 4-fluorophenyl.
31. A nitrogen hexacycle compound of any one of statements 1-30 wherein the P2 group is one of Formulas IA, ID, IE, IG, IH, IK, IK-sat, JO, IP, IS, IT, IU, IW, IX, IY and IZ-2 and the substituents R¹ through R⁶ and Rₙ are present, are delineated as given by any one of statements 1-30 and are positioned as given in Formula I:

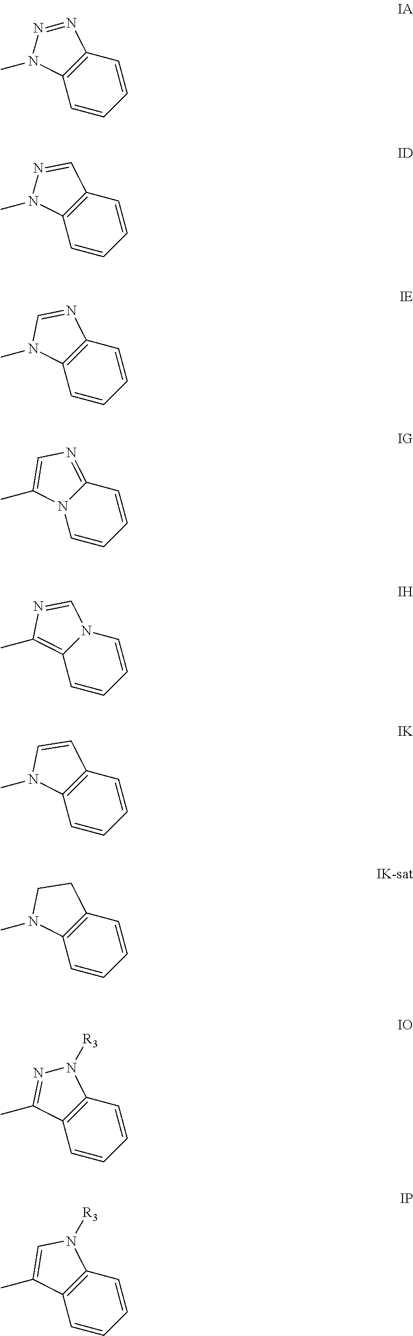

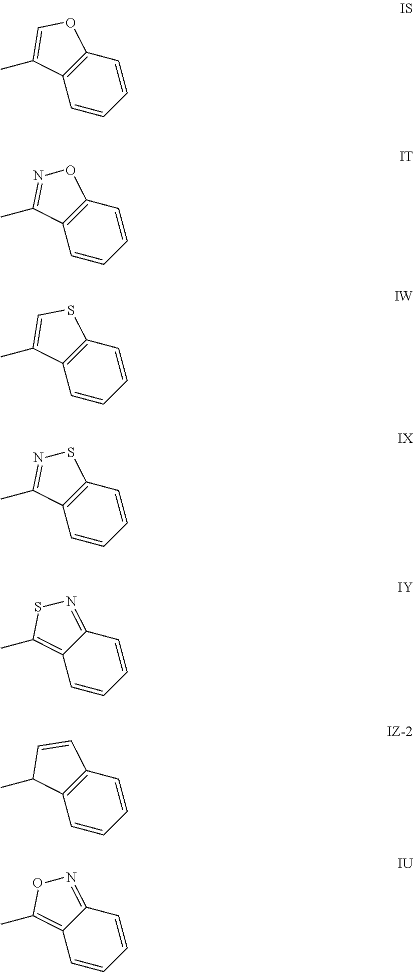

32. A nitrogen hexacycle compound of any one of statements 1-31 wherein Q¹ and Q²⁻ are both nitrogen as shown by formulas NT5 and NT6 wherein the upper right arrow designates the P2 group position and the bottom arrow designates the P4 group position:

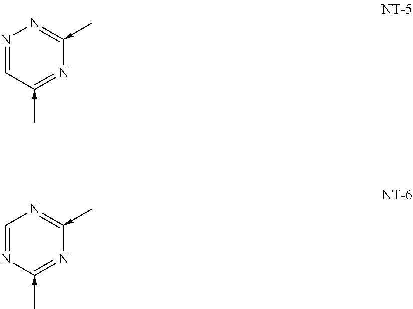

33. A pharmaceutical composition comprising a pharmaceutically acceptable carrier and an amount of a compound of any of statements 1 to 32 which is effective as an inhibitor of the AAA family member Valosin containing protein.
34. A pharmaceutical composition according to statement 33 wherein the Valosin containing protein is in a human cell.
35. A method of decreasing Valosin containing protein activity or decreasing degradation of a proteasome system substrate comprising administering to a patient an effective amount of a compound of any of statements 1 to 32.
36. A method of decreasing Valosin containing protein activity or degradation of a proteasome system substrate comprising administering to a patient an effective amount of a pharmaceutical composition of statements 33 or 34.
37. A method according to statement 35 or 36 wherein the patient is a human.
38. A method for treatment of a neoplastic malcondiction associated with Valosin containing protein comprising administering to a patient in need thereof an effective amount of a compound according to statements 1 to 32 or an effective amount of a pharmaceutical composition according to statements 33 or 34.

### Summary Statements

The inventions, examples, biological assays and results described and claimed herein have may attributes and embodiments include, but hot limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such paten, publication, scientific article, web site, electronically available information, text book or other referenced material or document.

The written description of this patent application includes all claims. All claims including all original claims are hereby incorporated by reference in their entirety into the written description portion of the specification and the right is reserved to physically incorporated into the written description or any of the portion of the application any an all such claims. Thus, for example, under no circumstances may the patent be interpreted as allegedly not providing a written description for a claim on the assertion that the precise wording of the claim is not set forth in haec verba in written description portion of the patent. All features disclosed in this specification may be combined in any order and in any combination with any of the formulas I, II and/or III.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

What is claimed is:

1. A nitrogen hexacycle compound of Formula I 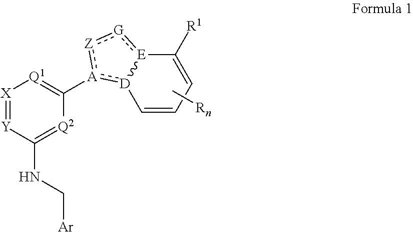
Wherein:
One of X and Y is nitrogen and the other is CR²;
One of O¹ and Q² is nitrogen and the other is CR^{2′}, or both of Q¹ and Q² are nitrogen;
The bicyclic ADEGZ group is a P2 group;
The five member ring of the P2 group is partially saturated or aromatic;
The six member ring of the P2 group is aromatic;
A, D and E each are independently C or nitrogen, the carbon of C being an sp² carbon and A additionally may be CR⁶, the carbon of CR⁶ being an sp³ carbon;
The squiggle bond between D and E is a single bond if one of either of D and E is nitrogen, and the squiggle bond between D and E is a double bond if both of D and E are carbon;
The dotted bonds of the five member ring may be single or double bonds depending upon the valence and electon configuration of the bonded atom(s).
G and Z are each independently nitrogen, NR³, CR⁴, C(R⁵)₂, oxygen or sulfur, the carbon of CR⁴ being an sp² carbon and the carbon of C(R⁵)₂ being an sp³ carbon, provided that:
when one of G and Z is oxygen or sulfur, each of A, D and E is an sp² carbon;
G and Z are not together a combination of oxygen and sulfur and are not both oxygen or both sulfur;
when G and Z are both C(R⁵)₂, A is CR⁶ or N and D and E are both sp² carbons;
when G and Z are both CR⁴, A is CR⁶ or N and D and E are both sp² carbons;
R¹ is selected from hydrogen, an aliphatic group optionally substituted by a functional group or a functional group;
R², R^{2′}, Rₙ, R⁴ and each of R⁵ are each independently selected from hydrogen, an aliphatic group optionally substituted by a functional group, an optionally substituted aromatic group and a functional group with one of R⁵ preferably being hydrogen;
R³ is hydrogen, an aliphatic group optionally substituted by a functional group and a functional group;
R⁶ is hydrogen or an alkyl group of 1 to 3 carbons;
n is 0 or an integer of 1 or 2;
Ar is an aryl group optionally substituted by a functional group or an aliphatic group;
The number of boronic acid or boronic ester groups bonded anywhere to Formula I is one, the boronic acid and boronic ester groups being specific groups of a functional group.

2. A nitrogen hexacycle compound according to claim 1 wherein A is nitrogen, G is CR⁴, D and E are sp² carbon, and Z is nitrogen or CR⁴.

3. A nitrogen hexacycle compound according to claim 1 wherein A and G both are nitrogen, D and E both are sp² carbon, and Z is nitrogen or CR⁴.

4. A nitrogen hexacycle compound according to claim 1 wherein A, D and E all are sp² carbon, G is NR³ or oxygen and Z is CR⁴

5. A nitrogen hexacycle compound according to claim 1 wherein A, D and E all are sp² carbon, G is NR³ or oxygen and Z is nitrogen.

6. A nitrogen hexacycle compound according to claim 1 wherein G and E both are nitrogen and A and D are both sp² carbon.

7. A nitrogen hexacycle compound according to claim 1 wherein G and D are nitrogen and A and E are both sp² carbon.

8. A nitrogen hexacycle compound according to claim 1 wherein Z and G are both CR⁴ or C(R⁵)₂, A is CR⁶ or N and D and E are both sp² carbon.

9. A nitrogen hexacycle compound according to claim 1 wherein A is CR⁶, Z is nitrogen, G, D and E are all sp² carbon.

10. A nitrogen hexacycle compound according to claim 1 wherein R¹ is not hydrogen but is a functional group or an aliphatic group optionally substituted by a functional group.

11. A nitrogen hexacycle compound according to claim 1 wherein R¹ is an aliphatic group selected from linear, branched or cyclic alkyl of 1 to 6 carbons, linear, branched or cyclic alkenyl of 2 to 6 carbons, linear, branched or cyclic alkoxyalkyl or alkoxyalkenyl of 2 to 6 carbons, the thia analog thereof, linear, branched or cyclic alkanoyl or alkenoyl of 2 to 6 carbons, linear, branched or cyclic alkanoylalkyl or alkenoylalkyl or 3 to 7 carbons, linear, branched or cyclic alkanoyloxy or alkanoyloxy of 2 to 6 carbons, or linear, branched or cyclic alkanoyloxyalkyl or alkenoyloxyalkyl of 3 to 7 carbons.

12. A nitrogen hexacycle compound according to claim 1 wherein R¹ is not hydrogen or an aliphatic group, but is a functional group that is polar and preferably is hydrogen bonding.

13. A nitrogen hexacycle compound according to claim 12 wherein R¹ is a polar, hydrogen bonding functional group.

14. A nitrogen hexacycle compound according to claim 13 wherein R¹ is selected from the group consisting of B(OH)₂, B(OR)₂ wherein R is an alkyl group of 1 to 6 carbons, OR^{d}, (CH₂)ₙOR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, —N(R^{d})C(O)N(R^{d})₂, N(R^{d})C(NR^{d})N(R^{d})₂, N(R^{d})S(O)ₜR^{d}, S(O)ₜOR^{d}, S(Oₜ)R^{d}, S(O)ₜN(R^{d})₂, N(R^{d})₂, (CH₂)ₙN(R^{d})₂, PO₃(R^{d})₂, C(O)R^{d} and CF₃; each n is independently an integer of 1, 2 or 3, preferably 1; each t is independently an integer of 1 or 2, preferably 2; each R^{d} is independently hydrogen, alkyl of 1 to 6 carbons, fluoroalkyl of 1 to 6 carbons, carbocyclyl of 3 to 10 carbons, carbocyclylalkyl of 4 to 12 carbons, aryl of 6 to 10 carbons, aralkyl, heterocyclyl, heterocyclylalkyl, heteroaryl, heteroarylalkyl, alkenyl of 2 to 6 carbons, alkynyl or 2 to 6 carbons or any combination thereof, and R^{d} preferably is hydrogen or alkyl of 1 to 4 carbons, more preferably hydrogen or methyl, most preferably hydrogen.

15. A nitrogen hexacycle compound according to claim 14 wherein R^{d} is hydrogen, phenyl or alkyl of 1 to 6 carbons, preferably 1 to 3 carbons, more preferably methyl or ethyl.

16. A nitrogen hexacycle compound according to claim 15 wherein R^{d} is hydrogen.

17. A nitrogen hexacycle compound according to claim 15 wherein R^{d} is methyl.

18. A nitrogen hexacycle compound according to claim 14 wherein R¹ is selected from the group consisting of boronic acid, boronic ester with the ester group being straight, branched or cyclic alkyl of 1 to 6 carbons, carboxylic acid, carboxyl ester with the ester group being straight, branched or cyclic alkyl of 1 to 6 carbons, carboxamide, N-alkyl carboxamide of 1 to 6 carbons in the straight, branched or cyclic alkyl group, sulfonic acid, sulfonyl ester with the ester group being straight, branched or cyclic alkyl of 1 to 6 carbons, sulfonamide, alkyl substituted sulfonamide with the alkyl group being straight, branched or cyclic of 1 to 6 carbons, amine (NH₂), mono or dialkyl amine with the alkyl being straight, branched or cyclic of 1 to 6 carbons, N-alkyl amino methyl with the alkyl group being straight, branched or cyclic of 1 to 6 carbons, nitrile, or straight, branched or cyclic alkoxy of 1 to 6 carbons.

19. A nitrogen hexacycle compound according to claim 14 wherein R¹ is selected from the group consisting of boronic acid, boronic alkyl ester of 1 to 3 carbons in the alkyl group, carboxyl, sulfonoxy, carboxamide, sulfonamide, aminocarbonyl alkyl, aminosulfonylalkyl, amine, monoalkyl amine, hydroxyl, hydroxyalkyl, alkoxy, cyano, nitro, carboxylic ester, sulfonic ester, methylenyl or ethylenyl carboxylic acid or sulfonic acid, methylenyl or ethylenyl carboxylic or sulfonic ester, methylenyl or ethylenyl carboxamide or sulfonamide.

20. A nitrogen hexacycle compound according to claim 14 wherein R¹ is selected from the group consisting of from OMe, OEt, CN, V(CH₂)ₘW, N(R^{a})₂, CO(NR^{a})₂, B(OH)₂, B(OR)₂ wherein R is an alkyl group of 1 to 6 carbons, SO₂R^{a} and SO₂N(R^{a})₂; wherein each R^{a} independently is H, Me, Et; n is independently 0 or 1; m is 0, 1, 2 or 3; W is amino, alkylamino, alkoxy, carboxy, carboxylic acid, carboxamide, carboxyl ester or N-alkyl carboxamide, sulfonic acid, sulfonamide, boronic acid, boronic Me or Et ester; and V is O, S, NH, CO₂, CONH and NH.

21. A nitrogen hexacycle compound according to claim 1 wherein R² and R^{2′} are each independently selected from the group consisting of hydrogen, linear, branched or cyclic alkyl or alkenyl of 1 to 6 carbons (2 minimum for alkenyl), halogen, B(OH)₂, B(OR^{g}) wherein R^{g} is an alkyl of 1 to 3 carbons, OR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, N(R^{d})C(O)N(R^{d})₂, N(R^{d})C(NR^{d})N(R^{d})₂, N(R^{d})S(O)ₜR^{d}, S(O)ₜOR^{d}, S(O)ₜN(R^{d})₂, N(R^{d})₂, (CH₂)ₜN(R^{d})₂ and PO₃(R^{d})₂ wherein each R^{d} is independently hydrogen, alkyl, fluoroalkyl, carbocyclyl, carbocyclylalkyl, aryl, aralkyl, heterocyclyl, heterocyclylalkyl, heteroaryl, heteroarylalkyl alkenyl, alkynyl or any combination thereof; each t is independently selected from the group of integers of 1 and 2; each q is independently an integer of 0, 1, 2 or 3; n is an integer of 0, 1 or 2, preferably 1, more preferably 0, and R^{d} preferably is hydrogen or alkyl of 1 to 4 carbons, more preferably hydrogen or methyl, most preferably hydrogen.

22. A nitrogen hexacycle compound according to claim 21 wherein R^{d} is hydrogen, phenyl or alkyl of 1 to 6 carbons, preferably 1 to 3 carbons, more preferably methyl or ethyl.

23. A nitrogen hexacycle compound according to claim 21 wherein R^{d} is hydrogen.

24. A nitrogen hexacycle compound according to claim 21 wherein R^{d} is methyl.

25. A nitrogen hexacycle compound according to claim 21 wherein R² and R^{2′} are each independently selected from the group consisting of hydrogen, halogen, straight, branched or cyclic alkyl of 1 to 6 carbons, B(OH)₂, B(OR)₂ wherein R is an alkyl group of 1 to 6 carbons, carboxylic acid, carboxyl ester with the ester group being straight, branched or cyclic alkyl of 1 to 6 carbons, N-alkyl amino methyl with the alkyl group being straight, branched or cyclic of 1 to 6 carbons, sulfonic acid, sulfonyl ester with the ester group being straight, branched or cyclic alkyl of 1 to 6 carbons, sulfonamide, amine (NH₂), mono, di or trialkyl amine with the alkyl being straight, branched or cyclic of 1 to 6 carbons, nitrile, carboxamide, N-alkyl carboxamide of 1 to 6 carbons in the straight, branched or cyclic alkyl group, perfluoroalkyl of 1 to 3 carbons, and straight, branched or cyclic alkoxy of 1 to 6 carbons.

26. A nitrogen hexacycle compound according to claim 1 wherein Rₙ, R⁴ and each of R⁵ are each independently selected from hydrogen, halogen, straight or branched alkyl of 1 to 6 carbons, carboxylic acid, carboxamide, substituted aminomethyl, sulfonic acid, sulfonamide, amine, mono, di or trialkyl amine of 1 to 6 carbons, nitrile, N-alkyl carboxamide of 1 to 6 carbons in the alkyl group, perfluoroalkyl of 1 to 3 carbons, alkoxy of 1 to 6 carbons, boronic acid or boronic ester having 1 to 3 carbons in the ester group.

27. A nitrogen hexacycle compound according to claim 26 wherein n of Rⁿ is 0, R³ is hydrogen or methyl, R⁴ and R⁵ are each independently hydrogen or methyl, and R² and R^{2′} are each independently selected from H, Me, Et, propyl (Pr), isopropyl (iPr), butyl (Bu), isobutyl (iBu), OMe, OEt, CN, OCH₂CH₂X, N(R^{a})₂, CO(NR^{a})₂, B(OH)₂, B(OR)₂ wherein R is an alkyl group of 1 to 6 carbons, SO₂R^{a}, SO₂N(R^{a})₂; R^{a} is H, Me, Et, Ph and when two R^{a}'s are present, each is selected independently; and X is amino, alkylamino, alkoxy, carboxy, carboxamide, carboxyl ester or N-alkyl carboxyamide.

28. A nitrogen hexacycle compound according to claim 27 wherein:
R¹ is B(OH)₂, B(OMe or Et)₂, CONH₂, CN, SO₂NH₂, COOH, COOMe, COOEt, CH₂NH₂, CH₂NHCOCH₃, CH₂NHSO₂CH₃ or CH₂OH; R² and R^{2′} are each independently H, Me, Et, COOH, CONH₂, SO₃H, SO₂NH₂, B(OH)₂, B(OMe or Et)₂, OMe, OEt, CN, F, Cl or Br, most especially, H or Me and of these two substituents, preferably H; R³ is H, Me, Et, COMe, COEt, SO₂Me or SO₂Et, most especially H or Me and of these two substituents, preferably H; R⁴ and each of R⁵ are each independently H, Me or Et, most especially H; Rₙ is excluded by n as 0; R⁶ is hydrogen.

29. A nitrogen hexacycle compound according to claim 1 wherein Ar is phenyl or substituted phenyl with the substituent being is fluoro, trifluoromethyl, boronic acid, boronic alkyl ester with a C1 to C6 alkyl, carboxylic acid, carboxylic alkyl ester with a C1 to C6 alkyl, carboxyamide, sulfonic acid, sulfonamide; preferably fluoro, boronic acid, boronic ester, carboxylic acid, carboxamid, sulfonic acid, sulfonic ester; more preferably fluoro, boronic acid, carboxylic acid, sulfonic acid; most preferred fluoro or boronic acid.

30. A nitrogen hexacycle compound according to claim 29 wherein Ar is phenyl, phenyl-4-boronic acid or 4-fluorophenyl.

31. A nitrogen hexacycle compound according to claim 1 wherein the P2 group is one of Formulas IA, ID, IE, IG, IH, IK, IK-sat, JO, IP, IS, IT, IU, IW, IX, IY and IZ-2 and the substituents R¹ through R⁶ and Rₙ are present, are delineated as given by any one of claims 1-30 and are positioned as given in Formula I:

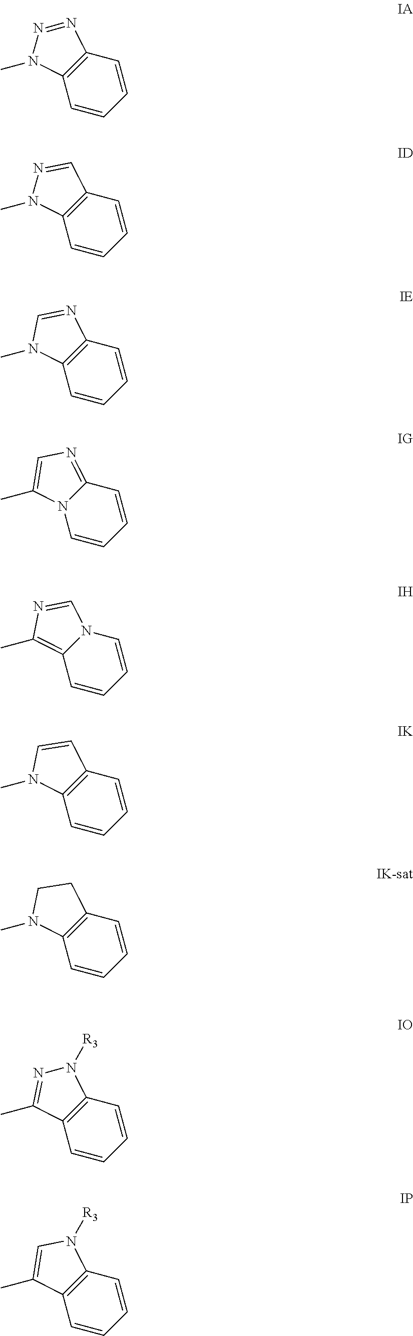

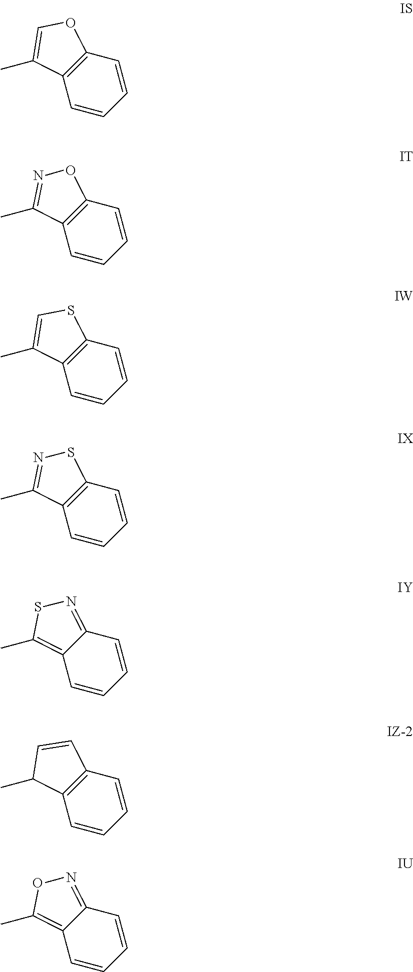

32. A nitrogen hexacycle compound according to claim 1 wherein Q¹ and Q²⁻ are both nitrogen as shown by formulas NT5 and NT6 wherein the upper right arrow designates the P2 group position and the bottom arrow designates the P4 group position:

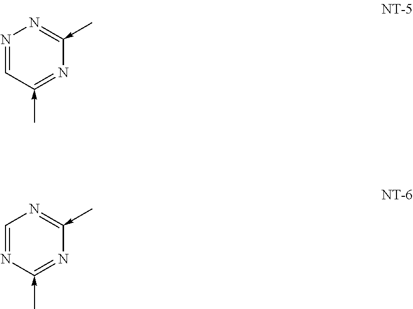

33. A pharmaceutical composition comprising a pharmaceutically acceptable carrier and an amount of a compound according to claim 1 which is effective as an inhibitor of the AAA family member Valosin containing protein.

34. A pharmaceutical composition according to claim 33 wherein the Valosin containing protein is in a human cell.

35. A method of decreasing Valosin containing protein activity or decreasing degradation of a proteasome system substrate comprising administering to a patient an effective amount of a compound according to claim 1.

36. A method of decreasing Valosin containing protein activity or degradation of a proteasome system substrate comprising administering to a patient an effective amount of a pharmaceutical composition of claim 33.

37. A method according to claim 35 wherein the patient is a human.

38. A method for treatment of a neoplastic malcondiction associated with Valosin containing protein comprising administering to a patient in need thereof an effective amount of a compound according to claim 1.

39. A method for treatment of a neoplastic malcondiction associated with Valosin containing protein comprising administering to a patient in need thereof an effective amount of a pharmaceutical composition according to claim 33.

40. A compound according to claim 1 having the IUPAC name:
1-(5-(benzylamino)-6-ethyl-1,2,4-triazin-3-yl)-1H-indazole-4-carboxamide
1-(5-(benzylamino)-6-methyl-1,2,4-triazin-3-yl)-1H-indazole-4-carboxamide
3-(4-((3-fluorobenzyl)amino)-6-ethyl-1,3,5-triazin-2-yl)-1H-indazole-7-carboxamide
3-(4-(benzylamino)-6-ethyl-1,3,5-triazin-2-yl)-1H-indazole-7-carboxamide
3-(4-((3-fluorobenzyl)amino)-6-methyl-1,3,5-triazin-2-yl)-1H-indazole-7-carboxamide
3-(4-(benzylamino)-6-methyl-1,3,5-triazin-2-yl)-1H-indazole-7-carboxamide
(1-(5-((3-fluorobenzyl)amino)-6-ethyl-1,2,4-triazin-3-yl)-2-methyl-1H-indole-4-yl)boronic acid
(1-(5-(benzylamino)-6-ethyl-1,2,4-triazin-3-yl)-2-methyl-1H-indole-4-yl)boronic acid
(3-(((3-(4-carbamoyl-2-methyl-1H-indol-1-yl)-6-ethyl-1,2,4-triazin-5-yl)amino)methyl)phenyl)boronic acid
1-(5-((3-fluorobenzyl)amino)-6-ethyl-1,2,4-triazin-3-yl)-2-methyl-1H-indole-4-carboxamide
1-(5-(benzylamino)-6-ethyl-1,2,4-triazin-3-yl)-2-methyl-1H-indole-4-carboxamide
(1-(5-((3-fluorobenzyl)amino)-6-methyl-1,2,4-triazin-3-yl)-2-methyl-1H-indole-4-yl)boronic acid
(1-(5-(benzylamino)-6-methyl-1,2,4-triazin-3-yl)-2-methyl-1H-indole-4-yl)boronic acid
(3-(((3-(4-carbamoyl-2-methyl-1H-indol-1-yl)-6-methyl-1,2,4-triazin-5-yl)amino)methyl)phenyl)boronic acid
1-(5-((3-fluorobenzyl)amino)-6-methyl-1,2,4-triazin-3-yl)-2-methyl-1H-indole-4-carboxamide
1-(5-(benzylamino)-6-methyl-1,2,4-triazin-3-yl)-2-methyl-1H-indole-4-carboxamide
(1-(4-((3-fluorobenzyl)amino)-6-ethyl-1,3,5-triazin-2-yl)-2-methyl-1H-indol-4-yl)boronic acid
(1-(4-(benzylamino)-6-ethyl-1,3,5-triazin-2-yl)-2-methyl-1H-indol-4-yl)boronic acid
(3-(((4-(4-carbamoyl-2-methyl-1H-indol-1-yl)-6-ethyl-1,3,5-triazin-2-yl)amino)methyl)phenyl)boronic acid
1-(4-((3-fluorobenzyl)amino)-6-ethyl-1,3,5-triazin-2-yl)-2-methyl-1H-indole-4-carboxamide
1-(4-(benzylamino)-6-ethyl-1,3,5-triazin-2-yl)-2-methyl-1H-indole-4-carboxamide
1-(4-((3-fluorobenzyl)amino)-6-ethyl-1,3,5-triazin-2-yl)-2-methyl-1H-indole-4-sulfonamide
1-(4-(benzylamino)-6-ethyl-1,3,5-triazin-2-yl)-2-methyl-1H-indole-4-sulfonamide
(1-(4-((3-fluorobenzyl)amino)-6-methyl-1,3,5-triazin-2-yl)-2-methyl-1H-indol-4-yl)boronic acid
(1-(4-(benzylamino)-6-methyl-1,3,5-triazin-2-yl)-2-methyl-1H-indol-4-yl)boronic acid
(3-(((4-(4-carbamoyl-2-methyl-1H-indol-1-yl)-6-methyl-1,3,5-triazin-2-yl)amino)methyl)phenyl)boronic acid
1-(4-((3-fluorobenzyl)amino)-6-methyl-1,3,5-triazin-2-yl)-2-methyl-1H-indole-4-carboxamide
1-(4-(benzylamino)-6-methyl-1,3,5-triazin-2-yl)-2-methyl-1H-indole-4-carboxamide
1-(4-((3-fluorobenzyl)amino)-6-methyl-1,3,5-triazin-2-yl)-2-methyl-1H-indole-4-sulfonamide
1-(4-(benzylamino)-6-methyl-1,3,5-triazin-2-yl)-2-methyl-1H-indole-4-sulfonamide
3-(5-(benzylamino)-6-ethyl-1,2,4-triazin-3-yl)-2-methyl-1H-indole-7-carboxamide
3-(5-(benzylamino)-6-methyl-1,2,4-triazin-3-yl)-2-methyl-1H-indole-7-carboxamide
3-(4-((3-fluorobenzyl)amino)-6-ethyl-1,3,5-triazin-2-yl)-2-methyl-1H-indole-7-carboxamide
3-(4-(benzylamino)-6-ethyl-1,3,5-triazin-2-yl)-2-methyl-1H-indole-7-carboxamide
3-(4-((3-fluorobenzyl)amino)-6-methyl-1,3,5-triazin-2-yl)-2-methyl-1H-indole-7-carboxamide
3-(4-(benzylamino)-6-methyl-1,3,5-triazin-2-yl)-2-methyl-1H-indole-7-carboxamide
1-(5-(benzylamino)-6-ethyl-1,2,4-triazin-3-yl)-2-methoxy-1H-benzo[d]imidazole-4-carboxamide
1-(5-(benzylamino)-6-methyl-1,2,4-triazin-3-yl)-2-methoxy-1H-benzo[d]imidazole-4-carboxamide
1-(4-((3-fluorobenzyl)amino)-6-ethyl-1,3,5-triazin-2-yl)-2-methoxy-1H-benzo[d]imidazole-4-carboxamide
1-(4-(benzylamino)-6-ethyl-1,3,5-triazin-2-yl)-2-methoxy-1H-benzo[d]imidazole-4-carboxamide
1-(4-((3-fluorobenzyl)amino)-6-methyl-1,3,5-triazin-2-yl)-2-methoxy-1H-benzo[d]imidazole-4-carboxamide, or
1-(4-(benzylamino)-6-methyl-1,3,5-triazin-2-yl)-2-methoxy-1H-benzo[d]imidazole-4-carboxamide.
